(19) United States
(12) Patent Application Publication
(10) Pub. No.: US 2016/0355475 A1
(43) Pub. Date: Dec. 8, 2016

(54) NOVEL INDOLE DERIVATIVE COMPOUND AND PHARMACEUTICAL COMPOSITION COMPRISING THE SAME

(71) Applicant: CHONG KUN DANG PHARMACEUTICAL CORP., Seoul (KR)

(72) Inventors: Changsik LEE, Gyeonggi-do (KR); Hyun-Mo YANG, Gyeonggi-do (KR); Dohoon KIM, Gyeonggi-do (KR); Miseon BAE, Gyeonggi-do (KR); Youngil CHOI, Gyeonggi-do (KR); Nina HA, Gyeonggi-do (KR)

(73) Assignee: CHONG KUN DANG PHARMACEUTICAL CORP., Seoul (KR)

(21) Appl. No.: 15/108,873

(22) PCT Filed: Jan. 2, 2015
(86) PCT No.: PCT/KR2015/000014

§ 371 Date: Jun. 29, 2016

(30) Foreign Application Priority Data
Jan. 3, 2014 (KR) 10-2014-0000802

(51) Int. Cl. C07D 209/20; C07D 401/08; C07D 471/04; C07D 417/14; C07D 401/14; C07D 403/06; C07D 405/14

(52) U.S. Cl. CPC C07D 209/20; C07D 403/06; C07D 401/08; C07D 405/14; C07D 417/14; C07D 401/14; C07D 471/04

(57) ABSTRACT

The present invention provides a novel indole derivative compound, an isomer thereof, a pharmaceutically acceptable salt thereof, or a hydrate or solvate thereof. The compound according to the present invention can selectively inhibit histone deacetylase (HDAC), and thus can be used to effectively treat a disease associated with histone deacetylase (HDAC) activity.

### TECHNICAL FIELD

The present invention relates to an indole derivative compound containing a carbon-carbon bond, a preparation method thereof and a pharmaceutical composition comprising the same. More specifically, the present invention relates to a novel indole derivative compound containing a carbon-carbon bond, which has histone deacetylase (HDAC) inhibitory activity, an isomer thereof, a pharmaceutically acceptable salt thereof, a hydrate thereof, or a solvate thereof, a preparation method thereof, the use thereof for the preparation of a pharmaceutical composition, a pharmaceutical composition containing the same, and a method of treating disease using the pharmaceutical composition.

### BACKGROUND ART

The cellular transcriptional regulation is a complex biological process. One of the basic principles is the post-translation modification of histone proteins H2A/B, H3 and H4 that form the octameric histone core complex. The complex N-terminal modifications at lysine residues by acetylation or methylation and at serine residues by phosphorylation constitute part of the so-called “histone code” (Stahl & Ellis, Nature 403, 41-45, 2000).

In a simple model, acetylation of positively charged lysine residues decreases affinity to negatively charged DNA, thus transcription factors may be easily entered.

Histone acetylation and deacetylation is catalysed by histone acetyltransferases (HATs) and histone deacetylases (HDACs), respectively. The HDAC is associated with transcriptional repressor complexes, switching chromatin to a transcriptionally inactive, silent structure (Marks et al. Nature Cancer Rev 1, 189-202, 2001). The opposite holds true for certain HATs which are associated with transcriptional activator complexes. Three different classes of HDACs, located in the nucleus, have been described so far, namely, class I (HDAC 1-3, 8; Mr=42-55 kDa) sensitive towards inhibition Trichostatin A (TSA), class II (HDAC 4-7, 9, 10; Mr=120-130 kDa) sensitive to TSA, and class III (Sir2) which are quite distinct by their NAB+ dependency and TSA insensitivity.

Inhibitors of histone deacetylases (HDACs) constitute a new class of anticancer drugs with differentiation and apoptosis activity. By targeting histone deacetylases (HDACs), the HDAC inhibitors affect histone (protein) acetylation and chromatin structure, inducing a complex transcriptional reprogramming, exemplified by reactivation of tumor suppressor genes and repression of oncogenes. In addition to generating acetylation of N-terminal lysine residue in core histone protein, there are non-histone targets important for cancer cell biology, including heat-shock-protein (HSP90), tubulin or p53 tumor suppressor protein. Thus, the medical use of HDAC inhibitors might not be restricted to cancer therapy, since efficacy in animal models for inflammatory diseases, rheumatoid arthritis and neurodegeneration has been shown.

HDAC inhibitors known up to now can be classified into four categories according to their structures: 1) short chain fatty acids (butyric acid, valproic acid); 2) hydroxamic acids (trichostatin A, SAHA, LBH-589); 3) cyclic peptides (desipeptide); and 4) benzamide (MS-275, MGCD-0103) (International Journal of Onocology 33, 637-646, 2008). These many HDAC inhibitors (SAHA, LBH-589, MS-275, etc.) effectively induce growth inhibition, differentiation, and apoptosis of various transformed cells in culture medium as well as in animal models (Marks, P. A et. al., Curr Opin Oncol. 2001. 13. 477-483), and some HDAC inhibitors such as SAHA, LBH-589, MS-275, etc. are clinically evaluated for the treatment of various cancers (Johnstone, R. W Nat. Rev. Drug Discov. 2002 1. 287-299). Typical examples of HDAC inhibitor compounds that are currently known include hydroxamate compounds, such as SAHA (U.S. Pat. No. 771,760, Zolinza, Vorinostat), PXD101 (WO 02/30879, Belinostat), LBH-589 (WO 02/22577, Panobinostat), and benzamide compounds such as MS-275 (EP8799) and MGCD0103 (WO 04/69823). Among these compounds, SAHA was approved on October 2006 and has been used for the treatment of CTCL (cutaneous T-cell lymphoma). Diseases for which medicine is efficacious have been additionally expanded, but it is known that there are drawbacks in terms of effectiveness and side effects (Cancer Res 2006, 66, 5781-5789).

Although many HDAC inhibitors have been reported to date, there has been a continued need for HDAC inhibitors that are more selectively, have less side effects and are more effective (Mol Cancer Res, 5, 981, 2007).

### DISCLOSURE

### Technical Problem

It is an object of the present invention to provide a novel indole derivative compound, an optical isomer thereof, a pharmaceutically acceptable salt thereof, or a hydrate or solvate thereof. Specifically, an object of the present invention is to provide an indole derivative containing a carbon-carbon bond and having histone deacetylase (HDAC) inhibitory activity, a pharmaceutically acceptable salt thereof, or a hydrate or solvate thereof.

Another object of the present invention is to provide a method for preparing the composition of the present invention, which has histone deacetylase (HDAC) inhibitory activity.

Still another object of the present invention is to provide a pharmaceutical composition comprising a novel indole derivative compound, an optical isomer thereof, a pharmaceutically acceptable salt thereof, or a hydrate or solvate thereof, together with a carrier.

Still another object of the present invention is to provide a pharmaceutical composition for inhibiting a disease associated with histone deacetylase (HDAC) activity, the composition comprising a pharmaceutical composition comprising a novel indole derivative compound, an optical isomer thereof, a pharmaceutically acceptable salt thereof, or a hydrate or solvate thereof.

Still another object of the present invention is to provide a method of inhibiting a histone deacetylase (HDAC) activity-associated disease using a pharmaceutical composition comprising a novel indole derivative compound, an optical isomer thereof, a pharmaceutically acceptable salt thereof, or a hydrate or solvate thereof.

Yet another object of the present invention is to provide the use of a pharmaceutical composition comprising a novel indole derivative compound, an optical isomer thereof, a pharmaceutically acceptable salt thereof, or a hydrate or solvate thereof, for inhibition of a disease associated with histone deacetylase (HDAC) activity.

### Technical Solution

To achieve the above objects, the present invention provides an indole derivative compound represented by the following formula I, an isomer, a pharmaceutically acceptable salt thereof, or a hydrate or solvate thereof:

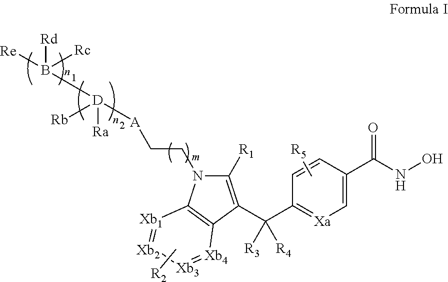

wherein

R₁ is hydrogen, halogen, a straight or branched C₁₋₅ alkyl, —NH₂, —OH, a straight or branched chain C₁₋₅ alkoxy, —CF₃, aryl, a 4- to 6-membered heteroaryl containing one or two heteroatoms selected from N, O and S,

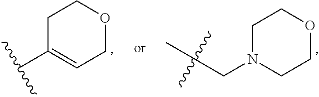 [SMILES: *C1=CCOCC1 ; "or" ; *CN1CCOCC1]

wherein the aryl and heteroaryl may each independently be unsubstituted or substituted with halogen, a straight or branched chain C₁₋₅ alkoxy or a straight or branched chain C₁₋₅ alkyl;

R₂ is hydrogen, halogen, a straight or branched chain C₁₋₅ alkyl, —NH₂, —OH, a straight or branched chain C₂₋₁₀ alkylalkoxy (—C₁₋₅—O—C₁₋₅ alkyl), a straight or branched chain C₁₋₅ alkoxy-OC₁₋₅ alkyl, —CF₃, a straight or branched C₁₋₅ alkyl-halogen, or a straight or branched chain C₁₋₅ alkyl hydroxide (—C₁₋₅ alkyl-OH);

R₃ and R₄ are each independently hydrogen or —OH;

R₅ is hydrogen, halogen, —CF₃, or a straight or branched chain C₁₋₃ alkyl;

n₁ and n₂ are each independently 0, 1 or 2;

A is

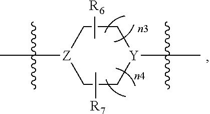

a 3- to 8-membered C₂₋₁₂ heterocycloalkyl containing one or two heteroatoms selected from N, O and S, or a 3- to 8-membered C₂₋₁₂ heteroaryl containing one or two heteroatoms selected from N, O and S, wherein Y is C or N, Z is C, O or N, R₆ and R₇ are each independently hydrogen, halogen, a straight or branched chain C₁₋₅ alkyl, —NH₂, —OH, a straight or branched chain C₂₋₁₀ alkylalkoxy (—C₁₋₅O—C₁₋₅ alkyl), a straight or branched chain C₁₋₅ alkoxy, —CF₃, a straight or branched chain C₁₋₅ alkyl-halogen, or a straight or branched chain C₁₋₅ alkyl hydroxide (—C₁₋₅ alkyl-OH), and n3 and n4 are each independently 0, 1 or 2;

Xa, Xb₁, Xb₂, Xb₃ and Xb₄ are each independently C or N;

B and D are each independently —H, C or halogen, and when B and D are H or halogen, Ra, Rb, Rc, Rd or Re, linked to B and D, does not exist;

m is 0, 1 or 2;

Ra is hydrogen, halogen, a straight or branched chain C₁₋₅ alkyl, a C₃₋₁₂ cycloalkyl, phenyl, —OH, a 5- or 6-membered heteroaryl containing one or two heteroatoms selected from N and O, or ═O;

Rb does not exist or is hydrogen, halogen, a straight or branched chain C₁₋₅ alkyl, —OH, a straight or branched chain C₁₋₅ alkoxy, a C₃₋₁₂ cycloalkyl, a 5- or 6-membered heteroaryl containing one or two heteroatoms selected from N and O, or phenyl;

the straight or branched chain C₁₋₅ alkoxy, C₃₋₁₂ cycloalkyl and phenyl in Ra or Rb may each independently be unsubstituted or substituted with halogen, —CN, thiazole, a straight or branched chain C₁₋₅ alkoxy or a straight or branched chain C₁₋₅ alkyl;

Rc is —H, halogen, a straight or branched chain —C₁₋₅ alkyl, a straight or branched chain C₁₋₅ alkoxy, —CO(O)C₁₋₅ alkyl, aryl, a 4- to 6-membered heteroaryl containing one or two heteroatoms selected from N, O and S, a C₃₋₁₂ cycloalkyl, —OH, or phenyl;

Rd is hydrogen, halogen, a straight or branched chain C₁₋₅ alkyl, a straight or branched chain C₁₋₅ alkoxy, a C₃₋₁₂ cycloalkyl, —OH, or phenyl;

Rc and Rd may be linked together to form a C₃₋₈ cycloalkyl or a 4- to 6-membered cycloheteroalkyl containing one or two heteroatoms selected from N, O and S;

Re is hydrogen, halogen, —CF₃, a straight or branched chain C₁₋₃ perfluoroalkyl, a straight or branched chain C₁₋₅ alkyl, a straight or branched chain C₁₋₅ alkoxy, a 4- to 6-membered heterocycloalkyl containing one or two heteroatoms selected from N, O and S, a C₃₋₁₂ cycloalkyl, aryl, a 4- to 6-membered heteroaryl containing one or two heteroatoms selected from N, O and S, NH₂, —OH, a straight or branched chain NHC₁₋₅ alkyl, —N-(straight or branched chain C₁₋₅ alkyl)₂, or an aryl substituted with a straight or branched chain C₁₋₅ alkyl;

wherein the straight or branched chain C₁₋₅ alkyl, C₃₋₁₂ cycloalkyl, 4- to 6-membered cycloheteroalkyl containing one or two heteroatoms selected from N, O and S, aryl, and 4- to 6-membered heteroaryl containing one or two heteroatoms selected from N, O and S in Rc, Rd or Re, and the cycloalkyl or cycloheteroalkyl formed by linkage between Rc and Rd, may each independently be unsubstituted or substituted with halogen, —CF₃, —CN, thiazole, a straight or branched chain C₁₋₅ alkoxy, a straight or branched chain C₁₋₅ alkyl, a straight or branched chain —C(═O)C₁₋₅ alkyl, a straight or branched chain —C(═O)OC₁₋₅ alkyl, a straight or branched chain —C(═O)NHC₁₋₅ alkyl, a straight or branched chain SO₂C₁₋₅ alkyl, —CF₃

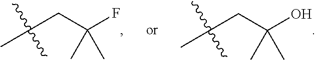

In the present invention, A in formula I may be

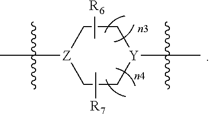

Z and Y in A may be the same or different. For example, Z and Y may all be carbon, or one of Z and Y may be N, and the other one may be carbon (C).

In the present invention, R₃ and R₄ in formula I may be the same or different. For example, R₃ and R₄ may all be —H, or one of R₃ and R₄ may be —H, and the other one may be —OH.

In the present invention, halogen may be F, Cl, Br or I, and preferably F.

In the present invention, R₁ in formula I may be —H, methyl, ethyl, propyl, butyl, phenyl,

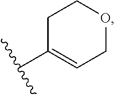 [SMILES: *C1=CCOCC1]

pyridinyl or pyrimidinyl, wherein the phenyl, pyridinyl or pyrimidinyl may be unsubstituted or substituted with one or two —F or —CF₃ groups.

In the present invention, B and D in formula I may be the same or different. For example, B and D may all be carbon (C).

In the present invention, the cycloheteroalkyl formed by linkage between Rc and Rd may be

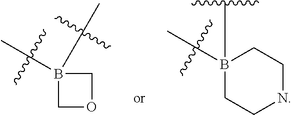

In the present invention, Xa, Xb1, Xb₂, Xb₃ and Xb₄ may all be carbon (C). Alternatively, Xa may be N, and Xb₁, Xb₂, Xb₃ and Xb₄ may be carbon (C).

As used herein, the term “pharmaceutically acceptable salt” means any salt that is generally used in the pharmaceutical field. Examples of the pharmaceutically acceptable salt include, but are not limited to, salts with inorganic ions such as calcium, potassium, sodium or magnesium ions, salts with inorganic acids such as hydrochloric acid, nitric acid, phosphoric acid, hydrobromic acid, iodic acid, perchloric acid, tartaric acid or sulfuric acid, salts with organic acids such as acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, gluconic acid, aspartic acid, ascorbic acid, carbonic acid, vanillic acid, hydroiodic acid, mandelic acid, mucic acid, nitric acid, pamoic acid, pantothenic acid, succinic acid or tartaric acid, salts with sulfonic acids such as methanesulfonic acid, ethanesulfonic acid, benzenesulfonic acid, p-toluenesulfonic acid, camphorsulfonic acid or naphthalenesulfonic acid, salts with amino acids such as glycine, arginine or lysine, and salts with amines such as trimethylamine, triethylamine, ammonia or picoline. For example, the pharmaceutically acceptable salt may be a salt with hydrochloric acid as inorganic acid, or a salt with methanesulfonic acid as organic acid.

As used herein, the term “isomer” is meant to include all structural isomers, cis-trans isomers, diastereomers, and optical isomers.

A hydrate of the compound represented by formula I according to the present invention, an isomer thereof, or a pharmaceutically acceptable salt thereof, may include a stoichiometric or non-stoichiometric amount of water bound by non-covalent intermolecular forces. The hydrate may include at least one equivalent (preferably one to five equivalents) of water. This hydrate may be prepared by crystallizing the compound represented by formula I, the above-listed compounds, an optical isomer thereof, or a pharmaceutically acceptable salt thereof, from water or a water-containing solvent.

A solvate of the compound represented by formula I, an isomer thereof, or a pharmaceutically acceptable salt thereof, may comprise a stoichiometric or non-stoichiometric amount of a solvent bound by non-covalent intermolecular forces. Preferred examples of the solvent include solvents that are non-volatile, non-toxic, and suitable for administration to humans. More specific examples include ethanol, methanol, propanol, methylene chloride and the like.

In the present invention, the compound represented by formula I may be selected from the group consisting of the following compounds:
N-hydroxy-4-((2-methyl-1-(2-morpholinoethyl)-1H-indol-3-yl)methyl)benzamide;
N-hydroxy-4-((2-methyl-1-(2-(4-methylpiperazin-1-yl)ethyl)-1H-indol-3-yl)methyl)benzamide;
N-hydroxy-4-((2-(morpholinomethyl)-1H-indol-3-yl)methyl)benzamide;
N-hydroxy-4-((1-(2-(4-isopropylpiperazin-1-yl)ethyl)-2-methyl-1H-indol-3-yl)methyl)benzamide;
N-hydroxy-4-((1-(2-(4-(2-hydroxyethyl)piperazin-1-yl)ethyl)-2-methyl-1H-indol-3-yl)methyl)benzamide;
N-hydroxy-4-((1-(2-(4-(2-methoxyethyl)piperazin-1-yl)ethyl)-2-methyl-1H-indol-3-yl)methyl)benzamide;
(S)—N-hydroxy-4-((1-(2-(2-(hydroxymethyl)pyrrolidin-1-yl)ethyl)-2-methyl-1H-indol-3-yl)methyl)benzamide;
(S)—N-hydroxy-4-((1-(2-(2-(methoxymethyl)pyrrolidin-1-yl)ethyl)-2-methyl-1H-indol-3-yl)methyl)benzamide;
N-hydroxy-4-((2-methyl-1-(2-(2-methyl-1H-imadazol-1-yl)ethyl)-1H-indol-3-yl)methyl)benzamide;
N-hydroxy-6-((2-methyl-1-(2-(4-methylpeperazin-1-yl)ethyl)-1H-indol-3-yl)methyl)nicotinamide;
N-hydroxy-6-((2-methyl-1-(2-morpholinoethyl)-1H-indol-3-yl)methyl)nocotinamide;
4-((1-((1-(2-fluoro-2-methylpropyl)piperidin-4-yl)methyl)-2-methyl-1H-indol-3-yl)methyl)-N-hydroxybenzamide;
4-((1-(2-(3-fluoropiperidin-1-yl)ethyl)-2-methyl-1H-indol-3-yl)methyl)-N-hydroxybenzamide;
(S)-4-((1-(2-(2-(fluoromethyl)pyrrolidin-1-yl)ethyl)-2-methyl-1H-indol-3-yl)methyl)-N-hydroxybenzamide;
4-((1-(2-(4-fluoropiperidin-1-yl)ethyl)-2-methyl-1H-indol-3-yl)methyl)-N-hydroxybenzamide;
4-((1-((1-benzylpiperidin-4-yl)methyl)-2-methyl-1H-indol-3-yl)methyl)-N-hydroxybenzamide;
4-((1-((1-butylpiperidin-4-yl)methyl)-2-methyl-1H-indol-3-yl)methyl)-N-hydroxybenzamide;
N-hydroxy-4-((2-methyl-1-((1-phenethylpiperidin-4-yl)methyl)-1H-indol-3-yl)methyl)benzamide;
3-fluoro-4-((1-((1-(2-fluoro-2-methylpropyl)piperidin-4-yl)methyl)-2-methyl-1H-indol-3-yl)methyl)-N-hydroxybenzamide;
4-((1-(2-(1-(2-fluoro-2-methylpropyl)piperidin-4-yl)ethyl)-2-methyl-1H-indol-3-yl)methyl)-N-hydroxybenzamide;
4-((1-(2-(4-(2-fluoro-2-methylpropyl)piperazin-1-yl)ethyl)-2-methyl-1H-indol-3-yl)methyl)-N-hydroxybenzamide;
4-((1-(2-(1-(2-ethyl-2-fluorobutyl)piperidin-4-yl)ethyl)-2-methyl-1H-indol-3-yl)methyl)-N-hydroxybenzamide;
4-((5-fluoro-1-((1-(2-hydroxy-2-methylpropyl)piperidin-4-yl)methyl)-2-methyl-1H-indol-3-yl)methyl)-N-hydroxybenzamide;
4-((1-((1-((4-fluoro-tetrahydro-2H-pyran-4-yl)methyl)piperidin-4-yl)methyl)-2-methyl-1H-indol-3-yl)methyl)-N-hydroxybenzamide;
4-((1-((1-((4-fluoro-tetrahydro-2H-pyran-4-yl)methyl)piperidin-4-yl)methyl)-2-methyl-1H-indol-3-yl)methyl)-N-hydroxybenzamide;
N-hydroxy-4-((1-(2-(3-(hydroxymethyl)pyrroldin-1-yl)ethyl)-2-methyl-1H-indol-3-yl)methyl)benzamide;
(S)-4-((5-fluoro-1-(2-(3-hydroxypyrrolidin-1-yl)ethyl)-2-methyl-1H-indol-3-yl)methyl)-N-hydroxybenzamide;
4-((5-fluoro-1-((1-(2-fluoro-2-methylpropyl)piperidin-4-yl)methyl)-2-methyl-1H-indol-3-yl)methyl)-N-hydroxybenzamide;
(S)-4-((5-fluoro-1-(2-(3-fluoropyrrolidin-1-yl)ethyl)-2-methyl-1H-indol-3-yl)methyl)-N-hydroxybenzamide;
4-((5-fluoro-1-(2-(4-fluoropiperidin-1-yl)ethyl)-2-methyl-1H-indol-3-yl)methyl)-N-hydroxybenzamide;
4-((1-(2-(3-(fluoromethyl)pyrrolidin-1-yl)ethyl)-2-methyl-1H-indol-3-yl)methyl)-N-hydroxybenzamide;
(S)—N-hydroxy-4-((1-(2-(3-hydroxypyrrolidin-1-yl)ethyl)-2-methyl-1H-indol-3-yl)methyl)benzamide;
(S)-4-((1-(2-(3-fluoropyrrolidin-1-yl)ethyl)-2-methyl-1H-indol-3-yl)methyl)-N-hydroxybenzamide;
N-hydroxy-4-((1-(2-(3-(hydroxymethyl)piperidin-1-yl)ethyl)-2-methyl-1H-indol-3-yl)methyl)benzamide;
4-((2-butyl-1-((1-(2-fluoro-2-methylpropyl)piperidin-4-yl)methyl)-1H-indol-3-yl)methyl)-N-hydroxybenzamide;
N-hydroxy-4-((1-((1-(2-hydroxy-2-methylpropyl)piperidin-4-yl)methyl)-2-propyl-1H-indol-3-yl)methyl)benzamide;
4-((1-((1-(2-fluoro-2-methylpropyl)piperidin-4-yl)methyl)-2-propyl-1H-indol-3-yl)methyl)-N-hydroxybenzamide;
N-hydroxy-4-((2-methyl-1-((1-(3-(thiazol-2-yl)benzyl)piperidin-4-yl)methyl)-1H-indol-3-yl)methyl)benzamide;
N-hydroxy-4-((1-((1-(2-hydroxy-2-methylpropyl)piperidin-4-yl)methyl)-2-methyl-1H-indol-3-yl)methyl)benzamide;
4-((1-((1-((3-fluoroxetan-3-yl)methyl)piperidin-4-yl)methyl)-2-methyl-1H-indol-3-yl)methyl)-N-hydroxybenzamide;
tert-butyl-4-fluoro-4-((4-((3-(4-(hydroxycarbamoyl)benzyl)-2-methyl-1H-indol-1-yl)methyl)piperidin-1-yl)methyl)piperidine-1-carboxylate;
4-((1-((1-((4-fluoropiperidin-4-yl)methyl)piperidin-4-yl)methyl)-2-methyl-1H-indol-3-yl)methyl)-N-hydroxybenzamide;
4-((1-((1-((1-acetyl-4-fluoropiperidin-4-yl)methyl)piperidin-4-yl)methyl)-2-methyl-1H-indol-3-yl)methyl)-N-hydroxybenzamide;
4-((1-((1-((4-fluoro-1-(2-hydroxy-2-methylpropyl)piperidin-4-yl)methyl)piperidine-4-yl)methyl)-2-methyl-1H-indol-3-yl)methyl)-N-hydroxybenzamide;
4-((1-((1-(2-fluoro-2-methylpropyl)piperidine-4-yl)methyl)-1H-indol-3-yl)methyl)-N-hydroxybenzamide;
4-((1-((1-((4-fluoro-1-(2-fluoro-2-methylpropyl)piperidine-4-yl)methyl)piperidin-4-yl)methyl)-2-methyl-1H-indol-3-yl)methyl)-N-hydroxybenzamide;
4-((1-((1-(2-fluoro-2-methylpropyl)piperidine-4-yl)methyl)-2-phenyl-1H-indol-3-yl)methyl)-N-hydroxybenzamide;
N-hydroxy-4-((2-methyl-1-((1-(2,4,5-trifluorobenzyl)piperidine-4-yl)methyl)-1H-indol-3-yl)methyl)benzamide;
4-((1-((1-(2-fluorobenzyl)piperidine-4-yl)methyl)-2-methyl-1H-indol-3-yl)methyl)-N-hydroxybenzamide;
N-hydroxy-4-((2-methyl-1-((1-(pyridin-4-ylmethyl)piperidine-4-yl)methyl)-1H-indol-3-yl)methyl)benzamide;
4-((1-((1-(4-fluorobenzyl)piperidine-4-yl)methyl)-2-methyl-1H-indol-3-yl)methyl)-N-hydroxybenzamide;
N-hydroxy-4-((2-methyl-1-((1-(pyridin-2-ylmethyl)piperidine-4-yl)methyl)-1H-indol-3-yl)methyl)benzamide;
4-((1-((1-(2-fluoro-2-methylpropyl)piperidine-4-yl)methyl)-2-(pyridin-4-yl)-1H-indol-3-yl)methyl)-N-hydroxybenzamide;
4-((1-((1-(2-fluoro-2-methylpropyl)piperidine-4-yl)methyl)-2-(pyrimidin-5-yl)-1H-indol-3-yl)methyl)-N-hydroxybenzamide;
4-((2-(3,5-difluorophenyl)-1-((1-(2-fluoro-2-methylpropyl)piperidine-4-yl)methyl)-1H-indol-3-yl)methyl)-N-hydroxybenzamide;
4-((2-(3,6-dihydro-2H-pyran-4-yl)-1-((1-(2-fluoro-2-methylpropyl)piperidine-4-yl)methyl)-1H-indol-3-yl)methyl)-N-hydroxybenzamide;
4-((1-((1-(3-fluorobenzyl)piperidine-4-yl)methyl)-2-methyl-1H-indol-3-yl)methyl)-N-hydroxybenzamide;
N-hydroxy-4-((2-methyl-1-((1-(pyridin-3-ylmethyl)piperidine-4-yl)methyl)-1H-indol-3-yl)methyl)benzamide;
4-((1-(2-((3S,5R)-4-(2-fluoro-2-methylpropyl)-3,5-dimethylpiperazin-1-yl)ethyl)-2-methyl-1H-indol-3-yl)methyl)-N-hydroxybenzamide;
4-((1-(4-(4-(2-fluoro-2-methylpropyl)piperazin-1-yl)butyl)-2-methyl-1H-indol-3-yl)methyl)-N-hydroxybenzamide;
4-((1-(3-(4-(2-fluoro-2-methylpropyl)piperazin-1-yl)propyl)-2-methyl-1H-indol-3-yl)methyl)-N-hydroxybenzamide;
N-hydroxy-4-((2-methyl-1-((1-((3-methyloxetan-3-yl)methyl)piperidine-4-yl)methyl)-1H-indol-3-yl)methyl)benzamide;
4-((1-((1-((4-fluoro-1-methylpiperidine-4-yl)methyl)piperidine-4-yl)methyl)-2-methyl-1H-indol-3-yl)methyl)-N-hydroxybenzamide;
4-((1-((1-((4-fluoro-1-isopropylpiperidin-4-yl)methyl)piperidin-4-yl)methyl)-2-methyl-1H-indol-3-yl)methyl)-N-hydroxybenzamide;
4-fluoro-4-((4-((3-(4-(hydroxycarbamoyl)benzyl)-2-methyl-1H-indol-1-yl)methyl)piperidin-1-yl)methyl)-N-isopropylpiperidine-1-carboxamide;
4-((1-((1-((4-fluoro-1-(methylsulfonyl)piperidin-4-yl)methyl)piperidin-4-yl)methyl)-2-methyl-1H-indol-3-yl)methyl)-N-hydroxybenzamide;
4-((1-((1-(3,5-bis(trifluoromethyl)benzyl)piperidin-4-yl)methyl)-2-methyl-1H-indol-3-yl)methyl)-N-hydroxybenzamide;
4-((1-((1-((1-fluorocyclohexyl)methyl)piperidin-4-yl)methyl)-2-methyl-1H-indol-3-yl)methyl)-N-hydroxybenzamide;
4-((4-fluoro-1-((1-(2-fluoro-2-methylpropyl)piperidin-4-yl)methyl)-1H-indol-3-yl)methyl)-N-hydroxybenzamide;
4-((6-fluoro-1-((1-(2-fluoro-2-methylpropyl)piperidin-4-yl)methyl)-1H-indol-3-yl)methyl)-N-hydroxybenzamide;
4-((1-((1-(2-fluoro-2-methylpropyl)pyrrolidin-3-yl)methyl)-2-methyl-1H-indol-3-yl)methyl)-N-hydroxybenzamide;
N-hydroxy-4-((2-methyl-1-((1-((1-(trifluoromethyl)cyclobutyl)methyl)piperidin-4-yl)methyl)-1H-indol-3-yl)methyl)benzamide;
4-((1-((1-(2-fluoro-2-methylpropyl)piperidin-4-yl)methyl)-1H-pyrrolo[2,3-b]pyridin-3-yl)(hydroxy)methyl)-N-hydroxybenzamide;
4-((1-((1-(2-fluoro-2-methylpropyl)piperidin-4-yl)methyl)-1H-pyrrolo[2,3-b]pyridin-3-yl)methyl)-N-hydroxybenzamide;
4-((1-((1-((3-fluorooxetan-3-yl)methyl)azetidin-3-yl)methyl)-2-methyl-1H-indol-3-yl)methyl)-N-hydroxybenzamide; and
4-((1-((1-(2-fluoro-2-methylpropyl)azetidin-3-yl)methyl)-2-methyl-1H-indol-3-yl)methyl)-N-hydroxybenzamide.

Preferably, the compound represented by formula I may be 4-((1-((1-(2-fluoro-2-methylpropyl)piperidin-4-yl)methyl)-2-methyl-1H-indol-3-yl)methyl)-N-hydroxybenzamide, or 4-((1-((1-((3-fluorooxetan-3-yl)methyl)piperidin-4-yl)methyl)-2-methyl-1H-indol-3-yl)methyl)-N-hydroxybenzamide.

Preparation of Compounds

In the present invention, the compound represented by formula I can be prepared by any one of the methods shown by the following reaction schemes 1 to 16:

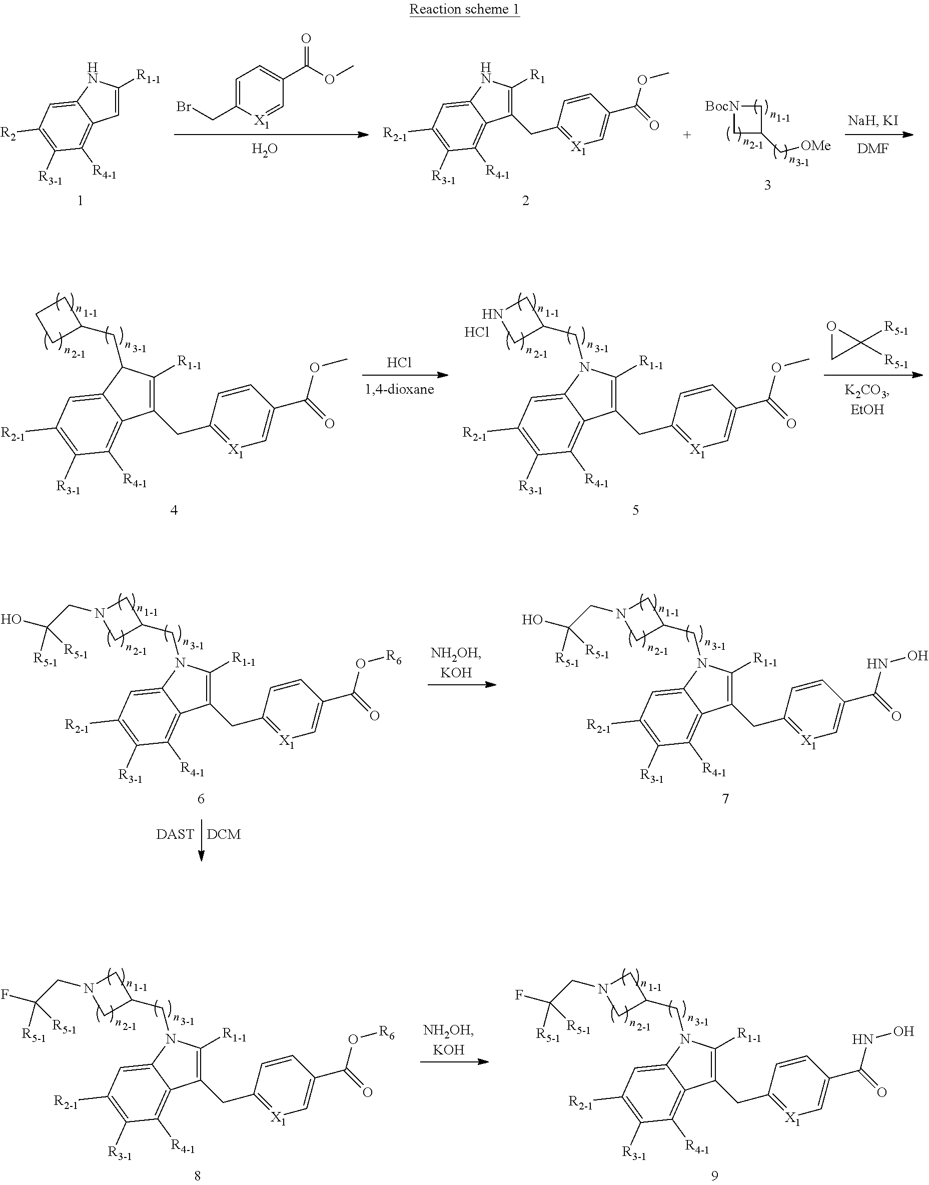

wherein
n₁₋₁, n₂₋₁ and n₃₋₁ may each independently be 1 or 2;
R₁₋₁ to R₅₋₁ may each independently be —H, methyl, n-propyl or n-butyl; and

### X₁ may be —CH—.

TABLE 1
Formula 7

| Com- | | | | | | | | | |
|---|---|---|---|---|---|---|---|---|---|
| pound | | | | | | | | | |
| No. | n₂₋₃ | n₂₋₁ | n₃₋₁ | R₁₋₁ | R₂₋₁ | R₃₋₁ | R₄₋₁ | R₅₋₁ | X₁ |
| 582 | 2 | 2 | 1 | Me | H | F | H | Me | CH |
| 601 | 2 | 2 | 1 | n-Pr | H | H | H | Me | CH |
| 608 | 2 | 2 | 1 | Me | H | H | H | Me | CH |

TABLE 2
Formula 9

| Com- | | | | | | | | | |
|---|---|---|---|---|---|---|---|---|---|
| pound | | | | | | | | | |
| No. | n₂₋₃ | n₂₋₁ | n₃₋₁ | R₁₋₁ | R₂₋₁ | R₃₋₁ | R₄₋₁ | R₅₋₁ | X₁ |
| 528 | 2 | 2 | 1 | Me | H | H | H | Me | CH |
| 563 | 2 | 2 | 2 | Me | H | H | H | Me | CH |
| 581 | 2 | 2 | 2 | Me | H | H | H | Et | CH |
| 588 | 2 | 2 | 1 | Me | H | F | H | Me | CH |
| 600 | 2 | 2 | 1 | n-Bu | H | H | H | Me | CH |
| 602 | 2 | 2 | 1 | n-Pr | H | H | H | Me | CH |
| 614 | 2 | 2 | 1 | H | H | H | H | Me | CH |
| 707 | 2 | 2 | 1 | H | H | H | F | Me | CH |
| 708 | 2 | 2 | 1 | H | F | H | H | Me | CH |
| 713 | 1 | 2 | 1 | Me | H | H | H | Me | CH |
| 750 | 1 | 1 | 1 | Me | H | H | H | Me | CH |

In reaction scheme 1 above, a compound of formula 1 is subjected to catalyst-free Friedel-Crafts alkylation [The European Journal of Organic Chemistry. 2010, 1029-1032] with methyl 4-(bromomethyl)benzoate or methyl 6-(bromomethyl)nicotinate in a microwave reactor to synthesize a compound of formula 2. The compound of formula 2 is subjected to a substitution reaction in the presence of sodium hydride to synthesize a compound of formula 4, which is then treated with hydrochloric acid to remove the Boc protecting group, thereby obtaining a compound of formula 5. The compound of formula 5 is reacted with various oxirane compounds to synthesize compounds of formula 6, and the hydroxyl group of the compounds of formula 6 is substituted with fluoride to synthesize compounds of formula 8. Each of the compounds of formula 6 and the compounds of formula 8 is reacted with an aqueous hydroxylamine solution and potassium hydroxide, thereby synthesizing compounds 582, 601, 608, 528, 563, 581, 588, 600, 602, 614, 707, 708, 713 and 750.

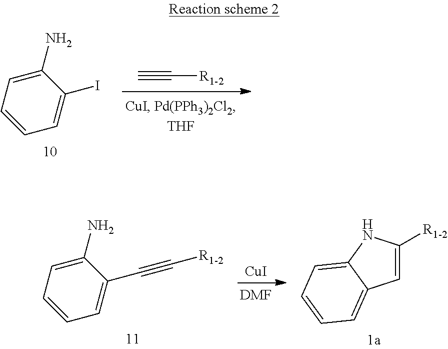

wherein R₁₋₂ is n-propyl or n-butyl.

Reaction formula 2 shows a method for synthesizing the intermediate used in reaction formula 1, in which a compound of formula 10 is subjected to Sonogashira coupling to synthesize a compound of formula 11 having a triple bond, and the compound of formula 11 is cyclized in the presence of copper iodide to synthesize an indole-type compound of formula 1a having a substituent at position 2.

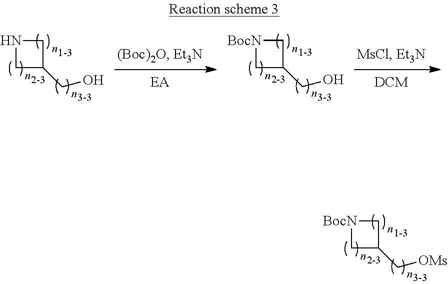

Reaction scheme 3 above shows a method for synthesizing the intermediate used in reaction schemes 1, 13, 15 and 16, in which an amine of formula 12 is protected with Boc, and the hydroxyl group is activated with methanesulfonyl chloride, thereby synthesizing a compound of formula 3.

In reaction scheme 3 above, n₁₋₃, n₂₋₃ and n₃₋₃ are each independently 1 or 2.

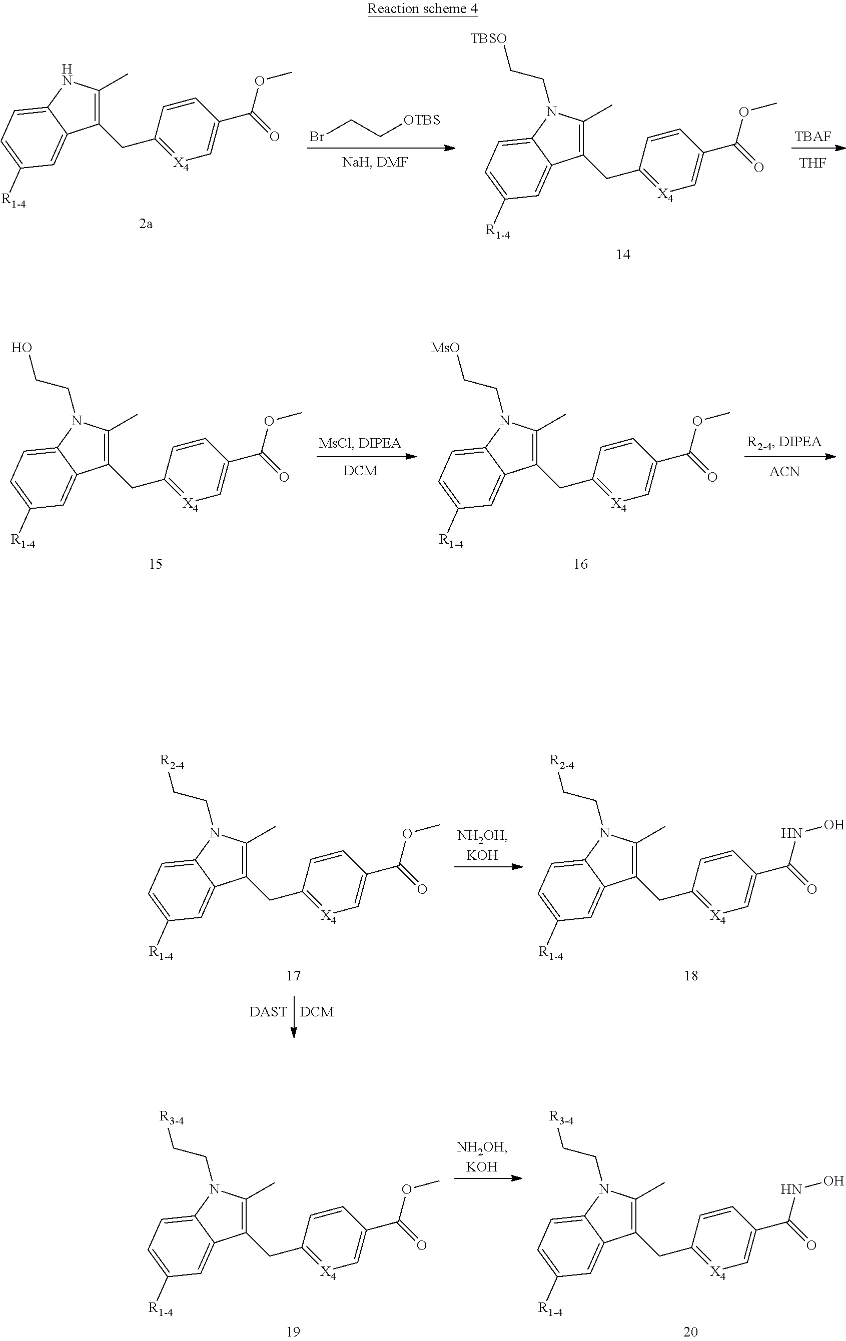

wherein R₁₋₄ is —H or —F;

R₂₋₄ may be morpholinyl, 4-methylpiperazin-1-yl, 4-isopropylpiperazin-1-yl, 4-(2-hydroxyethyl)piperazin-1-yl, 4-(2-methoxyethyl)piperazin-1-yl, (S)-2-(hydroxymethyl)pyrrolidin-1-yl, (S)-2-(methoxymethyl)pyrrolidin-1-yl, 2-methyl-1H-imidazol-1-yl, piperidin-1-yl, 3-(hydroxymethyl)pyrrolidin-1-yl, (S)-3-hydroxypyrrolidin-1-yl, or 3-(hydroxymethyl)piperidin-1-yl;

R₃₋₄ may be 3-fluoropiperidin-1-yl, (S)-2-(fluoromethyl)pyrrolidin-1-yl, 4-fluoropiperidin-1-yl, (S)-3-fluoropyrrolidin-1-yl, or 3-(fluoromethyl)pyrrolidin-1-yl, and

X₄ may be —CH— or —N—.

TABLE 3
Formula 18

| Compound No. | R₁₋₄ | R₂₋₄ | X₄ |
|---|---|---|---|
| 153 | H | morpholine | CH |
| 154 | H | 1-methylpiperazine | CH |
| 196 | H | 1-isopropylpiperazine | CH |
| 197 | H | 2-(piperazin-1-yl)ethanol | CH |
| 198 | H | 1-(2-methoxyethyl)piperazine | CH |
| 199 | H | (S)-pyrrolidin-2-ylmethanol | CH |
| 200 | H | (S)-2-(methoxymethyl)pyrrolidine | CH |
| 201 | H | 2-methyl-1H-imidazole | CH |
| 243 | H | 1-methylpiperazine | N |
| 244 | H | morpholine | N |
| 585 | H | piperidine | CH |
| 586 | H | pyrrolidin-3-ylmethanol | CH |
| 587 | F | (S)-pyrrolidin-3-ol | CH |
| 592 | H | (S)-pyrrolidin-3-ol | CH |
| 594 | H | piperidin-3-ylmethanol | CH |

TABLE 4
Formula 20

| Compound | | | |
|---|---|---|---|
| No. | R₁₋₄ | R₂₋₄ | X₄ |
| 550 | H | S-fluoropiperidine | CH |
| 551 | H | (S)-2-(fluoromethyl)pyrrolidine | CH |
| 553 | H | 4-fluoropiperidine | CH |
| 589 | F | (S)-3-fluoropyrrolidine | CH |
| 590 | F | 4-fluoropiperidine | CH |
| 591 | H | 3-(fluoromethyl)pyrrolidine | CH |
| 593 | H | (S)-3-fluoropyrrolidine | CH |

In reaction formula 4 above, a compound of formula 2a is substituted with (2-bromoethoxy)(tert-butyl)dimethylsilane) in the presence of sodium hydride to synthesize a compound of formula 14, which is then deprotected with fluoride to synthesize a compound of formula 15. The hydroxyl group of the compound of formula 15 is activated with methanesulfonyl chloride to synthesize a compound of formula 16, which is then substituted with various amines in a microwave reactor to synthesize compounds of formula 17, and the hydroxyl group of the compounds of formula 17 is substituted with fluoride to synthesize compounds of formula 19. Each of the compounds of formula 17 and the compounds of formula 19 is reacted with an aqueous hydroxylamine solution and potassium hydroxide, thereby synthesizing compounds 153, 154, 196, 197, 198, 199, 200, 201, 243, 244, 585, 586, 587, 592, 594, 550, 551, 553, 589, 590, 591 and 593.

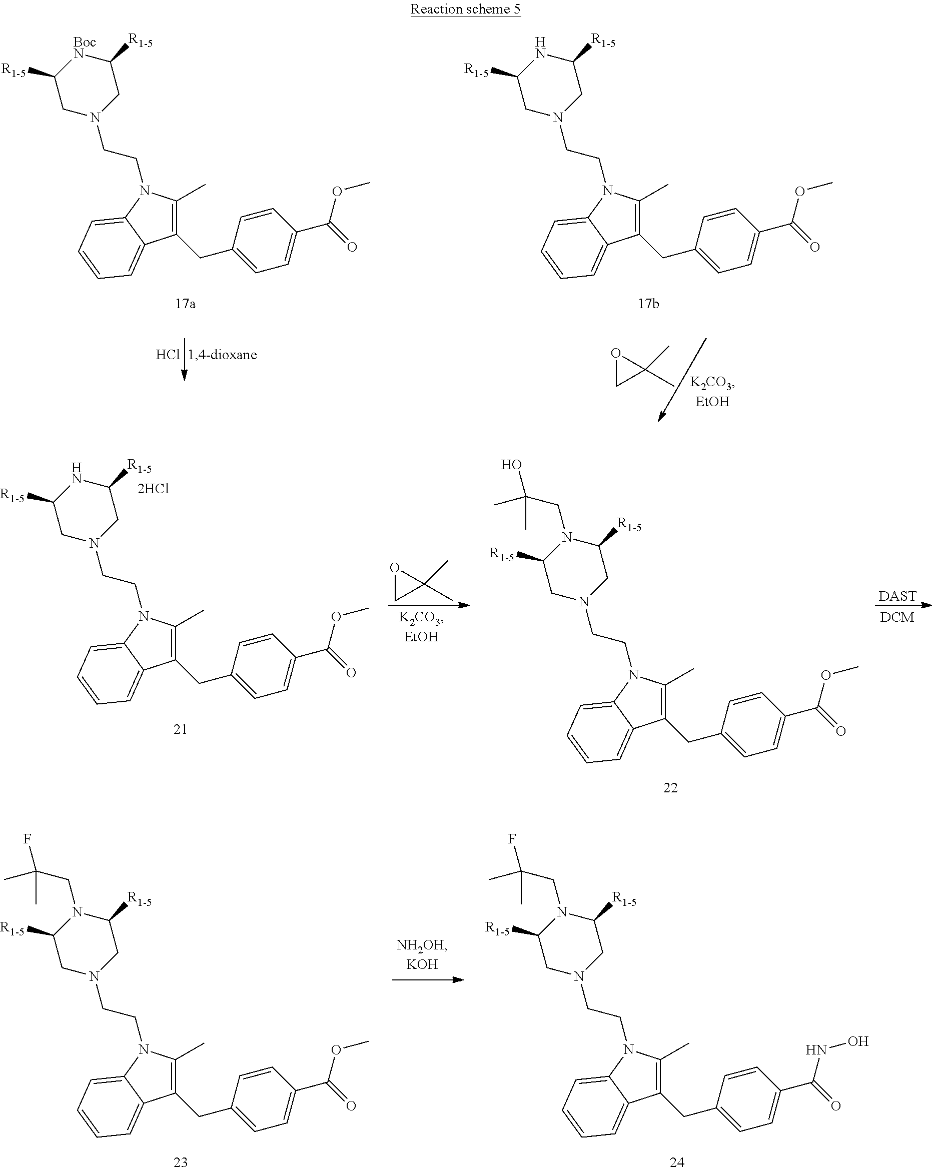

wherein R₁₋₅ may be H or methyl.

TABLE 5
Formula 24

| | | | |
|---|---|---|---|
| Compound 564 | R₁₋₅ = H | Compound 633 | R₁₋₅ = Me |

In Reaction scheme 5 above, a compound of formula 17a is treated with hydrochloric acid to remove the Boc protecting group, thereby synthesizing a compound of formula 21. Each of the compound of formula 21 and the compound of formula 17b is reacted with 2,2-dimethyloxirane to synthesize a compound of formula 22. Next, the hydroxyl group of the compound of formula 22 is substituted with fluoride, and the substituted compound is reacted with an aqueous hydroxylamine solution and potassium hydroxide, thereby synthesizing compounds 564 and 633.

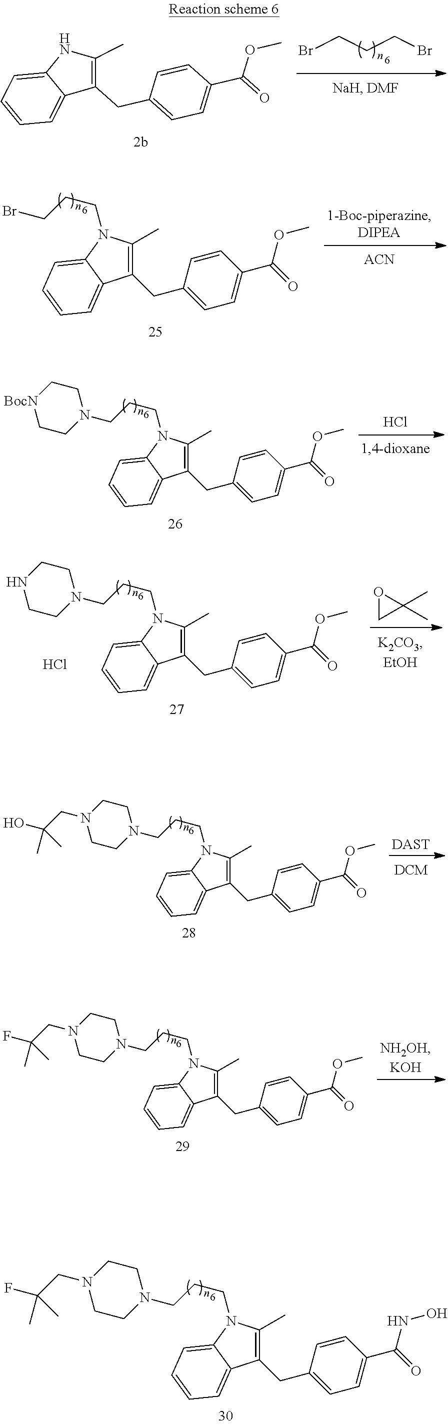

wherein n₆ may be 1 or 2.

TABLE 6
Formula 30

| | | | |
|---|---|---|---|
| Compound 639 | n₆ = 2 | Compound 640 | n₆ = 1 |

In reaction scheme 6 above, a compound of formula 2b is subjected to a substitution reaction with 1,3-dibromopropane or 1,4-dibromobutane to synthesize a compound of formula 25, which is then substituted with 1-Boc-piperazine and treated with hydrochloric acid to remove the Boc protecting group, thereby synthesizing a compound of formula 27. The compound of formula 27 is treated in the same manner as shown in reaction formula 5 above, thereby synthesizing compounds 639 and 640.

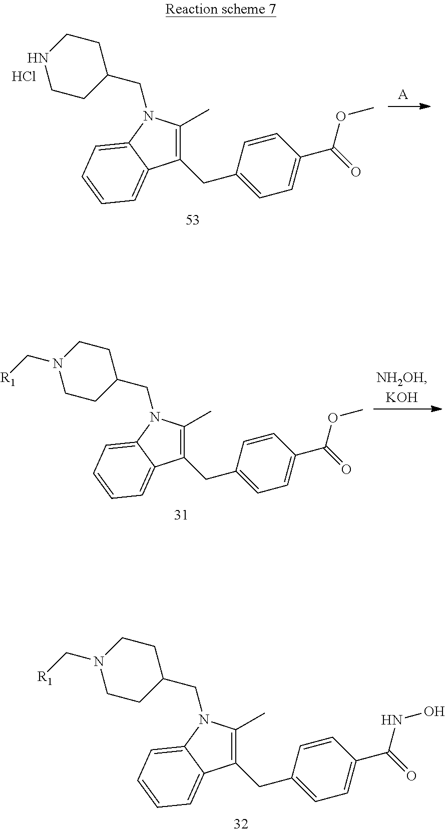

wherein reagent A may be a combination of the following compounds:
R₁₋₇CH₂Br and diisopropylethylamine;
R₁₋₇CHO, NaBH₃CN and acetic acid;
R₁₋₇CHO, NaBH(OAc)₃ and acetic acid; or
R₁₋₇CH₂OTs and diisopropylethylamine (TS=CH₃C₆H₄SO₂⁻).

In reaction scheme 7 and reagent A above, R₁₋₇ may be

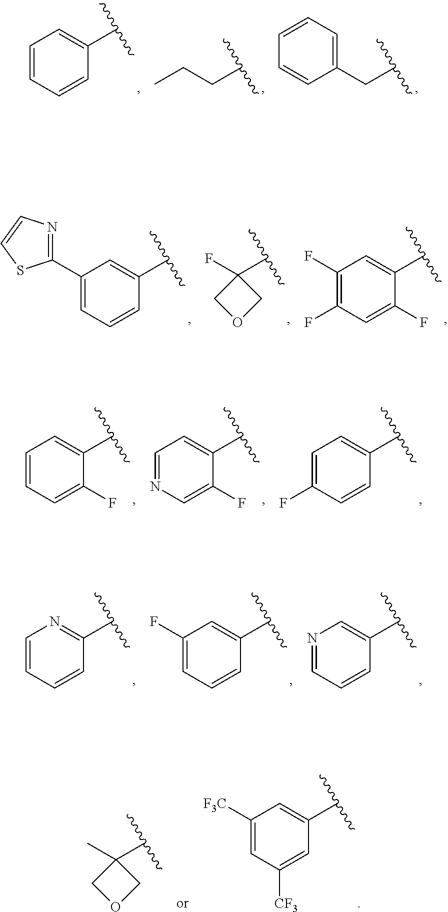

TABLE 7
Formula 32

| Compound | | |
|---|---|---|
| No. | Reagent (A) | R₁₋₇ |
| 556 | R₁₋₇CH₂Br, DIPEA | 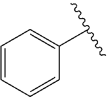 [SMILES: *c1ccccc1] |
| 558 | R₁₋₇CHO, NaBH₃CN, AcOH | 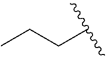 [SMILES: *CCC] |
| 559 | R₁₋₇CHO, NaBH₃CN, AcOH | 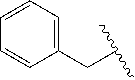 [SMILES: *Cc1ccccc1] |
| 603 | R₁₋₇CHO, NaBH₃CN, AcOH | 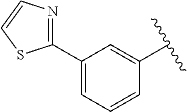 [SMILES: *c1cccc(-c2nccs2)c1] |
| 609 | R₁₋₇CH₂Br, DIPEA | 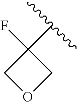 [SMILES: *C1(F)COC1] |
| 619 | R₁₋₇CH₂Br, DIPEA | 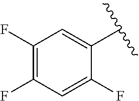 [SMILES: *c1cc(F)c(F)cc1F] |
| 620 | R₁₋₇CH₂Br, DIPEA | 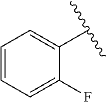 [SMILES: *c1ccccc1F] |
| 621 | R₁₋₇CHO, NaBH(OAc)₃, AcOH | 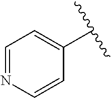 [SMILES: *c1ccncc1] |
| 622 | R₁₋₇CHO, NaBH(OAc)₃, AcOH | 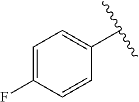 [SMILES: *c1ccc(F)cc1] |
| 623 | R₁₋₇CHO, NaBH(OAc)₃, AcOH | 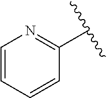 [SMILES: *c1ccccn1] |
| 631 | R₁₋₇CHO, NaBH(OAc)₃, AcOH | 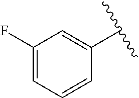 [SMILES: *c1cccc(F)c1] |
| 632 | R₁₋₇CHO, NaBH(OAc)₃, AcOH | 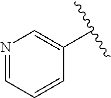 [SMILES: *c1cccnc1] |
| 643 | R₁₋₇CH₂OTs, DIPEA | 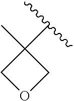 [SMILES: *C1(C)COC1] |
| 697 | R₁₋₇CH₂Br, DIPEA | 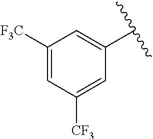 [SMILES: *c1cc(C(F)(F)F)cc(C(F)(F)F)c1] |

In reaction scheme 7 above, a compound of formula 5a is subjected either to a reductive amination reaction with an aldehyde-containing compound or to a substitution reaction with a compound containing a leaving group to synthesize a compound of formula 31, which is then reacted with an aqueous hydroxylamine solution and potassium hydroxide, thereby synthesizing compounds 556, 558, 559, 603, 609, 619, 620, 621, 622, 623, 631, 632, 643 and 697.

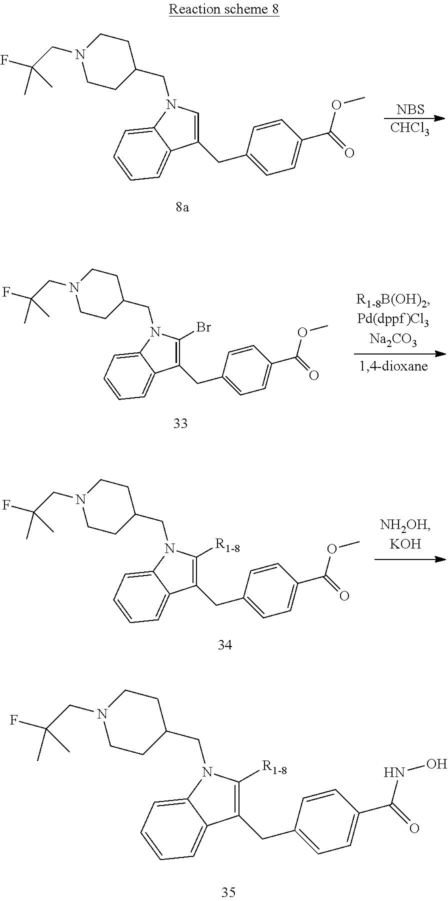

wherein R₁₋₈ may be phenyl, pyridin-4-yl, pyrimidin-5-yl, 3,5-difluorophenyl, or 3,6-dihydro-2H-pyran-4-yl.

TABLE 8
Formula 35

| Compound No. | R₁₋₈ |
|---|---|
| 616 | Phenyl |
| 624 | Pyridin-1-yl |
| 625 | Pyrimidin-5-yl |
| 626 | 3,5-difluorophenyl |
| 627 | 3,6-difluoro-2H-pyran- |
| | 4-yl |

In reaction scheme 8 above, a compound of formula 8a is reacted with N-bromosuccinimide (NBS) to synthesize a compound of formula 33 having a bromine substituent, and the compound of formula 33 is subjected to the Suzuki coupling reaction with various boronic acid compounds to synthesize compounds of formula 34, which are then reacted with an aqueous hydroxylamine solution and potassium hydroxide, thereby synthesizing compounds 616, 624, 625, 626 and 627.

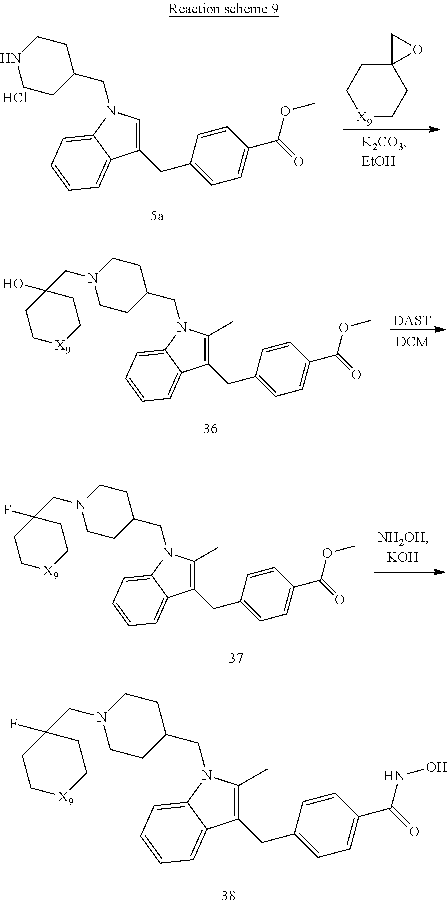

wherein X₉ may be —O—, —NBoc or —CH₂—.

TABLE 9
Formula 38

| Compound | X₉ = O | Compound | X₉ = NBoc | Com- | X₉ = CH₂ |
|---|---|---|---|---|---|
| 584 | | 610 | | pound | |
| | | | | 698 | |

In reaction scheme 9 above, a compound of formula 5a is reacted with various oxirane compounds to synthesize compounds of formula 36. The hydroxyl group of the compounds of the formula 36 is substituted with fluoride to synthesize compounds of formula 37, which are then reacted with an aqueous hydroxylamine solution and potassium hydroxide, thereby synthesizing compounds 610 and 698.

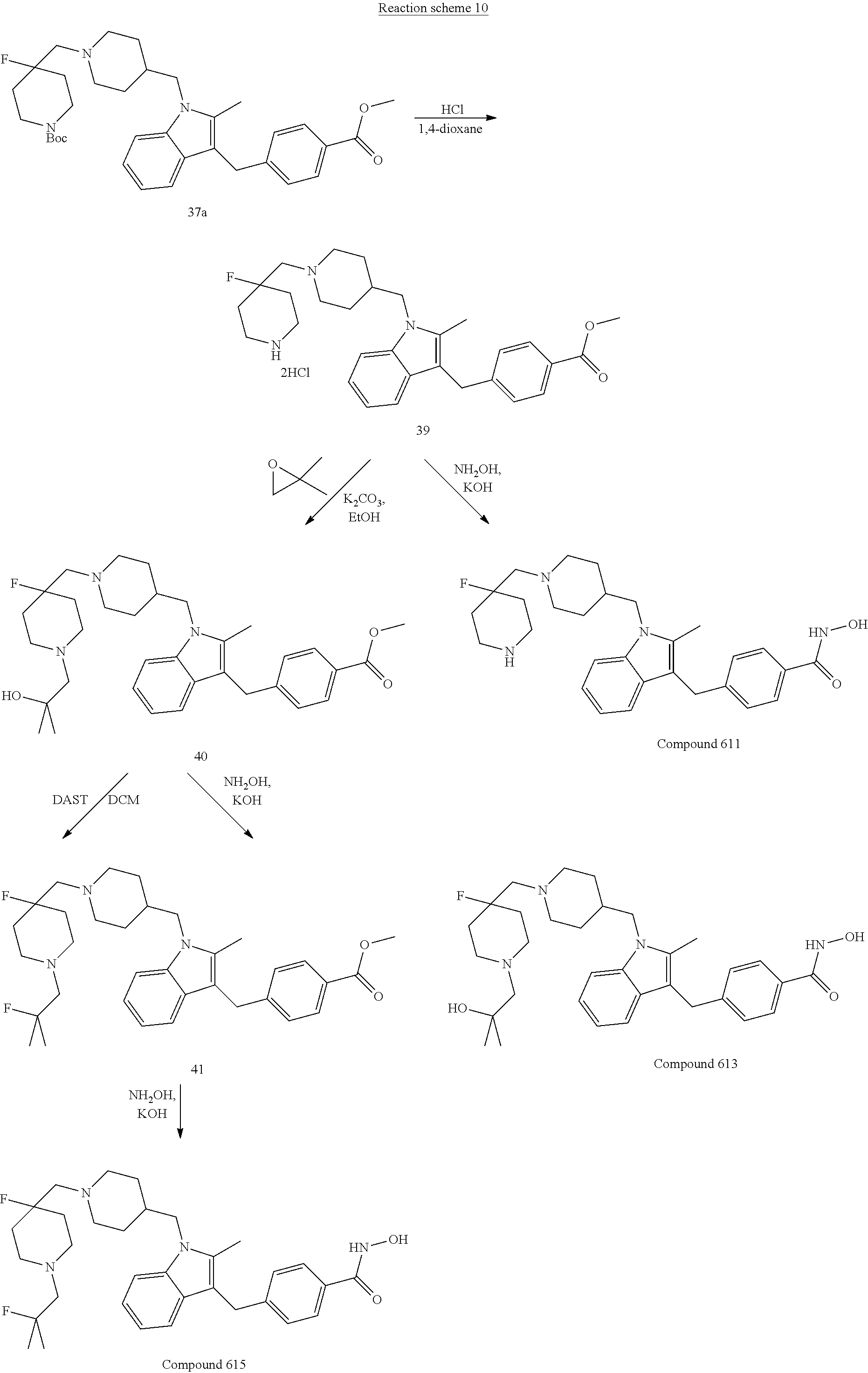

In reaction scheme 10 above, a compound of formula 37a is treated with hydrochloric acid to remove the Boc protecting group to thereby synthesize a compound of formula 39, which is then reacted with 2,2-dimethyloxirane to synthesize a compound of formula 40. The hydroxyl group of the compound of formula 40 is substituted with fluoride to synthesize a compound of formula 41. Each of the compounds of formulas 39, 40 and 41 is reacted with an aqueous hydroxylamine solution and potassium hydroxide, thereby synthesizing compounds 611, 613 and 615.

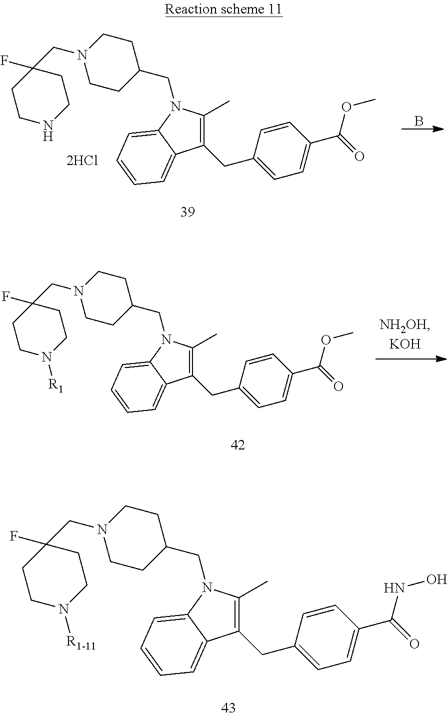

wherein reagent B may be a combination of the following compounds:

Acetic anhydride and diisopropylethylamine;

paraformaldehyde, NaBH₃CN and acetic acid (AcOH);

acetone, NaBH₃CN and acetic acid (AcOH);

isopropyl isocyanate and triethylamine; or

methanesulfonyl chloride and triethylamine.

In reaction scheme 11 above, R₁₋₁₁ may be

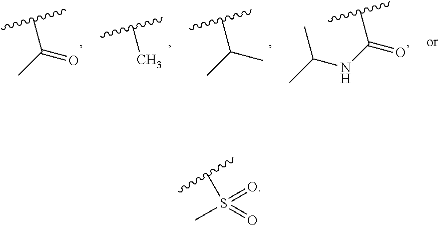

TABLE 10
Formula 47

| Compound | | |
|---|---|---|
| No. | Reagent (B) | R₁₋₁₁ |
| 612 | Acetic anhydride, DIPEA | 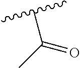 [SMILES: *C(C)=O] |
| 679 | Taraformaldehyde, NaBH₃CN, AcOH | 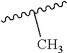 |
| 681 | Acetone, NaBH₃CN, AcOH | 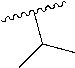 [SMILES: *C(C)C] |
| 695 | Isopropyl Isocyanate, Et₃N | 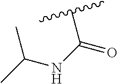 [SMILES: *C(=O)NC(C)C] |
| 696 | Methanesulfonyl chloride, Et₃N | 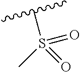 [SMILES: *S(C)(=O)=O] |

In reaction scheme 11, a secondary amine of formula 39 is reacted with reagent B to synthesize compounds of formula 42 having various substituents, which are then reacted with an aqueous hydroxylamine solution and potassium hydroxide, thereby synthesizing compounds 612, 679, 681, 695 and 696.

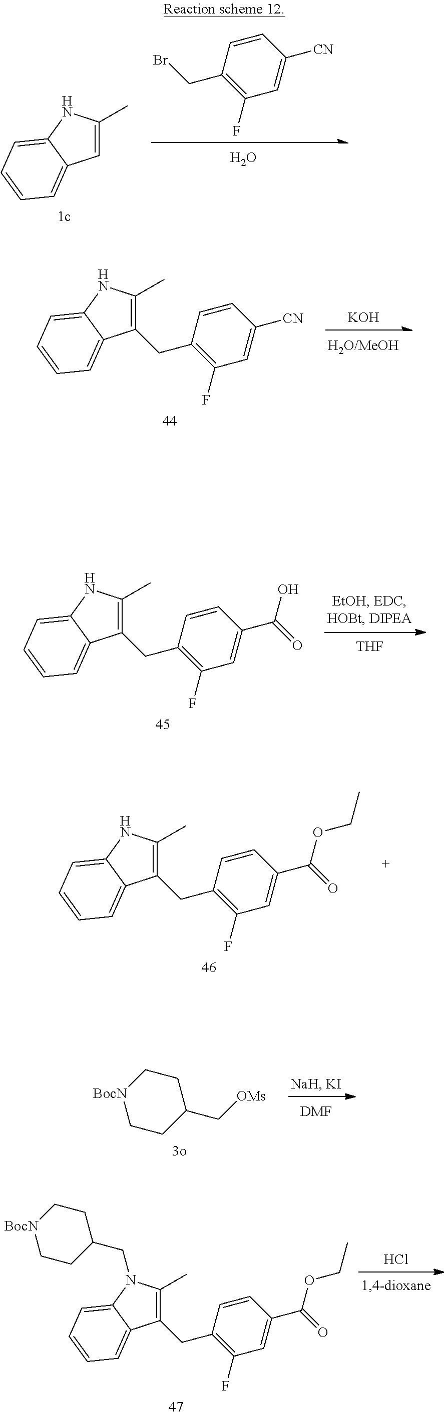

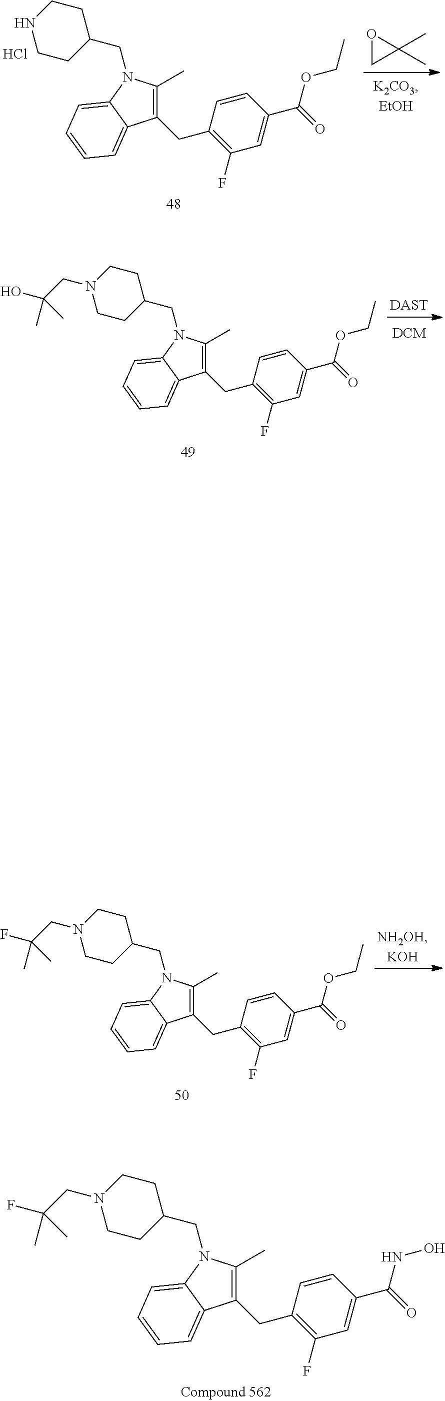

In reaction scheme 12 above, a compound of formula 1c is subjected to catalyst-free Friedel-Crafts alkylation with 4-(bromomethyl)-3-fluorobenzonitrile in a microwave reactor to synthesize a compound of formula 44, which is then hydrolyzed with potassium hydroxide and esterified with 1-ethyl-3-(3-dimethylaminopropyl)carbodiimide (EDC), thereby synthesizing a compound of formula 46. The compound of formula 47 is subjected to a substitution reaction with a compound of formula 3a to synthesize a compound of formula 47, which is then treated with hydrochloric acid to remove the Boc protecting group and is reacted with 2,2-dimethyloxirane to synthesize a compound of formula 49. The hydroxyl group of the compound of formula 49 is substituted with fluoride to synthesize a compound of formula 50, which is then reacted with an aqueous hydroxylamine solution and potassium hydroxide, thereby synthesizing compound 562.

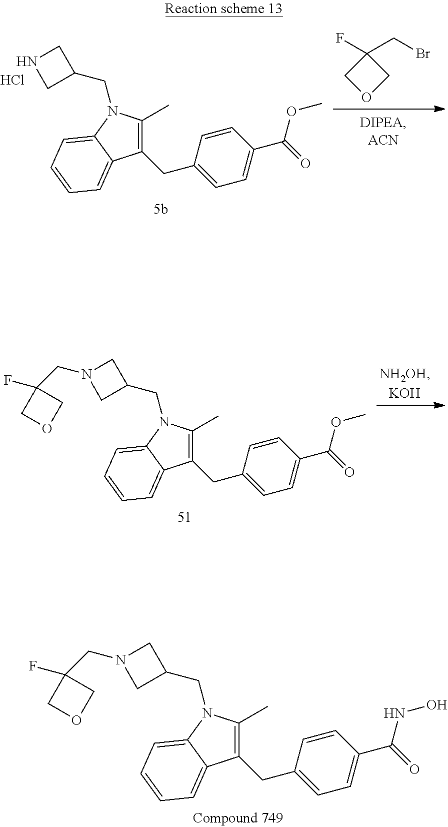

In reaction scheme 13 above, a secondary amine of formula 5b is substituted with 3-(bromomethyl)-3-fluorooxetane to synthesize a compound of formula 51, which is then reacted with an aqueous hydroxylamine solution and potassium hydroxide, thereby synthesizing compound 749.

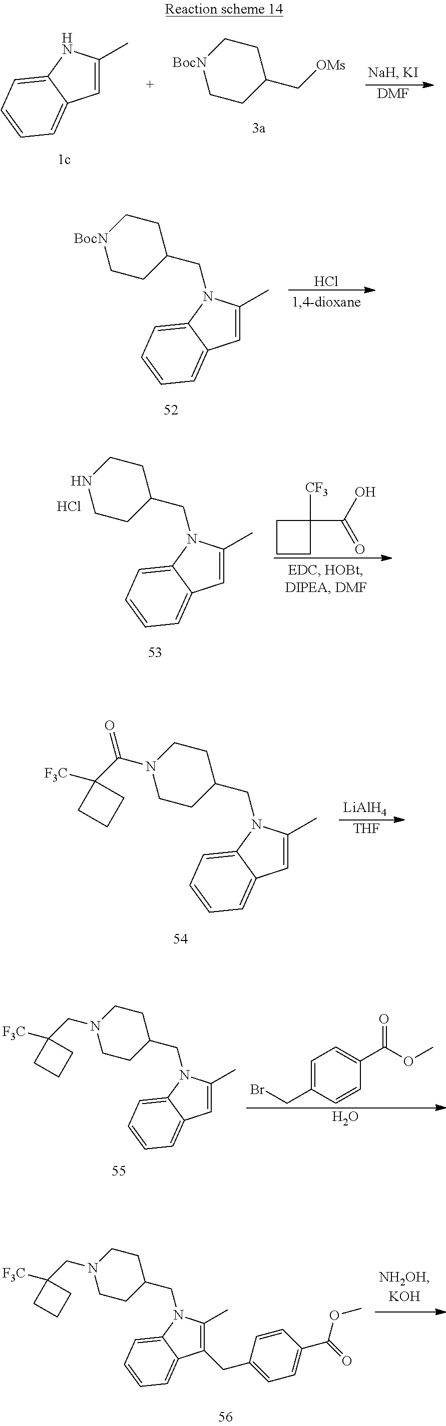

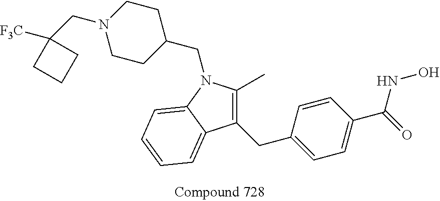 [SMILES: Cc1c(Cc2ccc(C(=O)NO)cc2)c2ccccc2n1CC1CCN(CC2(C(F)(F)F)CCC2)CC1]

In reaction scheme 14 above, a compound of formula 1c is subjected to a substitution reaction with a compound of formula 3a in the presence of sodium hydride to synthesize a compound of formula 52, which is then treated with hydrochloric acid to remove the Boc protecting group and is subjected to amide coupling with 1-(trifluoromethyl)cyclobutanecarboxylic acid and 1-ethyl-3-(3-dimethylaminopropyl)carbodiimide (EDC) to synthesize a compound of formula 54, which is then reduced with lithium aluminum hydride (LAH) to synthesize a compound of formula 55. The compound of formula 55 is subjected to catalyst-free Friedel-Crafts alkylation with methyl 4-(bromomethyl)benzoate in a microwave reactor to synthesize a compound of formula 56, which is then reacted with an aqueous hydroxylamine solution and potassium hydroxide, thereby synthesizing compound 728.

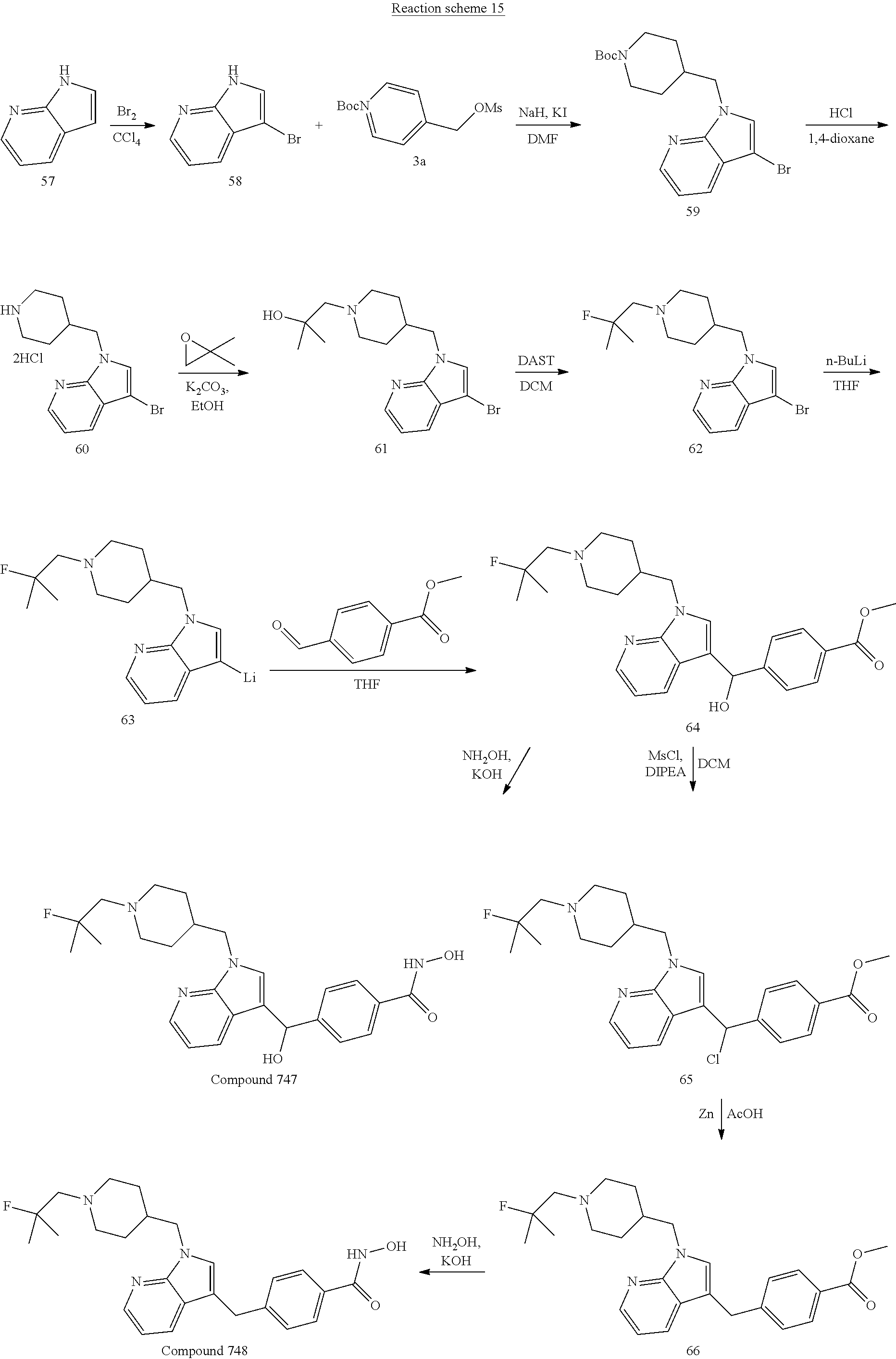

In reaction scheme 15 above, a compound of formula 57 is reacted with bromine to synthesize a compound of formula 58, which is then subjected to a substitution reaction with a compound of formula 3a in the presence of sodium hydride to synthesize a compound of formula 59. The compound of formula 59 is treated with hydrochloric acid to remove the Boc protecting group, and is reacted with 2,2-dimethyloxirane to synthesize a compound of formula 61. The hydroxyl group of the compound of formula 61 is substituted with fluoride to synthesize a compound of formula 62. The compound of formula 62 is reacted with n-butyllithium to substitute the bromine group with lithium, and reacted with methyl 4-formylbenzoate to synthesize a compound of formula 64. The compound of formula 64 is reacted with methanesulfonyl chloride to substitute the hydroxyl group with chloride to thereby synthesize a compound of formula 65, which is then treated with zinc to remove chloride, thereby synthesizing a compound of formula 66. Each of the compounds of formulas 64 and 66 is reacted with an aqueous hydroxylamine solution and potassium hydroxide, thereby synthesizing compounds 747 and 748.

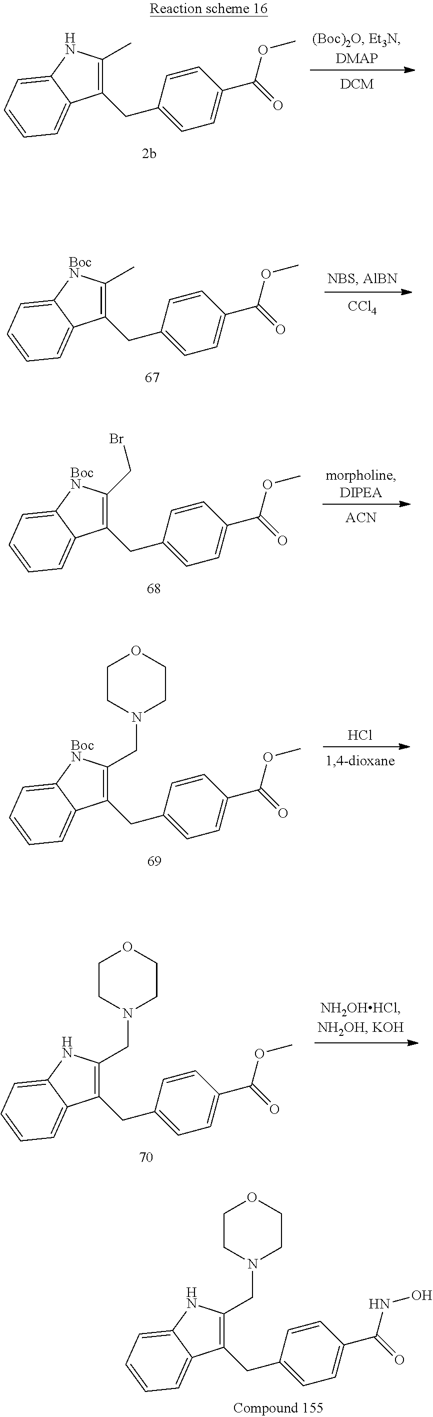

In reaction scheme 16 above, a compound of formula 2b is protected with Boc and reacted with N-bromosuccinimide (NBS) to introduce a bromine group therein to thereby synthesize a compound of formula 68, which is then substituted with morpholine to synthesize a compound of formula 69. The compound of formula 69 is treated with hydrochloric acid to remove the Boc protecting group and is reacted with an aqueous hydroxylamine solution and potassium hydroxide, thereby synthesizing compound 155.

The present invention also provides a pharmaceutical composition comprising the compound represented by formula I, or at least one of the above-listed compounds, an isomer thereof, a pharmaceutically acceptable salt thereof, or a hydrate or solvate thereof, together with a pharmaceutically acceptable carrier.

The pharmaceutical composition may be a composition for preventing or treating a disease associated with histone deacetylase (HDAC) activity.

The disease associated with histone deacetylase (HDAC) activity may be selected from among cell proliferative diseases such as cancer, autosomal dominant diseases such as Huntington's disease, genetic metabolic diseases such as fibrosis diseases, for example, cystic fibrosis, hepatic fibrosis, kidney fibrosis, pulmonary fibrosis and skin fibrosis, autoimmune diseases such as rheumatoid arthritis, diabetes, acute/chronic neurological diseases such as stroke, hypertrophy such as cardiac hypertrophy, congestive heart failure, amyotrophic lateral sclerosis, glaucoma, ocular diseases (associated with angiogenesis), and Alzheimer's disease.

In the pharmaceutical composition, the compound represented by formula I, I-1 or I-2, or at least one of the above-listed compounds, may be 4-((1-((1-(2-fluoro-2-methylpropyl)piperidin-4-yl)methyl)-2-methyl-1H-indol-3-yl)methyl)-N-hydroxybenzamide, or 4-((1-((1-((3-fluorooxetan-3-yl)methyl)piperidin-4-yl)methyl)-2-methyl-1H-indol-3-yl)methyl)-N-hydroxybenzamide.

The pharmaceutically acceptable carrier that is used in the composition of the present invention may be physiological saline, sterile water, Ringer's solution, buffered saline, dextrose solution, maltodextrin solution, glycerol, ethanol, and a mixture of two or more thereof. If necessary, the composition may contain other conventional additives such as an antioxidant, a buffer or a bacteriostatic agent.

The pharmaceutical composition of the present invention may be administered orally or parenterally (e.g., intravenously, subcutaneously, intraperitoneally or topically) depending on the intended use. The dose of the pharmaceutical composition varies depending on the patient's weight, age, sex, health conditions and diet, the time of administration, the mode of administration, the duration or interval of administration, excretion rate, idiosyncrasy, the nature of the formulation, the severity of the disease, and the like. The daily dose of the compound represented by formula I, I-1 or I-2, at least one of the above-listed compounds, an isomer thereof, a pharmaceutically acceptable salt thereof, or a hydrate or solvate thereof, may be about 0.01-100 mg/kg, preferably 0.1-30 mg/kg, and may be administered once to three times a day.

For administration, the pharmaceutical composition of the present invention may be prepared in various formulations. The pharmaceutical composition may be formulated in various forms using excipients. The excipient is any pharmaceutically acceptable solid, semi-solid or liquid excipient that is non-toxic and inert, and examples thereof include fillers, extenders, binders, wetting agents, disintegrants, dispersing agents, surfactants, and diluents.

The pharmaceutical composition of the present invention may be formulated as tablets, coated tablets, capsules, pills, granules, suppositories, liquids, suspensions, emulsions, pastes, ointments, gels, cream, lotion, powers or spray solutions. For example, for oral administration, the pharmaceutical composition may be formulated as solid preparations such as tablets, pills, powders, granules or capsules, or liquid preparations such as suspensions, solutions for internal use, emulsions, or syrups. For parenteral administration, the pharmaceutical composition may be formulated as injectable solutions, suspensions, emulsions, freeze-dried preparations, or suppositories. For example, the pharmaceutical composition may be formulated as microcapsules using the compound represented by formula I, an optical isomer thereof, a pharmaceutically acceptable salt thereof, a hydrate thereof, or a solvent thereof, together with at least one excipient.

The pharmaceutical composition of the present invention may comprise, as an active ingredient, the compound represented by formula I, at least one of the above-listed compounds, an isomer thereof, a pharmaceutically acceptable salt thereof, or a hydrate or solvate thereof, in an amount of about 0.1-99.5 wt %, and preferably about 0.5-95 wt %, based on the total weight of the composition.

The contents of excipients and additives, such as carriers, fillers, extenders, binders, wetting agents, disintegrants, dispersing agents, surfactants or diluents, which are added in the formulation of the pharmaceutical composition of the present invention, are not specifically limited, and may be suitably selected within content ranges that are used in conventional formulation.

The present invention also provides a method of preventing or treating a disease associated with histone deacetylase (HDAC) activity, for example, cell proliferative disease, autosomal dominant disease, fibrosis, autoimmune disease, diabetes, acute neurological disease, chronic neurological disease, hypertrophy, congestive heart failure, amyotrophic lateral sclerosis, glaucoma, angiogenesis-related ocular disease, or Alzheimer's disease, using the compound represented by formula I, at least one of the above-listed compounds, an isomer thereof, a pharmaceutically acceptable salt thereof, or a hydrate or solvate thereof.

The composition that is used in the method of preventing or treating a histone deacetylase (HDAC) activity-associated disease according to the present invention includes the pharmaceutical composition described in the specification.

In addition, subjects in need of the method of preventing or treating a histone deacetylase (HDAC) activity-associated disease according to the present invention include mammals, particularly humans.

The present invention also provides the use of the compound represented by formula I, at least one of the above-listed compounds, an isomer thereof, a pharmaceutically acceptable salt thereof, or a hydrate or solvate thereof, for preventing or treating a disease associated with histone deacetylase (HDAC) activity.

### Advantageous Effects

The present invention provides novel indole derivative compounds capable of inhibiting histone deacetylase (HDAC). Thus, the novel indole derivative compounds according to the present invention can be used to effectively treat a disease associated with histone deacetylase (HDAC) activity.

### DESCRIPTION OF DRAWINGS

FIG. 1 shows the inhibitory effect of a compound of the present invention against histone deacetylase 6.

FIG. 2 shows the distribution of a compound of the present invention in the brain after oral administration of the compound.

### MODE FOR INVENTION

Hereinafter, the present invention will be described in further detail with reference to examples and experimental examples. It is to be understood, however, that these examples are for illustrative purposes only and are not intended to limit the scope of the present invention.

In addition, the reagents and solvents used in the following examples are those purchased from Sigma-Aldrich Korea Co. or TCI Korea Co., unless specified otherwise. Purity was measured by area % of HPLC, and the HLPC system used was Alliance (Waters Corp.). ¹H NMR was measured using an Oxford NMR 400 spectrometer (Varian Instrument Co.). Mass spectra were measured using an LC/MSD SL mass spectrometer (Agilent Technologies, USA) equipped with an electrospray ionization source. For purification of compounds, MPLC was performed using CombiFlash Rf-200 (Teledyne ISCO, USA).

### EXAMPLES

### Example 1

### Synthesis of Compound 153

### Step 1: (Formula 17) Methyl 4-((2-methyl-1-(2-morpholinoethyl)-1H-indol-3-yl)methyl)benzoate

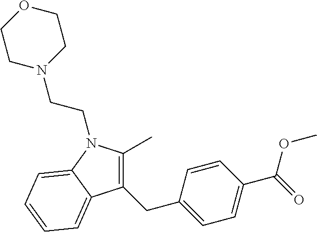 [SMILES: COC(=O)c1ccc(Cc2c(C)n(CCN3CCOCC3)c3ccccc23)cc1]

Methyl 4-((2-methyl-1-(2-(methylsulfonyloxy)ethyl)-1H-indol-3-yl)methyl)benzoate (0.160 g, 0.40 mmol) was dissolved in acetonitrile (3 mL), and morpholine (0.174 mL, 1.99 mmol) and diisopropylethylamine (0.348 mL, 1.99 mmol) were added thereto. The mixture was allowed to react in a microwave reactor at 120° C. for 1 hour. Then, the reaction solution was extracted with ethyl acetate and a saturated aqueous solution of sodium hydrogen carbonate, and the organic layer was dried with anhydrous magnesium sulfate and filtered. The filtrate was concentrated under reduced pressure, and the residue was purified by column chromatography (SiO₂; hexane/ethyl acetate=1/2) to afford the title compound (0.106 g, 68%) as a light yellow solid.

### Step 2: (Compound 153) N-hydroxy-4-((2-methyl-1-(2-morpholinoethyl)-1H-indol-3-yl)methyl)benzamide

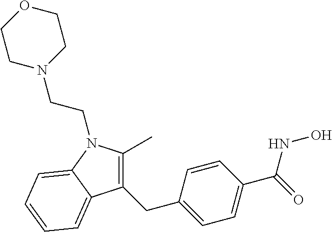 [SMILES: Cc1c(Cc2ccc(C(=O)NO)cc2)c2ccccc2n1CCN1CCOCC1]

Methyl 4-((2-methyl-1-(2-morpholinoethyl)-1H-indol-3-yl)methyl)benzoate (0.106 g, 0.27 mmol) was dissolved in methanol (5 mL)/tetrahydrofuran (2 mL), and hydroxylamine (50 wt % aqueous solution, 5.0 mL, 81.02 mmol), hydroxylamine hydrochloride (0.09 g, 1.35 mmol) and potassium hydroxide (0.15 g, 2.70 mmol) were sequentially added thereto, followed by stirring at room temperature for 16 hours. After completion of the reaction, the reaction solution was concentrated under reduced pressure to a volume of about 2-3 mL, and about 1-2 mL of a saturated aqueous solution of sodium hydrogen carbonate was added thereto, followed by stirring. The produced solid product was filtered, washed with water, and dried in a vacuum to afford compound 153 (0.045 g, 42%) as a white solid.

¹H NMR (400 MHz, DMSO-d₆) δ 11.11 (brs, 1H), 8.99 (brs, 1H), 7.59 (d, 2H, J=8.3 Hz), 7.35 (d, 2H, J=8.4 Hz), 7.23 (d, 2H, J=8.2 Hz), 7.03 (t, 1H, J=7.7 Hz), 6.91 (t, 1H, J=7.4 Hz), 4.17 (t, 2H, J=7.2 Hz), 4.05 (s, 2H), 3.54-3.52 (m, 4H), 2.55-2.53 (m, 2H), 2.39 (s, 3H), 2.32-2.31 (m, 4H); MS (ESI) m/z 394.1 (M⁺+H).

### Example 2

### Synthesis of Compound 154

### Step 1: (Formula 17) Methyl 4-((2-methyl-1-(2-(4-methylpiperazin-1-yl)ethyl)-1H-indol-3-yl)methyl)benzoate

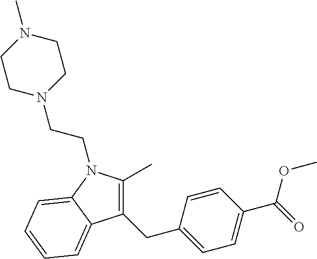 [SMILES: COC(=O)c1ccc(Cc2c(C)n(CCN3CCN(C)CC3)c3ccccc23)cc1]

Methyl 4-((2-methyl-1-(2-(methylsulfonyloxy)ethyl)-1H-indol-3-yl)methyl)benzoate (0.160 g, 0.40 mmol) was dissolved in acetonitrile (3 mL), and 1-methylpiperazine (0.221 mL, 1.99 mmol) and diisopropylethylamine (0.348 mL, 1.99 mmol) were added thereto. The mixture was allowed to react in a microwave reactor at 120° C. for 1 hour. Then, the reaction solution was extracted with ethyl acetate and a saturated aqueous solution of sodium hydrogen carbonate, and the organic layer was dried with anhydrous magnesium sulfate and filtered. The filtrate was concentrated under reduced pressure, and the residue was purified by column chromatography (SiO₂; methylene chloride/methanol=10/1) to afford the title compound (0.087 g, 54%) as a yellow solid.

### Step 2: (Compound 154) N-hydroxy-4-((2-methyl-1-(2-(4-methylpiperazin-1-yl)ethyl)-1H-indol-3-yl)methyl)benzamide

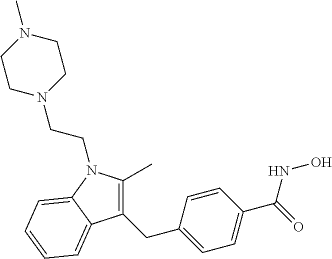 [SMILES: Cc1c(Cc2ccc(C(=O)NO)cc2)c2ccccc2n1CCN1CCN(C)CC1]

Methyl 4-((2-methyl-1-(2-(4-methylpiperazin-1-yl)ethyl)-1H-indol-3-yl)methyl)benzoate (0.087 g, 0.22 mmol) was dissolved in methanol (5 mL)/tetrahydrofuran (2 mL), and hydroxylamine (50 wt % aqueous solution, 5.2 mL, 85.81 mmol), hydroxylamine hydrochloride (0.07 g, 1.07 mmol) and potassium hydroxide (0.12 g, 2.15 mmol) were sequentially added, followed by stirring at room temperature for 16 hours. After completion of the reaction, the reaction solution was concentrated under reduced pressure to a volume of about 2-3 mL, and about 1-2 mL of a saturated aqueous solution of sodium hydrogen carbonate was added thereto, followed by stirring. The produced solid product was filtered, washed with water, and dried in a vacuum to afford compound 154 (0.023 g, 26%) as a light yellow solid.

¹H NMR (400 MHz, DMSO-d₆) δ 11.09 (brs, 1H), 9.00 (brs, 1H), 7.59 (d, 2H, J=8.2 Hz), 7.35-7.32 (m, 2H), 7.23 (d, 2H, J=8.0 Hz), 7.02 (t, 1H, J=7.2 Hz), 6.90 (t, 1H, J=7.1 Hz), 4.19 (t, 2H, J=6.9 Hz), 4.04 (s, 2H), 3.43-3.36 (m, 4H), 2.53-2.52 (m, 2H), 2.42 (s, 3H), 2.33-2.26 (m, 4H), 2.12 (s, 3H); MS (ESI) m/z 407.2 (M⁺+H).

### Example 3

### Synthesis of Compound 155

### Step 1: (Formula 67) Tert-butyl 3-(4-(methoxycarbonyl)benzyl)-2-methyl-1H-indole-1-carboxylate

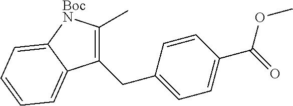 [SMILES: COC(=O)c1ccc(Cc2c(C)n(C(=O)OC(C)(C)C)c3ccccc23)cc1]

Methyl 4-((2-methyl-1H-indol-3-yl)methyl)benzoate (2.000 g, 7.16 mmol) was dissolved in methylene chloride (30 mL), and triethylamine (1.489 mL, 10.74 mmol), 4-dimethylaminopyridine (0.086 g, 0.72 mmol) and di-tert-butyl dicarbonate (1.875 g, 8.59 mmol) were added thereto, followed by stirring at room temperature for 3 hours. After completion of the reaction, the reaction solution was extracted with ethyl acetate and a saturated aqueous solution of sodium hydrogen carbonate, and the organic layer was dried with anhydrous magnesium sulfate and filtered. The filtrate was concentrated under reduced pressure, and the residue was purified by column chromatography (SiO₂; ethyl acetate/hexane=1/15) to afford the title compound (2.470 g, 91%) as a colorless liquid.

### Step 2: (Formula 68) Tert-butyl 2-(bromomethyl)-3-(4-(methoxycarbonyl)benzyl)-1H-indol-1-carboxylate

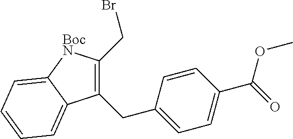 [SMILES: COC(=O)c1ccc(Cc2c(CBr)n(C(=O)OC(C)(C)C)c3ccccc23)cc1]

Tert-butyl 3-(4-(methoxycarbonyl)benzyl)-2-methyl-1H-indol-1-carboxylate (2.470 g, 6.51 mmol) was dissolved in tetrachloromethane (30 mL), and N-bromosuccinimide (1.217 g, 6.84 mmol) and 2,2′-azobis(2-methylpropionitrile) (0.107 g, 0.65 mmol) were added thereto, followed by stirring under reflux for 2 hours. Then, the reaction solution was cooled to room temperature, and then extracted with ethyl acetate and a saturated aqueous solution of sodium hydrogen carbonate, and the organic layer was dried with anhydrous magnesium sulfate and filtered. The filtrate was concentrated under reduced pressure, and the residue was purified by column chromatography (SiO₂; ethyl acetate/hexane=1/10) to afford the title compound (1.45 g, 49%) as a white solid.

### Step 3: (Formula 69) Tert-butyl 3-(4-(methoxycarbonyl)benzyl)-2-(morpholinomethyl)-1H-indole-1-carboxylate

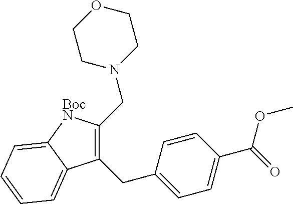 [SMILES: COC(=O)c1ccc(Cc2c(CN3CCOCC3)n(C(=O)OC(C)(C)C)c3ccccc23)cc1]

Tert-butyl 2-(bromomethyl)-3-(4-(methoxycarbonyl)benzyl)-1H-indole-1-carboxylate (0.200 g, 0.44 mmol) was dissolved in acetonitrile (10 mL), and morpholine (0.076 mL, 0.87 mmol) and diisopropylethylamine (0.169 g, 1.31 mmol) were added thereto, followed by stirring at room temperature for 16 hours. After completion of the reaction, the residue was purified by column chromatography (SiO₂; ethyl acetate/hexane=1/3) to afford the title compound (0.101 g, 50%) as a white solid.

### Step 4: (Formula 70) Methyl 4-((2-(morpholinomethyl)-1H-indol-3-yl)methyl)benzoate

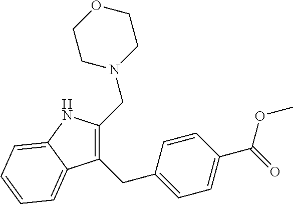 [SMILES: COC(=O)c1ccc(Cc2c(CN3CCOCC3)[nH]c3ccccc23)cc1]

Tert-butyl 3-(4-(methoxycarbonyl)benzyl)-2-(morpholinomethyl)-1H-indole-1-carboxylate (0.097 g, 0.21 mmol) was dissolved in 1,4-dioxane (3 mL), and a hydrochloric acid solution (4.0 M (dissolved in 1,4-dioxane solution, 3.132 mL, 12.53 mmol)) was added thereto, followed by stirring at room temperature for 16 hours. After completion of the reaction, the reaction solution was concentrated under reduced pressure and extracted with ethyl acetate and a saturated aqueous solution of sodium hydrogen carbonate, and the organic layer was dried with anhydrous magnesium sulfate and filtered. The filtrate was concentrated under reduced pressure, and the residue was purified by column chromatography (SiO₂; ethyl acetate/hexane=1/1) to afford the title compound (0.048 g, 63%) as a light yellow solid.

### Step 5: (Compound 155) N-hydroxy-4-((2-(morpholinomethyl)-1H-indol-3-yl)methyl)benzamide

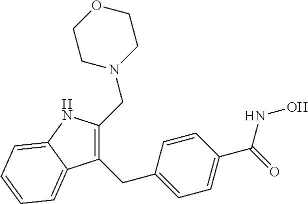 [SMILES: O=C(NO)c1ccc(Cc2c(CN3CCOCC3)[nH]c3ccccc23)cc1]

Methyl 4-((2-(morpholinomethyl)-1H-indol-3-yl)methyl)benzoate (0.048 g, 0.13 mmol) was dissolved in tetrahydrofuran (1 mL)/methanol (3 mL), and hydroxylamine (50 wt % aqueous solution, 2.014 mL, 65.85 mmol), hydroxylamine hydrochloride (0.046 g, 0.66 mmol) and potassium hydroxide (0.074 g, 1.32 mmol) were sequentially added thereto, followed by stirring at room temperature for 16 hours. After completion of the reaction, the reaction solution was concentrated under reduced pressure to a volume of about 2-3 mL, and about 1-2 mL of a saturated aqueous solution of sodium hydrogen carbonate was added thereto, followed by stirring. The produced solid product was filtered, washed with water, and dried in a vacuum to afford compound 155 (0.033 g, 69%) as a white solid.

¹H NMR (400 MHz, DMSO-d₆) δ 10.89 (s, 1H), 7.60 (d, 2H, J=8.2 Hz), 7.32-7.27 (m, 4H), 6.99 (t, 1H, J=7.1 Hz), 6.87 (t, 1H, J=7.7 Hz), 4.09 (s, 2H), 3.63 (s, 2H), 3.56-3.53 (m, 4H), 2.33-2.31 (m, 4H); MS (ESI) m/z 366.1 (M⁺+H).

### Example 4

### Synthesis of Compound 196

### Step 1: (Formula 17) Methyl 4-((1-(2-(4-isopropylpiperazin-1-yl)ethyl)-2-methyl-1H-indol-3-yl)methyl)benzoate)

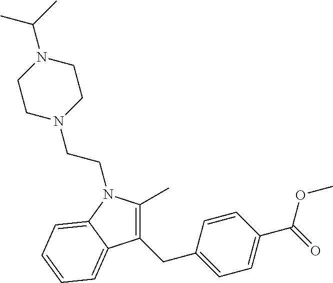 [SMILES: COC(=O)c1ccc(Cc2c(C)n(CCN3CCN(C(C)C)CC3)c3ccccc23)cc1]

Methyl 4-((2-methyl-1-(2-(methylsulfonyloxy)ethyl)-1H-indol-3-yl)methyl)benzoate (0.150 g, 0.37 mmol) was dissolved in acetonitrile (3 mL), and 1-isopropylpiperazine (0.144 g, 1.12 mmol) and diisopropylethylamine (0.326 mL, 1.87 mmol) were added thereto. The mixture was allowed to react in a microwave reactor at 120° C. for 3 hours. Then, the reaction solution was concentrated under reduced pressure, and the residue was purified by column chromatography (SiO₂; methylene chloride/methanol=10/1) to afford the title compound (0.082 g, 51%) as a light yellow solid.

### Step 2: (Compound 196) N-hydroxy-4-((1-(2-(4-isopropylpiperazin-1-yl)ethyl)-2-methyl-1H-indol-3-yl)methyl)benzamide

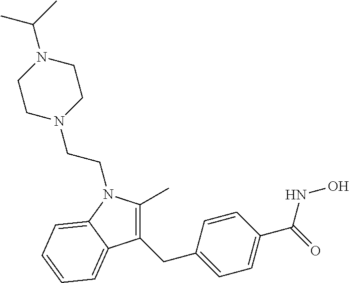 [SMILES: Cc1c(Cc2ccc(C(=O)NO)cc2)c2ccccc2n1CCN1CCN(C(C)C)CC1]

Methyl 4-((1-(2-(4-isopropylpiperazin-1-yl)ethyl)-2-methyl-1H-indol-3-yl)methyl)benzoate (0.082 g, 0.19 mmol) was dissolved in methanol (10 mL), and hydroxylamine (50 wt % aqueous solution, 3.470 mL, 56.74 mmol), hydroxylamine hydrochloride (0.066 g, 0.95 mmol) and potassium hydroxide (0.212 g, 3.78 mmol) were sequentially added thereto, followed by stirring a room temperature for 16 hours. After completion of the reaction, the reaction solution was concentrated under reduced pressure to a volume of about 2-3 mL, and about 1-2 mL of a saturated aqueous solution of sodium hydrogen carbonate was added thereto, followed by stirring. The produced solid product was filtered, washed with water, and dried in a vacuum to afford compound 196 (0.045 g, 55%) as a white solid.

¹H NMR (400 MHz, DMSO-d₆) δ 7.59 (d, 2H, J=8.3 Hz), 7.33 (t, 2H, J=7.1 Hz), 7.22 (d, 2H, J=8.2 Hz), 7.02 (t, 1H, J=7.1 Hz), 6.90 (t, 1H, J=7.4 Hz), 4.19 (t, 2H, J=6.8 Hz), 4.04 (s, 2H), 2.57-2.52 (m, 3H), 2.42 (s, 3H), 2.40-2.38 (m, 8H), 0.94 (s, 3H), 0.92 (s, 3H); MS (ESI) m/z 435.3 (M⁺+H).

### Example 5

### Synthesis of Compound 197

### Step 1: (Formula 17) Methyl 4-((1-(2-(4-(2-hydroxyethyl)piperazin-1-yl)ethyl)-2-methyl-1H-indol-3-yl)methyl)benzoate

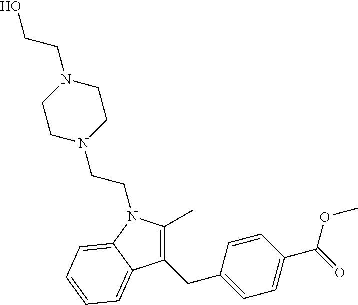 [SMILES: COC(=O)c1ccc(Cc2c(C)n(CCN3CCN(CCO)CC3)c3ccccc23)cc1]

Methyl 4-((2-methyl-1-(2-(methylsulfonyloxy)ethyl)-1H-indol-3-yl)methyl)benzoate (0.150 g, 0.37 mmol) was dissolved in acetonitrile (3 mL), and 2-(piperazin-1-yl)ethanol (0.146 g, 1.12 mmol) and diisopropylethylamine (0.326 mL, 1.87 mmol) were added thereto. The mixture was allowed to react in a microwave reactor at 120° C. for 3 hours. Then, the reaction solution was concentrated under reduced pressure, and the residue was purified by column chromatography (SiO₂; methylene chloride/methanol=10/1) to afford the title compound (0.057 g, 35%) as a yellow liquid.

### Step 2: (Compound 197) N-hydroxy-4-((1-(2-(4-(2-hydroxyethyl)piperazin-1-yl)ethyl)-2-methyl-1H-indol-3-yl)methyl)benzamide

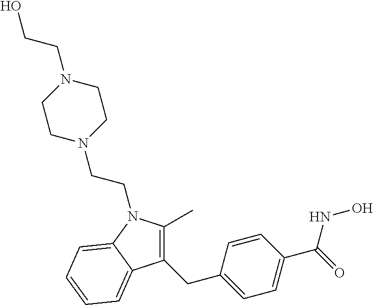 [SMILES: Cc1c(Cc2ccc(C(=O)NO)cc2)c2ccccc2n1CCN1CCN(CCO)CC1]

Methyl 4-((1-(2-(4-(2-hydroxyethyl)piperazin-1-yl)ethyl)-2-methyl-1H-indol-3-yl)methyl)benzoate (0.057 g, 0.13 mmol) was dissolved in methanol (10 mL), and hydroxylamine (50 wt % aqueous solution, 2.401 mL, 39.26 mmol), hydroxylamine hydrochloride (0.046 g, 0.65 mmol) and potassium hydroxide (0.147 g, 2.62 mmol) were sequentially added thereto, followed by stirring at room temperature for 16 hours.

After completion of the reaction, the reaction solution was concentrated under reduced pressure to a volume of about 2-3 mL, and about 1-2 mL of a saturated aqueous solution of sodium hydrogen carbonate was added thereto, followed by stirring. The produced solid product was filtered, washed with water, and dried in a vacuum to afford compound 197 (0.034 g, 60%) as a light green solid.

¹H NMR (400 MHz, DMSO-d₆) δ 11.09 (brs, 1H), 9.01 (brs, 1H), 7.59 (d, 2H, J=8.3 Hz), 7.36-7.30 (m, 2H), 7.24 (d, 2H, J=8.3 Hz), 7.01 (m, 1H), 6.91 (m, 1H), 4.19 (t, 2H, J=7.0 Hz), 4.04 (s, 2H), 3.46-3.44 (m, 2H), 3.40-3.38 (m, 2H), 2.53-2.51 (m, 2H), 2.44 (s, 3H), 2.44-2.32 (m, 9H); MS (ESI) m/z 437.2 (M⁺+H).

### Example 6

### Synthesis of Compound 198

### Step 1: (Formula 17) Methyl 4-((1-(2-(4-(2-methoxyethyl)piperazin-1-yl)ethyl)-2-methyl-1H-indol-3-yl)methyl)benzoate

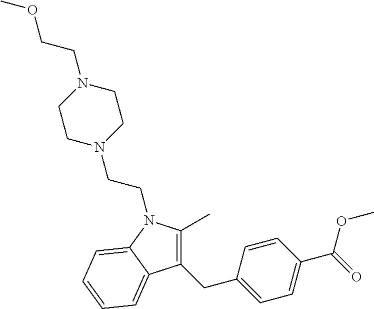

Methyl 4-((2-methyl-1-(2-(methylsulfonyloxy)ethyl)-1H-indol-3-yl)methyl)benzoate (0.150 g, 0.37 mmol) was dissolved in acetonitrile (3 mL), and 1-(2-methoxyethyl)piperazine (0.162 g, 1.12 mmol) and diisopropylethylamine (0.326 mL, 1.87 mmol) were added thereto. The mixture was allowed to react in a microwave reactor at 120° C. for 3 hours. Then, the reaction solution was concentrated under reduced pressure, and the residue was purified by column chromatography (SiO₂; methylene chloride/methanol=10/1) to afford the title compound (0.080 g, 48%) as a yellow liquid.

### Step 2: (Compound 198) N-hydroxy-4-((1-(2-(4-(2-methoxyethyl)piperazin-1-yl)ethyl)-2-methyl-1H-indol-3-yl)methyl)benzamide

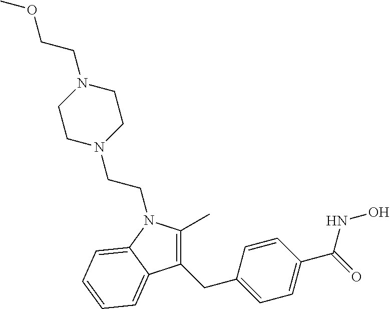 [SMILES: COCCN1CCN(CCn2c(C)c(Cc3ccc(C(=O)NO)cc3)c3ccccc32)CC1]

Methyl 4-((1-(2-(4-(2-methoxyethyl)piperazin-1-yl)ethyl)-2-methyl-1H-indol-3-yl)methyl)benzoate (0.080 g, 0.18 mmol) was dissolved in methanol (10 mL), and hydroxylamine (50% aqueous solution, 3.265 mL, 53.38 mmol), hydroxylamine hydrochloride (0.062 g, 0.89 mmol) and potassium hydroxide (0.200 g, 3.56 mmol) were sequentially added thereto, followed by stirring at room temperature for 16 hours. After completion of the reaction, the reaction solution was concentrated under reduced pressure to a volume of about 2-3 mL, and about 1-2 mL of a saturated aqueous solution of sodium hydrogen carbonate was added thereto, followed by stirring. The produced solid product was filtered, washed with water, and dried in a vacuum to afford compound 198 (0.052 g, 65%) as a light yellow solid.

¹H NMR (400 MHz, DMSO-d₆) δ 7.59 (d, 2H, J=8.2 Hz), 7.35-7.32 (m, 2H), 7.23 (d, 2H, J=8.0 Hz), 7.02 (m, 1H), 6.92 (m, 1H), 4.19-4.18 (m, 2H), 4.04 (s, 3H), 3.39-3.35 (m, 4H), 3.22-3.21 (m, 3H), 2.95-2.92 (m, 2H), 2.63-2.59 (m, 3H), 2.49-2.41 (m, 7H); MS (ESI) m/z 451.2 (M⁺+H).

### Example 7

### Synthesis of Compound 199

### Step 1: (Formula 17) (S)-methyl 4-((1-(2-(2-(hydroxymethyl)pyrrolidin-1-yl)ethyl)-2-methyl-1H-indol-3-yl)methyl)benzoate)

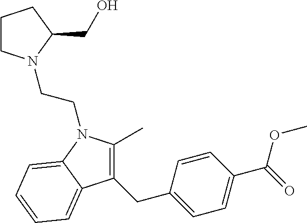 [SMILES: COC(=O)c1ccc(Cc2c(C)n(CCN3CCC[C@H]3CO)c3ccccc23)cc1]

Methyl 4-((2-methyl-1-(2-(methylsulfonyloxy)ethyl)-1H-indol-3-yl)methyl)benzoate (0.150 g, 0.37 mmol) was dissolved in acetonitrile (3 mL), and (S)-pyrrolidin-2-ylmethanol (0.113 g, 1.12 mmol) and diisopropylethylamine (0.326 mL, 1.87 mmol) were added thereto. The mixture was allowed to react in a microwave reactor at 120° C. for 3 hours. Then, the reaction solution was concentrated under reduced pressure, and the residue was purified by column chromatography (SiO₂; methylene chloride/methanol=15/1) to afford the title compound (0.079 g, 52%).

### Step 2: (Compound 199) (S)—N-hydroxy-4-((1-(2-(2-(hydroxymethyl)pyrrolidin-1-yl)ethyl)-2-methyl-1H-indol-3-yl)methyl)benzamide

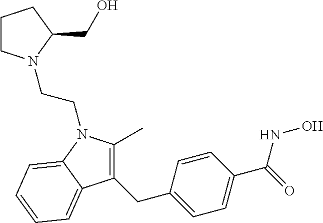 [SMILES: Cc1c(Cc2ccc(C(=O)NO)cc2)c2ccccc2n1CCN1CCC[C@H]1CO]

(S)-methyl 4-((1-(2-(2-(hydroxymethyl)pyrrolidin-1-yl)ethyl)-2-methyl-1H-indol-3-yl)methyl)benzoate (0.079 g, 0.19 mmol) was dissolved in methanol (10 mL), and hydroxylamine (50 wt % aqueous solution, 3.566 mL, 58.30 mmol), hydroxylamine hydrochloride (0.068 g, 0.97 mmol) and potassium hydroxide (0.218 g, 3.89 mmol) were added thereto, followed by stirring at room temperature for 16 hours. After completion of the reaction, the reaction solution was concentrated under reduced pressure to a volume of about 2-3 mL, and about 1-2 mL of a saturated aqueous solution of sodium hydrogen carbonate was added thereto, followed by stirring. The produced solid product was filtered, washed with water, and dried in a vacuum to afford compound 199 (0.052 g, 66%) as a light brown solid.

¹H NMR (400 MHz, DMSO-d₆) δ 7.58 (d, 2H, J=8.2 Hz), 7.33 (d, 2H, J=8.8 Hz), 7.19 (d, 2H, J=8.2 Hz), 7.00 (t, 1H, J=7.9 Hz), 6.90 (t, 1H, J=7.7 Hz), 4.36 (brs, 1H), 4.19-4.17 (m, 2H), 4.03 (s, 2H), 3.22 (m, 1H), 3.14 (m, 1H), 3.04-3.03 (m, 2H), 2.56 (m, 1H), 2.46 (m, 1H), 2.40 (s, 3H), 2.22 (m, 1H), 1.74 (m, 1H), 1.62-1.58 (m, 2H), 1.46 (m, 1H); MS (ESI) m/z 408.2 (M⁺+H).

### Example 8

### Synthesis of Compound 200

### Step 1: (Formula 17) (S)-methyl 4-((1-(2-(2-(methoxymethyl)pyrrolidin-1-yl)ethyl)-2-methyl-1H-indol-3-yl)methyl)benzoate)

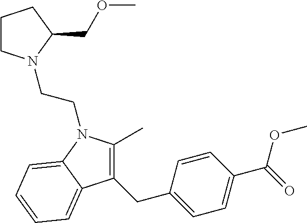 [SMILES: COC[C@@H]1CCCN1CCn1c(C)c(Cc2ccc(C(=O)OC)cc2)c2ccccc21]

Methyl 4-((2-methyl-1-(2-(methylsulfonyloxy)ethyl)-1H-indol-3-yl)methyl)benzoate (0.150 g, 0.37 mmol) was dissolved in acetonitrile (3 mL), and (S)-2-(methoxymethyl)pyrrolidine (0.129 g, 1.12 mmol) and diisopropylethylamine (0.326 mL, 1.87 mmol) were added thereto. The mixture was allowed to react in a microwave reactor at 120° C. for 3 hours. Then, the reaction solution was concentrated under reduced pressure, and the residue was purified by column chromatography (SiO₂; methylene chloride/methanol=20/1) to afford the title compound (0.097 g, 62%) as a light yellow liquid.

### Step 2: (Compound 200) (S)—N-hydroxy-4-((1-(2-(2-(methoxymethyl)pyrrolidin-1-yl)ethyl)-2-methyl-1H-indol-3-yl)methyl)benzamide

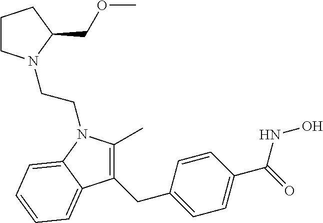 [SMILES: COC[C@@H]1CCCN1CCn1c(C)c(Cc2ccc(C(=O)NO)cc2)c2ccccc21]

(S)-methyl 4-((1-(2-(2-(methoxymethyl)pyrrolidin-1-yl)ethyl)-2-methyl-1H-indol-3-yl)methyl)benzoate (0.097 g, 0.23 mmol) was dissolved in methanol (10 mL), and hydroxylamine (50 wt % aqueous solution, 4.233 mL, 69.20 mmol), hydroxylamine hydrochloride (0.080 g, 1.15 mmol) and potassium hydroxide (0.259 g, 4.61 mmol) were sequentially added thereto, followed by stirring at room temperature for 16 hours. After completion of the reaction, the reaction solution was concentrated under reduced pressure to a volume of about 2-3 mL, and about 1-2 mL of a saturated aqueous solution of sodium hydrogen carbonate was added thereto, followed by stirring. The produced solid product was filtered, washed with water, and dried in a vacuum to afford compound 200 (0.072 g, 74%) as a light yellow solid.

¹H NMR (400 MHz, DMSO-d₆) δ 7.58 (d, 2H, J=8.2 Hz), 7.35-7.30 (m, 2H), 7.19 (d, 2H, J=8.2 Hz), 7.03 (t, 1H, J=7.6 Hz), 6.90 (t, 1H, J=7.4 Hz), 4.19-4.15 (m, 2H), 4.03 (s, 2H), 3.09 (s, 3H), 3.08-3.04 (m, 4H), 2.60-2.56 (m, 2H), 2.40 (s, 3H), 2.22 (m, 1H), 1.78 (m, 1H), 1.65-1.61 (m, 2H), 1.38 (m, 1H); MS (ESI) m/z 422.2 (M⁺+H).

### Example 9

### Synthesis of Compound 201

### Step 1: (Formula 17) Methyl 4-((2-methyl-1-(2-(2-methyl-1H-indazol-1-yl)ethyl)-1H-indol-3-yl)methyl)benzoate

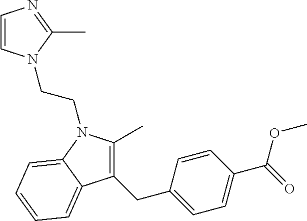 [SMILES: COC(=O)c1ccc(Cc2c(C)n(CCn3ccnc3C)c3ccccc23)cc1]

Methyl 4-((2-methyl-1-(2-(methylsulfonyloxy)ethyl)-1H-indol-3-yl)methyl)benzoate (0.150 g, 0.37 mmol) was dissolved in acetonitrile (3 mL), and 2-methyl-1H-imidazole (0.092 g, 1.12 mmol) and diisopropylethylamine (0.326 mL, 1.87 mmol) were added thereto. The mixture was allowed to react in a microwave reactor at 120° C. for 3 hours. Then, the reaction solution was concentrated under reduced pressure, and the residue was purified by column chromatography (SiO₂; methylene chloride/methanol=10/1) to afford the title compound (0.108 g, 75%) as a light yellow liquid.

### Step 2: (Compound 201) N-hydroxy-4-((2-methyl-1-(2-(2-methyl-1H-imidazol-1-yl)ethyl)-1H-indol-3-yl)methyl)benzamide

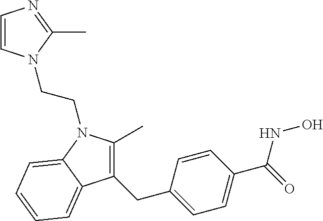 [SMILES: Cc1nccn1CCn1c(C)c(Cc2ccc(C(=O)NO)cc2)c2ccccc21]

Methyl 4-((2-methyl-1-(2-(2-methyl-1H-indazol-1-yl)ethyl)-1H-indol-3-yl)methyl)benzoate (0.108 g, 0.28 mmol) was dissolved in methanol (10 mL), and hydroxylamine (50 wt % aqueous solution, 5.115 mL, 83.62 mmol), hydroxylamine hydrochloride (0.097 g, 1.39 mmol) and potassium hydroxide (0.313 g, 5.58 mmol) were sequentially added thereto, followed by stirring at room temperature for 16 hours. After completion of the reaction, the reaction solution was concentrated under reduced pressure to a volume of about 2-3 mL, and about 1-2 mL of a saturated aqueous solution of sodium hydrogen carbonate was added thereto, followed by stirring. The produced solid product was filtered, washed with water, and dried in a vacuum to afford compound 201 (0.09 g, 82%) as a white solid.

¹H NMR (400 MHz, DMSO-d₆) δ 7.60 (d, 2H, J=8.2 Hz), 7.32 (t, 2H, J=7.3 Hz), 7.17 (d, 2H, J=6.1 Hz), 7.01 (t, 1H, J=6.1 Hz), 6.93-6.89 (m, 2H), 6.67 (d, 1H, J=0.9 Hz), 4.41 (t, 2H, J=4.1 Hz), 4.19 (t, 2H, J=4.0 Hz), 3.98 (s, 2H), 1.91 (s, 3H), 1.55 (s, 3H); MS (ESI) m/z 389.2 (M⁺+H).

### Example 10

### Synthesis of Compound 243

### Step 1: (Formula 2a) Methyl 6-((2-methyl-1H-indol-3-yl)methyl)nicotinate

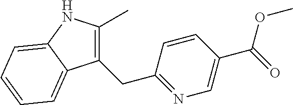 [SMILES: COC(=O)c1ccc(Cc2c(C)[nH]c3ccccc23)nc1]

2-Methylindole (0.500 g, 3.81 mmol) and methyl 6-(bromomethyl)nicotinate (0.877 g, 3.81 mmol) were added to water (5 mL) and allowed to react in a microwave reactor at 150° C. for 7 minutes. After completion of the reaction, the reaction solution was extracted with ethyl acetate and a saturated aqueous solution of sodium hydrogen carbonate, and the organic layer was dried with anhydrous magnesium sulfate and filtered. The filtrate was concentrated under reduced pressure, and the residue was purified by column chromatography (SiO₂; ethyl acetate/hexane=1/2) to afford the title compound (0.585 g, 55%) as a yellow solid.

### Step 2: (Formula 14) Methyl 6-((1-(2-(tert-butyldimethylsilyloxy)ethyl)-2-methyl-1H-indol-3-yl)methyl)nicotinate

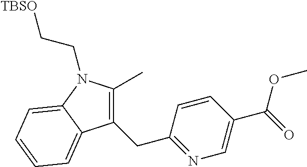 [SMILES: COC(=O)c1ccc(Cc2c(C)n(CCO[Si](C)(C)C(C)(C)C)c3ccccc23)nc1]

Methyl 6-((2-methyl-1H-indol-3-yl)methyl)nicotinate (0.500 g, 1.78 mmol) was dissolved in N,N-dimethylformamide (10 mL) and cooled to 0° C., and then sodium hydride (95%, 0.059 g, 2.32 mmol) was added thereto, followed by stirring for 5 minutes. Then, (2-bromoethoxy)(tert-butyl)dimethylsilane (0.457 mL, 2.14 mmol) was added thereto, and the mixture was warmed slowly and stirred at room temperature for 3 hours. After completion of the reaction, the reaction solution was extracted with ethyl acetate and a saturated aqueous solution of sodium hydrogen carbonate, and the organic layer was dried with anhydrous magnesium sulfate and filtered. The filtrate was concentrated under reduced pressure, and the residue was purified by column chromatography (SiO₂; ethyl acetate/hexane=1/3) to afford the title compound (0.490 g, 63%) as a yellow liquid.

### Step 3: (Formula 15) Methyl 6-((1-(2-hydroxyethyl)-2-methyl-1H-indol-3-yl)methyl)nicotinate

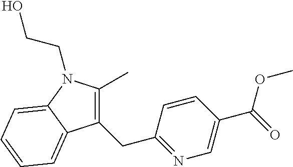 [SMILES: COC(=O)c1ccc(Cc2c(C)n(CCO)c3ccccc23)nc1]

Methyl 6-((1-(2-(tert-butyldimethylsilyloxy)ethyl)-2-methyl-1H-indol-3-yl)methyl)nicotinate (0.490 g, 1.12 mmol) was dissolved in tetrahydrofuran (10 mL), and tetrabutylammonium fluoride solution (1.0 M, dissolved in tetrahydrofuran, 3.351 mL, 3.35 mmol) was added thereto, followed by stirring at room temperature for 1 hour. After completion of the reaction, the reaction solution was extracted with ethyl acetate and a saturated aqueous solution of sodium hydrogen carbonate, and the organic layer was dried with anhydrous magnesium sulfate and filtered. The filtrate was concentrated under reduced pressure, and then dried in a vacuum to afford the title compound of formula 15 (0.360 g, 99%) as a yellow solid.

### Step 4: (Formula 16) Methyl 6-((2-methyl-1-(2-(methylsulfonyloxy)ethyl)-1H-indol-3-yl)methyl)nicotinate

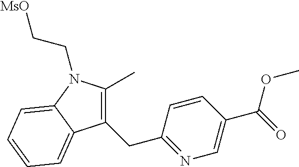 [SMILES: COC(=O)c1ccc(Cc2c(C)n(CCOS(C)(=O)=O)c3ccccc23)nc1]

Methyl 6-((1-(2-hydroxyethyl)-2-methyl-1H-indol-3-yl)methyl)nicotinate (0.360 g, 1.11 mmol) was dissolved in methylene chloride (10 mL) and cooled to 0° C., and triethylamine (0.231 mL, 1.67 mmol) and methanesulfonyl chloride (0.129 mL, 1.67 mmol) were sequentially added thereto, followed by stirring at the same temperature for 1 hour. After completion of the reaction, the reaction solution was extracted with ethyl acetate and a saturated aqueous solution of sodium hydrogen carbonate, and the organic layer was dried with anhydrous magnesium sulfate and filtered. The filtrate was concentrated under reduced pressure, and then dried in a vacuum to afford the title compound (0.350 g, 78%) as a yellow liquid.

### Step 5: (Formula 17) Methyl 6-((2-methyl-1-(2-(4-methylpiperazin-1-yl)ethyl)-1H-indol-3-yl)methyl)nicotinate

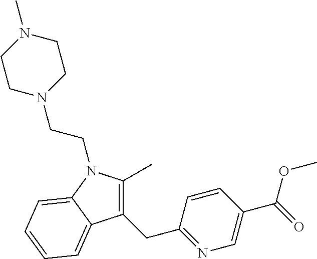 [SMILES: COC(=O)c1ccc(Cc2c(C)n(CCN3CCN(C)CC3)c3ccccc23)nc1]

Methyl 6-((2-methyl-1-(2-(methylsulfonyloxy)ethyl)-1H-indol-3-yl)methyl)nicotinate (0.170 g, 0.42 mmol) was dissolved in acetonitrile (3 mL), and 1-methylpiperazine (0.141 mL, 1.27 mmol) and diisopropylethylamine (0.221 mL, 1.27 mmol) were added thereto. The mixture was allowed to react in a microwave reactor at 120° C. for 3 hours. Then, the reaction solution was concentrated under reduced pressure, and the residue was purified by column chromatography (SiO₂; methylene chloride/methanol=10/1) to afford the title compound (0.076 g, 44%) as a yellow liquid.

### Step 6: (Compound 243) N-hydroxy-6-((2-methyl-1-(2-(4-methylpiperazin-1-yl)ethyl)-1H-indol-3-yl)methyl)nicotinamide

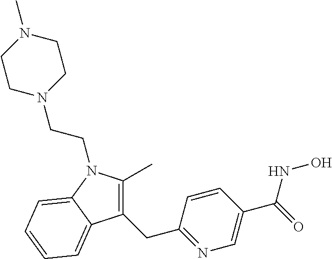 [SMILES: Cc1c(Cc2ccc(C(=O)NO)cn2)c2ccccc2n1CCN1CCN(C)CC1]

Methyl 6-((2-methyl-1-(2-(4-methylpiperazin-1-yl)ethyl)-1H-indol-3-yl)methyl)nicotinate (0.076 g, 0.19 mmol) was dissolved in methanol (5 mL), and hydroxylamine (50 wt % aqueous solution, 3.431 mL, 56.09 mmol), hydroxylamine hydrochloride (0.065 g, 0.94 mmol) and potassium hydroxide (0.210 g, 3.74 mmol) were sequentially added thereto, followed by stirring at room temperature for 1 hour. After completion of the reaction, the reaction solution was concentrated under reduced pressure to a volume of about 2-3 mL, and then extracted with a saturated aqueous solution of sodium hydrogen carbonate and ethyl acetate/tetrahydrofuran, and the organic layer was dried with anhydrous magnesium sulfate and filtered. The filtrate was concentrated under reduced pressure, and then dried in a vacuum to afford compound 243 (0.042 g, 55%) as a light brown solid.

¹H NMR (400 MHz, DMSO-d₆) δ 8.78 (d, 1H, J=1.0 Hz), 7.91 (dd, 1H, J=6.1, 1.5 Hz), 7.39 (d, 1H, J=5.8 Hz), 7.33 (d, 1H, J=6.0 Hz), 7.16 (d, 1H, J=6.1 Hz), 7.02 (t, 1H, J=5.6 Hz), 6.91 (t, 1H, J=5.4 Hz), 4.19 (s, 4H), 2.66-2.59 (m, 2H), 2.49-2.18 (m, 11H), 2.12 (s, 3H); MS (ESI) m/z 408.2 (M⁺+H).

### Example 11

### Synthesis of Compound 244

### Step 1: (Formula 17) Methyl 6-((2-methyl-1-(2-morpholinoethyl)-1H-indol-3-yl)methyl)nicotinate

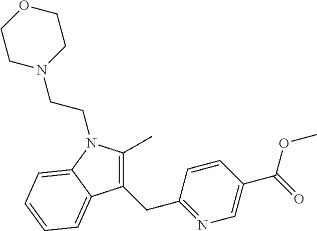 [SMILES: COC(=O)c1ccc(Cc2c(C)n(CCN3CCOCC3)c3ccccc23)nc1]

Methyl 6-((2-methyl-1-(2-(methylsulfonyloxy)ethyl)-1H-indol-3-yl)methyl)nicotinate (0.170 g, 0.42 mmol) was dissolved in acetonitrile (3 mL), and morpholine (0.110 mL, 1.27 mmol) and diisopropylethylamine (0.221 mL, 1.27 mmol) were added thereto. The mixture was allowed to react in a microwave reactor at 120° C. for 3 hours. Then, the reaction solution was concentrated under reduced pressure, and the residue was purified by column chromatography (SiO₂; methylene chloride/methanol=10/1) to afford the title compound (0.082 g, 49%) as a yellow liquid.

### Step 2: (Compound 244) N-hydroxy-6-((2-methyl-1-(2-morpholinoethyl)-1H-indol-3-yl)methyl)nicotinamide)

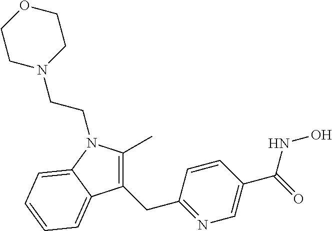 [SMILES: Cc1c(Cc2ccc(C(=O)NO)cn2)c2ccccc2n1CCN1CCOCC1]

Methyl 6-((2-methyl-1-(2-morpholinoethyl)-1H-indol-3-yl)methyl)nicotinate (0.082 g, 0.21 mmol) was dissolved in methanol (5 mL), and hydroxylamine (50 wt % aqueous solution, 3.824 mL, 62.52 mmol), hydroxylamine hydrochloride (0.072 g, 1.04 mmol) and potassium hydroxide (0.234 g, 4.17 mmol) were sequentially added thereto, followed by stirring at room temperature for 1 hour. After completion of the reaction, the reaction solution was concentrated under reduced pressure to a volume of about 2-3 mL, and then extracted with a saturated aqueous solution of sodium hydrogen carbonate and ethyl acetate/tetrahydrofuran, and the organic layer was dried with anhydrous magnesium sulfate and filtered. The filtrate was concentrated under reduced pressure, and then dried in a vacuum to afford compound 244 (0.027 g, 33%) as a white solid.

¹H NMR (400 MHz, DMSO-d₆) δ 11.28 (brs, 1H), 9.18 (brs, 1H), 8.77 (d, 1H, J=1.3 Hz), 7.91 (dd, 1H, J=6.1, 1.7 Hz), 7.39 (d, 1H, J=5.8 Hz), 7.34 (d, 1H, J=6.2 Hz), 7.17 (d, 1H, J=6.2 Hz), 7.03 (t, 1H, J=5.6 Hz), 6.91 (t, 1H, J=5.4 Hz), 4.23-4.19 (m, 4H), 3.54-3.52 (m, 4H), 2.54-2.52 (m, 2H), 2.44 (s, 3H), 2.41-2.39 (m, 4H); MS (ESI) m/z 395.2 (M⁺+H).

### Example 12

### Synthesis of Compound 528

### Step 1: (Formula 13) Tert-butyl 4-(hydroxymethyl)piperidine-1-carboxylate

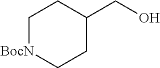 [SMILES: CC(C)(C)OC(=O)N1CCC(CO)CC1]

Piperidin-4-ylmethanol (30.000 g, 260.49 mmol) was dissolved in ethyl acetate (300 mL), and at 0° C., triethylamine (72.215 mL, 520.97 mmol) was added thereto, and then di-tert-butyl dicarbonate (62.536 g, 286.53 mmol) was slowly added thereto, followed by stirring at the same temperature for 30 minutes. Then, a saturated aqueous solution of ammonium chloride was added to the reaction mixture, followed by extraction with ethyl acetate. The organic layer was washed with a saturated aqueous solution of sodium hydrogen carbonate, dried with anhydrous magnesium sulfate, and then concentrated under reduced pressure. The product was used without additional purification (formula 13, 55.500 g, 99%, colorless liquid).

### Step 2: (Formula 3) Tert-butyl 4-((methylsulfonyloxy)methyl)piperidine-1-carboxylate

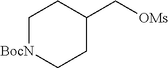 [SMILES: CC(C)(C)OC(=O)N1CCC(COS(C)(=O)=O)CC1]

Tert-butyl 4-(hydroxymethyl)piperidine-1-carboxylate (55.500 g, 257.79 mmol) was dissolved in methylene chloride (300 mL), and at 0° C., triethylamine (53.601 mL, 386.69 mmol) was added thereto, and then methanesulfonyl chloride (23.943 mL, 309.35 mmol) was slowly added thereto. Then, the mixture was warmed slowly to room temperature and stirred for 16 hours. Then, a saturated aqueous solution of ammonium chloride was added to the reaction mixture, followed by extraction with ethyl acetate. The organic layer was washed with a saturated aqueous solution of sodium hydrogen carbonate, dried with anhydrous magnesium sulfate, and then concentrated under reduced pressure. The concentrate was filtered through a silica gel pad using methylene chloride, and then concentrated under reduced pressure to afford the title compound (68.500 g, 91%) as a light yellow solid.

### Step 3: (Formula 2) Methyl 4-((2-methyl-1H-indol-3-yl)methyl)benzoate

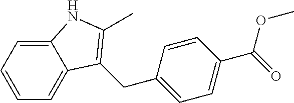 [SMILES: COC(=O)c1ccc(Cc2c(C)[nH]c3ccccc23)cc1]

2-methyl-1H-indole (4.000 g, 17.46 mmol) and methyl 4-(bromomethyl)benzoate (2.749 g, 20.95 mmol) were added to water (40 mL) and heated by microwave irradiation at 150° C. for 5 minutes, followed by cooling to room temperature. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 10% to 30%) and concentrated to afford the title compound (3.090 g, 63%) as a light brown solid.

### Step 4: (Formula 4) Tert-butyl 4-((3-(4-(methoxycarbonyl)benzyl)-2-methyl-1H-indol-1-yl)methyl)piperidine-1-carboxylate

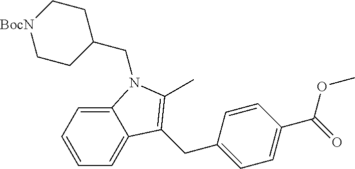 [SMILES: COC(=O)c1ccc(Cc2c(C)n(CC3CCN(C(=O)OC(C)(C)C)CC3)c3ccccc23)cc1]

Methyl 4-((2-methyl-1H-indol-3-yl)methyl)benzoate (1.000 g, 3.58 mmol) was dissolved in N,N-dimethylformamide (20 mL), and at 0° C., sodium hydride (0.112 g, 4.65 mmol) was slowly added dropwise thereto, followed by stirring for 5 minutes. Then, tert-butyl 4-((methylsulfonyloxy)methyl)piperidine-1-carboxylate (1.260 g, 4.30 mmol) and potassium iodide (0.713 g, 4.30 mmol) were added to the solution, followed by stirring at 60° C. for 2 hours. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 10% to 30%) and concentrated to afford the title compound (0.913 g, 54%) as a light yellow solid.

### Step 5: (Formula 5) Methyl 4-((2-methyl-1-(piperidin-4-ylmethyl)-1H-indol-3-yl)methyl)benzoate hydrochloride

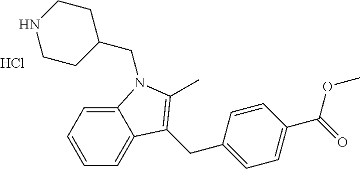 [SMILES: Cl ; COC(=O)c1ccc(Cc2c(C)n(CC3CCNCC3)c3ccccc23)cc1]

Tert-butyl 4-((3-(4-(methoxycarbonyl)benzyl)-2-methyl-1H-indol-1-yl)methyl)piperidine-1-carboxylate (0.913 g, 1.92 mmol) was dissolved in 1,4-dioxane (10 mL), and at room temperature, hydrochloric acid (4.0 M, 1,4-dioxane solution, 9.578 mL, 38.31 mmol) was added thereto, followed by stirring at the same temperature for 1 hour. Then, the reaction mixture was concentrated under reduced pressure. The concentrate was added to diethyl ether (50 mL) and stirred, and the precipitated solid was filtered and dried to afford the title compound (0.750 g, 95%) as a light pink solid.

### Step 6: (Formula 6) Methyl 4-((1-((1-(2-hydroxy-2-methylpropyl)piperidin-4-yl)methyl)-2-methyl-1H-indol-3-yl)methyl)benzoate

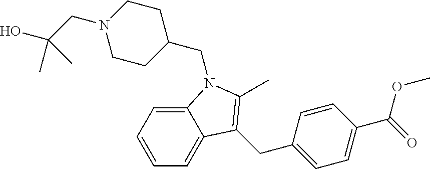 [SMILES: COC(=O)c1ccc(Cc2c(C)n(CC3CCN(CC(C)(C)O)CC3)c3ccccc23)cc1]

Methyl 4-((2-methyl-1-(piperidin-4-ylmethyl)-1H-indol-3-yl)methyl)benzoate hydrochloride (0.750 g, 1.82 mmol), 2,2-dimethyloxirane (1.310 g, 18.16 mmol) and potassium carbonate (2.510 g, 18.16 mmol) were added to ethanol (18 mL) and heated by microwave irradiation at 110° C. for 20 minutes, followed by cooling to room temperature. Then, the reaction mixture was concentrated under reduced pressure to remove the solvent. The concentrate was added to water, followed by extraction with ethyl acetate. The organic layer was washed with a saturated aqueous solution of sodium chloride, dried with anhydrous magnesium sulfate, and then concentrated under reduced pressure. The product was used without additional purification (formula 6, 0.750 g, 92%, light brown solid).

### Step 7: (Formula 8) Methyl 4-((1-((1-(2-fluoro-2-methylpropyl)piperidin-4-yl)methyl)-2-methyl-1H-indol-3-yl)methyl)benzoate

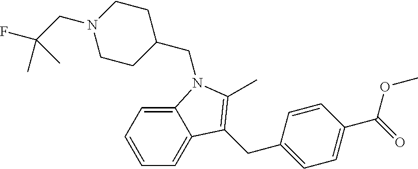 [SMILES: COC(=O)c1ccc(Cc2c(C)n(CC3CCN(CC(C)(C)F)CC3)c3ccccc23)cc1]

Methyl 4-((1-((1-(2-hydroxy-2-methylpropyl)piperidin-4-yl)methyl)-2-methyl-1H-indol-3-yl)methyl)benzoate (0.750 g, 1.67 mmol) was dissolved in methylene chloride (20 mL), and at room temperature, diethylaminosulfur trifluoride (0.218 mL, 1.84 mmol) was added thereto, followed by stirring at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 30% to 60%) and concentrated to afford the title compound (0.510 g, 68%) as a yellow solid.

### Step 8: (Compound 528) 4-((1-((1-(2-fluoro-2-methylpropyl)piperidin-4-yl)methyl)-2-methyl-1H-indol-3-yl)methyl)-N-hydroxybenzamide

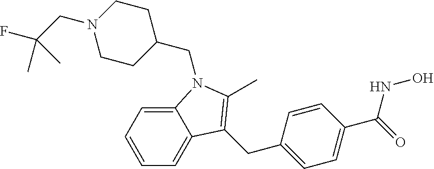 [SMILES: Cc1c(Cc2ccc(C(=O)NO)cc2)c2ccccc2n1CC1CCN(CC(C)(C)F)CC1]

Methyl 4-((1-((1-(2-fluoro-2-methylpropyl)piperidin-4-yl)methyl)-2-methyl-1H-indol-3-yl)methyl)benzoate (0.510 g, 1.132 mmol) was dissolved in methanol (10 mL)/tetrahydrofuran (3 mL), and at room temperature, hydroxylamine (50 wt % aqueous solution, 3.462 mL, 56.59 mmol) was added thereto, and then potassium hydroxide (0.635 g, 11.32 mmol) was added thereto, followed by stirring at the same temperature for 30 minutes. Next, the reaction mixture was concentrated under reduced pressure to a volume of about 5 mL, and a saturated aqueous solution of sodium hydrogen carbonate was added to the concentrate. The precipitated solid was filtered, and then dried to afford compound 528 (0.470 g, 92%) as a light yellow solid.

¹H NMR (400 MHz, DMSO-d₆) δ 7.59 (d, 2H, J=7.9 Hz), 7.38-7.32 (m, 2H), 7.19 (d, 2H, J=8.0 Hz), 7.01 (t, 1H, J=7.6 Hz), 6.89 (t, 1H, J=7.5 Hz), 4.03 (s, 2H), 3.99 (d, 2H, J=7.3 Hz), 2.85-2.82 (m, 2H), 2.38-2.32 (m, 6H), 1.97-1.92 (m, 2H), 1.72 (m, 1H), 1.37-1.34 (m, 3H), 1.29 (s, 3H), 1.24 (s, 3H); MS (ESI) m/z 452.3 (M⁺+H).

### Example 13

### Synthesis of Compound 550

### Step 1: (Formula 17) Methyl 4-((1-(2-(3-hydroxypiperidin-1-yl)ethyl)-2-methyl-1H-indol-3-yl)methyl)benzoate

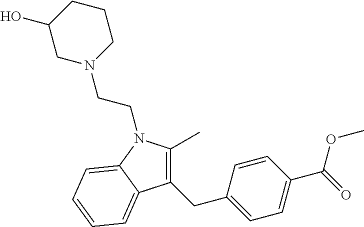 [SMILES: COC(=O)c1ccc(Cc2c(C)n(CCN3CCCC(O)C3)c3ccccc23)cc1]

Methyl 4-((2-methyl-1-(2-(methylsulfonyloxy)ethyl)-1H-indol-3-yl)methyl)benzoate (0.300 g, 0.75 mmol), piperidin-3-ol hydrochloride (0.514 g, 3.74 mmol) and diisopropylethylamine (1.323 mL, 7.47 mmol) were added to acetonitrile (3 mL) and heated by microwave irradiation at 120° C. for 1 hour, followed by cooling to room temperature. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; methanol/methylene chloride=from 0% to 20%) and concentrated to afford the title compound (0.047 g, 16%) as a yellow liquid.

### Step 2: (Formula 19) Methyl 4-((1-(2-(3-fluoropiperidin-1-yl)ethyl)-2-methyl-1H-indol-3-yl)methyl)benzoate

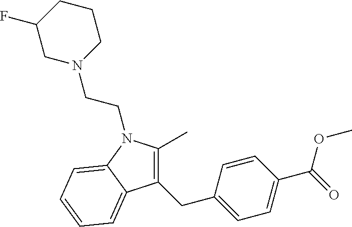 [SMILES: COC(=O)c1ccc(Cc2c(C)n(CCN3CCCC(F)C3)c3ccccc23)cc1]

Methyl 4-((1-(2-(3-hydroxypiperidin-1-yl)ethyl)-2-methyl-1H-indol-3-yl)methyl)benzoate (0.047 g, 0.12 mmol) was dissolved in methylene chloride (5 mL), and at room temperature, diethylaminosulfur trifluoride (0.016 mL, 0.14 mmol) was added thereto, followed by stirring at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 30% to 60%) and concentrated to afford the title compound (0.014 g, 30%) as a light yellow liquid.

### Step 3: (Compound 550) 4-((1-(2-(3-Fluoropiperidin-1-yl)ethyl)-2-methyl-1H-indol-3-yl)methyl)-N-hydroxybenzamide

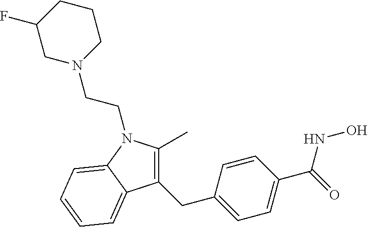 [SMILES: Cc1c(Cc2ccc(C(=O)NO)cc2)c2ccccc2n1CCN1CCCC(F)C1]

Methyl 4-((1-(2-(3-fluoropiperidin-1-yl)ethyl)-2-methyl-1H-indol-3-yl)methyl)benzoate (0.014 g, 0.03 mmol) was dissolved in methanol (10 mL). At room temperature, hydroxylamine (50 wt % aqueous solution, 0.629 mL, 10.28 mmol) was added to the solution, and then potassium hydroxide (0.019 g, 0.34 mmol) was added thereto, followed by stirring at the same temperature for 1 hour. Then, the reaction mixture was concentrated under reduced pressure to remove the solvent, and a saturated aqueous solution of sodium hydrogen carbonate was added to the concentrate and stirred. The precipitated solid was filtered, washed with water, and then dried to afford compound 550 (0.012 g, 86%) as a light brown solid.

¹H NMR (400 MHz, DMSO-d₆) δ 7.59 (d, 2H, J=8.2 Hz), 7.38-7.33 (m, 2H), 7.19 (d, 2H, J=7.8 Hz), 7.03 (t, 1H, J=7.5 Hz), 6.92 (t, 1H, J=7.2 Hz), 4.60 (m, 1H), 4.28-4.20 (m, 2H), 4.03 (s, 2H), 2.80 (m, 1H), 2.60-2.55 (m, 2 H), 2.46-2.42 (m, 5H), 2.31 (m, 1H), 1.86-1.63 (m, 2H), 1.50-1.42 (m, 2H); MS (ESI) m/z 410.2 (M⁺+H).

### Example 14

### Synthesis of Compound 551

### Step 1: (Formula 17) (S)-methyl 4-((1-(2-(2-(hydroxymethyl)pyrrolidin-1-yl)ethyl)-2-methyl-1H-indol-3-yl)methyl)benzoate

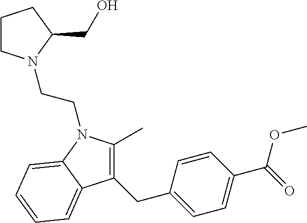 [SMILES: COC(=O)c1ccc(Cc2c(C)n(CCN3CCC[C@H]3CO)c3ccccc23)cc1]

Methyl 4-((2-methyl-1-(2-(methylsulfonyloxy)ethyl)-1H-indol-3-yl)methyl)benzoate (0.300 g, 0.75 mmol), (5)-pyrrolidin-2-ylmethanol (0.378 g, 3.74 mmol) and diisopropylethylamine (0.662 mL, 3.74 mmol) were added to acetonitrile (3 mL) and heated by microwave irradiation at 120° C. for 1 hour, followed by cooling to room temperature. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; methanol/methylene chloride=from 0% to 20%) and concentrated to afford the title compound (0.151 g, 50%) as a yellow liquid.

### Step 2: (Formula 19) (S)-methyl 4-((1-(2-(2-(fluoromethyl)pyrrolidin-1-yl)ethyl)-2-methyl-1H-indol-3-yl)methyl)benzoate

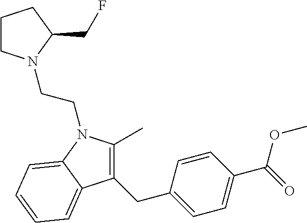 [SMILES: COC(=O)c1ccc(Cc2c(C)n(CCN3CCC[C@H]3CF)c3ccccc23)cc1]

(S)-methyl 4-((1-(2-(2-(hydroxymethyl)pyrrolidin-1-yl)ethyl)-2-methyl-1H-indol-3-yl)methyl)benzoate (0.151 g, 0.37 mmol) was dissolved in methylene chloride (5 mL). At room temperature, diethylaminosulfur trifluoride (0.053 mL, 0.45 mmol) was added to the solution, followed by stirring at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 30% to 60%) and concentrated to afford the title compound (0.115 g, 76%) as a light yellow liquid.

### Step 3: (Compound 551) (S)-4-((1-(2-(2-(fluoromethyl)pyrrolidin-1-yl)ethyl)-2-methyl-1H-indol-3-yl)methyl)-N-hydroxybenzamide

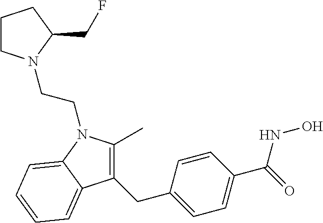 [SMILES: Cc1c(Cc2ccc(C(=O)NO)cc2)c2ccccc2n1CCN1CCC[C@H]1CF]

(S)-methyl 4-((1-(2-(2-(fluoromethyl)pyrrolidin-1-yl)ethyl)-2-methyl-1H-indol-3-yl)methyl)benzoate (0.115 g, 0.28 mmol) was dissolved in methanol (10 mL). At room temperature, hydroxylamine (50 wt % aqueous solution, 2.583 mL, 42.23 mmol) was added to the solution, and then potassium hydroxide (0.158 g, 2.82 mmol) was added thereto, followed by stirring at the same temperature for 1 hour. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 551 (0.100 g, 87%) as a white solid.

¹H NMR (400 MHz, DMSO-d₆) δ 7.55 (d, 2H, J=8.0 Hz), 7.32-7.29 (m, 2H), 7.17 (d, 2H, J=8.0 Hz), 6.99 (t, 1H, J=7.6 Hz), 6.88 (t, 1H, J=7.8 Hz), 4.54 (m, 1H), 4.20-4.12 (m, 2H), 4.03 (s, 2H), 2.63 (m, 1H), 2.56-2.52 (m, 2H), 2.42-2.38 (m, 5H), 2.25 (m, 1H), 1.78-1.63 (m, 2H), 1.46-1.36 (m, 2H); MS (ESI) m/z 410.2 (M⁺+H).

### Example 15

### Synthesis of Compound 553

### Step 1: (Formula 17) methyl 4-((1-(2-(4-hydroxypiperidin-1-yl)ethyl)-2-methyl-1H-indol-3-yl)methyl)benzoate

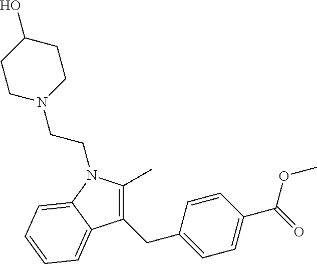 [SMILES: COC(=O)c1ccc(Cc2c(C)n(CCN3CCC(O)CC3)c3ccccc23)cc1]

Methyl 4-((2-methyl-1-(2-(methylsulfonyloxy)ethyl)-1H-indol-3-yl)methyl)benzoate (0.300 g, 0.75 mmol), piperidin-4-ol (0.378 g, 3.74 mmol) and diisopropylethylamine (0.662 mL, 3.74 mmol) were added to acetonitrile (3 mL) and heated by microwave irradiation at 120° C. for 1 hour, followed by cooling to room temperature. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; methanol/methylene chloride=from 0% to 20%) and concentrated to afford the title compound (0.258 g, 85%) as a yellow solid.

### Step 2: (Formula 19) Methyl 4-((1-(2-(4-fluoropiperidin-1-yl)ethyl)-2-methyl-1H-indol-3-yl)methyl)benzoate

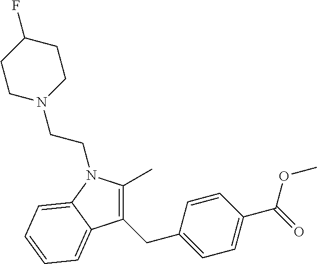 [SMILES: COC(=O)c1ccc(Cc2c(C)n(CCN3CCC(F)CC3)c3ccccc23)cc1]

Methyl 4-((1-(2-(4-hydroxypiperidin-1-yl)ethyl)-2-methyl-1H-indol-3-yl)methyl)benzoate (0.258 g, 0.64 mmol) was dissolved in methylene chloride (5 mL). At room temperature, diethylaminosulfur trifluoride (0.090 mL, 0.76 mmol) was added to the solution, followed by stirring at the same temperature for 2 hour. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 50% to 80%) and concentrated to afford the title compound (0.176 g, 68%) as a light yellow liquid.

### Step 3: (Compound 553) 4-((1-(2-(4-Fluoropiperidin-1-yl)ethyl)-2-methyl-1H-indol-3-yl)methyl)-N-hydroxybenzamide

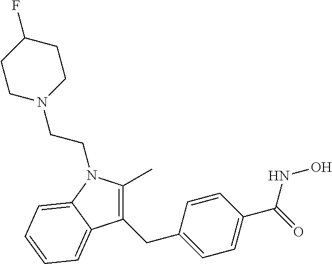 [SMILES: Cc1c(Cc2ccc(C(=O)NO)cc2)c2ccccc2n1CCN1CCC(F)CC1]

Methyl 4-((1-(2-(4-fluoropiperidin-1-yl)ethyl)-2-methyl-1H-indol-3-yl)methyl)benzoate (0.176 g, 0.43 mmol) was added in methanol (10 mL). At room temperature, hydroxylamine (50 wt % aqueous solution, 2.635 mL, 43.08 mmol) was added to the solution, and then potassium hydroxide (0.242 g, 4.31 mmol) was added thereto, followed by stirring at the same temperature for 1 hour. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 553 (0.155 g, 88%) as a white solid.

¹H NMR (400 MHz, DMSO-d₆) δ 7.56 (d, 2H, J=8.4 Hz), 7.31 (d, 2H, J=8.0 Hz), 7.19 (d, 2H, J=8.0 Hz), 7.00 (t, 1H, J=7.6 Hz), 6.88 (t, 1H, J=7.2 Hz), 4.62 (m, 1H), 4.17 (t, 2H, J=6.8 Hz), 4.01 (s, 2H), 2.52-2.50 (m, 4H), 2.39 (s, 3H), 2.32-2.28 (m, 2H), 1.82-1.73 (m, 2H), 1.68-1.63 (m, 2H); MS (ESI) m/z 410.2 (M⁺+H).

### Example 16

### Synthesis of Compound 556

### Step 1: (Formula 31) Methyl 4-((1-((1-benzylpiperidin-4-yl)methyl)-2-methyl-1H-indol-3-yl)methyl)benzoate

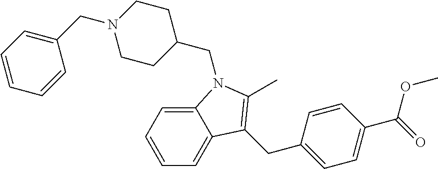 [SMILES: COC(=O)c1ccc(Cc2c(C)n(CC3CCN(Cc4ccccc4)CC3)c3ccccc23)cc1]

Methyl 4-((2-methyl-1-(piperidin-4-ylmethyl)-1H-indol-3-yl)methyl)benzoate hydrochloride (0.100 g, 0.24 mmol), benzyl bromide (0.035 mL, 0.29 mmol) and diisopropylethylamine (0.125 mL, 0.73 mmol) were dissolved in methylene chloride (5 mL) at room temperature. The solution was stirred at the same temperature for 2 hours. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=10% to 50%) and concentrated to afford the title compound (0.100 g, 88%) as a yellow liquid.

### Step 2: (Compound 556) 4-((1-((1-Benzylpiperidin-4-yl)methyl)-2-methyl-1H-indol-3-yl)methyl)-N-hydroxybenzamide

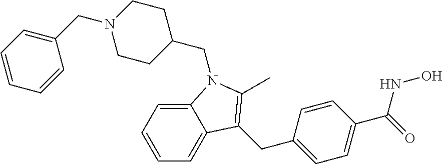 [SMILES: Cc1c(Cc2ccc(C(=O)NO)cc2)c2ccccc2n1CC1CCN(Cc2ccccc2)CC1]

Methyl 4-((1-((1-benzylpiperidin-4-yl)methyl)-2-methyl-1H-indol-3-yl)methyl)benzoate (0.100 g, 0.21 mmol), hydroxylamine (50 wt % aqueous solution, 0.918 mL, 15.00 mmol) and potassium hydroxide (0.120 g, 2.14 mmol) were dissolved in methanol (3 mL) at room temperature, and the solution was stirred at the same temperature for 0.5 hours. Then, the reaction mixture was concentrated under reduced pressure to remove the solvent, and a saturated aqueous solution of sodium hydrogen carbonate was added to the concentrate. The precipitated solid was filtered, and then dried to afford desired compound 556 (0.088 g, 88%) as a light yellow solid.

¹H-NMR (400 MHz, DMSO-d₆) δ 9.65 (brs, 1H), 7.58 (d, 2H, J=8.1 Hz), 7.39-7.21 (m, 7H), 7.12 (d, 2H, J=8.1 Hz), 7.01 (t, 1H, J=7.1 Hz), 6.90 (t, 1H, J=7.4 Hz), 4.02 (s, 2H), 4.00 (s, 2H), 2.69 (d, 2H, J=11.3 Hz), 2.37 (s, 3H), 1.81 (t, 2H, J=10.8 Hz), 1.76-1.71 (m, 1H), 1.43 (d, 2H, J=10.1 Hz), 1.34 (t, 2H, J=10.6 Hz), 1.29-1.24 (m, 2H); MS (ESI) m/z 468.3 (M⁺+1).

### Example 17

### Synthesis of Compound 558

### Step 1: (Formula 31) Methyl 4-((1-((1-butylpiperidin-4-yl)methyl)-2-methyl-1H-indol-3-yl)methyl)benzoate

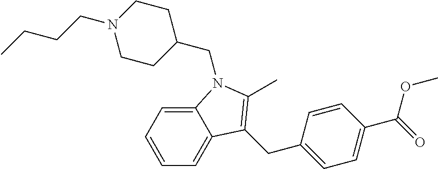 [SMILES: CCCCN1CCC(Cn2c(C)c(Cc3ccc(C(=O)OC)cc3)c3ccccc32)CC1]

Methyl 4-((2-methyl-1-(piperidin-4-ylmethyl)-1H-indol-3-yl)methyl)benzoate hydrochloride (0.150 g, 0.363 mmol) was dissolved in methanol (10 mL). At room temperature, butyl aldehyde (0.039 mL, 0.44 mmol) and acetic acid (0.031 mL, 0.55 mmol) were added to the solution, followed by stirring for 1 hour. Sodium cyanoborohydride (0.027 g, 0.44 mmol) was added to the reaction solution, followed by stirring at the same temperature for 1 hour. Then, the reaction mixture was concentrated under reduced pressure to remove the solvent, and a saturated aqueous solution of sodium hydrogen carbonate was added to the concentrate, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 10%) and concentrated to afford the title compound (0.069 g, 44%) as a yellow liquid.

### Step 2: (Compound 558) 4-((1-((1-Butylpiperidin-4-yl)methyl)-2-methyl-1H-indol-3-yl)methyl)-N-hydroxybenzamide

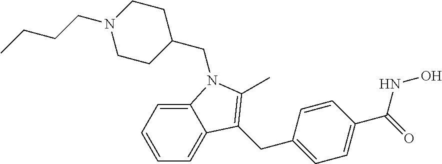 [SMILES: CCCCN1CCC(Cn2c(C)c(Cc3ccc(C(=O)NO)cc3)c3ccccc32)CC1]

Methyl 4-((1-((1-butylpiperidin-4-yl)methyl)-2-methyl-1H-indol-3-yl)methyl)benzoate (0.069 g, 0.16 mmol), hydroxylamine (50 wt % aqueous solution, 0.683 mL, 11.17 mmol) and potassium hydroxide (0.089 g, 1.60 mmol) were dissolved in methanol (2 mL) at room temperature, and the solution was stirred at the same temperature for 0.5 hours. Then, the reaction mixture was concentrated under reduced pressure to remove the solvent, and a saturated aqueous solution of sodium hydrogen carbonate was added to the concentrate. The precipitated solid was filtered, and then dried to afford desired compound 558 (0.017 g, 24%) as an ivory solid.

¹H-NMR (400 MHz, DMSO-d₆) δ 7.57 (d, 2H, J=8.2 Hz), 7.36 (t, 2H, J=7.3 Hz), 7.10 (d, 2H, J=8.0 Hz), 7.02 (t, 1H, J=7.8 Hz), 6.90 (t, 1H, J=7.5 Hz), 4.00 (s, 2H), 3.99 (s, 2H), 2.81 (d, 2H, J=10.5 Hz), 2.39 (s, 3H), 2.19 (t, 2H, J=7.3 Hz), 1.74-1.69 (m, 3H), 1.43-1.22 (m, 8H), 0.86 (t, 3H, J=7.3 Hz); MS (ESI) m/z 434.3 (M⁺+1).

### Example 18

### Synthesis of Compound 559

### Step 1: (Formula 31) Methyl 4-((2-methyl-1-((1-phenethylpiperidin-4-yl)methyl)-1H-indol-3-yl)methyl)benzoate

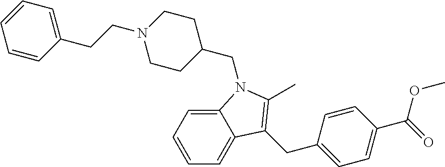 [SMILES: COC(=O)c1ccc(Cc2c(C)n(CC3CCN(CCc4ccccc4)CC3)c3ccccc23)cc1]

Methyl 4-((2-methyl-1-(piperidin-4-ylmethyl)-1H-indol-3-yl)methyl)benzoate hydrochloride (0.150 g, 0.36 mmol) was dissolved in methanol (10 mL). At room temperature, 2-phenylacetaldehyde (0.051 mL, 0.44 mmol) and acetic acid (0.031 mL, 0.55 mmol) were added to the solution and stirred for 1 hour. Then, sodium cyanoborohydride (0.027 g, 0.44 mmol) was added to the reaction solution, followed by stirring at the same temperature for 1 hour. Then, the reaction mixture was concentrated under reduced pressure to remove the solvent, and a saturated aqueous solution of sodium hydrogen carbonate was added to the concentrate, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 10%) and concentrated to afford the title compound (0.044 g, 25%) as a yellow liquid.

### Step 2: (Compound 559) N-hydroxy-4-((2-methyl-1-((1-phenethylpiperidin-4-yl)methyl)-1H-indol-3-yl)methyl)benzamide

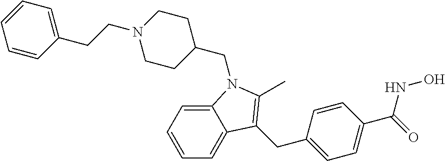 [SMILES: Cc1c(Cc2ccc(C(=O)NO)cc2)c2ccccc2n1CC1CCN(CCc2ccccc2)CC1]

Methyl 4-((2-methyl-1-((1-phenethylpiperidin-4-yl)methyl)-1H-indol-3-yl)methyl)benzoate (0.044 g, 0.09 mmol), hydroxylamine (50 wt % aqueous solution, 0.392 mL, 6.41 mmol) and potassium hydroxide (0.051 g, 0.92 mmol) were dissolved in methanol (2 mL) at room temperature, and the solution was stirred at the same temperature for 0.5 hours. Then, the reaction mixture was concentrated under reduced pressure to remove the solvent, and a saturated aqueous solution of sodium hydrogen carbonate was added to the concentrated. The precipitated solid was filtered, and then dried to afford desired compound 559 (0.031 g, 38%) as an ivory solid.

¹H-NMR (400 MHz, DMSO-d₆) δ 7.57 (d, 2H, J=8.1 Hz), 7.38 (t, 2H, J=7.7 Hz), 7.26 (t, 2H, J=7.4 Hz), 7.20 (d, 2H, J=7.2 Hz), 7.17 (d, 2H, J=7.4 Hz), 7.10 (d, 2H, J=8.1 Hz), 7.02 (t, 1H, J=7.0 Hz), 6.91 (t, 1H, J=7.4 Hz), 4.02 (s, 2H), 4.00 (s, 2H), 2.91 (d, 2H, J=11.6 Hz), 2.69 (t, 2H, J=7.7 Hz), 2.40 (s, 3H), 1.83 (t, 2H, J=10.6 Hz), 1.78-1.72 (m, 1H), 1.44 (d, 2H, J=9.5 Hz), 1.36-1.24 (m, 4H); MS (ESI) m/z 482.3 (M⁺+1).

### Example 19

### Synthesis of Compound 562

### Step 1: (Formula 44) 3-Fluoro-4-((2-methyl-1H-indol-3-yl)methyl)benzonitrile

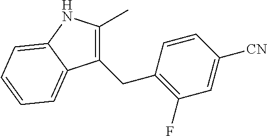 [SMILES: Cc1[nH]c2ccccc2c1Cc1ccc(C#N)cc1F]

2-methylindole (1.000 g, 7.62 mmol) and 4-(bromomethyl)-3-fluorobenzonitrile (1.632 g, 7.62 mmol) were added to water (10 mL) and allowed to react in a microwave reactor at 150° C. for 7 minutes. After completion of the reaction, the reaction solution was extracted with ethyl acetate and a saturated aqueous solution of sodium hydride carbonate, and the organic layer was dried with anhydrous magnesium sulfate, and then filtered. The filtrate was concentrated under reduced pressure, and the filtrate was purified by column chromatography (SiO₂; ethyl acetate/hexane=1/4) to afford the title compound (1.610 g, 80%) as a yellow solid.

### Step 2: (Formula 45) 3-Fluoro-4-((2-methyl-1H-indol-3-yl)methyl)benzoic acid

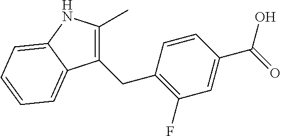 [SMILES: Cc1[nH]c2ccccc2c1Cc1ccc(C(=O)O)cc1F]

3-Fluoro-4-((2-methyl-1H-indol-3-yl)methyl)benzonitrile (1.160 g, 4.39 mmol) and potassium hydroxide (2.463 g, 43.89 mmol) were mixed with water (10 mL)/methanol (10 mL), and the reaction mixture was heated under reflux for 6 hours, and then cooled to room temperature. Then, the reaction mixture was concentrated under reduced pressure to remove the solvent, and a 1 N aqueous solution of hydrochloric acid was added to the concentrate, followed by stirring. The precipitated solid was filtered and dried to afford the title compound (1.190 g, 96%) as a light brown solid.

### Step 3: (Formula 46) Ethyl 3-fluoro-4-((2-methyl-1H-indol-3-yl)methyl)benzoate

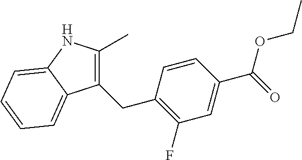 [SMILES: CCOC(=O)c1ccc(Cc2c(C)[nH]c3ccccc23)c(F)c1]

3-Fluoro-4-((2-methyl-1H-indol-3-yl)methyl)benzoic acid (1.190 g, 4.20 mmol), ethanol (2.450 mL, 42.01 mmol) and diisopropylethylamine (2.231 mL, 12.60 mmol) were dissolved in tetrahydrofuran (30 mL). At room temperature, 1-ethyl-3-(3-dimethylaminopropyl)carbodiimide (1.610 g, 8.40 mmol) and 1-hydroxybenzotriazole anhydride (1.135 g, 8.40 mmol) were added to the solution, followed by stirring at the same temperature for 16 hours. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 10% to 30%) and concentrated to afford the title compound (1.210 g, 93%) as a yellow solid.

### Step 4: (Formula 47) Tert-butyl 4-((3-(4-(ethoxycarbonyl)-2-fluorobenzyl)-2-methyl-1H-indol-1-yl)methyl)piperidine-1-carboxylate

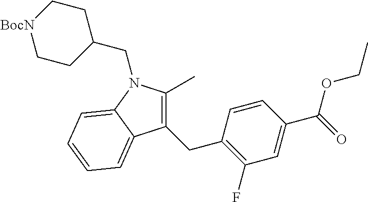 [SMILES: CCOC(=O)c1ccc(Cc2c(C)n(CC3CCN(C(=O)OC(C)(C)C)CC3)c3ccccc23)c(F)c1]

Ethyl 3-fluoro-4-((2-methyl-1H-indol-3-yl)methyl)benzoate (1.200 g, 3.85 mmol) was dissolved in N,N-dimethylformamide (10 mL). At room temperature, sodium hydride (0.120 g, 5.01 mmol) was added to the solution, followed by stirring for 10 minutes. Then, tert-butyl 4-((methylsulfonyloxy)methyl)piperidine-1-carboxylate (1.357 g, 4.63 mmol) and potassium iodide (0.768 g, 4.63 mmol) were added to the reaction solution, followed by stirring at 60° C. for 2 hours. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 10% to 30%) and concentrated to afford the title compound (0.698 g, 36%) as a light brown solid.

### Step 5: (Formula 48) Ethyl 3-fluoro-4-((2-methyl-1-(piperidin-4-ylmethyl)-1H-indol-3-yl)methyl)benzoate hydrochloride

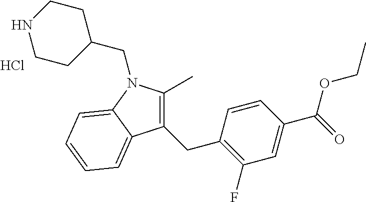 [SMILES: Cl ; CCOC(=O)c1ccc(Cc2c(C)n(CC3CCNCC3)c3ccccc23)c(F)c1]

Tert-butyl 4-((3-(4-(ethoxycarbonyl)-2-fluorobenzyl)-2-methyl-1H-indol-1-yl)methyl)piperidine-1-carboxylate (0.698 g, 1.37 mmol) was dissolved in 1,4-dioxane (5 mL). At room temperature, hydrochloric acid (4.0 M, 1,4-dioxane solution, 5.146 mL, 20.59 mmol) was added to the solution, followed by stirring at the same temperature for 1 hour. Then, the reaction mixture was concentrated under reduced pressure. Methylene chloride (3 mL) and hexane (30 mL) were added to the concentrated, followed by stirring. The precipitated solid was filtered and dried to afford the title compound (0.580 g, 95%) as a light brown solid.

### Step 6: (Formula 49) Ethyl 3-fluoro-4-((1-((1-(2-hydroxy-2-methylpropyl)piperidin-4-yl)methyl)-2-methyl-1H-indol-3-yl)methyl)benzoate

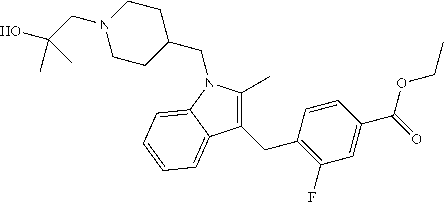 [SMILES: CCOC(=O)c1ccc(Cc2c(C)n(CC3CCN(CC(C)(C)O)CC3)c3ccccc23)c(F)c1]

Ethyl 3-fluoro-4-((2-methyl-1-(piperidin-4-ylmethyl)-1H-indol-3-yl)methyl)benzoate hydrochloride (0.350 g, 0.79 mmol), 2,2-dimethyloxirane (0.567 g, 7.87 mmol) and potassium carbonate (1.087 g, 7.87 mmol) were added to ethanol (10 mL) and heated by microwave irradiation at 110° C. for 20 minutes, followed by cooling to room temperature. Then, the reaction mixture was concentrated under reduced pressure to remove the solvent, and water was added to the concentrate, followed by extraction with ethyl acetate. The organic layer was washed with a saturated aqueous solution of sodium chloride, dried with anhydrous magnesium sulfate, and then concentrated under reduced pressure. The product was used without additional purification (formula 49, 0.374 g, 99%, brown liquid).

### Step 7: (Formula 50) Ethyl 3-fluoro-4-((1-((1-(2-fluoro-2-methylpropyl)piperidin-4-yl)methyl)-2-methyl-1H-indol-3-yl)methyl)benzoate

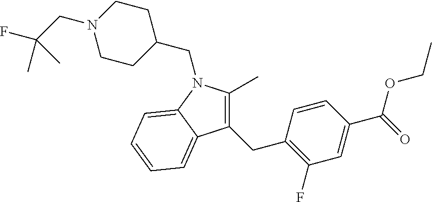 [SMILES: CCOC(=O)c1ccc(Cc2c(C)n(CC3CCN(CC(C)(C)F)CC3)c3ccccc23)c(F)c1]

Ethyl 3-fluoro-4-((1-((1-(2-hydroxy-2-methylpropyl)piperidin-4-yl)methyl)-2-methyl-1H-indol-3-yl)methyl)benzoate (0.374 g, 0.78 mmol) was dissolved in methylene chloride (10 mL). At room temperature, diethylaminosulfur trifluoride (0.111 mL, 0.93 mmol) was added to the solution, followed by stirring at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 30% to 70%) and concentrated to afford the title compound (0.215 g, 57%) as a yellow liquid.

### Step 8: (Compound 562) 3-Fluoro-4-((1-((1-(2-fluoro-2-methylpropyl)piperidin-4-yl)methyl)-2-methyl-1H-indol-3-yl)methyl)-N-hydroxybenzamide

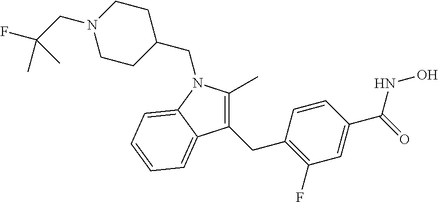 [SMILES: Cc1c(Cc2ccc(C(=O)NO)cc2F)c2ccccc2n1CC1CCN(CC(C)(C)F)CC1]

Ethyl 3-fluoro-4-((1-((1-(2-fluoro-2-methylpropyl)piperidin-4-yl)methyl)-2-methyl-1H-indol-3-yl)methyl)benzoate (0.215 g, 0.45 mmol) was dissolved in methanol (10 mL)/tetrahydrofuran (3 mL). At room temperature, hydroxylamine (50% aqueous solution, 2.725 mL, 44.55 mmol) was added to the solution, and then potassium hydroxide (0.250 g, 4.46 mmol) was added thereto, followed by stirring at the same temperature for 3 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 562 (0.201 g, 96%) as a white solid.

¹H NMR (400 MHz, DMSO-d₆) δ 7.44-7.30 (m, 4H), 7.09 (t, 1H, J=7.8 Hz), 6.99 (m, 1H), 6.87 (t, 1H, J=7.2 Hz), 4.00 (s, 2H), 3.97 (d, 2H, J=7.2 Hz), 2.82-2.80 (m, 2H), 2.36-2.30 (m, 5H), 1.94-1.89 (m, 2H), 1.66 (m, 1H), 1.37-1.31 (m, 4H), 1.27 (s, 3H), 1.22 (s, 3H); MS (ESI) m/z 470.3 (M⁺+H).

### Example 20

### Synthesis of Compound 563

### Step 1: (Formula 3) Tert-butyl 4-(2-(methylsulfonyloxy)ethyl)piperidine-1-carboxylate

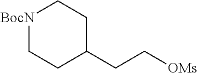 [SMILES: CC(C)(C)OC(=O)N1CCC(CCOS(C)(=O)=O)CC1]

Tert-butyl 4-(2-hydroxyethyl)piperidine-1-carboxylate (0.950 g, 4.14 mmol) was dissolved in methylene chloride (30 mL). At 0° C., methanesulfonyl chloride (0.385 mL, 4.97 mmol) and diisopropylethylamine (1.100 mL, 6.21 mmol) were added to the solution, followed by stirring at room temperature for 1 hour. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The product was used without addition purification (formula 3, 1.160 g, 91%, light brown solid).

### Step 2: (Formula 4) Tert-butyl 4-(2-(3-(4-(methoxycarbonyl)benzyl)-2-methyl-1H-indol-1-yl)ethyl)piperidine-1-carboxylate

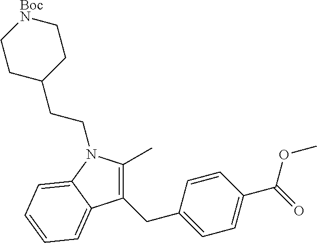 [SMILES: COC(=O)c1ccc(Cc2c(C)n(CCC3CCN(C(=O)OC(C)(C)C)CC3)c3ccccc23)cc1]

Methyl 4-((2-methyl-1H-indole-3-yl)methyl)benzoate (0.850 g, 3.04 mmol) was dissolved in N,N-dimethylformamide (10 mL). At room temperature, sodium hydride (0.095 g, 3.96 mmol) was added to the solution, followed by stirring for 10 minutes. Then, tert-butyl 4-(2-(methylsulfonyloxy)ethyl)piperidine-1-carboxylate (1.123 g, 3.65 mmol) and potassium iodide (0.606 g, 3.65 mmol) were added to the reaction solution, followed by stirring at 60° C. for 2 hours. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 10% to 30%) and concentrated to afford the title compound (0.744 g, 50%) as a light yellow solid.

### Step 3: (Formula 5) Methyl 4-((2-methyl-1-(2-(piperidin-4-yl)ethyl)-1H-indol-3-yl)methyl)benzoate hydrochloride

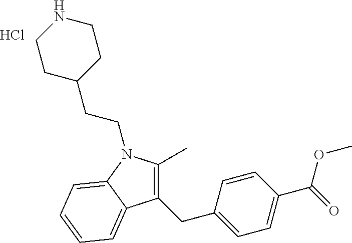 [SMILES: Cl ; COC(=O)c1ccc(Cc2c(C)n(CCC3CCNCC3)c3ccccc23)cc1]

Tert-butyl 4-(2-(3-(4-(methoxycarbonyl)benzyl)-2-methyl-1H-indol-1-yl)ethyl)piperidine-1-carboxylate (0.744 g, 1.52 mmol) was dissolved in 1,4-dioxane (5 mL). At room temperature, hydrochloric acid (4.0 M, 1,4-dioxane solution, 5.69 mL, 22.75 mmol) was added to the solution, followed by stirring at the same temperature for 1 hour. Then, the reaction mixture was concentrated. Methylene chloride (3 mL) and hexane (30 mL) were added to the concentrate, followed by stirring. The precipitated solid was filtered and dried to afford the title compound (0.620 g, 96%) as a light purple solid.

### Step 4: (Formula 6) Methyl 4-((1-(2-(1-(2-hydroxy-2-methylpropyl)piperidin-4-yl)ethyl)-2-methyl-1H-indol-3-yl)methyl)benzoate

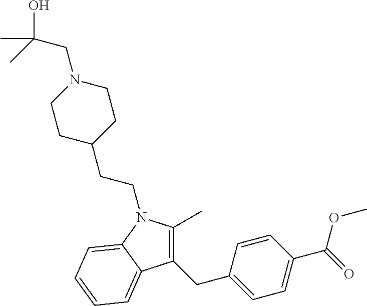 [SMILES: COC(=O)c1ccc(Cc2c(C)n(CCC3CCN(CC(C)(C)O)CC3)c3ccccc23)cc1]

Methyl 4-((2-methyl-1-(2-(piperidin-4-yl)ethyl)-1H-indol-3-yl)methyl)benzoate hydrochloride (0.350 g, 0.82 mmol), 2,2-dimethyloxirane (0.591 g, 8.20 mmol) and potassium carbonate (1.133 g, 8.20 mmol) were added to ethanol (10 mL) and heated by microwave irradiation at 110° C. for 20 minutes, followed by cooling to room temperature. Then, the reaction mixture was concentrated under reduced pressure to remove the solvent, and water was added to the concentrate, followed by extraction with ethyl acetate. The organic layer was washed with a saturated aqueous solution of sodium chloride, dried with anhydrous magnesium sulfate, and then concentrated under reduced pressure. The product was used without additional purification (formula 6, 0.375 g, 99%, brown liquid).

### Step: (Formula 8) Methyl 4-((1-(2-(1-(2-fluoro-2-methylpropyl)piperidin-4-yl)ethyl)-2-methyl-1H-indol-3-yl)methyl)benzoate

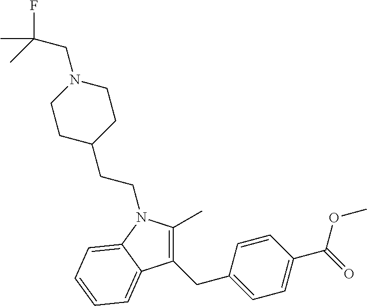 [SMILES: COC(=O)c1ccc(Cc2c(C)n(CCC3CCN(CC(C)(C)F)CC3)c3ccccc23)cc1]

Methyl 4-((1-(2-(1-(2-hydroxy-2-methylpropyl)piperidin-4-yl)ethyl)-2-methyl-1H-indol-3-yl)methyl)benzoate (0.375 g, 0.81 mmol) was dissolved in methylene chloride (10 mL). At room temperature, diethylaminosulfur trifluoride (0.115 mL, 0.97 mmol) was added to the solution, followed by stirring at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 30% to 70%) and concentrated to afford the title compound (0.225 g, 60%) as a yellow liquid.

### Step 6: (Compound 563) 4-((1-(2-(1-(2-Fluoro-2-methylpropyl)piperidin-4-yl)ethyl)-2-methyl-1H-indol-3-yl)methyl)-N-hydroxybenzamide

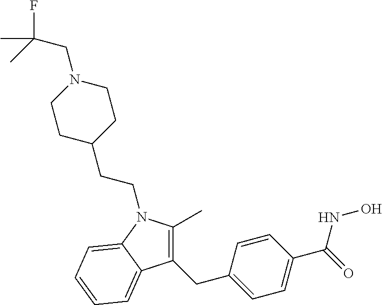 [SMILES: Cc1c(Cc2ccc(C(=O)NO)cc2)c2ccccc2n1CCC1CCN(CC(C)(C)F)CC1]

Methyl 4-((1-(2-(1-(2-fluoro-2-methylpropyl)piperidin-4-yl)ethyl)-2-methyl-1H-indol-3-yl)methyl)benzoate (0.225 g, 0.48 mmol) was dissolved in methanol (10 mL)/tetrahydrofuran (3 mL). At room temperature, hydroxylamine (50 wt % aqueous solution, 2.962 mL, 48.43 mmol) was added to the solution, and then potassium hydroxide (0.272 g, 4.84 mmol) was added thereto, followed by stirring at the same temperature for 3 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 563 (0.146 g, 65%) as a light yellow solid.

¹H NMR (400 MHz, DMSO-d₆) δ 7.56 (d, 2H, J=8.4 Hz), 7.33-7.27 (m, 2H), 7.17 (d, 2H, J=8.0 Hz), 6.99 (t, 1H, J=7.4 Hz), 6.87 (t, 1H, J=7.2 Hz), 4.07 (t, 2H, J=7.8 Hz), 4.00 (s, 2H), 2.84-2.81 (m, 2H), 2.38-2.32 (m, 5H), 2.02-1.96 (m, 2H), 1.65-1.62 (m, 2H), 1.52-1.47 (m, 2H), 1.28-1.15 (m, 9H); MS (ESI) m/z 466.3 (M⁺+H).

### Example 21

### Synthesis of Compound 564

### Step 1: (Formula 14) Methyl 4-((1-(2-(tert-butyldimethylsilyloxy)ethyl)-2-methyl-1H-indol-3-yl)methyl)benzoate

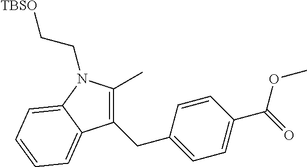 [SMILES: COC(=O)c1ccc(Cc2c(C)n(CCO[Si](C)(C)C(C)(C)C)c3ccccc23)cc1]

Methyl 4-((2-methyl-1H-indol-3-yl)methyl)benzoate (3.000 g, 10.74 mmol) was dissolved in N,N-dimethylformamide (20 mL). At 0° C., sodium hydride (0.309 g, 12.89 mmol) was slowly added dropwise to the solution, followed by stirring for 10 minutes. Then, (2-bromoethoxy)(tert-butyl)dimethylsilane (3.441 mL, 16.11 mmol) was added to the reaction solution, followed by stirring at room temperature for 3 hours. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 10% to 30%) and concentrated to afford the title compound (4.280 g, 91%) as a yellow liquid.

### Step 2: (Formula 15) Methyl 4-((1-(2-hydroxyethyl)-2-methyl-1H-indol-3-yl)methyl)benzoate

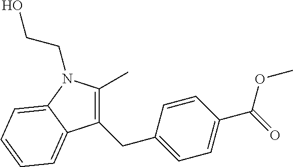 [SMILES: COC(=O)c1ccc(Cc2c(C)n(CCO)c3ccccc23)cc1]

Methyl 4-((1-(2-(tert-butyldimethylsilyloxy)ethyl)-2-methyl-1H-indol-3-yl)methyl)benzoate (4.280 g, 9.78 mmol) was dissolved in tetrahydrofuran (50 mL). At room temperature, tetrabutylammonium fluoride (1.0 M tetrahydrofuran solution, 11.735 mL, 11.74 mmol) was added to the solution, followed by stirring at the same temperature for 1 hour. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 40% to 70%) and concentrated to afford the title compound (3.100 g, 98%) as a yellow solid.

### Step 3: (Formula 16) Methyl 4-((2-methyl-1-(2-(methylsulfonyloxy)ethyl)-1H-indol-3-yl)methyl)benzoate

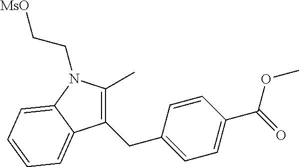 [SMILES: COC(=O)c1ccc(Cc2c(C)n(CCOS(C)(=O)=O)c3ccccc23)cc1]

Methyl 4-((1-(2-hydroxyethyl)-2-methyl-1H-indol-3-yl)methyl)benzoate (3.100 g, 9.59 mmol) was dissolved in methylene chloride (30 mL). At room temperature, methanesulfonyl chloride (1.113 mL, 14.38 mmol) and diisopropylethylamine (2.546 mL, 14.38 mmol) were added to the solution, followed by stirring at the same temperature for 1 hour. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 20% to 50%) and concentrated to afford the title compound (3.420 g, 89%) as a brown solid.

### Step 4: (Formula 17a) Tert-butyl 4-(2-(3-(4-(methoxycarbonyl)benzyl)-2-methyl-1H-indol-1-yl)ethyl)piperazine-1-carboxylate

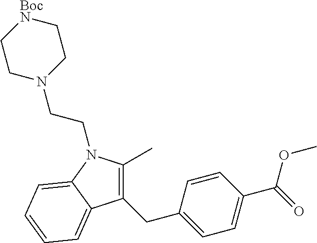 [SMILES: COC(=O)c1ccc(Cc2c(C)n(CCN3CCN(C(=O)OC(C)(C)C)CC3)c3ccccc23)cc1]

Methyl 4-((2-methyl-1-(2-(methylsulfonyloxy)ethyl)-1H-indol-3-yl)methyl)benzoate (0.420 g, 1.05 mmol), 1-Boc-piperazine (0.974 g, 5.23 mmol) and diisopropylethylamine (0.676 g, 5.23 mmol) were added to acetonitrile (3 mL) and heated by microwave irradiation at 120° C. for 2 hours, followed by cooling to room temperature. Then, the reaction mixture was concentrated under reduced pressure. The concentrate was purified by column chromatography (SiO₂, 12 g cartridge; ethyl acetate/hexane=from 30% to 70%) and concentrated to afford the title compound (0.253 g, 49%) as a light yellow solid.

### Step 5: (Formula 21) Methyl 4-((2-methyl-1-(2-(piperazin-1-yl)ethyl)-1H-indol-3-yl)methyl)benzoate hydrochloride

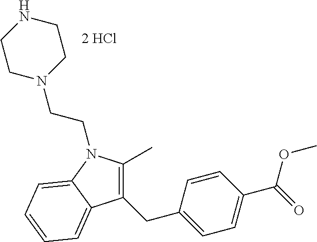 [SMILES: COC(=O)c1ccc(Cc2c(C)n(CCN3CCNCC3)c3ccccc23)cc1]

Tert-butyl 4-(2-(3-(4-(methoxycarbonyl)benzyl)-2-methyl-1H-indol-1-yl)ethyl)piperazine-1-carboxylate (0.253 g, 0.52 mmol) was dissolved in 1,4-dioxane (5 mL). At room temperature, hydrochloric acid (4.0 M, 1,4-dioxane solution, 6.433 mL, 25.73 mmol) was added to the solution, followed by stirring at the same temperature for 1 hour. Then, the reaction mixture was concentrated under reduced pressure. The product was used without additional purification (formula 21, 0.238 g, 100%, light brown solid).

### Step 6: (Formula 22) Ethyl 4-((1-(2-(4-(2-hydroxy-2-methylpropyl)piperazin-1-yl)ethyl)-2-methyl-1H-indol-3-yl)methyl)benzoate

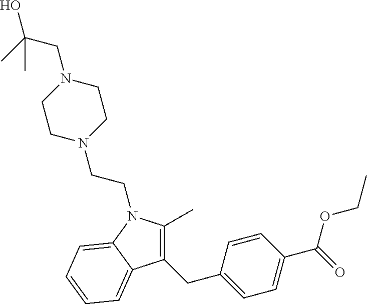 [SMILES: CCOC(=O)c1ccc(Cc2c(C)n(CCN3CCN(CC(C)(C)O)CC3)c3ccccc23)cc1]

Methyl 4-((2-methyl-1-(2-(piperazin-1-yl)ethyl)-1H-indol-3-yl)methyl)benzoate hydrochloride (0.238 g, 0.51 mmol), 2,2-dimethyloxirane (0.370 g, 5.13 mmol) and potassium carbonate (0.708 g, 5.13 mmol) were added to ethanol (10 mL) and heated by microwave irradiation at 110° C. for 20 minutes, followed by cooling to room temperature. Then, the reaction mixture was concentrated under reduced pressure to remove the solvent, and water was added to the concentrate, followed by extraction with ethyl acetate. The organic layer was washed with a saturated aqueous solution of sodium chloride, dried with anhydrous magnesium sulfate, and then concentrated under reduced pressure. The product was used without additional purification (formula 22, 0.213 g, 87%, yellow liquid).

### Step 7: (Formula 23) Ethyl 4-((1-(2-(4-(2-fluoro-2-methylpropyl)piperazin-1-yl)ethyl)-2-methyl-1H-indol-3-yl)methyl)benzoate

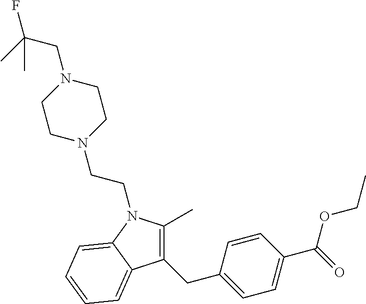 [SMILES: CCOC(=O)c1ccc(Cc2c(C)n(CCN3CCN(CC(C)(C)F)CC3)c3ccccc23)cc1]

Ethyl 4-((1-(2-(4-(2-hydroxy-2-methylpropyl)piperazin-1-yl)ethyl)-2-methyl-1H-indol-3-yl)methyl)benzoate (0.213 g, 0.45 mmol) was dissolved in methylene chloride (10 mL). At room temperature, diethylaminosulfur trifluoride (0.063 mL, 0.54 mmol) was added to the solution, followed by stirring at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 50% to 100%) and concentrated to afford the title compound (0.123 g, 56%) as a yellow liquid.

### Step 8: (Compound 564) 4-((1-(2-(4-(2-Fluoro-2-methylpropyl)piperazin-1-yl)ethyl)-2-methyl-1H-indol-3-yl)methyl)-N-hydroxybenzamide

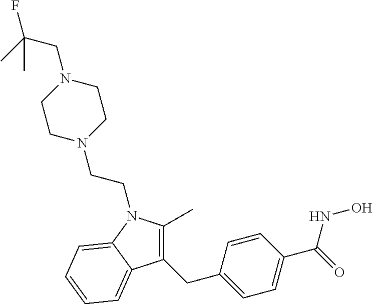 [SMILES: Cc1c(Cc2ccc(C(=O)NO)cc2)c2ccccc2n1CCN1CCN(CC(C)(C)F)CC1]

Ethyl 4-((1-(2-(4-(2-fluoro-2-methylpropyl)piperazin-1-yl)ethyl)-2-methyl-1H-indol-3-yl)methyl)benzoate (0.123 g, 0.26 mmol) was dissolved in methanol (10 mL)/tetrahydrofuran (3 mL). At room temperature, hydroxylamine (50 wt % aqueous solution, 3.137 mL, 51.29 mmol) was added to the solution, and then potassium hydroxide (0.144 g, 2.56 mmol) was added thereto, followed by stirring at the same temperature for 1 hour. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 564 (0.092 g, 77%) as a white solid.

¹H NMR (400 MHz, DMSO-d₆) δ 7.58 (d, 2H, J=8.3 Hz), 7.36-7.33 (m, 2H), 7.16-7.13 (m, 2H), 7.03 (m, 1H), 6.91 (m, 1H), 4.22-4.19 (m, 2H), 4.01 (s, 2H), 2.55-2.53 (m, 2H), 2.46-2.43 (m, 11H), 2.38-2.33 (m, 2H), 1.35 (m, 3H), 1.29 (s, 3H); MS (ESI) m/z 467.3 (M⁺+H).

### Example 22

### Synthesis of Compound 581

### Step 1: (Formula 6) Methyl 4-((1-(2-(1-(2-ethyl-2-hydroxybutyl)piperidin-4-yl)ethyl)-2-methyl-1H-indol-3-yl)methyl)benzoate

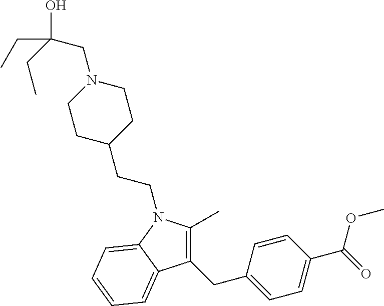 [SMILES: CCC(O)(CC)CN1CCC(CCn2c(C)c(Cc3ccc(C(=O)OC)cc3)c3ccccc32)CC1]

Methyl 4-((2-methyl-1-(2-(piperidin-4-yl)ethyl)-1H-indol-3-yl)methyl)benzoate hydrochloride (0.270 g, 0.63 mmol), 2,2-diethyloxirane (0.633 g, 6.32 mmol) and potassium carbonate (0.874 g, 6.32 mmol) were added to ethanol (10 mL) and heated by microwave irradiation at 110° C. for 20 minutes, followed by cooling to room temperature. Then, the reaction mixture was concentrated under reduced pressure to remove the solvent, and water was added to the concentrate, followed by extraction with ethyl acetate. The organic layer was washed with a saturated aqueous solution of sodium chloride, dried with anhydrous magnesium sulfate, and then concentrated under reduced pressure. The product was used without additional purification (formula 6, 0.260 g, 84%, brown liquid).

### Step 2: (Formula 8) Methyl 4-((1-(2-(1-(2-ethyl-2-fluorobutyl)piperidin-4-yl)ethyl)-2-methyl-1H-indol-3-yl)methyl)benzoate

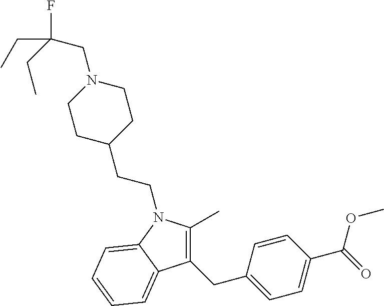 [SMILES: CCC(F)(CC)CN1CCC(CCn2c(C)c(Cc3ccc(C(=O)OC)cc3)c3ccccc32)CC1]

Methyl 4-((1-(2-(1-(2-ethyl-2-hydroxybutyl)piperidin-4-yl)ethyl)-2-methyl-1H-indol-3-yl)methyl)benzoate (0.260 g, 0.53 mmol) was dissolved in methylene chloride (10 mL). At room temperature, diethylaminosulfur trifluoride (0.075 mL, 0.64 mmol) was added to the solution, followed by stirring at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 20% to 50%) and concentrated to afford the title compound (0.074 g, 28%) as a yellow liquid.

### Step 3: (Compound 581) 4-((1-(2-(1-(2-Ethyl-2-fluorobutyl)piperidin-4-yl)ethyl)-2-methyl-1H-indol-3-yl)methyl)-N-hydroxybenzamide

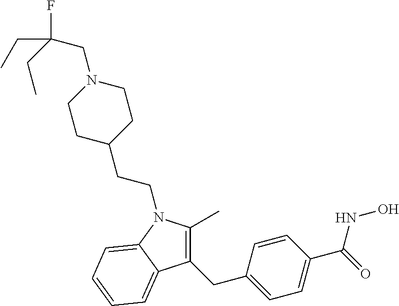 [SMILES: CCC(F)(CC)CN1CCC(CCn2c(C)c(Cc3ccc(C(=O)NO)cc3)c3ccccc32)CC1]

Methyl 4-((1-(2-(1-(2-ethyl-2-fluorobutyl)piperidin-4-yl)ethyl)-2-methyl-1H-indol-3-yl)methyl)benzoate (0.074 g, 0.15 mmol) was dissolved in methanol (5 mL). At room temperature, hydroxylamine (50 wt % aqueous solution, 2.756 mL, 45.06 mmol) was added to the solution, and then potassium hydroxide (0.169 g, 3.00 mmol) was added thereto, followed by stirring at the same temperature for 3 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 581 (0.036 g, 49%) as a light yellow solid.

¹H NMR (400 MHz, DMSO-d₆) δ 7.60 (d, 2H, J=8.3 Hz), 7.37-7.31 (m, 2H), 7.21 (d, 2H, J=8.2 Hz), 7.03 (t, 1H, J=7.6 Hz), 6.91 (t, 1H, J=7.4 Hz), 4.11 (t, 2H, J=7.8 Hz), 4.04 (s, 2H), 2.88-2.85 (m, 2H), 2.43-2.37 (m, 5H), 2.03-1.97 (m, 2H), 1.70-1.52 (m, 8H), 1.27-1.21 (m, 3H), 0.84-0.79 (m, 6H); MS (ESI) m/z 494.3 (M⁺+H).

### Example 23

### Synthesis of Compound 582

4-((5-Fluoro-1-((1-(2-hydroxy-2-methylpropyl)piperidin-4-yl)methyl)-2-methyl-1H-indol-3-yl)methyl)-N-hydroxybenzamide

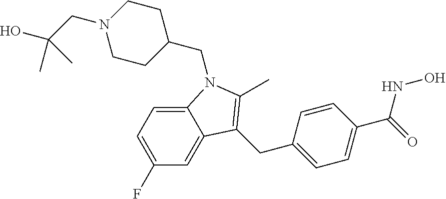 [SMILES: Cc1c(Cc2ccc(C(=O)NO)cc2)c2cc(F)ccc2n1CC1CCN(CC(C)(C)O)CC1]

Ethyl 4-((5-fluoro-1-((1-(2-hydroxy-2-methylpropyl)piperidin-4-yl)methyl)-2-methyl-1H-indol-3-yl)methyl)benzoate (0.140 g, 0.29 mmol) was dissolved in methanol (10 mL). At room temperature, hydroxylamine (50 wt % aqueous solution, 1.782 mL, 29.13 mmol) was added to the solution, and then potassium hydroxide (0.163 g, 2.91 mmol) was added thereto, followed by stirring at the same temperature for 16 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 582 (0.130 g, 95%) as a white solid.

¹H NMR (400 MHz, DMSO-d₆) δ 7.61 (d, 2H, J=8.2 Hz), 7.40 (m, 1H), 7.24 (d, 2H, J=8.1 Hz), 7.11 (dd, 1H, J=9.9, 2.4 Hz), 6.85 (td, 1H, J=9.2, 2.3 Hz), 4.03 (s, 2H), 4.01 (s, 2H), 3.99 (s, 1H), 2.90-2.87 (m, 2H), 2.39 (s, 3H), 2.13 (s, 2H), 1.99-1.94 (m, 2H), 1.68 (m, 1H), 1.34-1.31 (m, 4H), 1.05 (s, 6H); MS (ESI) m/z 468.4 (M⁺+H).

### Example 24

### Synthesis of Compound 584

### Step 1: (Formula 36) Methyl 4-((1-((1-((4-hydroxy-tetrahydro-2H-pyran-4-yl)methyl)piperidin-4-yl)methyl)-2-methyl-1H-indol-3-yl)methyl)benzoate

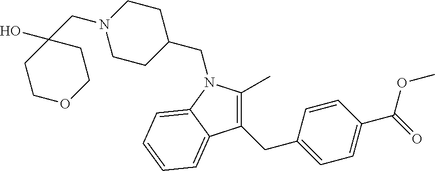 [SMILES: COC(=O)c1ccc(Cc2c(C)n(CC3CCN(CC4(O)CCOCC4)CC3)c3ccccc23)cc1]

Methyl 4-((2-methyl-1-(piperidin-4-ylmethyl)-1H-indol-3-yl)methyl)benzoate hydrochloride (2.000 g, 4.84 mmol), 1,6-dioxaspiro[2,5]octane (4.849 g, 48.43 mmol) and potassium carbonate (6.694 g, 48.43 mmol) were added to ethanol (10 mL), and heated by microwave irradiation at 110° C. for 20 minutes, followed by cooling to room temperature. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The title compound was used without additional purification (1.023 g, 43%, brown liquid).

### Step 2: (Formula 37) Methyl 4-((1-((1-((4-fluoro-tetrahydro-2H-pyran-4-yl)methyl)piperidin-4-yl)methyl)-2-methyl-1H-indol-3-yl)methyl)benzoate

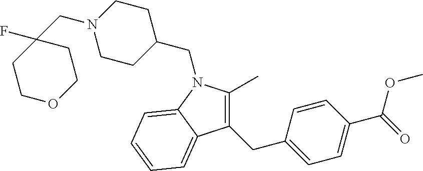 [SMILES: COC(=O)c1ccc(Cc2c(C)n(CC3CCN(CC4(F)CCOCC4)CC3)c3ccccc23)cc1]

Methyl 4-((1-((1-((4-hydroxy-tetrahydro-2H-pyran-4-yl)methyl)piperidin-4-yl)methyl)-2-methyl-1H-indol-3-yl)methyl)benzoate (1.023 g, 2.09 mmol) and diethylaminosulfur trifluoride (0.301 mL, 2.29 mmol) were dissolved in methylene chloride (5 mL) at room temperature. The solution was stirred at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 30% to 70%) and concentrated to afford the title compound (0.180 g, 18%) as a yellow liquid.

### Step 3: (Compound 584) 4-((1-((1-((4-Fluoro-tetrahydro-2H-pyran-4-yl)methyl)piperidin-4-yl)methyl)-2-methyl-1H-indol-3-yl)methyl)-N-hydroxybenzamide

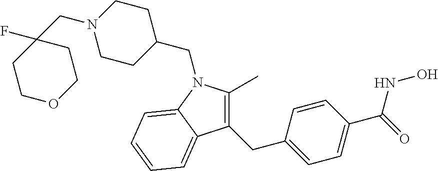 [SMILES: Cc1c(Cc2ccc(C(=O)NO)cc2)c2ccccc2n1CC1CCN(CC2(F)CCOCC2)CC1]

Methyl 4-((1-((1-((4-fluoro-tetrahydro-2H-pyran-4-yl)methyl)piperidin-4-yl)methyl)-2-methyl-1H-indol-3-yl)methyl)benzoate (0.180 g, 0.37 mmol), hydroxylamine (50 wt % aqueous solution, 1.561 mL, 25.52 mmol) and potassium hydroxide (0.205 g, 3.65 mmol) were dissolved in methanol (2 mL) at room temperature, and the solution was stirred at the same temperature 0.5 hours. Then, the reaction mixture was concentrated under reduced pressure to remove the solvent, and a saturated aqueous solution of sodium hydrogen carbonate was added to the concentrate. The precipitated solid was filtered, and then dried to afford desired compound 584 (0.073 g, 40%) as an ivory solid.

¹H-NMR (400 MHz, DMSOd6) d 9.66 (s, 1H), 7.59 (d, 2H, J=8.2 Hz), 7.37 (d, 1H, J=8.3 Hz), 7.35 (d, 1H, J=8.0 Hz), 7.17 (d, 2H, J=8.1 Hz), 7.02 (t, 1H, J=7.4 Hz), 6.91 (t, 1H, J=7.2 Hz), 4.03 (s, 2H), 4.00 (d, 2H, J=7.2 Hz), 3.67-3.64 (m, 2H), 3.57-3.51 (m, 2H), 2.85 (d, 2H, J=11.4 Hz), 2.40 (s, 3H), 1.99 (t, 2H, J=10.2 Hz), 1.72-1.71 (m, 4H), 1.68-1.64 (m, 2H), 1.41-1.29 (m, 4H), 1.25-1.17 (m, 1H); MS (ESI) m/z 494.2 (M⁺+1).

### Example 25

### Synthesis of Compound 585

### Step 1: (Formula 17) Methyl 4-((2-methyl-1-(2-(piperidin-1-yl)ethyl)-1H-indol-3-yl)methyl)benzoate

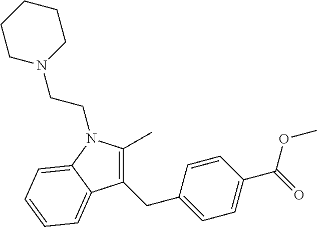 [SMILES: COC(=O)c1ccc(Cc2c(C)n(CCN3CCCCC3)c3ccccc23)cc1]

Methyl 4-((2-methyl-1-(2-(methylsulfonyloxy)ethyl)-1H-indol-3-yl)methyl)benzoate (0.300 g, 0.75 mmol), piperidine (0.221 mL, 2.24 mmol) and diisopropylethylamine (0.651 mL, 3.74 mmol) were added to acetonitrile (3 mL), and heated by microwave irradiation at 120° C. for 1 hour, followed by cooling to room temperature. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 10% to 30%) and concentrated to afford the title compound (0.238 g, 81%) as a yellow liquid.

### Step 2: (Compound 585) 4-((1-((1-((4-Fluoro-tetrahydro-2H-pyran-4-yl)methyl)piperidin-4-yl)methyl)-2-methyl-1H-indol-3-yl)methyl)-N-hydroxybenzamide

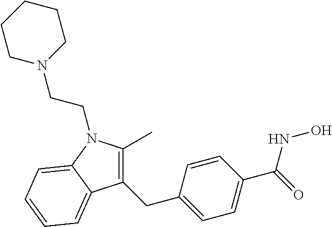 [SMILES: Cc1c(Cc2ccc(C(=O)NO)cc2)c2ccccc2n1CCN1CCCCC1]

Methyl 4-((2-methyl-1-(2-(piperidin-1-yl)ethyl)-1H-indol-3-yl)methyl)benzoate (0.238 g, 0.61 mmol), hydroxylamine (50 wt % aqueous solution, 2.605 mL, 42.58 mmol) and potassium hydroxide (0.341 g, 6.08 mmol) were dissolved in methanol (2 mL) at room temperature, and the solution was stirred at the same temperature for 0.5 hours. Then, the reaction mixture was concentrated under reduced pressure to remove the solvent, and a saturated aqueous solution of sodium hydrogen carbonate was added to the concentrate, followed by filtration. Methylene chloride (1 mL) and diethyl ether (3 mL) were added to the filtrate, followed by stirring. The precipitated solid was filtered and dried to afford compound 585 (0.146 g, 61%) as a white solid.

¹H-NMR (400 MHz, DMSO-d₆) δ 7.24 (d, 2H, J=6.2 Hz), 7.18 (t, 2H, J=6.6 Hz), 7.03 (t, 1H, J=7.6 Hz), 6.89 (t, 1H, J=7.3 Hz), 6.75 (d, 2H, J=6.1 Hz), 4.03 (s, 2H), 3.68 (s, 2H), 2.45 (t, 2H, J=7.3 Hz), 2.36 (s, 4H), 2.14 (s, 3H), 1.56-1.53 (m, 4H), 1.42-1.39 (m, 2H); MS (ESI) m/z 392.2 (M⁺+1).

### Example 26

### Synthesis of Compound 586

Sep 1: (Formula 17) Methyl 4-((1-(2-(3-(hydroxymethyl)pyrrolidin-1-yl)ethyl)-2-methyl-1H-indol-3-yl)methyl)benzoate

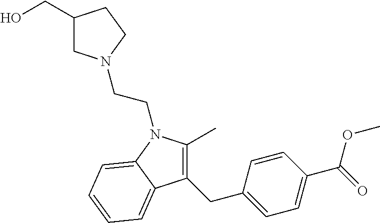 [SMILES: COC(=O)c1ccc(Cc2c(C)n(CCN3CCC(CO)C3)c3ccccc23)cc1]

Methyl 4-((2-methyl-1-(2-(methylsulfonyloxy)ethyl)-1H-indol-3-yl)methyl)benzoate (0.650 g, 1.62 mmol), pyrrolidin-3-ylmethanol (0.491 g, 4.86 mmol) and diisopropylethylamine (1.414 mL, 8.10 mmol) were added to acetonitrile (5 mL), and heated by microwave irradiation at 120° C. for 1 hour, followed by cooling to room temperature. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; methanol/methylene chloride=from 0% to 10%) and concentrated to afford the title compound (0.524 g, 80%) as a yellow liquid.

### Step 2: (Compound 586) N-hydroxy-4-((1-(2-(3-(hydroxymethyl)pyrrolidin-1-yl)ethyl)-2-methyl-1H-indol-3-yl)methyl)benzamide

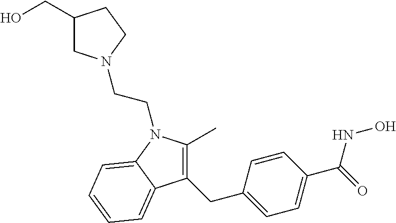 [SMILES: Cc1c(Cc2ccc(C(=O)NO)cc2)c2ccccc2n1CCN1CCC(CO)C1]

Methyl 4-((1-(2-(3-(hydroxymethyl)pyrrolidin-1-yl)ethyl)-2-methyl-1H-indol-3-yl)methyl)benzoate (0.200 g, 0.49 mmol), hydroxylamine (50 wt % aqueous solution, 2.107 mL, 34.44 mmol) and potassium hydroxide (0.276 g, 4.92 mmol) were dissolved in methanol (2 mL) at room temperature, and the solution was stirred at the same temperature for 0.5 hours. Then, the reaction mixture was concentrated under reduced pressure to remove the solvent, and a saturated aqueous solution of sodium hydrogen carbonate was added to the concentrate, followed by filtration. Methylene chloride (1 mL) and diethyl ether (3 mL) were added to the filtrate, followed by stirring. The precipitated solid was filtered and dried to afford desired compound (0.057 g, 28%) as an ivory solid.

¹H-NMR (400 MHz, DMSO-d₆) δ 7.59 (d, 2H, J=8.2 Hz), 7.35, 7.34 (ABq, 2H, J=7.7, 8.0 Hz), 7.19 (d, 2H, J=8.2 Hz), 7.03 (t, 1H, J=7.5 Hz), 6.92 (t, 1H, J=7.4 Hz), 4.20 (t, 2H, J=7.0 Hz), 4.03 (s, 2H), 3.27 (dd, 1H, J=10.2, 3.6 Hz), 2.63 (t, 2H, J=7.1 Hz), 2.57 (t, 1H, J=8.4 Hz), 2.46 (t, 2H, J=7.0 Hz), 2.42 (s, 3H), 2.33 (dd, 1H, J=10.2, 3.6 Hz), 2.21-2.13 (m, 1H), 1.82-1.70 (m, 1H), 1.39-1.30 (m, 1H); MS (ESI) m/z 408.3 (M⁺+1).

### Example 27

### Synthesis of Compound 587

### (S)-4-((5-fluoro-1-(2-(3-hydroxypyrrolidin-1-yl)ethyl)-2-methyl-1H-indol-3-yl)methyl)-N-hydroxybenzamide

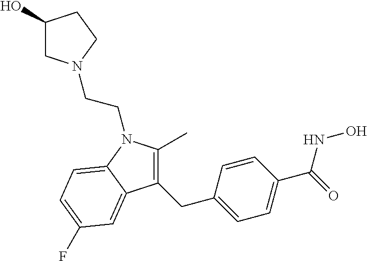 [SMILES: Cc1c(Cc2ccc(C(=O)NO)cc2)c2cc(F)ccc2n1CCN1CC[C@H](O)C1]

(S)-methyl 4-((5-fluoro-1-(2-(3-hydroxypyrrolidin-1-yl)ethyl)-2-methyl-1H-indol-3-yl)methyl)benzoate (0.100 g, 0.24 mmol) was dissolved in methanol (10 mL). At room temperature, hydroxylamine (50 wt % aqueous solution, 1.490 mL, 24.36 mmol) was added to the solution, and then potassium hydroxide (0.137 g, 2.44 mmol) was added thereto, followed by stirring at the same temperature for 16 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 587 (0.041 g, 41%) as a white solid.

¹H NMR (400 MHz, DMSO-d₆) δ 7.61 (d, 2H, J=8.1 Hz), 7.34 (m, 1H), 7.21 (d, 2H, J=7.4 Hz), 7.11 (m, 1H), 6.87 (m, 1H), 4.71 (m, 1H), 4.20-4.17 (m, 3H), 4.01 (s, 2H), 2.73 (m, 1H), 2.70-2.56 (m, 4H), 2.41 (s, 3H), 2.35 (m, 1H), 1.93 (m, 1H), 1.52 (m, 1H); MS (ESI) m/z 412.3 (M⁺+H).

### Example 28

### Synthesis of Compound 588

### Step 1: (Formula 2) methyl 4-((5-fluoro-2-methyl-1H-indol-3-yl)methyl)benzoate

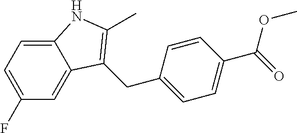 [SMILES: COC(=O)c1ccc(Cc2c(C)[nH]c3ccc(F)cc23)cc1]

Methyl 4-(bromomethyl)benzoate (6.000 g, 26.19 mmol) and 5-fluoro-2-methylindole (4.688 g, 31.43 mmol) were added to water (45 mL), and heated by microwave irradiation at 150° C. for 5 minutes, followed by cooling to room temperature. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 80 g cartridge; ethyl acetate/hexane=from 20% to 50%) and concentrated to afford the title compound (4.640 g, 60%) as a brown solid.

### Step 2: (Formula 4) Tert-butyl 4-((5-fluoro-3-(4-(methoxycarbonyl)benzyl)-2-methyl-1H-indol-1-yl)methyl)piperidine-1-carboxylate

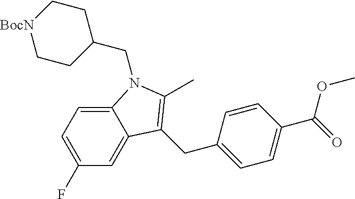 [SMILES: COC(=O)c1ccc(Cc2c(C)n(CC3CCN(C(=O)OC(C)(C)C)CC3)c3ccc(F)cc23)cc1]

Methyl 4-((5-fluoro-2-methyl-1H-indol-3-yl)methyl)benzoate (2.300 g, 7.74 mmol) was dissolved in N,N-dimethylformamide (20 mL). At 0° C., sodium hydride (0.241 g, 10.06 mmol) was slowly added dropwise thereto, followed by stirring for 10 minutes. Then, tert-butyl 4-((methylsulfonyloxy)methyl)piperidine-1-carboxylate (2.723 g, 9.28 mmol) and potassium iodide (1.541 g, 9.28 mmol) were added to the reaction solution, followed by stirring at 60° C. for 2 hours. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 10% to 40%) and concentrated to afford the title compound (2.450 g, 64%) as a light yellow solid.

### Step 3: (Formula 5) Methyl 4-((5-fluoro-2-methyl-1-(piperidin-4-ylmethyl)-1H-indol-3-yl)methyl)benzoate hydrochloride

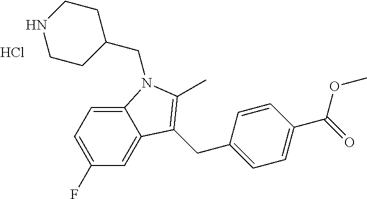 [SMILES: Cl ; COC(=O)c1ccc(Cc2c(C)n(CC3CCNCC3)c3ccc(F)cc23)cc1]

Tert-butyl 4-((5-fluoro-3-(4-(methoxycarbonyl)benzyl)-2-methyl-1H-indol-1-yl)methyl)piperidine-1-carboxylate (2.450 g, 4.95 mmol) was dissolved in 1,4-dioxane (5 mL). At room temperature, hydrochloric acid (4.0 M, 1,4-dioxane solution, 18.576 mL, 74.30 mmol) was added to the solution, followed by stirring at the same temperature for 1 hour. Then, the reaction mixture was concentrated under reduce pressure. Hexane (100 mL) was added to the concentrate, followed by stirring. The precipitated solid was filtered and dried to afford the title compound (2.130 g, 100%) as a light brown solid.

### Step 4: (Formula 6) Ethyl 4-((5-fluoro-1-((1-(2-hydroxy-2-methylpropyl)piperidin-4-yl)methyl)-2-methyl-1H-indol-3-yl)methyl)benzoate

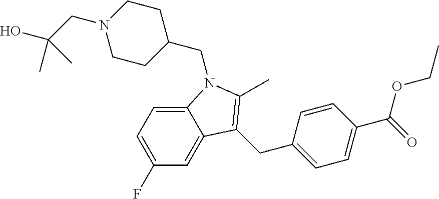 [SMILES: CCOC(=O)c1ccc(Cc2c(C)n(CC3CCN(CC(C)(C)O)CC3)c3ccc(F)cc23)cc1]

Methyl 4-((5-fluoro-2-methyl-1-(piperidin-4-ylmethyl)-1H-indol-3-yl)methyl)benzoate hydrochloride (1.000 g, 2.32 mmol), 2,2-dimethyloxirane (1.673 g, 23.21 mmol) and potassium carbonate (3.207 g, 23.21 mmol) were added to ethanol (15 mL), and heated by microwave irradiation at 110° C. for 20 minutes, followed by cooling to room temperature. Then, the reaction mixture was concentrated under reduced pressure to remove the solvent, and water was added to the concentrate, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 50% to 100%) and concentrated to afford the title compound (0.844 g, 76%) as a light yellow solid.

### Step 5: (Formula 8) Ethyl 4-((5-fluoro-1-((1-(2-fluoro-2-methylpropyl)piperidin-4-yl)methyl)-2-methyl-1H-indol-3-yl)methyl)benzoate

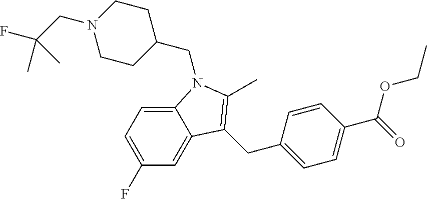 [SMILES: CCOC(=O)c1ccc(Cc2c(C)n(CC3CCN(CC(C)(C)F)CC3)c3ccc(F)cc23)cc1]

Ethyl 4-((5-fluoro-1-((1-(2-hydroxy-2-methylpropyl)piperidin-4-yl)methyl)-2-methyl-1H-indol-3-yl)methyl)benzoate (0.700 g, 1.46 mmol) was dissolved in methylene chloride (10 mL). At room temperature, diethylaminosulfur trifluoride (0.207 mL, 1.75 mmol) was added to the solution, followed by stirring at the same temperature for 2 hours. Then, a saturated aqueous solution of sodium hydrogen carbonate was added to the reaction mixture, followed by extraction with methylene chloride. The organic layer was dried with anhydrous magnesium sulfate, and then concentrated under reduced pressure. The concentrate was purified by column chromatography (SiO₂, 40 g cartridge; ethyl acetate/hexane=from 20% to 60%) and concentrated to afford the title compound (0.480 g, 68%) as a yellow solid.

### Step 6: (Compound 588) 4-((5-Fluoro-1-((1-(2-fluoro-2-methylpropyl)piperidin-4-yl)methyl)-2-methyl-1H-indol-3-yl)methyl)-N-hydroxybenzamide

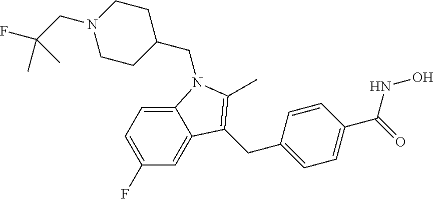 [SMILES: Cc1c(Cc2ccc(C(=O)NO)cc2)c2cc(F)ccc2n1CC1CCN(CC(C)(C)F)CC1]

Ethyl 4-((5-fluoro-1-((1-(2-fluoro-2-methylpropyl)piperidin-4-yl)methyl)-2-methyl-1H-indol-3-yl)methyl)benzoate (0.480 g, 1.00 mmol) was dissolved in methanol (20 mL)/tetrahydrofuran (5 mL). At room temperature, hydroxylamine (50 wt % aqueous solution, 6.084 mL, 99.46 mmol) was added to the solution, and then potassium hydroxide (0.558 g, 9.95 mmol) was added thereto, followed by stirring at the same temperature for 16 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 588 (0.423 g, 91%) as a light yellow solid.

¹H NMR (400 MHz, DMSO-d₆) δ 7.61 (d, 2H, J=8.2 Hz), 7.40 (m, 1H), 7.21 (d, 2H, J=8.2 Hz), 7.11 (dd, 1H, J=9.9, 2.5 Hz), 6.85 (td, 1H, J=9.2, 2.4 Hz), 4.01 (s, 2H), 4.00 (s, 2H), 2.86-2.83 (m, 2H), 2.39-2.33 (m, 5H), 1.98-1.92 (m, 2H), 1.67 (m, 1H), 1.37-1.31 (m, 4H), 1.25 (s, 3H), 1.20 (s, 3H); MS (ESI) m/z 470.4 (M⁺+H).

### Example 29

### Synthesis of Compound 589

### Step 1: (Formula 14) Methyl 4-((1-(2-(tert-butyldimethylsilyloxy)ethyl)-5-fluoro-2-methyl-1H-indol-3-yl)methyl)benzoate

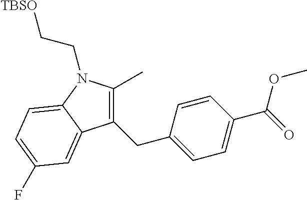 [SMILES: COC(=O)c1ccc(Cc2c(C)n(CCO[Si](C)(C)C(C)(C)C)c3ccc(F)cc23)cc1]

Methyl 4-((5-fluoro-2-methyl-1H-indol-3-yl)methyl)benzoate (2.300 g, 7.74 mmol) was dissolved in N,N-dimethylformamide (10 mL). At 0° C., sodium hydride (0.223 g, 9.28 mmol) was slowly added dropwise to the solution, followed by stirring for 10 minutes. Then, (2-bromoethoxy)(tert-butyl)dimethylsilane (2.479 mL, 11.60 mmol) was added to the reaction solution, followed by stirring at room temperature for 16 hours. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 0% to 30%) and concentrated to afford the title compound (2.770 g, 79%) as a yellow liquid.

### Step 2: (Formula 15) Methyl 4-((5-fluoro-1-(2-hydroxyethyl)-2-methyl-1H-indol-3-yl)methyl)benzoate

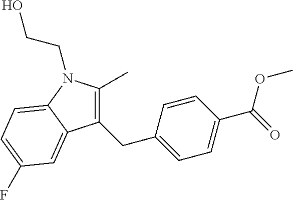 [SMILES: COC(=O)c1ccc(Cc2c(C)n(CCO)c3ccc(F)cc23)cc1]

Methyl 4-((1-(2-(tert-butyldimethylsilyloxy)ethyl)-5-fluoro-2-methyl-1H-indol-3-yl)methyl)benzoate (2.770 g, 6.08 mmol) was dissolved in tetrahydrofuran (20 mL). At room temperature, tetrabutylammonium fluoride (1.0 M tetrahydrofuran solution, 7.295 mL, 7.30 mmol) was added to the solution, followed by stirring at the same temperature for 1 hour. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 40% to 70%) and concentrated to afford the title compound (1.800 g, 87%) as a light yellow solid.

### Step 3: (Formula 16) Methyl 4-((5-fluoro-2-methyl-1-(2-(methylsulfonyloxy)ethyl)-1H-indol-3-yl)methyl)benzoate

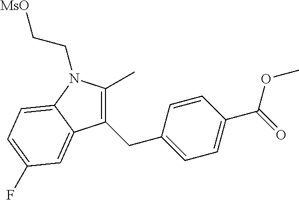 [SMILES: COC(=O)c1ccc(Cc2c(C)n(CCOS(C)(=O)=O)c3ccc(F)cc23)cc1]

Methyl 4-((5-fluoro-1-(2-hydroxyethyl)-2-methyl-1H-indol-3-yl)methyl)benzoate (1.800 g, 5.27 mmol) was dissolved in methylene chloride (30 mL). A room temperature, methanesulfonyl chloride (0.531 mL, 6.86 mmol) and diisopropylethylamine (1.214 mL, 6.86 mmol) were added to the solution, followed by stirring at the same temperature for 1 hour. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The product was used without additional purification (formula 16, 2.180 g, 99%, light yellow solid).

### Step 4: (Formula 17) (S)-methyl 4-((5-fluoro-1-(2-(3-hydroxypyrrolidin-1-yl)ethyl)-2-methyl-1H-indol-3-yl)methyl)benzoate

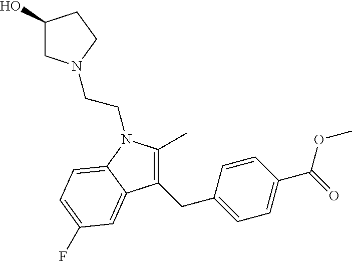 [SMILES: COC(=O)c1ccc(Cc2c(C)n(CCN3CC[C@H](O)C3)c3ccc(F)cc23)cc1]

Methyl 4-((5-fluoro-2-methyl-1-(2-(methylsulfonyloxy)ethyl)-1H-indol-3-yl)methyl)benzoate (0.500 g, 1.19 mmol), (S)-pyrrolidin-3-ol (0.519 g, 5.96 mmol) and diisopropylethylamine (1.055 mL, 5.96 mmol) were added to acetonitrile (4 mL), and heated by microwave irradiation at 120° C. for 2 hours, followed by cooling to room temperature. Then, a saturated aqueous solution of sodium hydrogen carbonate was added to the reaction mixture, followed by extraction with ethyl acetate. The organic layer was dried with anhydrous magnesium sulfate, and then concentrated under reduced pressure. The concentrate was purified by column chromatography (SiO₂, 12 g cartridge; methanol/methylene chloride=from 0% to 10%) and concentrated to afford the title compound (0.411 g, 84%) as a light yellow solid.

### Step 5: (Formula 19) (S)-methyl 4-((5-fluoro-1-(2-(3-fluoropyrrolidin-1-yl)ethyl)-2-methyl-1H-indol-3-yl)methyl)benzoate

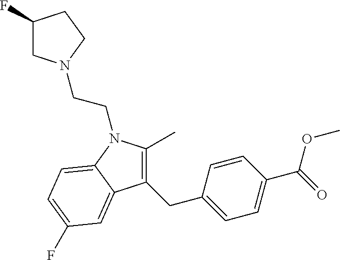 [SMILES: COC(=O)c1ccc(Cc2c(C)n(CCN3CC[C@H](F)C3)c3ccc(F)cc23)cc1]

(S)-methyl 4-((5-fluoro-1-(2-(3-hydroxypyrrolidin-1-yl)ethyl)-2-methyl-1H-indol-3-yl)methyl)benzoate (0.300 g, 0.73 mmol) was dissolved in methylene chloride (10 mL). At room temperature, diethylaminosulfur trifluoride (0.104 mL, 0.88 mmol) was added to the solution, followed by stirring at the same temperature for 2 hours. Then, a saturated aqueous solution of sodium hydrogen carbonate was added to the reaction mixture, followed by extraction with methylene chloride. The organic layer was dried with anhydrous magnesium sulfate, and then concentrated under reduced pressure. The concentrate was purified by column chromatography (SiO₂, 12 g cartridge; ethyl acetate/hexane=from 50% to 80%) and concentrated to afford the title compound (0.198 g, 66%) as a yellow liquid.

### Step 6: (Compound 589) (S)-4-((5-fluoro-1-(2-(3-fluoropyrrolidin-1-yl)ethyl)-2-methyl-1H-indol-3-yl)methyl)-N-hydroxybenzamide

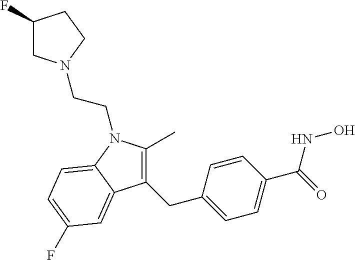 [SMILES: Cc1c(Cc2ccc(C(=O)NO)cc2)c2cc(F)ccc2n1CCN1CC[C@H](F)C1]

(S)-methyl 4-((5-fluoro-1-(2-(3-fluoropyrrolidin-1-yl)ethyl)-2-methyl-1H-indol-3-yl)methyl)benzoate (0.198 g, 0.48 mmol) was dissolved in methanol (10 mL). At room temperature, hydroxylamine (50 wt % aqueous solution, 2.936 mL, 48.00 mmol) was added to the solution, and then potassium hydroxide (0.269 g, 4.80 mmol) was added thereto, followed by stirring at the same temperature for 16 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 589 (0.167 g, 84%) as a white solid.

¹H NMR (400 MHz, DMSO-d₆) δ 7.62 (d, 2H, J=8.2 Hz), 7.37 (m, 1H), 7.25 (d, 2H, J=8.2 Hz), 7.13 (dd, 1H, J=10.0, 2.4 Hz), 6.87 (td, 1H, J=9.2, 2.5 Hz), 5.25-5.08 (m, 1H), 4.23 (t, 2H, J=7.0 Hz), 4.05 (s, 2H), 2.91-2.80 (m, 2H), 2.70-2.67 (m, 2H), 2.60 (m, 1H), 2.43 (s, 3H), 2.29 (m, 1H), 2.11 (m, 1H), 1.83 (m, 1H); MS (ESI) m/z 414.1 (M⁺+H).

### Example 30

### Synthesis of Compound 590

### Step 1: (Formula 17) Methyl 4-((5-fluoro-1-(2-(4-hydroxypiperidin-1-yl)ethyl)-2-methyl-1H-indol-3-yl)methyl)benzoate

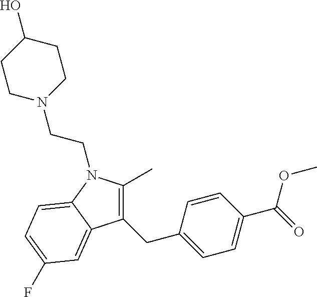 [SMILES: COC(=O)c1ccc(Cc2c(C)n(CCN3CCC(O)CC3)c3ccc(F)cc23)cc1]

Methyl 4-((5-fluoro-2-methyl-1-(2-(methylsulfonyloxy)ethyl)-1H-indol-3-yl)methyl)benzoate (0.500 g, 1.19 mmol), piperidin-4-ol (0.603 g, 5.96 mmol) and diisopropylethylamine (1.055 mL, 5.96 mmol) were added to acetonitrile (4 mL), and heated by microwave irradiation at 120° C. for 2 hours, followed by cooling to room temperature. Then, a saturated aqueous solution of sodium hydrogen carbonate was added to the reaction mixture, followed by extraction with ethyl acetate. The organic layer was dried with anhydrous magnesium sulfate, and then concentrated under reduced pressure. The concentrate was purified by column chromatography (SiO₂, 12 g cartridge; methanol/methylene chloride=from 0% to 10%) and concentrated to afford the title compound (0.423 g, 84%) as a light yellow solid.

### Step 2: (Formula 19) Methyl 4-((5-fluoro-1-(2-(4-fluoropepiridin-1-yl)ethyl)-2-methyl-1H-indol-3-yl)methyl)benzoate

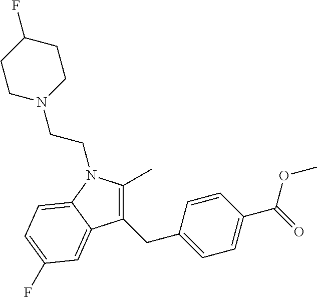 [SMILES: COC(=O)c1ccc(Cc2c(C)n(CCN3CCC(F)CC3)c3ccc(F)cc23)cc1]

Methyl 4-((5-fluoro-1-(2-(4-hydroxypiperidin-1-yl)ethyl)-2-methyl-1H-indol-3-yl)methyl)benzoate (0.323 g, 0.76 mmol) was dissolved in methylene chloride (10 mL). At room temperature, diethylaminosulfur trifluoride (0.108 mL, 0.91 mmol) was added to the solution, followed by stirring at the same temperature for 2 hours. Then, a saturated aqueous solution of sodium hydrogen carbonate was added to the reaction mixture, followed by extraction with methylene chloride. The organic layer was dried with anhydrous magnesium sulfate, and then concentrated under reduced pressure. The concentrate was purified by column chromatography (SiO₂, 12 g cartridge; ethyl acetate/hexane=from 50% to 80%) and concentrated to afford the title compound (0.233 g, 72%) as a light yellow liquid.

### Step 3: (Compound 590) 4-((5-Fluoro-1-(2-(4-fluoropiperidin-1-yl)ethyl)-2-methyl-1H-indol-3-yl)methyl)-N-hydroxybenzamide

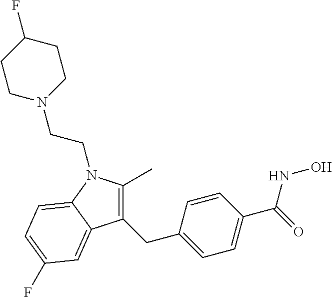 [SMILES: Cc1c(Cc2ccc(C(=O)NO)cc2)c2cc(F)ccc2n1CCN1CCC(F)CC1]

Methyl 4-((5-fluoro-1-(2-(4-fluoropiperidin-1-yl)ethyl)-2-methyl-1H-indol-3-yl)methyl)benzoate (0.233 g, 0.55 mmol) was dissolved in methanol (10 mL). At room temperature, hydroxylamine (50 wt % aqueous solution, 3.342 mL, 54.63 mmol) was added to the solution, and then potassium hydroxide (0.307 g, 5.46 mmol) was added thereto, followed by stirring at the same temperature for 16 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 590 (0.209 g, 90%) as a white solid.

¹H NMR (400 MHz, DMSO-d₆) δ 7.61 (d, 2H, J=8.2 Hz), 7.36 (m, 1H), 7.23 (d, 2H, J=8.2 Hz), 7.12 (dd, 1H, J=9.9, 2.5 Hz), 6.86 (td, 1H, J=9.1, 2.4 Hz), 4.73-4.59 (m, 1H), 4.21 (t, 2H, J=6.8 Hz), 4.02 (s, 2H), 2.55-2.52 (m, 4H), 2.42 (s, 3H), 2.33-2.31 (m, 2H), 1.86-1.76 (m, 2H), 1.68-1.63 (m, 2H); MS (ESI) m/z 428.2 (M⁺+H).

### Example 31

### Synthesis of Compound 591

### Step 1: (Compound 19) Methyl 4-((1-(2-(3-(fluoromethyl)pyrrolidin-1-yl)ethyl)-2-methyl-1H-indol-3-yl)methyl)benzoate

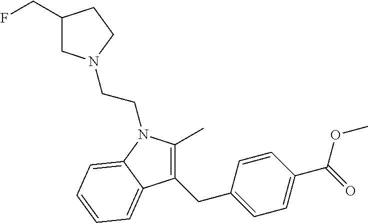 [SMILES: COC(=O)c1ccc(Cc2c(C)n(CCN3CCC(CF)C3)c3ccccc23)cc1]

Methyl 4-((1-(2-(3-(hydroxymethyl)pyrrolidin-1-yl)ethyl)-2-methyl-1H-indol-3-yl)methyl)benzoate (0.324 g, 0.80 mmol) and diethylaminosulfur trifluoride (0.115 mL, 0.88 mmol) were dissolved in methylene chloride (3 mL) at room temperature, and the solution was stirred at the same temperature for 2 hours. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 10% to 50%) and concentrated to afford the title compound (0.062 g, 19%) as a colorless liquid.

### Step 2: (Compound 591) 4-((1-(2-(3-(Fluoromethyl)pyrrolidin-1-yl)ethyl)-2-methyl-1H-indol-3-yl)methyl)-N-hydroxybenzamide

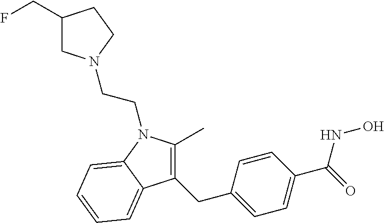 [SMILES: Cc1c(Cc2ccc(C(=O)NO)cc2)c2ccccc2n1CCN1CCC(CF)C1]

Methyl 4-((1-(2-(3-(fluoromethyl)pyrrolidin-1-yl)ethyl)-2-methyl-1H-indol-3-yl)methyl)benzoate (0.062 g, 0.15 mmol), hydroxylamine (50 wt % aqueous solution, 0.651 mL, 10.64 mmol) and potassium hydroxide (0.085 g, 1.52 mmol) were dissolved in tetrahydrofuran (40 mL) at room temperature, and the solution was stirred at the same temperature for 0.5 hours. Then, the reaction mixture was concentrated under reduced pressure to remove the solvent, and a saturated aqueous solution of sodium hydrogen carbonate was added to the concentrate, followed by filtration. Methylene chloride (1 mL) and diethyl ether (3 mL) were added to the filtrate, followed by stirring. The precipitated solid was filtered and dried to afford compound 591 (0.061 g, 97%) as a white solid.

¹H-NMR (400 MHz, DMSO-d₆) δ 9.39 (brs, 1H), 7.58 (d, 2H, J=8.1 Hz), 7.35 (t, 2H, J=8.5 Hz), 7.13 (d, 2H, J=8.1 Hz), 7.03 (t, 1H, J=7.4 Hz), 6.92 (t, 1H, J=7.5 Hz), 4.21 (t, 2H, J=10.7 Hz), 4.01 (s, 2H), 2.70-2.63 (m, 2H), 2.60-2.54 (m, 3H), 2.47-2.45 (m, 2H), 2.42 (s, 3H), 2.40-2.37 (m, 2H), 1.88-1.77 (m, 2H), 1.41-1.33 (m, 2H); MS (ESI) m/z 410.3 (M⁺+1).

### Example 32

### Synthesis of Compound 592

### Step 1: (Formula 17) (S)-methyl 4-((1-(2-(3-hydroxypyrrolidin-1-yl)ethyl)-2-methyl-1H-indol-3-yl)methyl)benzoate

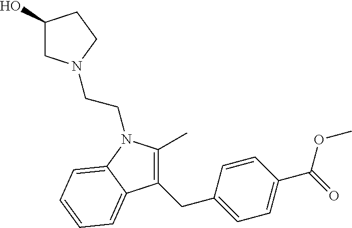 [SMILES: COC(=O)c1ccc(Cc2c(C)n(CCN3CC[C@H](O)C3)c3ccccc23)cc1]

Methyl 4-((2-methyl-1-(2-(methylsulfonyloxy)ethyl)-1H-indol-3-yl)methyl)benzoate (0.600 g, 1.49 mmol), (S)-pyrrolidin-3-ol (0.361 mL, 4.48 mmol) and diisopropylethylamine (1.305 mL, 7.47 mmol) were added to acetonitrile (3 mL), and heated by microwave irradiation at 120° C. for 1 hour, followed by cooling to room temperature. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; methanol/methylene chloride=from 0% to 10%) and concentrated to afford the title compound (0.487 g, 83%) as a light yellow solid.

### Step 2: (Compound 592) (S)—N-hydroxy-4-((1-(2-(3-hydroxypyrrolidin-1-yl)ethyl)-2-methyl-1H-indol-3-yl)methyl)benzamide

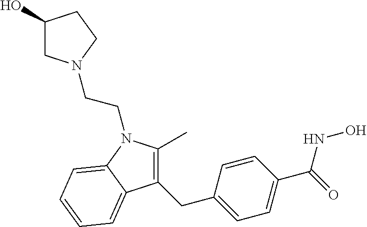 [SMILES: Cc1c(Cc2ccc(C(=O)NO)cc2)c2ccccc2n1CCN1CC[C@H](O)C1]

(S)-methyl 4-((1-(2-(3-hydroxypyrrolidin-1-yl)ethyl)-2-methyl-1H-indol-3-yl)methyl)benzoate (0.202 g, 0.51 mmol), hydroxylamine (50 wt % aqueous solution, 2.203 mL, 36.01 mmol) and potassium hydroxide (0.289 g, 5.14 mmol) were dissolved in methanol (2 mL) at room temperature, and the solution was stirred at the same temperature for 0.5 hours. Then, the reaction mixture was concentrated under reduced pressure to remove the solvent, and a saturated aqueous solution of sodium hydrogen carbonate was added to the concentrate, followed by filtration. Methylene chloride (1 mL) and diethyl ether (3 mL) were added to the filtrate, followed by stirring. The precipitated solid was filtered and dried to afford desired compound 592 (0.202 g, 100%) as a yellow solid.

¹H-NMR (400 MHz, DMSO-d₆) δ 7.55 (d, 2H, J=8.0 Hz), 7.35 (d, 1H, J=7.8 Hz), 7.31 (d, 1H, J=8.1 Hz), 7.07 (d, 2H, J=8.0 Hz), 7.02 (t, 1H, J=7.5 Hz), 6.90 (t, 1H, J=7.4 Hz), 4.18 (t, 2H, J=7.0 Hz), 3.97 (s, 2H), 2.73 (q, 1H, J=5.2 Hz), 2.65-2.55 (m, 4H), 2.41 (s, 3H), 2.37-2.34 (m, 2H), 1.96-1.91 (m, 1H), 1.52-1.51 (m, 1H); MS (ESI) m/z 394.3 (M⁺+1).

### Example 33

### Synthesis of Compound 593

### Step 1: (Formula 19) (S)-methyl 4-((1-(2-(3-fluoropyrrolidin-1-yl)ethyl)-2-methyl-1H-indol-3-yl)methyl)benzoate

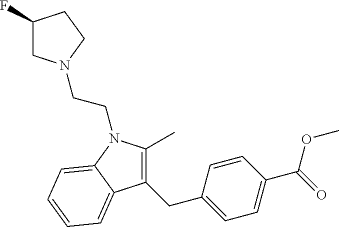 [SMILES: COC(=O)c1ccc(Cc2c(C)n(CCN3CC[C@H](F)C3)c3ccccc23)cc1]

(S)-methyl 4-((1-(2-(3-hydroxypyrrolidin-1-yl)ethyl)-2-methyl-1H-indol-3-yl)methyl)benzoate (0.202 g, 0.51 mmol) and diethylaminosulfur trifluoride (0.101 mL, 0.77 mmol) were dissolved in methylene chloride (5 mL) at room temperature, and the solution was stirred at the same temperature for 2 hours. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; methanol/methylene chloride=from 0% to 10%) and concentrated to afford the title compound (0.196 g, 97%) as a brown liquid.

### Step 2: (Compound 593) (S)-4-((1-(2-(3-fluoropyrrolidin-1-yl)ethyl)-2-methyl-1H-indol-3-yl)methyl)-N-hydroxybenzamide

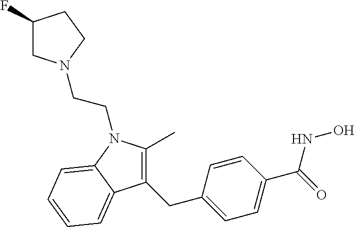 [SMILES: Cc1c(Cc2ccc(C(=O)NO)cc2)c2ccccc2n1CCN1CC[C@H](F)C1]

(S)-methyl 4-((1-(2-(3-fluoropyrrolidin-1-yl)ethyl)-2-methyl-1H-indol-3-yl)methyl)benzoate (0.105 g, 0.27 mmol), hydroxylamine (50 wt % aqueous solution, 1.145 mL, 18.73 mmol) and potassium hydroxide (0.150 g, 2.68 mmol) were dissolved in methanol (2 mL) at room temperature, and the solution was stirred at the same temperature for 0.5 stirred. Then, the reaction mixture was concentrated under reduced pressure to remove the solvent, and a saturated aqueous solution of sodium hydrogen carbonate was added to the concentrate, followed by filtration. Methylene chloride (1 mL) and diethyl ether (3 mL) were added to the filtrate, followed by stirring. The precipitated solid was filtered and dried to afford desired compound 593 (0.071 g, 67%) as a light orange solid.

¹H-NMR (400 MHz, DMSO-d₆) δ 7.57 (d, 2H, J=8.2 Hz), 7.32 (t, 2H, J=7.7 Hz), 7.18 (d, 2H, J=8.3 Hz), 7.01 (td, 1H, J=7.5, 0.9 Hz), 6.89 (t, 1H, J=7.4 Hz), 4.18 (t, 2H, J=6.8 Hz), 4.01 (s, 2H), 3.28-3.15 (m, 2H), 2.40 (s, 3H), 1.90 (t, 1H, J=9.9 Hz), 1.69 (t, 1H, J=10.4 Hz), 1.60-1.54 (m, 3H), 1.43-1.34 (m, 1H), 0.88-0.80 (m, 1H); MS (ESI) m/z 396.2 (M⁺+1).

### Example 34

### Synthesis of Compound 594

### Step 1: (Formula 17) Methyl 4-((1-(2-(3-(hydroxymethyl)piperidin-1-yl)ethyl)-2-methyl-1H-indol-3-yl)methyl)benzoate

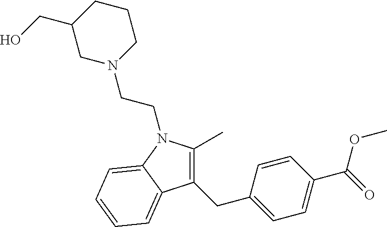 [SMILES: COC(=O)c1ccc(Cc2c(C)n(CCN3CCCC(CO)C3)c3ccccc23)cc1]

Methyl 4-((2-methyl-1-(2-(methylsulfonyloxy)ethyl)-1H-indol-3-yl)methyl)benzoate (0.300 g, 0.76 mmol), 3-piperidinemethanol (0.256 mL, 2.28 mmol) and diisopropylethylamine (0.664 mL, 3.80 mmol) were added to acetonitrile (5 mL), and heated by microwave irradiation at 120° C. for 1 hour, followed by cooling to room temperature. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; methanol/methylene chloride=from 0% to 10%) and concentrated to afford the title compound (0.269 g, 84%) as a brown solid.

### Step 2: (Compound 594) N-hydroxy-4-((1-(2-(3-(hydroxymethyl)piperidin-1-yl)ethyl)-2-methyl-1H-indol-3-yl)methyl)benzamide

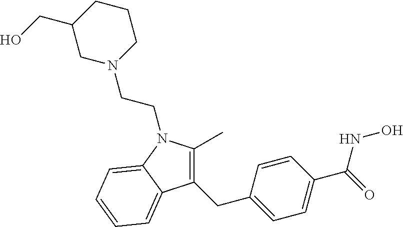 [SMILES: Cc1c(Cc2ccc(C(=O)NO)cc2)c2ccccc2n1CCN1CCCC(CO)C1]

Methyl 4-((1-(2-(3-(hydroxymethyl)piperidin-1-yl)ethyl)-2-methyl-1H-indol-3-yl)methyl)benzoate (0.055 g, 0.13 mmol), hydroxylamine (50 wt % aqueous solution, 0.560 mL, 9.16 mmol) and potassium hydroxide (0.073 g, 1.31 mmol) were dissolved in methanol (2 mL) at room temperature, and the solution was stirred at the same temperature for 0.5 hours. Then, the reaction mixture was concentrated under reduced pressure to remove the solvent, and a saturated aqueous solution of sodium hydrogen carbonate was added to the concentrate, followed by filtration. Methylene chloride (1 mL) and diethyl ether (3 mL) were added to the filtrate, followed by stirring. The precipitated solid was filtered and dried to afford compound 594 (0.039 g, 70%) as a white solid.

¹H-NMR (400 MHz, DMSO-d₆) δ 7.58 (d, 2H, J=8.1 Hz), 7.35 (d, 2H, J=8.1 Hz), 7.18 (d, 2H, J=8.0 Hz), 7.03 (t, 1H, J=7.7 Hz), 6.91 (t, 1H, J=7.4 Hz), 4.22 (t, 2H, J=7.0 Hz), 4.02 (s, 2H), 2.82 (t, 2H, J=6.8 Hz), 2.69 (t, 2H, J=7.1 Hz), 2.55-2.54 (m, 1H), 2.46-2.44 (m, 2H), 2.42 (s, 3H), 2.31 (q, 2H, J=7.8 Hz), 2.20-2.02 (m, 2H), 1.91-1.74 (m, 2H); MS (ESI) m/z 422.2 (M⁺+1).

### Example 35

### Synthesis of Compound 600

### Step 1: (Formula 11) 2-(1-hexynyl)benzeneamine

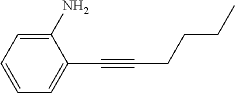

2-iodoaniline (5.000 g, 22.83 mmol), 1-hexyn (3.907 mL, 34.24 mmol), PdCl₂(PPh₃)₂ (1.602 g, 2.28 mmol), copper iodide (0.435 g, 2.28 mmol) and triethylamine (9.493 mL, 68.49 mmol) were dissolved in tetrahydrofuran (40 mL) at room temperature, and the solution was stirred at the same temperature for 3 hours. Then, the reaction mixture was filtered through a celite pad to remove solids, and the filtrate was concentrated under reduced pressure. The concentrate was purified by column chromatography (SiO₂, 80 g cartridge; ethyl acetate/hexane=from 0% to 5%) and concentrated to the title compound (2.760 g, 70%) as a brown liquid.

### Step 2: (Formula 1a, 2-butyl-1H-indole)

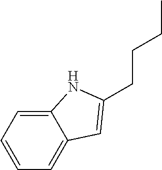

2-(1-hexynyl)benzeneamine (2.760 g, 15.93 mmol) and copper iodide (1.001 g, 5.26 mmol) were dissolved in N,N-dimethylformamide (60 mL) at 200° C., and the solution was stirred at 200° C. for 4 hours, and then stirred at room temperature for 16 hours. Then, the reaction mixture was filtered through a celite pad to remove solids, and the filtrate was concentrated under reduced pressure. The concentrate was purified by column chromatography (SiO₂, 40 g cartridge; ethyl acetate/hexane=from 0% to 5%) and concentrated to afford the title compound (1.406 g, 51%) as a brown liquid.

### Step 3: (Formula 2) Methyl 4-((2-butyl-1H-indol-3-yl)methyl)benzoate

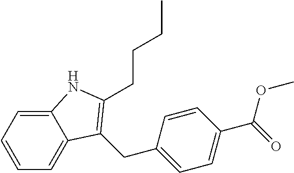 [SMILES: CCCCc1[nH]c2ccccc2c1Cc1ccc(C(=O)OC)cc1]

2-butyl-1H-indole (1.200 g, 6.93 mmol) and methyl 4-(bromomethyl)benzoate (1.428 g, 6.23 mmol) were added to water (5 mL), and heated by microwave irradiation at 140° C. for 5 minutes, followed by cooling to room temperature. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 0% to 10%) and concentrated to afford the title compound (1.403 g, 63%) as an ivory solid.

### Step 4: (Formula 4) Tert-butyl 4-((2-butyl-3-(4-(methoxycarbonyl)benzyl)-1H-indol-1-yl)methyl)piperidine-1-carboxylate

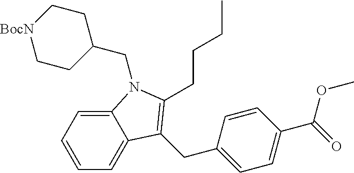 [SMILES: CCCCc1c(Cc2ccc(C(=O)OC)cc2)c2ccccc2n1CC1CCN(C(=O)OC(C)(C)C)CC1]

Methyl 4-((2-butyl-1H-indol-3-yl)methyl)benzoate (0.980 g, 3.05 mmol), tert-butyl 4-((methylsulfonyloxy)methyl)piperidine-1-carboxylate (1.073 g, 3.66 mmol), sodium hydride (60%, 0.159 g, 3.96 mmol) and potassium iodide (0.607 g, 3.66 mmol) were dissolved in N,N-dimethylformamide (20 mL) at 60° C., and the solution was stirred at the same temperature for 2 hours. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 0% to 20%) and concentrated to afford the title compound (0.556 g, 35%) as a yellow solid.

### Step 5: (Formula 5) Methyl 4-((2-butyl-1-(piperidin-4-ylmethyl)-1H-indol-3-yl)methyl)benzoate hydrochloride

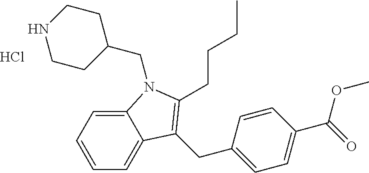 [SMILES: Cl ; CCCCc1c(Cc2ccc(C(=O)OC)cc2)c2ccccc2n1CC1CCNCC1]

Tert-butyl 4-((2-butyl-3-(4-(methoxycarbonyl)benzyl)-1H-indol-1-yl)methyl)piperidine-1-carboxylate (0.556 g, 1.07 mmol) and hydrochloric acid (4.0 M, 1,4-dioxane solution, 2.680 mL, 10.72 mmol) were dissolved in 1,4-dioxane (5 mL) at room temperature, and the solution was stirred at the same temperature for 1 hour. Then, the reaction mixture was concentrated under reduced pressure to remove the solvent, and the concentrate was washed with diethyl ether. The precipitated solid was filtered and dried to afford the title compound (0.461 g, 95%) as a light purple solid.

### Step 6: (Formula 6) Methyl 4-((2-butyl-1-((1-(2-hydroxy-2-methylpropyl)piperidin-4-yl)methyl)-1H-indol-3-yl)methyl)benzoate

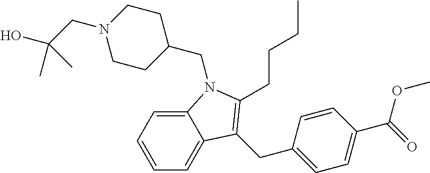 [SMILES: CCCCc1c(Cc2ccc(C(=O)OC)cc2)c2ccccc2n1CC1CCN(CC(C)(C)O)CC1]

Methyl 4-((2-butyl-1-(piperidin-4-ylmethyl)-1H-indol-3-yl)methyl)benzoate hydrochloride (0.461 g, 1.01 mmol), 2,2-dimethyloxirane (0.902 mL, 10.13 mmol) and potassium carbonate (1.400 g, 10.13 mmol) were added to ethanol (5 mL), and heated by microwave irradiation at 110° C. for 20 minutes, followed by cooling to room temperature. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 0% to 20%) and concentrated to afford the title compound (0.302 g, 61%) as a yellow liquid.

### Step 7: (Formula 8) Methyl 4-((2-butyl-1-((1-(2-fluoro-2-methylpropyl)piperidin-4-yl)methyl)-1H-indol-3-yl)methyl)benzoate

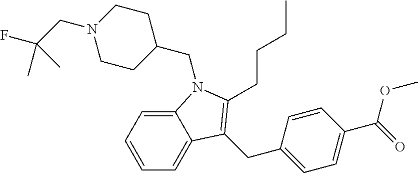 [SMILES: CCCCc1c(Cc2ccc(C(=O)OC)cc2)c2ccccc2n1CC1CCN(CC(C)(C)F)CC1]

Methyl 4-((2-butyl-1-((1-(2-hydroxy-2-methylpropyl)piperidin-4-yl)methyl)-1H-indol-3-yl)methyl)benzoate (0.300 g, 0.61 mmol) and diethylaminosulfur trifluoride (0.120 mL, 0.92 mmol) were dissolved in methylene chloride (3 mL) at room temperature, and the solution was stirred at the same temperature for 2 hours. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 0% to 20%) and concentrated to afford the title compound (0.172 g, 57%) as a colorless liquid.

### Step 8: (Compound 600) 4-((2-Butyl-1-((1-(2-fluoro-2-methylpropyl)piperidin-4-yl)methyl)-1H-indol-3-yl)methyl)-N-hydroxybenzamide

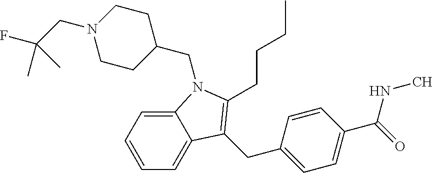 [SMILES: CCCCc1c(Cc2ccc(C(=O)NC)cc2)c2ccccc2n1CC1CCN(CC(C)(C)F)CC1]

Methyl 4-((2-butyl-1-((1-(2-fluoro-2-methylpropyl)piperidin-4-yl)methyl)-1H-indol-3-yl)methyl)benzoate (0.132 g, 0.27 mmol), hydroxylamine (50 wt % aqueous solution, 1.147 mL, 18.76 mmol) and potassium hydroxide (0.150 g, 2.68 mmol) were dissolved in methanol (2 mL)/tetrahydrofuran (2 mL) at room temperature, and the solution was stirred at the same temperature for 16 hours. Then, the reaction mixture was concentrated under reduced pressure to remove the solvent, and a saturated aqueous solution of sodium hydrogen carbonate was added to the concentrate. The precipitated solid was filtered and dried to afford compound 600 (0.050 g, 38%) as an ivory solid.

¹H-NMR (400 MHz, DMSO-d₆) δ 7.68 (d, 1H, J=8.0 Hz), 7.58 (d, 1H, J=8.2 Hz), 7.39 (d, 1H, J=8.2 Hz), 7.29 (d, 1H, J=7.8 Hz), 7.12 (d, 1H, J=8.1 Hz), 7.03 (t, 2H, J=7.7 Hz), 6.90 (t, 1H, J=7.4 Hz), 4.03 (s, 2H), 4.01 (t, 2H, J=3.5 Hz), 2.85 (d, 2H, J=11.4 Hz), 2.77 (t, 2H, J=6.6 Hz), 2.39 (s, 1H), 2.33 (s, 1H), 1.97-1.91 (m, 2H), 1.72-1.66 (m, 1H), 1.41-1.33 (m, 8H), 1.31 (s, 3H), 1.25 (s, 3H), 0.86 (t, 3H, J=7.0 Hz); MS (ESI) m/z 494.3 (M⁺+1).

### Example 36

### Synthesis of Compound 601

### Step 1: (Formula 11) 2-(1-Pentynyl)benzeneamine)

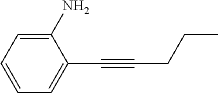

2-iodoaniline (5.000 g, 22.83 mmol), 1-pentyn (3.381 mL, 34.24 mmol), PdCl₂(ppd₃)₂ (1.602 g, 2.28 mmol), copper iodide (0.435 g, 2.28 mmol) and triethylamine (9.493 mL, 68.49 mmol) were dissolved in tetrahydrofuran (40 mL) at room temperature, and the solution was stirred at the same temperature for 3 hours. Then, the reaction mixture was concentrated under reduced pressure. The concentrate was purified by column chromatography (SiO₂, 40 g cartridge; ethyl acetate/hexane=from 0% to 10%) and concentrated to afford the title compound (2.855 g, 79%) as a red liquid.

### Step 2: (Formula 1a) 2-Propyl-1H-indole

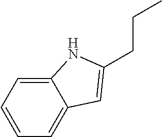

2-(1-pentynyl)benzeneamine (2.855 g, 17.93 mmol) and copper iodide (1.127 g, 5.92 mmol) were dissolved in N,N-dimethylformamide (60 mL) at 200° C., and the solution was stirred at the same temperature for 4 hours, and then stirred at room temperature for 16 hours. The reaction mixture was concentrated under reduced pressure, and the concentrate was purified by column chromatography (SiO₂, 40 g cartridge; ethyl acetate/hexane=from 0% to 5%) and concentrated to afford the title compound (2.437 g, 85%) as a red liquid.

### Step 3: (Formula 2) Methyl 4-((2-propyl-1H-indole-3-yl)methyl)benzoate

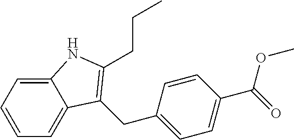 [SMILES: CCCc1[nH]c2ccccc2c1Cc1ccc(C(=O)OC)cc1]

2-propyl-1H-indole (2.437 g, 15.31 mmol) and methyl 4-(bromomethyl)benzoate (3.155 g, 13.77 mmol) were added to water (10 mL), and heated by microwave irradiation at 150° C. for 5 minutes, followed by cooling to room temperature. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 0% to 15%) and concentrated to afford the title compound (1.762 g, 38%) as a yellow solid.

### Step 4: (Formula 4) Tert-butyl 4-((3-(4-(methoxycarbonyl)benzyl)-2-propyl-1H-indol-1-yl)methyl)piperidine-1-carboxylate

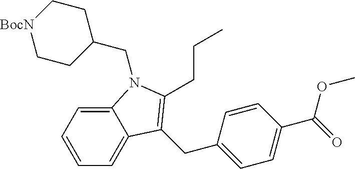 [SMILES: CCCc1c(Cc2ccc(C(=O)OC)cc2)c2ccccc2n1CC1CCN(C(=O)OC(C)(C)C)CC1]

Methyl 4-((2-propyl-1H-indol-3-yl)methyl)benzoate (1.642 g, 5.34 mmol) was dissolved in N,N-dimethylformamide (5 mL). At 0° C., sodium hydride (60%, 0.278 g, 6.94 mmol) and potassium iodide (1.064 g, 6.41 mmol) were slowly added dropwise to the solution, followed by stirring for 10 minutes. Then, tert-butyl 4-((methylsulfonyloxy)methyl)piperidine-1-carboxylate (1.881 g, 6.41 mmol) was added to the reaction solution, followed by stirring at 60° C. for 16 hours. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 80 g cartridge; ethyl acetate/hexane=from 0% to 30%) and concentrated to afford the title compound (0.708 g, 26%) as a yellow solid.

### Step 5: (Formula 5) Methyl 4-((1-(piperidin-4-ylmethyl)-2-propyl-1H-indol-3-yl)methyl)benzoate hydrochloride

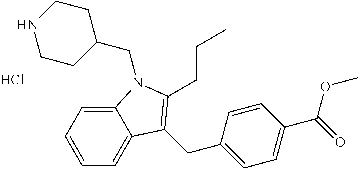 [SMILES: Cl ; CCCc1c(Cc2ccc(C(=O)OC)cc2)c2ccccc2n1CC1CCNCC1]

Tert-butyl 4-((3-(4-(methoxycarbonyl)benzyl)-2-propyl-1H-indol-1-yl)methyl)piperidine-1-carboxylate (0.700 g, 1.39 mmol) and hydrochloric acid (4.0 M, 1,4-dioxane solution, 3.468 mL, 13.87 mmol) were dissolved in 1,4-dioxane (2 mL) at room temperature, and the solution was stirred at the same temperature for 1 hour. Then, the reaction mixture was concentrated under reduced pressure to remove the solvent, and the concentrate was washed with diethyl ether. The precipitated solid was filtered and dried to afford the title compound (0.710 g, 116%) as a brown solid.

### Step 6: (Formula 6) Methyl 4-((1-((1-(2-hydroxy-2-methylpropyl)piperidin-4-yl)methyl)-2-propyl-1H-indol-3-yl)methyl)benzoate

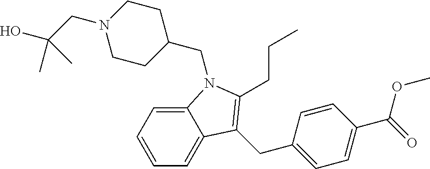 [SMILES: CCCc1c(Cc2ccc(C(=O)OC)cc2)c2ccccc2n1CC1CCN(CC(C)(C)O)CC1]

Methyl 4-((1-(piperidin-4-ylmethyl)-2-propyl-1H-indol-3-yl)methyl)benzoate chloride (0.710 g, 1.61 mmol), 2,2-dimethyloxirane (1.433 mL, 16.10 mmol) and potassium carbonate (2.225 g, 16.10 mmol) were added to ethanol (10 mL), and heated by microwave irradiation at 110° C. for 20 minutes, followed by cooling to room temperature. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; methylene chloride/methanol=from 0% to 5%) and concentrated to afford the title compound (0.599 g, 78%) as a yellow liquid.

### Step 7: (Compound 601) N-hydroxy-4-((1-((1-(2-hydroxy-2-methylpropyl)piperidin-4-yl)methyl)-2-propyl-1H-indol-3-yl)methyl)benzamide

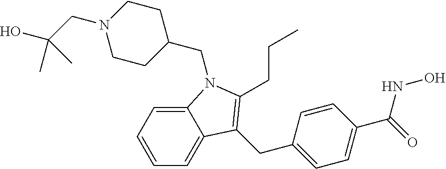 [SMILES: CCCc1c(Cc2ccc(C(=O)NO)cc2)c2ccccc2n1CC1CCN(CC(C)(C)O)CC1]

Methyl 4-((1-((1-(2-hydroxy-2-methylpropyl)piperidin-4-yl)methyl)-2-propyl-1H-indol-3-yl)methyl)benzoate (0.130 g, 0.27 mmol), hydroxylamine (50 wt % aqueous solution, 1.168 mL, 19.09 mmol) and potassium hydroxide (0.153 g, 2.73 mmol) were dissolved in tetrahydrofuran (1 mL)/methanol (2 mL) at room temperature, and the solution was stirred at the same temperature for 1 hour. Then, the reaction mixture was concentrated under reduced pressure to remove the solvent, and a saturated aqueous solution of sodium hydrogen carbonate was added to the concentrate. The precipitated solid was filtered and dried to afford compound 601 (0.080 g, 61%) as a yellow solid.

¹H-NMR (400 MHz, DMSO-d₆) δ 7.59 (d, 2H, J=8.2 Hz), 7.39 (d, 1H, J=8.1 Hz), 7.28 (d, 1H, J=7.8 Hz), 7.14 (d, 2H, J=8.1 Hz), 7.03 (t, 1H, J=7.6 Hz), 6.90 (t, 1H, J=7.4 Hz), 4.05 (s, 2H), 4.01 (s, 2H), 4.00 (s, 1H), 2.89 (d, 2H, J=11.2 Hz), 2.76 (t, 2H, J=7.7 Hz), 2.12 (s, 2H), 1.96-1.92 (m, 2H), 1.72-1.69 (m, 1H), 1.45 (q, 2H, J=7.5 Hz), 1.38-1.31 (m, 4H), 1.05 (s, 6H), 0.91 (t, 3H, J=7.3 Hz); MS (ESI) m/z 478.3 (M⁺+1).

### Example 37

### Synthesis of Compound 602

### Step 1: (Formula 8) Methyl 4-((1-((1-(2-fluoro-2-methylpropyl)piperidin-4-yl)methyl)-2-propyl-1H-indol-3-yl)methyl)benzoate

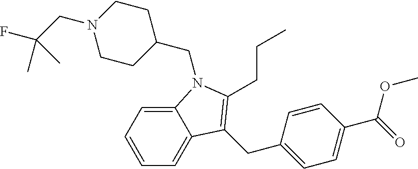 [SMILES: CCCc1c(Cc2ccc(C(=O)OC)cc2)c2ccccc2n1CC1CCN(CC(C)(C)F)CC1]

Methyl 4-((1-((1-(2-hydroxy-2-methylpropyl)piperidin-4-yl)methyl)-2-propyl-1H-indol-3-yl)methyl)benzoate (0.460 g, 0.97 mmol) and diethylaminosulfur trifluoride (0.190 mL, 1.45 mmol) were dissolved in methylene chloride (3 mL) at room temperature, and the solution was stirred at the same temperature for 2 hours. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 0% to 20%) and concentrated to afford the title compound (0.189 g, 41%) as a yellow liquid.

### Step 2: (Compound 602) 4-((1-((1-(2-fluoro-2-methylpropyl)piperidin-4-yl)methyl)-2-propyl-1H-indol-3-yl)methyl)-N-hydroxybenzamide

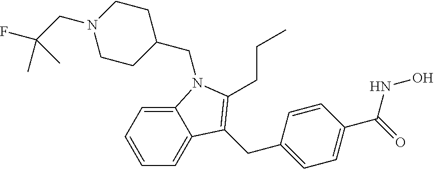 [SMILES: CCCc1c(Cc2ccc(C(=O)NO)cc2)c2ccccc2n1CC1CCN(CC(C)(C)F)CC1]

Methyl 4-((1-((1-(2-fluoro-2-methylpropyl)piperidin-4-yl)methyl)-2-propyl-1H-indol-3-yl)methyl)benzoate (0.189 g, 0.40 mmol), hydroxylamine (50 wt % aqueous solution, 1.691 mL, 27.64 mmol) and potassium hydroxide (0.222 g, 3.95 mmol) were dissolved in tetrahydrofuran (1 mL)/methanol (2 mL) at room temperature, and the solution was stirred at the same temperature for 1 hour. Then, the reaction mixture was concentrated under reduced pressure to remove the solvent, and a saturated aqueous solution of sodium hydrogen carbonate was added to the concentrate. The precipitated solid was filtered, and then dried to afford compound 602 (0.180 g, 95%) as an ivory solid.

¹H-NMR (400 MHz, DMSO-d₆) δ 7.57 (d, 2H, J=8.2 Hz), 7.39 (d, 1H, J=8.2 Hz), 7.29 (d, 1H, J=7.8 Hz), 7.09 (d, 2H, J=8.2 Hz), 7.03 (td, 1H, J=7.6, 0.9 Hz), 6.90 (t, 1H, J=7.1 Hz), 4.00 (s, 2H), 3.98 (s, 2H), 2.85 (d, 2H, J=11.3 Hz), 2.76 (t, 2H, J=7.8 Hz), 2.39 (s, 1H), 2.33 (s, 1 H), 1.97-1.90 (m, 2H), 1.77-1.66 (m, 1H), 1.44 (q, 2H, J=7.6 Hz), 1.37-1.33 (m, 4H), 1.29 (s, 3H), 1.23 (s, 3H), 0.92 (t, 3H, J=7.3 Hz); MS (ESI) m/z 480.2 (M⁺+1).

### Example 38

### Synthesis of Compound 603

### Step 1: (Formula 31) Methyl 4-((2-methyl-1-((1-(3-(thiazol-2-yl)benzyl)piperidin-4-yl)methyl)-1H-indol-3-yl)methyl)benzoate

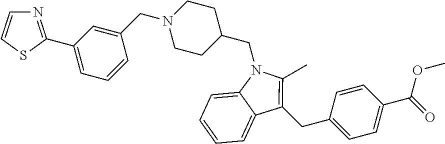 [SMILES: COC(=O)c1ccc(Cc2c(C)n(CC3CCN(Cc4cccc(-c5nccs5)c4)CC3)c3ccccc23)cc1]

Methyl 4-((2-methyl-1-(piperidin-4-ylmethyl)-1H-indol-3-yl)methyl)benzoate hydrochloride (0.300 g, 0.73 mmol), 3-(thiazol-2-yl)benzaldehyde (0.165 g, 0.87 mmol) and acetic acid (0.083 mL, 1.45 mmol) were dissolved in methanol (10 mL), and stirred at room temperature for 30 minutes. Then, sodium cyanoborohydride (0.068 g, 1.09 mmol) was added to the reaction solution, followed by stirring at the same temperature for 16 hours. Then, the reaction mixture was concentrated under reduced pressure to remove the solvent, and a saturated aqueous solution of sodium hydrogen carbonate was added to the concentrate, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 70% to 100%) and concentrated to afford the title compound (0.096 g, 24%) as a white solid.

### Step 2: (Compound 603) N-hydroxy-4-((2-methyl-1-((1-(3-(thiazol-2-yl)benzyl)piperidin-4-yl)methyl)-1H-indol-3-yl)methyl)benzamide

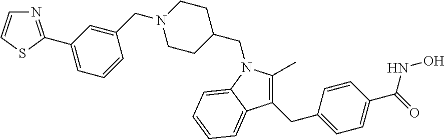 [SMILES: Cc1c(Cc2ccc(C(=O)NO)cc2)c2ccccc2n1CC1CCN(Cc2cccc(-c3nccs3)c2)CC1]

Methyl 4-((2-methyl-1-((1-(3-(thiazol-2-yl)benzyl)piperidin-4-yl)methyl)-1H-indol-3-yl)methyl)benzoate (0.096 g, 0.18 mmol) was dissolved in methanol (10 mL)/tetrahydrofuran (3 mL). At room temperature, hydroxylamine (50 wt % aqueous solution, 3.204 mL, 52.39 mmol) was added to the solution, and then potassium hydroxide (0.098 g, 1.75 mmol) was added thereto, followed by stirring at the same temperature for 3 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 603 (0.088 g, 92%) as a white solid.

¹H NMR (400 MHz, DMSO-d₆) δ 7.93 (d, 1H, J=3.2 Hz), 7.88 (s, 1H), 7.83-7.79 (m, 2H), 7.59 (d, 2H, J=8.2 Hz), 7.47 (t, 1H, J=5.8 Hz), 7.39-7.33 (m, 3H), 7.20 (d, 2H, J=8.2 Hz), 7.02 (t, 1H, J=7.6 Hz), 6.90 (t, 1H, J=7.5 Hz), 4.03-3.99 (m, 4H), 3.54 (s, 2H), 2.83-2.80 (m, 2H), 2.39 (s, 3H), 1.88-1.85 (m, 2H), 1.77 (m, 1H), 1.50-1.34 (m, 4H); MS (ESI) m/z 551.2 (M⁺+H).

### Example 39

### Synthesis of Compound 608

### N-hydroxy-4-((1-((1-(2-hydroxy-2-methylpropyl)piperidin-4-yl)methyl)-2-methyl-1H-indol-3-yl)methyl)benzamide

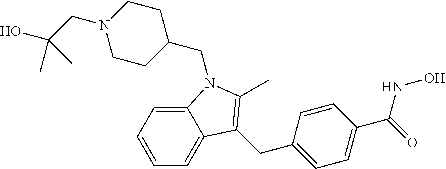 [SMILES: Cc1c(Cc2ccc(C(=O)NO)cc2)c2ccccc2n1CC1CCN(CC(C)(C)O)CC1]

Methyl 4-((1-((1-(2-hydroxy-2-methylpropyl)piperidin-4-yl)methyl)-2-methyl-1H-indol-3-yl)methyl)benzoate (0.050 g, 0.11 mmol) was dissolved in methanol (5 mL). At room temperature, hydroxylamine (50 wt % aqueous solution, 1.364 mL, 22.29 mmol) was added to the solution, and then potassium hydroxide (0.063 g, 1.12 mmol) was added thereto, followed by stirring at the same temperature for 3 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 608 (0.036 g, 72%) as a white solid.

¹H NMR (400 MHz, DMSO-d₆) δ 7.61 (d, 2H, J=8.1 Hz), 7.39-7.34 (m, 2H), 7.23 (d, 2H, J=7.8 Hz), 7.02 (t, 1H, J=7.5 Hz), 6.91 (t, 1H, J=7.4 Hz), 4.05 (s, 2H), 4.00 (s, 2 H), 3.99 (s, 1H), 2.91-2.88 (m, 2H), 2.40 (s, 3H), 2.13 (s, 2H), 2.00-1.94 (m, 2H), 1.70 (m, 1H), 1.37-1.32 (m, 4H), 1.05 (s, 6H); MS (ESI) m/z 450.2 (M⁺+H).

### Example 40

### Synthesis of Compound 609

### Step 1: (Formula 31) methyl 4-((1-((1-((3-fluorooxetan-3-yl)methyl)piperidin-4-yl)methyl)-2-methyl-1H-indol-3-yl)methyl)benzoate

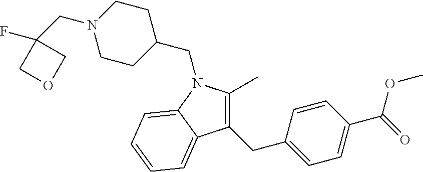 [SMILES: COC(=O)c1ccc(Cc2c(C)n(CC3CCN(CC4(F)COC4)CC3)c3ccccc23)cc1]

Methyl 4-((2-methyl-1-(piperidin-4-ylmethyl)-1H-indol-3-yl)methyl)benzoate hydrochloride (0.200 g, 0.48 mmol), 3-(bromomethyl)-3-fluorooxetane (0.409 g, 2.42 mmol) and diisopropylethylamine (0.429 mL, 2.42 mmol) were added to acetonitrile (3 mL), and heated by microwave irradiation at 120° C. for 6 hours, followed by cooling to room temperature. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 20% to 50%) and concentrated to afford the title compound (0.215 g, 96%) as a light yellow solid.

### Step 2: (Compound 609) 4-((1-((1-((3-Fluorooxetan-3-yl)methyl)piperidin-4-yl)methyl)-2-methyl-1H-indol-3-yl)methyl)-N-hydroxybenzamide

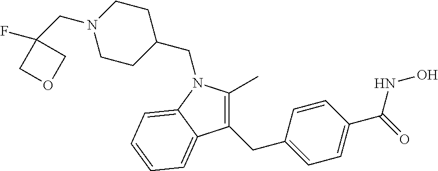 [SMILES: Cc1c(Cc2ccc(C(=O)NO)cc2)c2ccccc2n1CC1CCN(CC2(F)COC2)CC1]

Methyl 4-((1-((1-((3-fluorooxetan-3-yl)methyl)piperidin-4-yl)methyl)-2-methyl-1H-indol-3-yl)methyl)benzoate (0.215 g, 0.46 mmol) was dissolved in methanol (10 mL). At room temperature, hydroxylamine (50 wt % aqueous solution, 2.831 mL, 46.28 mmol) was added to the solution, and then potassium hydroxide (0.260 g, 4.63 mmol) was added thereto, followed by stirring at the same temperature for 16 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford desired compound 609 (0.206 g, 96%) as a white solid.

¹H NMR (400 MHz, DMSO-d₆) δ 7.60 (d, 2H, J=8.2 Hz), 7.39-7.34 (m, 2H), 7.21 (d, 2H, J=8.3 Hz), 7.02 (t, 1H, J=7.1 Hz), 6.91 (t, 1H, J=7.4 Hz), 4.61-4.50 (m, 4H), 4.04 (s, 2H), 4.00 (d, 2H, J=7.4 Hz), 2.83-2.81 (m, 3H), 2.75 (s, 1H), 2.40 (s, 3H), 2.02-1.97 (m, 2H), 1.73 (m, 1H), 1.41-1.31 (m, 4H); MS (ESI) m/z 466.2 (M⁺+H).

### Example 41

### Synthesis of Compound 610

### Step 1: (Formula 36) Tert-butyl 4-hydroxy-4-((4-((3-(4-(methoxycarbonyl)benzyl)-2-methyl-1H-indol-1-yl)methyl)piperidin-1-yl)methyl)piperidine-1-carboxylate

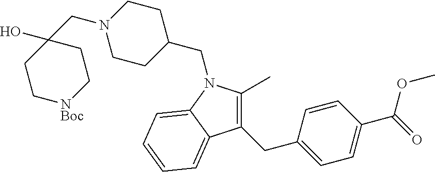 [SMILES: COC(=O)c1ccc(Cc2c(C)n(CC3CCN(CC4(O)CCN(C(=O)OC(C)(C)C)CC4)CC3)c3ccccc23)cc1]

Methyl 4-((2-methyl-1-(piperidin-4-ylmethyl)-1H-indol-3-yl)methyl)benzoate hydrochloride (1.000 g, 2.42 mmol), tert-butyl 1-oxa-6-azaspiro[2.5]octane-6-carboxylate (1.549 g, 7.27 mmol) and potassium carbonate (1.673 g, 12.11 mmol) were added to ethanol (15 mL), and heated by microwave irradiation at 110° C. for 20 minutes, followed by cooling to room temperature. The reaction mixture was concentrated under reduced pressure to remove the solvent, and water was added to the concentrate, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 50% to 80%) and concentrated to afford the title compound (1.120 g, 78%) as a white solid.

### Step 2: (Formula 37) Tert-butyl 4-fluoro-4-((4-((3-(4-(methoxycarbonyl)benzyl)-2-methyl-1H-indol-1-yl)methyl)piperidin-1-yl)methyl)piperidine-1-carboxylate

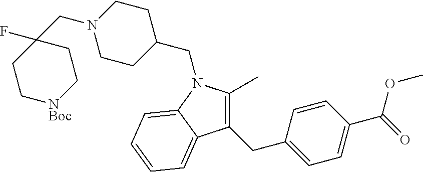 [SMILES: COC(=O)c1ccc(Cc2c(C)n(CC3CCN(CC4(F)CCN(C(=O)OC(C)(C)C)CC4)CC3)c3ccccc23)cc1]

Tert-butyl 4-hydroxy-4-((4-((3-(4-(methoxycarbonyl)benzyl)-2-methyl-1H-indol-1-yl)methyl)piperidin-1-yl)methyl)piperidine-1-carboxylate (1.120 g, 1.90 mmol) was dissolved in methylene chloride (30 mL). At room temperature, diethylaminosulfur trifluoride (0.278 mL, 2.28 mmol) was added to the solution, followed by stirring at the same temperature. Then, a saturated aqueous solution of sodium hydrogen carbonate was added to the reaction mixture, followed by extraction with methylene chloride. The organic layer was dried with anhydrous magnesium sulfate, and then concentrated under reduced pressure. The concentrate was purified by column chromatography (SiO₂, 40 g cartridge; ethyl acetate/hexane=from 20% to 60%) and concentrated to afford the title compound (1.050 g, 93%) as a light yellow solid.

### Step 3: (Compound 610) Tert-butyl 4-fluoro-4-((4-((3-(4-(hydroxycarbamoyl)benzyl)-2-methyl-1H-indol-1-yl)methyl)piperidin-1-yl)methyl)piperidine-1-carboxylate

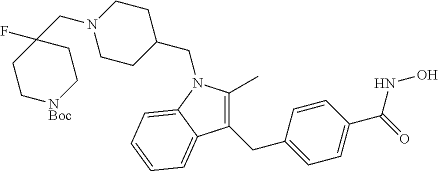 [SMILES: Cc1c(Cc2ccc(C(=O)NO)cc2)c2ccccc2n1CC1CCN(CC2(F)CCN(C(=O)OC(C)(C)C)CC2)CC1]

Tert-butyl 4-fluoro-4-((4-((3-(4-(methoxycarbonyl)benzyl)-2-methyl-1H-indol-1-yl)methyl)piperidin-1-yl)methyl)piperidine-1-carboxylate (0.050 g, 0.08 mmol) was dissolved in methanol (7 mL)/tetrahydrofuran (3 mL). At room temperature, hydroxylamine (50 wt % aqueous solution, 1.550 mL, 25.35 mmol) was added to the solution, and then potassium hydroxide (0.047 g, 0.85 mmol) was added thereto, followed by stirring at the same temperature for 3 hours. Then, the reaction mixture was concentrated under reduced pressure to remove the solvent, and a saturated aqueous solution of sodium hydrogen carbonate was added to the concentrate, followed by stirring. The precipitated solid was filtered, washed with water, and then dried to afford compound 610 (0.042 g, 84%) as a white solid.

¹H NMR (400 MHz, DMSO-d₆) δ 7.60 (d, 2H, J=8.1 Hz), 7.38-7.34 (m, 2H), 7.19 (d, 2H, J=8.4 Hz), 7.02 (t, 1H, J=7.9 Hz), 6.90 (t, 1H, J=7.5 Hz), 4.03-3.99 (m, 4H), 3.71-3.67 (m, 2H), 3.03-2.98 (m, 2H), 2.86-2.83 (m, 2H), 2.41-2.40 (m, 4H), 2.00-1.95 (m, 2H), 1.79-1.73 (m, 3H), 1.61-1.47 (m, 2H), 1.40-1.32 (m, 14H); MS (ESI) m/z 593.4 (M⁺+H).

### Example 42

### Synthesis of Compound 611

### 4-((1-((1-((4-fluoropiperidin-4-yl)methyl)piperidin-4-yl)methyl)-2-methyl-1H-indol-3-yl)methyl)-N-hydroxybenzamide

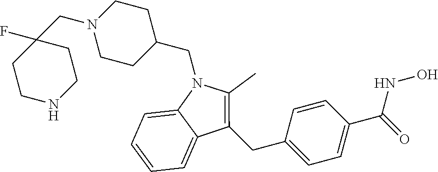 [SMILES: Cc1c(Cc2ccc(C(=O)NO)cc2)c2ccccc2n1CC1CCN(CC2(F)CCNCC2)CC1]

Methyl 4-((1-((1-((4-fluoropiperidin-4-yl)methyl)piperidin-4-yl)methyl)-2-methyl-1H-indole-3-yl)methyl)benzoate hydrochloride (0.050 g, 0.09 mmol) was mixed with methanol (10 mL). Hydroxylamine (50 wt % aqueous solution, 1.083 mL, 17.71 mmol) was added to the mixture, and then potassium hydroxide (0.050 g, 0.89 mmol) was added thereto, followed by stirring at the same temperature for 16 hours. Then, the reaction mixture was concentrated under reduced pressure to remove the solvent, and 5 mL of a saturated aqueous solution of sodium hydrogen carbonate was added to the concentrate, followed by stirring. The precipitated solid was filtered, washed with water, and then dried to afford desired compound 611 (0.021 g, 48%) as a white solid.

¹H NMR (400 MHz, DMSO-d₆) δ 7.61 (d, 2H, J=8.0 Hz), 7.39-7.34 (m, 2H), 7.25 (d, 2H, J=8.2 Hz), 7.02 (t, 1H, J=7.5 Hz), 6.91 (t, 1H, J=7.9 Hz), 4.06 (s, 2H), 4.00 (d, 2H, J=6.9 Hz), 2.86-2.83 (m, 2H), 2.70-2.66 (m, 4H), 2.42-2.37 (m, 5H), 1.98-1.93 (m, 2H), 1.74-1.67 (m, 3H), 1.64-1.59 (m, 2H), 1.58-1.52 (m, 5H); MS (ESI) m/z 493.3 (M⁺+H).

### Example 43

### Synthesis of Compound 612

### Step 1: (Formula 42) Methyl 4-((1-((1-((1-acetyl-4-fluoropiperidin-4-yl)methyl)piperidin-4-yl)methyl)-2-methyl-1H-indol-3-yl)methyl)benzoate

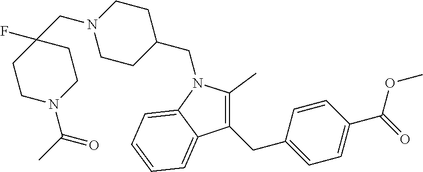 [SMILES: COC(=O)c1ccc(Cc2c(C)n(CC3CCN(CC4(F)CCN(C(C)=O)CC4)CC3)c3ccccc23)cc1]

Methyl 4-((1-((1-((4-fluoropiperidin-4-yl)methyl)piperidin-4-yl)methyl)-2-methyl-1H-indol-3-yl)methyl)benzoate hydrochloride (0.050 g, 0.09 mmol) and diisopropylethylamine (0.047 mL, 0.27 mmol) were dissolved in methylene chloride (10 mL). At room temperature, acetic anhydride (0.017 mL, 0.18 mmol) was added to the solution, followed by stirring at the same temperature for 1 hour. Then, a saturated aqueous solution of sodium hydrogen carbonate was added to the reaction mixture, followed by extraction with methylene chloride. The organic layer was dried with anhydrous magnesium sulfate, and then concentrated under reduced pressure. The concentrate was purified by column chromatography (SiO₂, 12 g cartridge; methanol/methylene chloride=from 0% to 15%) and concentrated to afford the title compound (0.046 g, 97%) as a brown liquid.

### Step 2: (Compound 612) 4-((1-((1-((1-acetyl-4-fluoropiperidin-4-yl)methyl)piperidin-4-yl)methyl)-2-methyl-1H-indol-3-yl)methyl)-N-hydroxybenzamide

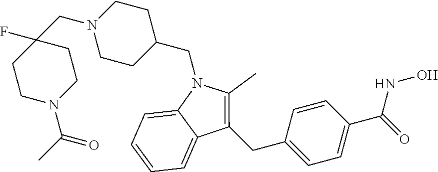 [SMILES: CC(=O)N1CCC(F)(CN2CCC(Cn3c(C)c(Cc4ccc(C(=O)NO)cc4)c4ccccc43)CC2)CC1]

Methyl 4-((1-((1-((1-acetyl-4-fluoropiperidin-4-yl)methyl)piperidin-4-yl)methyl)-2-methyl-1H-indol-3-yl)methyl)benzoate (0.046 g, 0.09 mmol) was mixed with tetrahydrofuran (2 mL)/methanol (5 mL) at room temperature. To the mixture, hydroxylamine (50 wt % aqueous solution, 1.582 mL, 25.86 mmol) was added, and then potassium hydroxide (0.048 g, 0.86 mmol) was added, followed by stirring at the same temperature for 16 hours. Then, the reaction mixture was concentrated under reduced pressure. To the concentrate, a saturated aqueous solution of sodium hydrogen carbonate (3 mL) was added, followed by stirring. The precipitated solid was filtered, washed with water, and then dried to afford compound 612 (0.030 g, 65%) as a light brown solid.

¹H NMR (400 MHz, DMSO-d₆) δ 11.10 (brs, 1H), 8.96 (brs, 1H), 7.61 (d, 2H, J=8.0 Hz), 7.39-7.34 (m, 2H), 7.25 (d, 2H, J=7.8 Hz), 7.03 (t, 1H, J=7.2 Hz), 6.91 (t, 1H, J=7.6 Hz), 4.10-4.00 (m, 5H), 3.62 (m, 1H), 3.57-3.54 (m, 4H), 3.24 (m, 1H), 2.89-2.86 (m, 2H), 2.40 (s, 3H), 2.07-2.00 (m, 5H), 1.83-1.65 (m, 4H), 1.39-1.24 (m, 4H); MS (ESI) m/z 535.3 (M⁺+H).

### Example 44

### Synthesis of Compound 613

### 4-((1-((1-((4-fluoro-1-(2-hydroxy-2-methylpropyl)piperidin-4-yl)methyl)piperidin-4-yl)methyl)-2-methyl-1H-indol-3-yl)methyl)-N-hydroxybenzamide

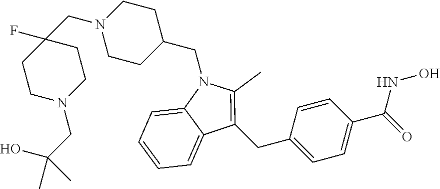 [SMILES: Cc1c(Cc2ccc(C(=O)NO)cc2)c2ccccc2n1CC1CCN(CC2(F)CCN(CC(C)(C)O)CC2)CC1]

4-((1-((1-((1-acetyl-4-fluoropiperidin-4-yl)methyl)piperidin-4-yl)methyl)-2-methyl-1H-indol-3-yl)methyl)-N-hydroxybenzamide (0.050 g, 0.09 mmol) was mixed with tetrahydrofuran (3 mL)/methanol (5 mL) at room temperature. To the mixture, hydroxylamine (50 wt % aqueous solution, 1.627 mL, 26.61 mmol) was added, and then potassium hydroxide (0.050 g, 0.89 mmol) was added, followed by stirring at the same temperature for 16 hours. Then, the reaction mixture was concentrated under reduced pressure. To the concentrate, a saturated aqueous solution of sodium hydrogen carbonate (3 mL) was added, followed by stirring. The precipitated solid was filtered, washed with water, and then dried to afford compound 613 (0.021 g, 42%) as a light yellow solid.

¹H NMR (400 MHz, DMSO-d₆) δ 11.08 (brs, 1H), 8.96 (brs, 1H), 7.61 (d, 2H, J=8.0 Hz), 7.39-7.32 (m, 2H), 7.25 (d, 2H, J=7.4 Hz), 7.02 (t, 1H, J=7.5 Hz), 6.91 (t, 1H, J=7.6 Hz), 4.06-4.00 (m, 5H), 3.41-3.39 (m, 2H), 2.86-2.83 (m, 2H), 2.67-2.64 (m, 2H), 2.43-2.34 (m, 6H), 1.98-1.93 (m, 2H), 1.72-1.56 (m, 6H), 1.38-1.25 (m, 4H), 1.07 (s, 6H); MS (ESI) m/z 565.4 (M⁺+H).

### Example 45

### Synthesis of Compound 614

### Step 1: (Formula 2) Methyl 4-((1H-indol-3-yl)methyl)benzoate

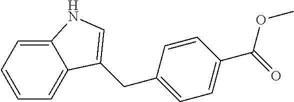 [SMILES: COC(=O)c1ccc(Cc2c[nH]c3ccccc23)cc1]

Methyl 4-(bromomethyl)benzoate (8.000 g, 34.92 mmol) and indole (4.910 g, 41.91 mmol) were added to water (60 mL), and heated by microwave irradiation at 150° C. for 5 minutes, followed by cooling to room temperature. Then, a saturated aqueous solution of sodium hydrogen carbonate was added to the reaction mixture, followed by extraction with methylene chloride. The organic layer was dried with anhydrous magnesium sulfate, and then concentrated under reduced pressure. The concentrate was purified by column chromatography (SiO₂, 80 g cartridge; ethyl acetate/hexane=from 10% to 30%) and concentrated to afford the title compound (4.110 g, 44%) as a light yellow solid.

### Step 2: (Formula 4) Tert-butyl 4-((3-(4-(methoxycarbonyl)benzyl)-1H-indol-1-yl)methyl)piperidine-1-carboxylate

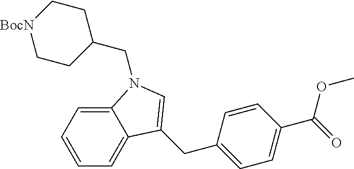 [SMILES: COC(=O)c1ccc(Cc2cn(CC3CCN(C(=O)OC(C)(C)C)CC3)c3ccccc23)cc1]

Methyl 4-((1H-indol-3-yl)methyl)benzoate (4.110 g, 15.49 mmol) was dissolved in N,N-dimethylformamide (50 mL). At room temperature, sodium hydride (95%, 0.470 g, 18.59 mmol) was slowly added dropwise thereto, followed by stirring for 10 minutes. Then, tert-butyl 4-((methylsulfonyloxy)methyl)piperidine-1-carboxylate (5.454 g, 18.59 mmol) and potassium iodide (3.086 g, 18.59 mmol) were added to the reaction solution, followed by stirring at 60° C. for hours. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 80 g cartridge; ethyl acetate/hexane=from 10% to 40%) and concentrated to afford the title compound (formula 4, 4.620 g, 65%) as a light yellow solid.

### Step 3: (Formula 5) Methyl 4-((1-(piperidin-4-ylmethyl)-1H-indol-3-yl)methyl)benzoate hydrochloride

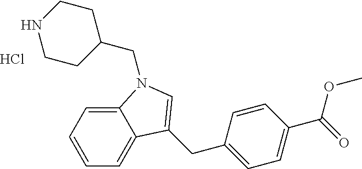 [SMILES: Cl ; COC(=O)c1ccc(Cc2cn(CC3CCNCC3)c3ccccc23)cc1]

Tert-butyl 4-((3-(4-(methoxycarbonyl)benzyl)-1H-indol-1-yl)methyl)piperidine-1-carboxylate (4.620 g, 9.99 mmol) was dissolved in 1,4-dioxane (15 mL). At room temperature, hydrochloric acid (4.0 M, 1,4-dioxane solution, 12.484 mL, 49.94 mmol) was added to the solution, followed by stirring at the same temperature for 3 hours. Then, the reaction mixture was concentrated under reduced pressure. To the concentrate, ethyl acetate (300 mL) was added, followed by stirring. The precipitated solid was filtered and dried to afford the title compound (3.860 g, 97%) as a light brown solid.

### Step 4: (Formula 6) Methyl 4-((1-((1-(2-hydroxy-2-methylpropyl)piperidin-4-yl)methyl)-1H-indol-3-yl)methyl)benzoate

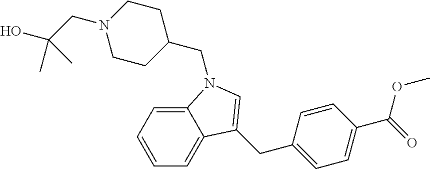 [SMILES: COC(=O)c1ccc(Cc2cn(CC3CCN(CC(C)(C)O)CC3)c3ccccc23)cc1]

Methyl 4-((1-(piperidin-4-ylmethyl)-1H-indol-3-yl)methyl)benzoate hydrochloride (3.860 g, 9.68 mmol), 2,2-dimethyloxirane (3.489 g, 48.38 mmol) and potassium carbonate (13.373 g, 96.76 mmol) were added to ethanol (60 mL), and heated by microwave irradiation at 110° C. for 20 minutes, followed by cooling to room temperature. Then, the reaction mixture was concentrated under reduced pressure to remove the solvent, and water was added to the concentrate, followed by extraction with ethyl acetate. The organic layer was washed with a saturated aqueous solution of sodium chloride, dried with anhydrous magnesium sulfate, and then concentrated under reduced pressure. The product was used without additional purification (formula 6, 4.160 g, 99%, brown liquid).

### Step 5: (Formula 8) Methyl 4-((1-((1-(2-fluoro-2-methylpropyl)piperidin-4-yl)methyl)-1H-indol-3-yl)methyl)benzoate

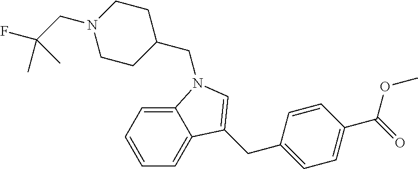 [SMILES: COC(=O)c1ccc(Cc2cn(CC3CCN(CC(C)(C)F)CC3)c3ccccc23)cc1]

Methyl 4-((1-((1-(2-hydroxy-2-methylpropyl)piperidin-4-yl)methyl)-1H-indol-3-yl)methyl)benzoate (4.160 g, 9.57 mmol) was dissolved in methylene chloride (50 mL). At room temperature, diethylaminosulfur trifluoride (1.403 mL, 11.49 mmol) was added to the solution, followed by stirring at the same temperature for 2 hours. Then, a saturated aqueous solution of sodium hydrogen carbonate was added to the reaction mixture, followed by extraction with methylene chloride. The organic layer was dried with anhydrous magnesium sulfate, and then concentrated under reduced pressure. The concentrate was purified by column chromatography (SiO₂, 40 g cartridge; ethyl acetate/hexane=from 20% to 50%) and concentrated to afford the title compound (2.620 g, 63%) as a light yellow liquid.

### Step 6: (Compound 614) 4-((1-((1-(2-Fluoro-2-methylpropyl)piperidin-4-yl)methyl)-1H-indol-3-yl)methyl)-N-hydroxybenzamide

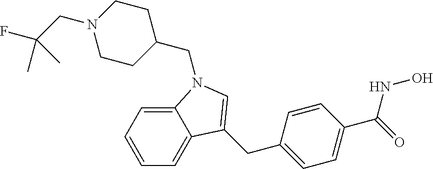 [SMILES: CC(C)(F)CN1CCC(Cn2cc(Cc3ccc(C(=O)NO)cc3)c3ccccc32)CC1]

Methyl 4-((1-((1-(2-fluoro-2-methylpropyl)piperidin-4-yl)methyl)-1H-indol-3-yl)methyl)benzoate (0.100 g, 0.23 mmol) was mixed with tetrahydrofuran (3 mL)/methanol (10 mL) at room temperature. To the mixture, hydroxylamine (50 wt % aqueous solution, 2.102 mL, 34.36 mmol) was added, and then potassium hydroxide (0.129 g, 2.29 mmol) was added, followed by stirring at the same temperature for 16 hours. Then, the reaction mixture was concentrated under reduced pressure. To the concentrate, a saturated aqueous solution of sodium hydrogen carbonate (3 mL) was added, followed by stirring. The precipitated solid was filtered, washed with water, and then dried to afford compound 614 (0.086 g, 86%) as a white solid.

¹H NMR (400 MHz, DMSO-d₆) δ 7.64 (d, 2H, J=8.2 Hz), 7.45-7.39 (m, 2H), 7.32 (d, 2H, J=8.1 Hz), 7.19 (s, 1H), 7.09 (t, 1H, J=7.2 Hz), 6.94 (t, 1H, J=7.4 Hz), 4.06 (s, 2H), 4.01 (d, 2H, J=7.0 Hz), 2.86-2.83 (m, 2H), 2.36 (d, 2H, J=22.8 Hz), 1.99-1.94 (m, 2H), 1.72 (m, 1H), 1.41-1.39 (m, 2H), 1.30-1.25 (m, 8H); MS (ESI) m/z 438.2 (M⁺+H).

### Example 46

### Synthesis of Compound 615

### Step 1: (Formula 39) Methyl 4-((1-((1-((4-fluoropiperidin-4-yl)methyl)piperidin-4-yl)methyl)-2-methyl-1H-indol-3-yl)methyl)benzoate hydrochloride

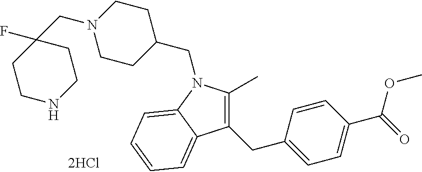 [SMILES: COC(=O)c1ccc(Cc2c(C)n(CC3CCN(CC4(F)CCNCC4)CC3)c3ccccc23)cc1]

Tert-butyl 4-fluoro-4-((4-((3-(4-(methoxycarbonyl)benzyl)-2-methyl-1H-indol-1-yl)methyl)piperidin-1-yl)methyl)piperidine-1-carboxylate (1.000 g, 1.69 mmol) was dissolved in 1,4-dioxane (5 mL). At room temperature, hydrochloric acid (4.0 M, 1,4-dioxane solution, 4.225 mL, 16.90 mmol) was added to the solution, followed by stirring at the same temperature for 1 hour. Then, the reaction mixture was concentrated under reduced pressure. To the concentrate, ethyl acetate (10 mL) and hexane (10 mL) were added, followed by stirring. The precipitated solid was filtered and dried to afford the title compound (0.455 g, 48%) as a light purple solid.

### Step 2: (Formula 40) Methyl 4-((1-((1-((4-fluoro-1-(2-hydroxy-2-methylpropyl)piperidin-4-yl)methyl)piperidin-4-yl)methyl)-2-methyl-1H-indol-3-yl)methyl)benzoate

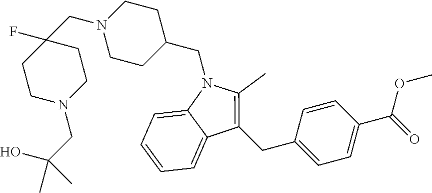 [SMILES: COC(=O)c1ccc(Cc2c(C)n(CC3CCN(CC4(F)CCN(CC(C)(C)O)CC4)CC3)c3ccccc23)cc1]

Methyl 4-((1-((1-((4-fluoropiperidin-4-yl)methyl)piperidin-4-yl)methyl)-2-methyl-1H-indol-3-yl)methyl)benzoate hydrochloride (0.250 g, 0.44 mmol), 2,2-dimethyloxirane (0.160 g, 2.21 mmol) and potassium carbonate (0.612 g, 4.43 mmol) were added to ethanol (15 mL), and heated by microwave irradiation at 110° C. for 20 minutes, followed by cooling to room temperature. Then, the reaction mixture was concentrated under reduced pressure to remove the solvent. To the concentrate, water was added, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; methanol/methylene chloride=from 0% to 10%) and concentrated to afford the title compound (0.231 g, 93%) as a yellow liquid.

### Step 3: (Formula 41) Methyl 4-((1-((1-((4-fluoro-1-(2-fluoro-2-methylpropyl)piperidin-4-yl)methyl)piperidin-4-yl)methyl)-2-methyl-1H-indol-3-yl)methyl)benzoate

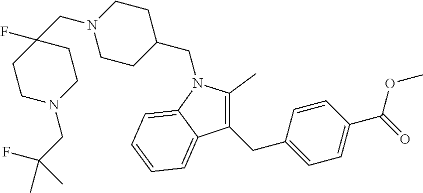 [SMILES: COC(=O)c1ccc(Cc2c(C)n(CC3CCN(CC4(F)CCN(CC(C)(C)F)CC4)CC3)c3ccccc23)cc1]

Methyl 4-((1-((1-((4-fluoro-1-(2-hydroxy-2-methylpropyl)piperidin-4-yl)methyl)piperidin-4-yl)methyl)-2-methyl-1H-indol-3-yl)methyl)benzoate (0.180 g, 0.32 mmol) was mixed with methylene chloride (10 mL) at room temperature. To the mixture, diethylaminosulfur trifluoride (0.047 mL, 0.38 mmol) was added, followed by stirring at the same temperature for 2 hours. Then, a saturated aqueous solution of sodium hydrogen carbonate was added to the reaction mixture, followed by extraction with methylene chloride. The organic layer was dried with anhydrous magnesium sulfate, and then concentrated under reduced pressure. The concentrate was purified by column chromatography (SiO₂, 12 g cartridge; ethyl acetate/hexane=from 40% to 80%) and concentrated to afford the title compound (0.092 g, 51%) as a yellow liquid.

### Step 4: (Compound 615) 4-((1-((1-((4-Fluoro-1-(2-fluoro-2-methylpropyl)piperidin-4-yl)methyl)piperidin-4-yl)methyl)-2-methyl-1H-indol-3-yl)methyl)-N-hydroxybenzamide

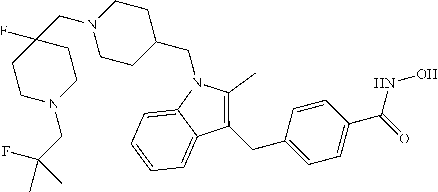 [SMILES: Cc1c(Cc2ccc(C(=O)NO)cc2)c2ccccc2n1CC1CCN(CC2(F)CCN(CC(C)(C)F)CC2)CC1]

Methyl 4-((1-((1-((4-fluoro-1-(2-fluoro-2-methylpropyl)piperidin-4-yl)methyl)piperidin-4-yl)methyl)-2-methyl-1H-indol-3-yl)methyl)benzoate (0.092 g, 0.16 mmol) was mixed with tetrahydrofuran (3 mL)/methanol (7 mL) at room temperature. To the mixture, hydroxylamine (50 wt % aqueous solution, 1.492 mL, 24.39 mmol) was added, and then potassium hydroxide (0.091 g, 1.63 mmol) was added, followed by stirring at the same temperature for 16 hours. Then, the reaction mixture was concentrated under reduced pressure. To the concentrate, a saturated aqueous solution of sodium hydrogen carbonate (3 mL) was added, followed by stirring. The precipitated solid was filtered, washed with water, and then dried to afford compound 615 (0.078 g, 85%) as a white solid.

¹H NMR (400 MHz, DMSO-d₆) δ 7.60 (d, 2H, J=8.1 Hz), 7.39-7.34 (m, 2H), 7.22 (d, 2H, J=8.2 Hz), 7.02 (t, 1H, J=7.4 Hz), 6.91 (t, 1H, J=7.4 Hz), 4.04 (s, 2H), 4.00 (d, 2H, J=7.2 Hz), 2.86-2.83 (m, 2H), 2.64-2.61 (m, 2H), 2.45-2.43 (m, 2H), 2.40-2.31 (m, 7H), 1.98-1.93 (m, 2H), 1.74-1.65 (m, 4H), 1.62 (m, 1H), 1.38-1.32 (m, 7H), 1.27 (s, 3H); MS (ESI) m/z 567.3 (M⁺+H).

### Example 47

### Synthesis of Compound 616

### Step 1: (Formula 33) Methyl 4-((2-bromo-1-((1-(2-fluoro-2-methylpropyl)piperidin-4-yl)methyl)-1H-indol-3-yl)methyl)benzoate

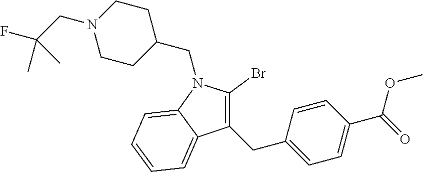 [SMILES: COC(=O)c1ccc(Cc2c(Br)n(CC3CCN(CC(C)(C)F)CC3)c3ccccc23)cc1]

Methyl 4-((1-((1-(2-fluoro-2-methylpropyl)piperidin-4-yl)methyl)-1H-indol-3-yl)methyl)benzoate (0.265 g, 0.61 mmol) was mixed with chloroform (10 mL) at 0° C. To the mixture, N-bromosuccinimide (0.119 g, 0.67 mmol) was added, followed by stirring at room temperature for 16 hours. Then, a saturated aqueous solution of sodium hydrogen carbonate was added to the reaction mixture, followed by extraction with methylene chloride. The organic layer was dried with anhydrous magnesium sulfate, and then concentrated under reduced pressure. The concentrate was purified by column chromatography (SiO₂, 12 g cartridge; ethyl acetate/hexane=from 20% to 50%) and concentrated to afford the title compound (0.075 g, 24%) as a light yellow solid.

### Step 2: (Formula 34) Methyl 4-((1-((1-(2-fluoro-2-methylpropyl)piperidin-4-yl)methyl)-2-phenyl-1H-indol-3-yl)methyl)benzoate

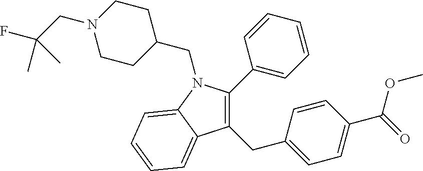 [SMILES: COC(=O)c1ccc(Cc2c(-c3ccccc3)n(CC3CCN(CC(C)(C)F)CC3)c3ccccc23)cc1]

Methyl 4-((2-bromo-1-((1-(2-fluoro-2-methylpropyl)piperidin-4-yl)methyl)-1H-indol-3-yl)methyl)benzoate (0.075 g, 0.15 mmol), phenylboronic acid (0.023 g, 0.19 mmol) and Pd(dppf)Cl₂ (0.012 g, 0.02 mmol) were added to 1,4-dioxane (3 mL) at room temperature. To the mixture, sodium carbonate (2.0 M aqueous solution, 1.091 mL, 2.18 mmol) was added, followed by stirring at 110° C. for 16 hours. Then, a saturated aqueous solution of sodium hydrogen carbonate was added to the reaction mixture, followed by extraction with ethyl acetate. The organic layer was dried with anhydrous magnesium sulfate, and then concentrated under reduced pressure. The concentrate was purified by column chromatography (SiO₂, 4 g cartridge; ethyl acetate/hexane=from 10% to 50%) and concentrated to afford the title compound (0.046 g, 62%) as a light yellow solid.

### Step 3: (Compound 616) 4-((1-((1-(2-Fluoro-2-methylpropyl)piperidin-4-yl)methyl)-2-phenyl-1H-indol-3-yl)methyl)-N-hydroxybenzamide

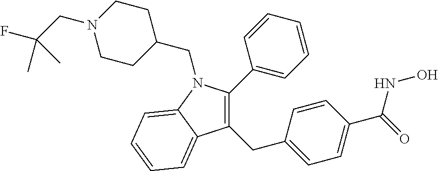 [SMILES: CC(C)(F)CN1CCC(Cn2c(-c3ccccc3)c(Cc3ccc(C(=O)NO)cc3)c3ccccc32)CC1]

Methyl 4-((1-((1-(2-fluoro-2-methylpropyl)piperidin-4-yl)methyl)-2-phenyl-1H-indol-3-yl)methyl)benzoate (0.046 g, 0.09 mmol) was mixed with tetrahydrofuran (3 mL)/methanol (7 mL) at room temperature. To the mixture, hydroxylamine (50 wt % aqueous solution, 1.647 mL, 26.92 mmol) was added, and then potassium hydroxide (0.050 g, 0.90 mmol) was added, followed by stirring at the same temperature for 16 hours. Then, the reaction mixture was concentrated under reduced pressure. To the concentrate, a saturated aqueous solution of sodium hydrogen carbonate (3 mL) was added, followed by stirring. The precipitated solid was filtered, washed with water, and then dried to afford compound 616 (0.014 g, 30%) as a light brown solid.

¹H NMR (400 MHz, DMSO-d₆) δ 7.53-7.42 (m, 6H), 7.37-7.35 (m, 2H), 7.15 (m, 1H), 7.03-6.97 (m, 3H), 4.03 (d, 2H, J=7.0 Hz), 3.96 (s, 2H), 2.69-2.67 (m, 2H), 2.29-2.23 (m, 2H), 1.79-1.77 (m, 2H), 1.46 (m, 1H), 1.23 (s, 3H), 1.18-1.13 (m, 6H), 0.98-0.96 (m, 2H); MS (ESI) m/z 514.2 (M⁺+H).

### Example 48

### Synthesis of Compound 619

### Step 1: (Formula 31) Methyl 4-((2-methyl-1-((1-(2,4,5-trifluorobenzyl)piperidin-4-yl)methyl)-1H-indol-3-yl)methyl)benzoate

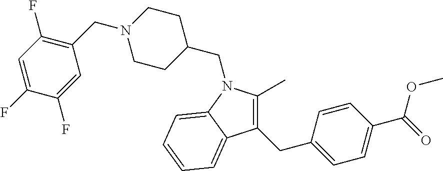 [SMILES: COC(=O)c1ccc(Cc2c(C)n(CC3CCN(Cc4cc(F)c(F)cc4F)CC3)c3ccccc23)cc1]

Methyl 4-((2-methyl-1-(piperidin-4-ylmethyl)-1H-indol-3-yl)methyl)benzoate hydrochloride (0.150 g, 0.36 mmol), 2,4,5-trifluorobenzyl bromide (0.057 mL, 0.44 mmol) and diisopropylethylamine (0.188 mL, 1.09 mmol) were dissolved in methylene chloride (2 mL) at room temperature, and the solution was stirred at the same temperature for 0.5 hours. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 0% to 60%) and concentrated to afford the title compound (0.137 g, 72%) as a colorless liquid.

### Step 2: (Compound 619) N-hydroxy-4-((2-methyl-1-((1-(2,4,5-trifluorobenzyl)piperidin-4-yl)methyl)-1H-indol-3-yl)methyl)benzamide

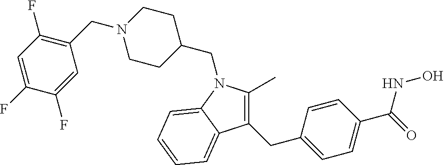 [SMILES: Cc1c(Cc2ccc(C(=O)NO)cc2)c2ccccc2n1CC1CCN(Cc2cc(F)c(F)cc2F)CC1]

Methyl 4-((2-methyl-1-((1-(2,4,5-trifluorobenzyl)piperidin-4-yl)methyl)-1H-indol-3-yl)methyl)benzoate (0.155 g, 0.30 mmol), hydroxylamine (50 wt % aqueous solution, 1.275 mL, 20.84 mmol) and potassium hydroxide (0.167 g, 2.98 mmol) were dissolved in methanol (1 mL)/tetrahydrofuran (1 mL) at room temperature, and the solution was stirred at the same temperature for 1 hour. Then, the reaction mixture was concentrated under reduced pressure to remove the solvent, and a saturated aqueous solution of sodium hydrogen carbonate was added to the concentrate. The precipitated solid was filtered and dried to afford compound 619 (0.136 g, 87%) as an ivory solid.

¹H-NMR (400 MHz, DMSO-d₆) δ 7.57 (d, 2H, J=8.2 Hz), 7.53-7.41 (m, 2H), 7.37 (d, 1H, J=8.2 Hz), 7.34 (d, 1H, J=7.8 Hz), 7.12 (d, 2H, J=8.1 Hz), 7.01 (t, 1H, J=7.6 Hz), 6.90 (t, 1H, J=7.4 Hz), 4.01 (s, 2H), 4.00 (s, 2H), 3.44 (s, 2H), 2.77 (d, 2H, J=11.1 Hz), 2.39 (s, 3H), 1.88 (t, 2H, J=10.6 Hz), 1.79-1.69 (m, 1H), 1.44-1.24 (m, 4H); MS (ESI) m/z 522.2 (M⁺+1).

### Example 49

### Synthesis of Compound 620

### Step 1: (Formula 31) Methyl 4-((1-((1-(2-fluorobenzyl)piperidin-4-yl)methyl)-2-methyl-1H-indol-3-yl)methyl)benzoate

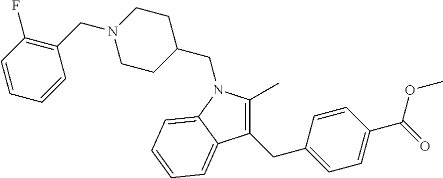 [SMILES: COC(=O)c1ccc(Cc2c(C)n(CC3CCN(Cc4ccccc4F)CC3)c3ccccc23)cc1]

Methyl 4-((2-methyl-1-(piperidin-4-ylmethyl)-1H-indol-3-yl)methyl)benzoate hydrochloride (0.150 g, 0.36 mmol), 2-fluorobenzyl bromide (0.053 mL, 0.44 mmol) and diisopropylethylamine (0.188 mL, 1.09 mmol) were dissolved in methylene chloride (2 mL) at room temperature, and the solution was stirred at the same temperature for 0.5 hours. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 0% to 60%) and concentrated to afford the title compound (0.079 g, 45%) as a colorless liquid.

### Step 2: (Compound 620) 4-((1-((1-(2-Fluorobenzyl)piperidin-4-yl)methyl)-2-methyl-1H-indol-3-yl)methyl)-N-hydroxybenzamide

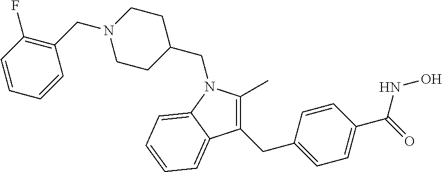 [SMILES: Cc1c(Cc2ccc(C(=O)NO)cc2)c2ccccc2n1CC1CCN(Cc2ccccc2F)CC1]

Methyl 4-((1-((1-(2-fluorobenzyl)piperidin-4-yl)methyl)-2-methyl-1H-indol-3-yl)methyl)benzoate (0.102 g, 0.21 mmol), hydroxylamine (50 wt % aqueous solution, 0.901 mL, 14.73 mmol) and potassium hydroxide (0.118 g, 2.11 mmol) were dissolved in methanol (1 mL)/tetrahydrofuran (1 mL) at room temperature, and the solution was stirred at the same temperature for 1 hour. Then, the reaction mixture was concentrated under reduced pressure to remove the solvent, and a saturated aqueous solution of sodium hydrogen carbonate was added to the concentrate. The precipitated solid was filtered, and then dried to afford compound 620 (0.093 g, 91%) as an ivory solid.

¹H-NMR (400 MHz, DMSO-d₆) δ 7.57 (d, 2H, J=8.2 Hz), 7.40-7.27 (m, 4H), 7.18-7.11 (m, 4H), 7.01 (td, 1H, J=7.6, 0.9 Hz), 6.90 (t, 1H, J=7.8 Hz), 4.01 (s, 2H), 4.00 (s, 2H), 3.46 (s, 2H), 2.79 (d, 2H, J=11.1 Hz), 2.39 (s, 3H), 1.87 (t, 2H, J=10.6 Hz), 1.80-1.67 (m, 1H), 1.44-1.24 (m, 4H); MS (ESI) m/z 486.2 (M⁺+1).

### Example 50

### Synthesis of Compound 621

### Step 1: (Formula 31) Methyl 4-((2-methyl-1-((1-(pyridin-4-ylmethyl)piperidin-4-yl)methyl)-1H-indol-3-yl)methyl)benzoate

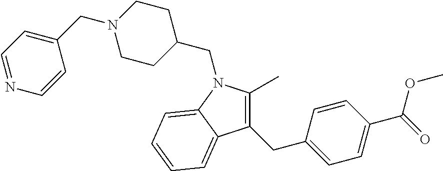 [SMILES: COC(=O)c1ccc(Cc2c(C)n(CC3CCN(Cc4ccncc4)CC3)c3ccccc23)cc1]

Methyl 4-((2-methyl-1-(piperidin-4-ylmethyl)-1H-indol-3-yl)methyl)benzoate hydrochloride (0.150 g, 0.36 mmol), 4-(bromomethyl)pyridine (0.110 g, 0.44 mmol) and diisopropylethylamine (0.188 mL, 1.09 mmol) were dissolved in methylene chloride (0.5 mL) at room temperature, and the solution was stirred at the same temperature for 0.5 hours. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 20%) and concentrated to afford the title compound (0.048 g, 28%) as a light yellow liquid.

### Step 2: (Compound 621) N-hydroxy-4-((2-methyl-1-((1-(pyridin-4-ylmethyl)piperidin-4-yl)methyl)-1H-indol-3-yl)methyl)benzamide

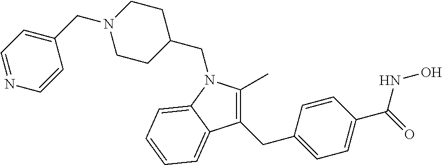 [SMILES: Cc1c(Cc2ccc(C(=O)NO)cc2)c2ccccc2n1CC1CCN(Cc2ccncc2)CC1]

Methyl 4-((2-methyl-1-((1-(pyridin-4-ylmethyl)piperidin-4-yl)methyl)-1H-indol-3-yl)methyl)benzoate (0.048 g, 0.10 mmol), hydroxylamine (50 wt % aqueous solution, 0.440 mL, 7.19 mmol) and potassium hydroxide (0.058 g, 1.03 mmol) were dissolved in methanol (1 mL)/tetrahydrofuran (1 mL) at room temperature, and the solution was stirred at the same temperature for 1 hour. Then, the reaction mixture was concentrated under reduced pressure to remove the solvent, and a saturated aqueous solution of sodium hydrogen carbonate was added to the concentrate. The precipitated solid was filtered and dried to afford desired compound 621 (0.032 g, 67%) as an ivory solid.

¹H-NMR (400 MHz, DMSO-d₆) δ 8.49 (d, 2H, J=5.8 Hz), 7.57 (d, 2H, J=8.1 Hz), 7.38 (d, 1H, J=8.2 Hz), 7.35 (d, 1H, J=8.0 Hz), 7.30 (d, 2H, J=5.6 Hz), 7.12 (d, 2H, J=8.0 Hz), 7.02 (t, 1H, J=7.5 Hz), 6.90 (t, 1H, J=7.4 Hz), 4.08 (s, 2H), 4.00 (s, 2H), 3.45 (s, 2H), 2.76 (d, 2H, J=11.3 Hz), 2.40 (s, 3H), 1.87 (t, 2H, J=10.6 Hz), 1.79-1.69 (m, 1H), 1.46-1.32 (m, 4H); MS (ESI) m/z 469.2 (M⁺+1).

### Example 51

### Synthesis of Compound 622

### Step 1: (Formula 31) Methyl 4-((1-((1-(4-fluorobenzyl)piperidin-4-yl)methyl)-2-methyl-1H-indol-3-yl)methyl)benzoate

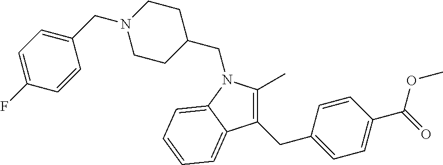 [SMILES: COC(=O)c1ccc(Cc2c(C)n(CC3CCN(Cc4ccc(F)cc4)CC3)c3ccccc23)cc1]

Methyl 4-((2-methyl-1-(piperidin-4-ylmethyl)-1H-indol-3-yl)methyl)benzoate hydrochloride (0.150 g, 0.36 mmol) was dissolved in methanol (3 mL). At 60° C., 4-fluorobenzaldehyde (0.047 mL, 0.44 mmol) and acetic acid (0.031 mL, 0.55 mmol) were added to the solution, followed by stirring for 1 hour. Sodium triacetoxyborohydride (0.092 g, 0.44 mmol) was added to the reaction solution, followed by stirring at the same temperature for 15 hours. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 30% to 70%) and concentrated to afford the title compound (0.052 g, 30%) as a colorless liquid.

### Step 2: (Compound 622) 4-((1-((1-(4-Fluorobenzyl)piperidin-4-yl)methyl)-2-methyl-1H-indol-3-yl)methyl)-N-hydroxybenzamide

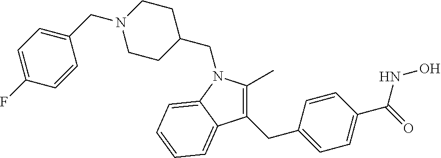 [SMILES: Cc1c(Cc2ccc(C(=O)NO)cc2)c2ccccc2n1CC1CCN(Cc2ccc(F)cc2)CC1]

Methyl 4-((1-((1-(4-fluorobenzyl)piperidin-4-yl)methyl)-2-methyl-1H-indol-3-yl)methyl)benzoate (0.052 g, 0.11 mmol), hydroxylamine (50 wt % aqueous solution, 0.459 mL, 7.51 mmol) and potassium hydroxide (0.060 g, 1.07 mmol) were dissolved in methanol (1 mL)/tetrahydrofuran (1 mL) at room temperature, and the solution was stirred at the same temperature for 1 hour. Then, the reaction mixture was concentrated under reduced pressure to remove the solvent, and a saturated aqueous solution of sodium hydrogen carbonate was added to the concentrate. The precipitated solid was filtered and dried to afford compound 622 (0.013 g, 25%) as an ivory solid.

¹H-NMR (400 MHz, DMSO-d₆) δ 7.56 (d, 2H, J=8.0 Hz), 7.35 (t, 2H, J=9.1 Hz), 7.29 (dd, 2H, J=10.2, 3.6 Hz), 7.14-7.09 (m, 4H), 7.00 (t, 1H, J=7.6 Hz), 6.89 (t, 1H, J=7.4 Hz), 4.05 (s, 2H), 4.01 (s, 2H), 3.49 (s, 2H), 2.75 (d, 2H, J=11.5 Hz), 2.39 (s, 3H), 1.83-1.74 (m, 3H), 1.50-1.23 (m, 4H); MS (ESI) m/z 486.2 (M⁺+1).

### Example 52

### Synthesis of Compound 623

### Step 1: (Formula 31) Methyl 4-((2-methyl-1-((1-(pyridin-2-ylmethyl)piperidin-4-yl)methyl)-1H-indol-3-yl)methyl)benzoate

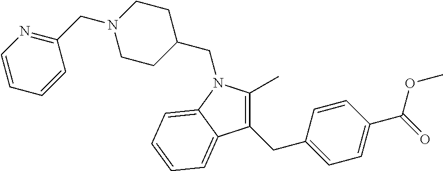 [SMILES: COC(=O)c1ccc(Cc2c(C)n(CC3CCN(Cc4ccccn4)CC3)c3ccccc23)cc1]

Methyl 4-((2-methyl-1-(piperidin-4-ylmethyl)-1H-indol-3-yl)methyl)benzoate hydrochloride (0.150 g, 0.36 mmol) was dissolved in methanol (3 mL). At room temperature, 2-pyridinecarboxylaldehyde (0.041 mL, 0.44 mmol) and acetic acid (0.031 mL, 0.55 mmol) were added to the solution, followed by stirring for 1 hour. Sodium triacetoxyborohydride (0.092 g, 0.44 mmol) was added to the reaction solution, followed by stirring at 60° C. for 15 hours. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methylene chloride/methanol=from 0% to 20%) and concentrated to afford the title compound (0.095 g, 56%) as a colorless liquid.

### Step 2: (Compound 623) N-hydroxy-4-((2-methyl-1-((1-(pyridin-2-ylmethyl)piperidin-4-yl)methyl)-1H-indol-3-yl)methyl)benzamide

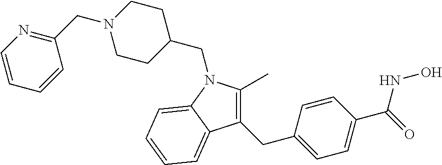 [SMILES: Cc1c(Cc2ccc(C(=O)NO)cc2)c2ccccc2n1CC1CCN(Cc2ccccn2)CC1]

Methyl 4-((2-methyl-1-((1-(pyridin-2-ylmethyl)piperidin-4-yl)methyl)-1H-indol-3-yl)methyl)benzoate (0.033 g, 0.07 mmol), hydroxylamine (50 wt % aqueous solution, 0.302 mL, 4.94 mmol) and potassium hydroxide (0.040 g, 0.71 mmol) were dissolved in methanol (1 mL)/tetrahydrofuran (1 mL) at room temperature, and the solution was stirred at the same temperature for 1 hour. Then, the reaction mixture was concentrated under reduced pressure to remove the solvent, and a saturated aqueous solution of sodium hydrogen carbonate was added to the concentrate. The precipitated solid was filtered, then dried to afford desired compound 623 (0.091 g, 96%) as a light yellow solid.

¹H-NMR (400 MHz, DMSO-d₆) d 8.47-8.45 (m, 1H), 7.75 (td, 1H, J=7.7, 0.9 Hz), 7.59 (d, 2H, J=8.2 Hz), 7.40 (dd, 2H, J=10.2, 3.6 Hz), 7.35 (d, 1H, J=7.7 Hz), 7.23 (dt, 1H, J=7.4, 4.9, 1.1 Hz), 7.14 (d, 2H, J=8.3 Hz), 7.02 (td, 1H, J=7.6, 0.9 Hz), 6.90 (td, 1H, J=7.4, 0.9 Hz), 4.03 (s, 2H), 4.01 (s, 2H), 3.53 (s, 2H), 2.79 (d, 2H, J=11.4 Hz), 2.36 (s, 3H), 1.90 (t, 2H, J=10.5 Hz), 1.79-1.74 (m, 1H), 1.45-1.30 (m, 4H); MS (ESI) m/z 469.2 (M⁺+1).

### Example 53

### Synthesis of Compound 624

### Step 1: (Formula 34) Methyl 4-((1-((1-(2-fluoro-2-methylpropyl)piperidin-4-yl)methyl)-2-(pyridin-4-yl)-1H-indol-3-yl)methyl)benzoate)

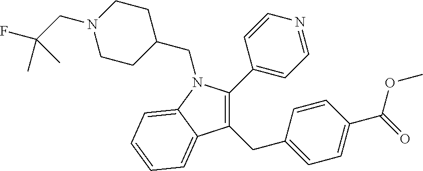 [SMILES: COC(=O)c1ccc(Cc2c(-c3ccncc3)n(CC3CCN(CC(C)(C)F)CC3)c3ccccc23)cc1]

Methyl 4-((2-bromo-1-((1-(2-fluoro-2-methylpropyl)piperidin-4-yl)methyl)-1H-indol-3-yl)methyl)benzoate (0.130 g, 0.25 mmol), 4-pyridineboronic acid (0.040 g, 0.33 mmol) and Pd(dppf)Cl₂ (0.021 g, 0.03 mmol) were mixed with 1,4-dioxane (3 mL) at room temperature. To the mixture, sodium carbonate (2.0 M aqueous solution, 1.009 mL, 2.02 mmol) was added, followed by stirring at 110° C. for 16 hours. Then, a saturated aqueous solution of sodium hydrogen carbonate was added to the reaction mixture, followed by extraction with ethyl acetate. The organic layer was dried with anhydrous magnesium sulfate, and then concentrated under reduced pressure. The concentrate was purified by column chromatography (SiO₂, 12 g cartridge; ethyl acetate/hexane=from 50% to 100%) and concentrated to afford the title compound (0.072 g, 56%) as a yellow liquid.

### Step 2: (Compound 624) 4-((1-((1-(2-Fluoro-2-methylpropyl)piperidin-4-yl)methyl)-2-(pyridin-4-yl)-1H-indol-3-yl)methyl)-N-hydroxybenzamide

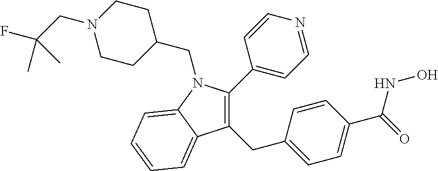 [SMILES: CC(C)(F)CN1CCC(Cn2c(-c3ccncc3)c(Cc3ccc(C(=O)NO)cc3)c3ccccc32)CC1]

Methyl 4-((1-((1-(2-fluoro-2-methylpropyl)piperidin-4-yl)methyl)-2-(pyridin-4-yl)-1H-indol-3-yl)methyl)benzoate (0.072 g, 0.14 mmol) was mixed with tetrahydrofuran (3 mL)/methanol (7 mL) at room temperature. To the mixture, hydroxylamine (50 wt % aqueous solution, 1.286 mL, 21.03 mmol) was added, and then potassium hydroxide (0.079 g, 1.40 mmol) was added, followed by stirring at the same temperature for 16 hours. Then, the reaction mixture was concentrated under reduced pressure. To the concentrate, a saturated aqueous solution of sodium hydrogen carbonate (3 mL) was added, followed by stirring. The precipitated solid was filtered, washed with water, and then dried to afford compound 624 (0.057 g, 79%) as a brown solid.

¹H NMR (400 MHz, DMSO-d₆) δ 8.73 (d, 2H, J=5.6 Hz), 7.61-7.55 (m, 3H), 7.48 (d, 2H, J=5.7 Hz), 7.41 (d, 1H, J=8.2 Hz), 7.20 (t, 1H, J=7.5 Hz), 7.06-7.00 (m, 3H), 4.09 (d, 2H, J=7.3 Hz), 4.04 (s, 2H), 2.70-2.67 (m, 2H), 2.26 (d, 2H, J=23.0 Hz), 1.81-1.75 (m, 2H), 1.45 (m, 1H), 1.23 (s, 3H), 1.21-1.13 (m, 5H), 0.96-0.91 (m, 2H); MS (ESI) m/z 515.2 (M⁺+H).

### Example 54

### Synthesis of Compound 625

### Step 1: (Formula 34) Methyl 4-((1-((1-(2-fluoro-2-methylpropyl)piperidin-4-yl)methyl)-2-(pyrimidin-5-yl)-1H-indol-3-yl)methyl)benzoate

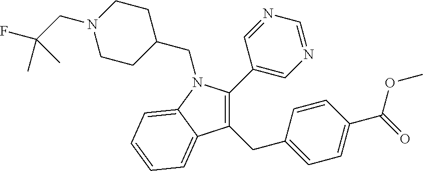 [SMILES: COC(=O)c1ccc(Cc2c(-c3cncnc3)n(CC3CCN(CC(C)(C)F)CC3)c3ccccc23)cc1]

Methyl 4-((2-bromo-1-((1-(2-fluoro-2-methylpropyl)piperidin-4-yl)methyl)-1H-indol-3-yl)methyl)benzoate (0.130 g, 0.25 mmol), 5-pyrimidineboronic acid (0.041 g, 0.33 mmol) and Pd(dppf)Cl₂ (0.021 g, 0.03 mmol) were added to 1,4-dioxane (3 mL) at room temperature. To the mixture, sodium carbonate (2.0 M aqueous solution, 1.009 mL, 2.02 mmol) was added, followed by stirring at 110° C. for 16 hours. Then, a saturated aqueous solution of sodium hydrogen carbonate was added to the reaction mixture, followed by extraction with ethyl acetate. The organic layer was dried with anhydrous magnesium sulfate, and then concentrated under reduced pressure. The concentrate was purified by column chromatography (SiO₂, 12 g cartridge; ethyl acetate/hexane=from 50% to 100%) and concentrated to afford the title compound (0.086 g, 66%) as a yellow solid.

### Step 2: (Compound 625) 4-((1-((1-(2-Fluoro-2-methylpropyl)piperidin-4-yl)methyl)-2-(pyrimidin-5-yl)-1H-indol-3-yl)methyl)-N-hydroxybenzamide

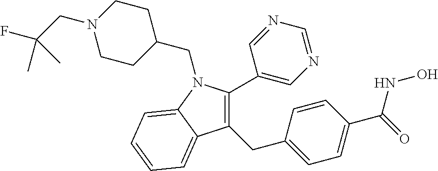 [SMILES: CC(C)(F)CN1CCC(Cn2c(-c3cncnc3)c(Cc3ccc(C(=O)NO)cc3)c3ccccc32)CC1]

Methyl 4-((1-((1-(2-fluoro-2-methylpropyl)piperidin-4-yl)methyl)-2-(pyrimidin-5-yl)-1H-indol-3-yl)methyl)benzoate (0.086 g, 0.17 mmol) was mixed with tetrahydrofuran (3 mL)/methanol (7 mL) at room temperature. To the mixture, hydroxylamine (50 wt % aqueous solution, 1.533 mL, 25.07 mmol) was added, and then potassium hydroxide (0.094 g, 1.67 mmol) was added, followed by stirring at the same temperature for 16 hours. Then, the reaction mixture was concentrated under reduced pressure. To the concentrate, a saturated aqueous solution of sodium hydrogen carbonate (3 mL) was added, followed by stirring. The precipitated solid was filtered, washed with water, and then dried to afford compound 625 (0.072 g, 84%) as a light brown solid.

¹H NMR (400 MHz, DMSO-d₆) δ 9.30 (s, 1H), 8.93 (s, 2H), 7.61 (d, 1H, J=8.2 Hz), 7.55 (d, 2H, J=7.9 Hz), 7.47 (d, 1H, J=8.0 Hz), 7.22 (t, 1H, J=7.7 Hz), 7.06-7.02 (m, 3H), 4.03-4.01 (m, 4H), 2.72-2.69 (m, 2H), 2.28 (d, 2H, J=22.8 Hz), 1.83-1.78 (m, 2H), 1.47 (m, 1H), 1.25 (s, 3H), 1.22-1.19 (m, 5H), 1.01-0.98 (m, 2H); MS (ESI) m/z 516.2 (M⁺+H).

### Example 55

### Synthesis of Compound 626

### Step 1: (Formula 34) Methyl 4-((2-(3,5-difluorophenyl)-1-((1-(2-fluoro-2-methylpropyl)piperidin-4-yl)methyl)-1H-indol-3-yl)methyl)benzoate

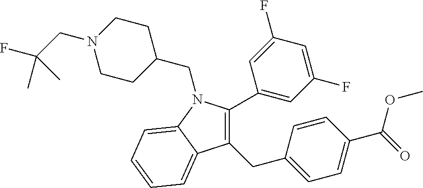 [SMILES: COC(=O)c1ccc(Cc2c(-c3cc(F)cc(F)c3)n(CC3CCN(CC(C)(C)F)CC3)c3ccccc23)cc1]

Methyl 4-((2-bromo-1-((1-(2-fluoro-2-methylpropyl)piperidin-4-yl)methyl)-1H-indol-3-yl)methyl)benzoate (0.130 g, 0.25 mmol), 3,5-difluorophenylboronic acid (0.052 g, 0.33 mmol) and Pd(dppf)Cl₂ (0.021 g, 0.03 mmol) were added to 1,4-dioxane (3 mL) at room temperature. To the mixture, sodium carbonate (2.0 M aqueous solution, 1.009 mL, 2.02 mmol) was added, followed by stirring at 110° C. for 16 hours. Then, a saturated aqueous solution of sodium hydrogen carbonate was added to the reaction mixture, followed by extraction with ethyl acetate. The organic layer was dried with anhydrous magnesium sulfate, and then concentrated under reduced pressure. The concentrate was purified by column chromatography (SiO₂, 12 g cartridge; ethyl acetate/hexane=from 20% to 50%) and concentrated to afford the title compound (0.082 g, 59%) as a yellow liquid.

### Step 2: (Compound 626) 4-((2-(3,5-Difluorophenyl)-1-((1-(2-fluoro-2-methylpropyl)piperidin-4-yl)methyl)-1H-indol-3-yl)methyl)-N-hydroxybenzamide

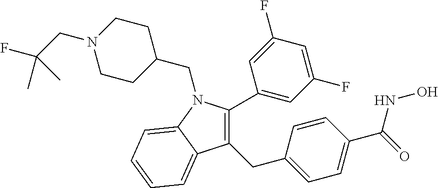 [SMILES: CC(C)(F)CN1CCC(Cn2c(-c3cc(F)cc(F)c3)c(Cc3ccc(C(=O)NO)cc3)c3ccccc32)CC1]

Methyl 4-((2-(3,5-difluorophenyl)-1-((1-(2-fluoro-2-methylpropyl)piperidin-4-yl)methyl)-1H-indol-3-yl)methyl)benzoate (0.082 g, 0.15 mmol) was mixed with tetrahydrofuran (3 mL)/methanol (7 mL) at room temperature. To the mixture, hydroxylamine (50% aqueous solution, 1.371 mL, 22.42 mmol) was added, and then potassium hydroxide (0.084 g, 1.50 mmol) was added, followed by stirring at the same temperature for 16 hours. Then, the reaction mixture was concentrated under reduced pressure. To the concentrate, a saturated aqueous solution of sodium hydrogen carbonate (3 mL) was added, followed by stirring. The precipitated solid was filtered, washed with water, and then dried to afford compound 626 (0.069 g, 84%) as a light brown solid.

¹H NMR (400 MHz, DMSO-d₆) δ 7.58-7.55 (m, 3H), 7.41-7.37 (m, 2H), 7.22-7.17 (m, 3H), 7.04-6.99 (m, 3H), 4.05 (d, 2H, J=7.4 Hz), 4.01 (s, 2H), 2.72-2.69 (m, 2H), 2.28 (d, 2H, J=23.0 Hz), 1.83-1.77 (m, 2H), 1.47 (m, 1H), 1.25 (s, 3H), 1.19-1.13 (m, 5H), 1.01-0.93 (m, 2H); MS (ESI) m/z 550.2 (M⁺+H).

### Example 56

### Synthesis of Compound 627

### Step 1: (Formula 34) Methyl 4-((2-(3,6-dihydro-2H-pyran-4-yl)-1-((1-(2-fluoro-2-methylpropyl)piperadin-4-yl)methyl)-1H-indol-3-yl)methyl)benzoate

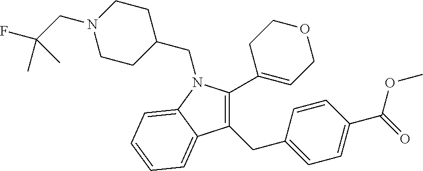 [SMILES: COC(=O)c1ccc(Cc2c(C3=CCOCC3)n(CC3CCN(CC(C)(C)F)CC3)c3ccccc23)cc1]

Methyl 4-((2-bromo-1-((1-(2-fluoro-2-methylpropyl)piperidin-4-yl)methyl)-1H-indol-3-yl)methyl)benzoate (0.130 g, 0.25 mmol), 2-(3,6-dihydro-2H-pyran-4-yl)-4,4,5,5-tetramethyl-1,3,2-dioxaborolan (0.069 g, 0.33 mmol) and Pd(dppf)Cl₂ (0.021 g, 0.03 mmol) were added to 1,4-dioxane (3 mL) at room temperature. To the mixture, sodium carbonate (2.0 M aqueous solution, 1.009 mL, 2.02 mmol) was added, followed by stirring at 110° C. for 16 hours. Then, a saturated aqueous solution of sodium hydrogen carbonate was added to the reaction mixture, followed by extraction with ethyl acetate. The organic layer was dried with anhydrous magnesium sulfate, and then concentrated under reduced pressure. The concentrate was purified by column chromatography (SiO₂, 12 g cartridge; ethyl acetate/hexane=from 20% to 60%) and concentrated to afford the title compound (0.105 g, 80%) as a yellow liquid.

### Step 2: (Compound 627) 4-((2-(3,6-Dihydro-2H-pyran-4-yl)-1-((1-(2-fluoro-2-methylpropyl)piperidin-4-yl)methyl)-1H-indol-3-yl)methyl)-N-hydroxybenzamide

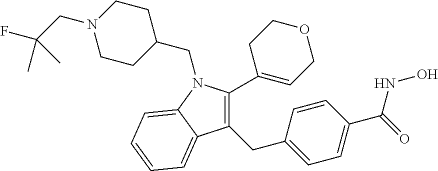 [SMILES: CC(C)(F)CN1CCC(Cn2c(C3=CCOCC3)c(Cc3ccc(C(=O)NO)cc3)c3ccccc32)CC1]

Methyl 4-((2-(3,6-dihydro-2H-pyran-4-yl)-1-((1-(2-fluoro-2-methylpropyl)piperidin-4-yl)methyl)-1H-indol-3-yl)methyl)benzoate (0.105 g, 0.20 mmol) was mixed with tetrahydrofuran (3 mL)/methanol (7 mL) at room temperature. To the mixture, hydroxylamine (50 wt % aqueous solution, 1.857 mL, 30.37 mmol) was added, and then potassium hydroxide (0.114 g, 2.02 mmol) was added, followed by stirring at the same temperature for 16 hours. Then, the reaction mixture was concentrated under reduced pressure. To the concentrate, a saturated aqueous solution of sodium hydrogen carbonate (3 mL) was added, followed by stirring. The precipitated solid was filtered, washed with water, and then dried to afford compound 627 (0.092 g, 88%) as a light brown solid.

¹H NMR (400 MHz, DMSO-d₆) δ 7.60 (d, 2H, J=8.0 Hz), 7.45 (d, 1H, J=8.1 Hz), 7.33 (d, 1H, J=7.8 Hz), 7.17 (d, 2H, J=7.6 Hz), 7.09 (t, 1H, J=7.3 Hz), 6.94 (t, 1H, J=7.5 Hz), 5.92 (s, 1H), 4.25 (s, 2H), 4.03 (s, 2H), 3.99 (d, 2H, J=7.4 Hz), 3.80 (t, 2H, J=4.8 Hz), 2.84-2.81 (m, 2H), 2.35 (d, 2H, J=22.9 Hz), 2.23 (brs, 2H), 1.94-1.89 (m, 2H), 1.73 (m, 1H), 1.30-1.15 (m, 10H); MS (ESI) m/z 520.2 (M⁺+H).

### Example 57

### Synthesis of Compound 631

### Step 1: (Formula 31) Methyl 4-((1-((1-(3-fluorobenzyl)piperidin-4-yl)methyl)-2-methyl-1H-indol-3-yl)methyl)benzoate

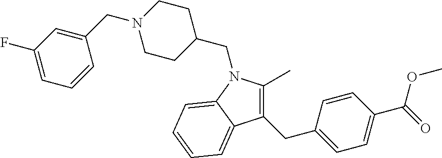 [SMILES: COC(=O)c1ccc(Cc2c(C)n(CC3CCN(Cc4cccc(F)c4)CC3)c3ccccc23)cc1]

Methyl 4-((2-methyl-1-(piperidin-4-ylmethyl)-1H-indol-3-yl)methyl)benzoate hydrochloride (0.150 g, 0.36 mmol), 1-(bromomethyl)-3-fluorobenzene (0.082 g, 0.44 mmol) and diisopropylethylamine (0.141 g, 1.09 mmol) were added to methylene chloride (2 mL) at room temperature, and the mixture was stirred at the same temperature for 0.5 hours. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 0% to 20%) and concentrated to afford the title compound (0.109 g, 62%) as a colorless liquid.

### Step 2: (Compound 631) 4-((1-((1-(3-Fluorobenzyl)piperidin-4-yl)methyl)-2-methyl-1H-indol-3-yl)methyl)-N-hydroxybenzamide

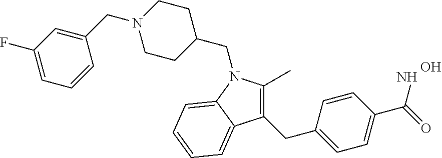

Methyl 4-((1-((1-(3-fluorobenzyl)piperidin-4-yl)methyl)-2-methyl-1H-indol-3-yl)methyl)benzoate (0.109 g, 0.23 mmol), hydroxylamine (50 wt % aqueous solution, 0.963 mL, 15.75 mmol) and potassium hydroxide (0.126 g, 2.25 mmol) were added to methanol (1 mL)/tetrahydrofuran (1 mL) at room temperature, and the mixture was stirred at the same temperature for 1 hour. Then, the reaction mixture was concentrated under reduced pressure to remove the solvent, and a saturated aqueous solution of sodium hydrogen carbonate was added to the concentrate. The precipitated solid was filtered, and then dried to afford compound 631 (0.105 g, 96%) as a white solid.

¹H-NMR (400 MHz, DMSO-d₆) δ 7.57 (d, 2H, J=7.8 Hz), 7.39-7.32 (m, 3H), 7.14-6.99 (m, 6H), 6.90 (t, 1H, J=7.4 Hz), 4.02 (s, 2H), 4.01 (s, 2H), 3.43 (s, 2H), 2.77 (d, 2H, J=10.8 Hz), 2.40 (s, 3H), 1.84 (t, 2H, J=11.1 Hz), 1.81-1.72 (m, 1H), 1.45-1.24 (m, 4H); MS (ESI) m/z 486.2 (M⁺+1).

### Example 58

### Synthesis of Compound 632

### Step 1: (Formula 31) Methyl 4-((2-methyl-1-((1-(pyridin-3-ylmethyl)piperidin-4-yl)methyl)-1H-indol-3-yl)methyl)benzoate

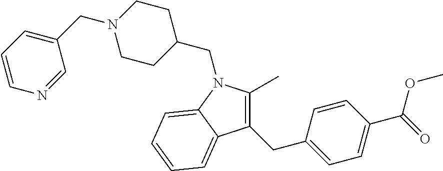 [SMILES: COC(=O)c1ccc(Cc2c(C)n(CC3CCN(Cc4cccnc4)CC3)c3ccccc23)cc1]

Methyl 4-((2-methyl-1-(piperidin-4-ylmethyl)-1H-indol-3-yl)methyl)benzoate hydrochloride (0.150 g, 0.36 mmol) and 3-(chloromethyl)pyridine hydrochloride (0.071 g, 0.44 mmol) were added to methylene chloride (2 mL) at room temperature, and the mixture was stirred at the same temperature for 0.5 hours. Then, water was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 10%) and concentrated to afford the title compound (0.058 g, 34%) as a yellow liquid.

### Step 2: (Compound 632) N-hydroxy-4-((2-methyl-1-((1-(pyridin-3-ylmethyl)piperidin-4-yl)methyl)-1H-indol-3-yl)methyl)benzamide

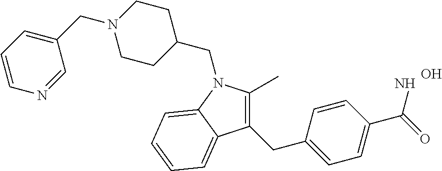

Methyl 4-((2-methyl-1-((1-(pyridin-3-ylmethyl)piperidin-4-yl)methyl)-1H-indol-3-yl)methyl)benzoate (0.058 g, 0.12 mmol), hydroxylamine (50 wt % aqueous solution, 0.531 mL, 8.68 mmol) and potassium hydroxide (0.070 g, 1.24 mmol) were added to methanol (1 mL)/tetrahydrofuran (1 mL) at room temperature, and the mixture was stirred at the same temperature for 1 hour. Then, the reaction mixture was concentrated under reduced pressure to remove the solvent, and a saturated aqueous solution of sodium hydrogen carbonate was added to the concentrate, followed by stirring. The precipitated solid was filtered and dried to afford compound 632 (0.048 g, 83%) as a light yellow solid.

¹H-NMR (400 MHz, DMSO-d₆) δ 8.45 (d, 2H, J=6.3 Hz), 7.67 (d, 1H, J=7.8 Hz), 7.58 (d, 2H, J=8.2 Hz), 7.38-7.30 (m, 3H), 7.15 (d, 2H, J=8.2 Hz), 7.01 (t, 1H, J=7.3 Hz), 6.90 (t, 1H, J=7.4 Hz), 4.01 (s, 2H), 4.00 (s, 2H), 3.44 (s, 2H), 2.39 (s, 3H), 1.84 (t, 2H, J=10.5 Hz), 1.79-1.72 (m, 1H), 1.45-1.24 (m, 4H); MS (ESI) m/z 469.2 (M⁺+1).

### Example 59

### Synthesis of Compound 633

### Step 1: (Formula 17b) Methyl 4-((1-(2-((3S,5R)-3,5-dimethylpiperazin-1-yl)ethyl)-2-methyl-1H-indol-3-yl)methyl)benzoate

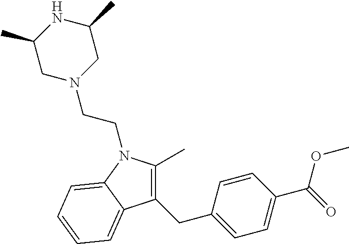

Methyl 4-((2-methyl-1-(2-(methylsulfonyloxy)ethyl)-1H-indol-3-yl)methyl)benzoate (1.000 g, 2.49 mmol), (2S,6R)-tert-butyl 2,6-dimethylpiperazine-1-carboxylate (2.669 g, 12.45 mmol) and diisopropylethylamine (2.205 mL, 12.45 mmol) were added to acetonitrile (5 mL), and heated by microwave irradiation at 120° C. for 2 hours, followed by cooling to room temperature. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; methylene chloride/methanol=from 0% to 10%) and concentrated to afford the title compound (1.250 g, 119%) as a yellow liquid.

### Step 2: (Formula 22) Methyl 4-((1-(2-((3S,5R)-4-(2-hydroxy-2-methylpropyl)-3,5-dimethylpiperazin-1-yl)ethyl)-2-methyl-1H-indol-3-yl)methyl)benzoate

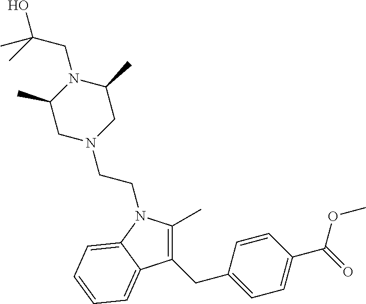 [SMILES: COC(=O)c1ccc(Cc2c(C)n(CCN3C[C@@H](C)N(CC(C)(C)O)[C@@H](C)C3)c3ccccc23)cc1]

Methyl 4-((1-(2-((3S,5R)-3,5-dimethylpiperazin-1-yl)ethyl)-2-methyl-1H-indol-3-yl)methyl)benzoate (0.100 g, 0.22 mmol), 2,2-dimethyloxirane (0.158 g, 2.19 mmol) and potassium carbonate (0.303 g, 2.19 mmol) were added to ethanol (4 mL), and heated by microwave irradiation at 110° C. for 1 hour, followed by cooling to room temperature. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 0% to 10%) and concentrated to afford the title compound (0.094 g, 85%) as a light yellow liquid.

### Step 3: (Formula 23) Methyl 4-((1-(2-((3S,5R)-4-(2-fluoro-2-methylpropyl)-3,5-dimethylpiperazin-1-yl)ethyl)-2-methyl-1H-indol-3-yl)methyl)benzoate

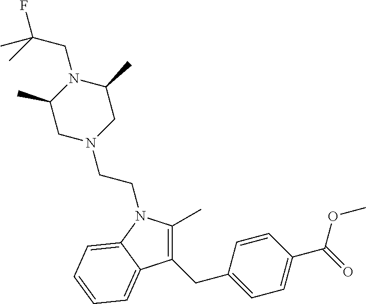 [SMILES: COC(=O)c1ccc(Cc2c(C)n(CCN3C[C@@H](C)N(CC(C)(C)F)[C@@H](C)C3)c3ccccc23)cc1]

4-((1-(2-((3S,5R)-4-(2-hydroxy-2-methylpropyl)-3,5-dimethylpiperazin-1-yl)ethyl)-2-methyl-1H-indol-3-yl)methyl)benzoate (0.094 g, 0.19 mmol) and diethylaminosulfur trifluoride (0.037 mL, 0.28 mmol) were added to methylene chloride (2 mL) at room temperature, and the mixture was stirred at the same temperature for 1 hour. Then, water was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 10%) and concentrated to afford the title compound (0.019 g, 20%) as a colorless liquid.

### Step 4: (Compound 633) 4-((1-(2-((3S,5R)-4-(2-fluoro-2-methylpropyl)-3,5-dimethylpiperazin-1-yl)ethyl)-2-methyl-1H-indol-3-yl)methyl)-N-hydroxybenzamide

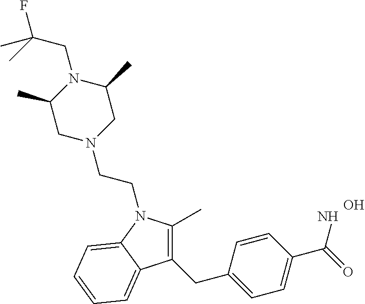

Methyl 4-((1-(2-((3S,5R)-4-(2-fluoro-2-methylpropyl)-3,5-dimethylpiperazin-1-yl)ethyl)-2-methyl-1H-indol-3-yl)methyl)benzoate (0.019 g, 0.04 mmol), hydroxylamine (50 wt % aqueous solution, 0.160 mL, 2.62 mmol) and potassium hydroxide (0.021 g, 0.37 mmol) were added to methanol (1 mL)/tetrahydrofuran (1 mL) at room temperature, and the mixture was stirred at the same temperature for 1 hour. Then, the reaction mixture was concentrated under reduced pressure to remove the solvent, and a saturated aqueous solution of sodium hydrogen carbonate was added to the concentrate. The precipitated solid was filtered, and then dried to afford compound 633 (0.014 g, 76%) as a white solid.

¹H-NMR (400 MHz, DMSO-d₆) δ 7.57 (d, 2H, J=8.2 Hz), 7.34 (dd, 2H, J=7.6, 5.4 Hz), 7.12 (d, 2H, J=8.0 Hz), 7.03 (t, 1H, J=7.5 Hz), 6.91 (t, 1H, J=7.4 Hz), 4.21 (t, 2H, J=6.8 Hz), 4.00 (s, 2H), 2.68-2.63 (m, 4H), 2.57-2.45 (m, 4H), 2.43 (s, 3H), 1.88 (t, 2H, J=10.1 Hz), 1.29 (s, 3H), 1.24 (s, 3H), 0.95 (d, 6H, J=6.2 Hz); MS (ESI) m/z 495.2 (M⁺+1).

### Example 60

### Synthesis of Compound 639

### Step 1: (Formula 25) Methyl 4-((1-(4-bromobutyl)-2-methyl-1H-indol-3-yl)methyl)benzoate

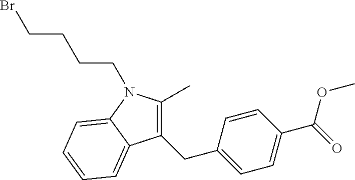 [SMILES: COC(=O)c1ccc(Cc2c(C)n(CCCCBr)c3ccccc23)cc1]

1,4-dibromobutane (1.283 mL, 10.74 mmol) was mixed with N,N-dimethylformamide (10 mL) at room temperature. To the mixture, methyl 4-((2-methyl-1H-indol-3-yl)methyl)benzoate (1.000 g, 3.58 mmol) and sodium hydride (60%, 0.143 g, 3.58 mmol) were added, followed by stirring at the same temperature for 1 hour. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 0% to 20%) and concentrated to afford the title compound (1.240 g, 84%) as a yellow liquid.

### Step 2: (Formula 26) Tert-butyl 4-(4-(3-(4-(methoxycarbonyl)benzyl)-2-methyl-1H-indol-1-yl)butyl)piperazine-1-carboxylate

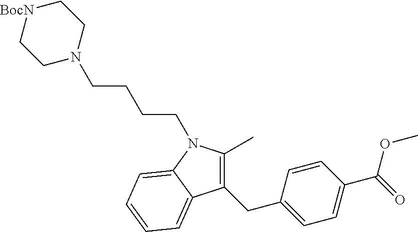 [SMILES: COC(=O)c1ccc(Cc2c(C)n(CCCCN3CCN(C(=O)OC(C)(C)C)CC3)c3ccccc23)cc1]

Methyl 4-((1-(4-bromobutyl)-2-methyl-1H-indol-3-yl)methyl)benzoate (0.500 g, 1.21 mmol), tert-butyl piperazine-1-carboxylate (0.337 g, 1.81 mmol) and diisopropylethylamine (0.624 mL, 3.62 mmol) were added to acetonitrile (5 mL) at room temperature, and the mixture was stirred at the same temperature for 1 hour. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 0% to 60%) and concentrated to afford the title compound (0.443 g, 71%) as a colorless liquid.

### Step 3: (Formula 27) Methyl 4-((2-methyl-1-(4-(piperazin-1-yl)butyl)-1H-indol-3-yl)methyl)benzoate hydrochloride

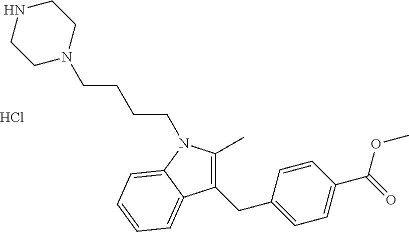 [SMILES: Cl ; COC(=O)c1ccc(Cc2c(C)n(CCCCN3CCNCC3)c3ccccc23)cc1]

Tert-butyl 4-(4-(3-(4-(methoxycarbonyl)benzyl)-2-methyl-1H-indol-1-yl)butyl)piperazine-1-carboxylate (0.443 g, 0.85 mmol) and hydrochloric acid (4.0 M, 1,4-dioxane solution, 2.131 mL, 8.53 mmol) were added to 1,4-dioxane (1 mL) at room temperature, and the mixture was stirred at the same temperature for 1 hour. Then, the reaction mixture was concentrated under reduced pressure. To the concentrate, diethyl ether (10 mL) was added, followed by stirring. The precipitated solid was filtered, and then dried to afford compound (0.288 g, 69%) as a light pink solid.

### Step 4: (Formula 28) Methyl 4-((1-(4-(4-(2-hydroxy-2-methylpropyl)piperazin-1-yl)butyl)-2-methyl-1H-indol-3-yl)methyl)benzoate

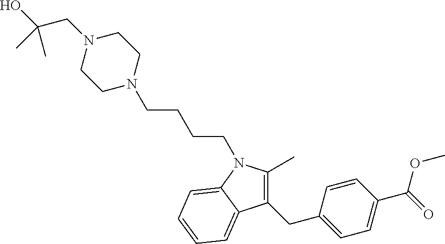 [SMILES: COC(=O)c1ccc(Cc2c(C)n(CCCCN3CCN(CC(C)(C)O)CC3)c3ccccc23)cc1]

Methyl 4-((2-methyl-1-(4-(piperazin-1-yl)butyl)-1H-indol-3-yl)methyl)benzoate hydrochloride (0.288 g, 0.59 mmol), 2,2-dimethyloxirane (0.521 mL, 5.85 mmol) and potassium carbonate (0.808 g, 5.85 mmol) were added to ethanol (3 mL), and heated by microwave irradiation at 120° C. for 20 minutes, followed by cooling to room temperature. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 10%) and concentrated to afford the title compound (0.229 g, 80%) as a yellow liquid.

### Step 5: (Formula 29) Methyl 4-((1-(4-(4-(2-fluoro-2-methylpropyl)piperazin-1-yl)butyl)-2-methyl-1H-indol-3-yl)methyl)benzoate

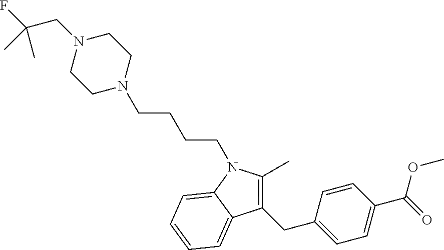 [SMILES: COC(=O)c1ccc(Cc2c(C)n(CCCCN3CCN(CC(C)(C)F)CC3)c3ccccc23)cc1]

Methyl 4-((1-(4-(4-(2-hydroxy-2-methylpropyl)piperazin-1-yl)butyl)-2-methyl-1H-indol-3-yl)methyl)benzoate (0.150 g, 0.31 mmol) and diethylaminosulfur trifluoride (0.060 mL, 0.46 mmol) were added to methylene chloride (2 mL) at room temperature, and the mixture was stirred at the same temperature for 2 hours. Then, water was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 10%) and concentrated to afford the title compound (0.095 g, 63%) as a yellow liquid.

### Step 6: (Compound 639) 4-((1-(4-(4-(2-Fluoro-2-methylpropyl)piperazin-1-yl)butyl)-2-methyl-1H-indol-3-yl)methyl)-N-hydroxybenzamide

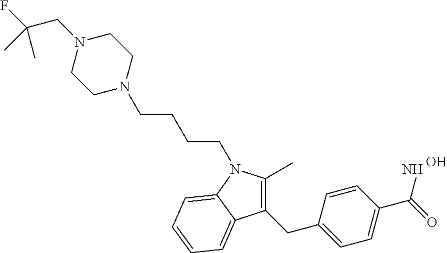

Methyl 4-((1-(4-(4-(2-fluoro-2-methylpropyl)piperazin-1-yl)butyl)-2-methyl-1H-indol-3-yl)methyl)benzoate (0.095 g, 0.19 mmol), hydroxylamine (50 wt % aqueous solution 0.824 mL, 13.47 mmol) and potassium hydroxide (0.108 g, 1.92 mmol) were added to tetrahydrofuran (1 mL)/methanol (1 mL) at room temperature, and the mixture was stirred at the same temperature for 1 hour. Then, the reaction mixture was concentrated under reduced pressure. To the concentrate, a saturated aqueous solution of sodium hydrogen carbonate (5 mL) was added, followed by stirring. The precipitated solid was filtered, and then dried to afford compound 639 (0.095 g, 100%) as an ivory solid.

¹H-NMR (400 MHz, DMSO-d₆) δ 7.59 (d, 2H, J=8.2 Hz), 7.39 (d, 1H, J=8.2 Hz), 7.36 (d, 1H, J=7.6 Hz), 7.20 (d, 2H, J=8.2 Hz), 7.03 (t, 1H, J=7.1 Hz), 6.91 (t, 1H, J=7.0 Hz), 4.12 (t, 2H, J=7.4 Hz), 4.04 (s, 2H), 2.44-2.42 (m, 4H), 2.41 (s, 3H), 2.36-2.29 (m, 6H), 2.25 (t, 2H, J=6.9 Hz), 1.69-1.61 (m, 2H), 1.47-1.40 (m, 2H), 1.32 (s, 3H), 1.26 (s, 3H); MS (ESI) m/z 495.2 (M⁺+1).

### Example 61

### Synthesis of Compound 640

### Step 1: (Formula 25) Methyl 4-((1-(3-bromopropyl)-2-methyl-1H-indol-3-yl)methyl)benzoate

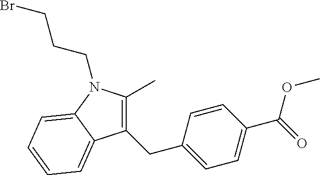 [SMILES: COC(=O)c1ccc(Cc2c(C)n(CCCBr)c3ccccc23)cc1]

1,3-dibromopropane (1.090 mL, 10.74 mmol) was dissolved in N,N-dimethylformamide (5 mL). At room temperature, sodium hydride (60%, 0.143 g, 3.58 mmol) was slowly added dropwise to the solution, and a starting material (1.000 g, 3.58 mmol) was added thereto, followed by stirring at the same temperature for hour. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 0% to 10%) and concentrated to afford the title compound (0.188 g, 13%) as a yellow liquid.

### Step 2: (Formula 26) Tert-butyl 4-(3-(3-(4-(methoxycarbonyl)benzyl)-2-methyl-1H-indol-1-yl)propyl)piperazine-1-carboxylate

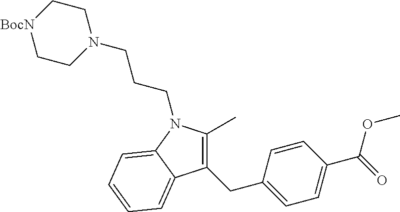 [SMILES: COC(=O)c1ccc(Cc2c(C)n(CCCN3CCN(C(=O)OC(C)(C)C)CC3)c3ccccc23)cc1]

Methyl 4-((1-(3-bromopropyl)-2-methyl-1H-indol-3-yl)methyl)benzoate (0.188 g, 0.47 mmol), tert-butyl piperazine-1-carboxylate (0.131 g, 0.70 mmol) and diisopropylethylamine (0.243 mL, 1.41 mmol) were added to acetonitrile (5 mL) at room temperature, and the mixture was stirred at the same temperature for 1 hour. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 0% to 20%) and concentrated to afford the title compound (0.206 g, 87%) as a yellow liquid.

### Step 3: (Formula 27) Methyl 4-((2-methyl-1-(3-(piperazin-1-yl)propyl)-1H-indol-3-yl)methyl)benzoate hydrochloride

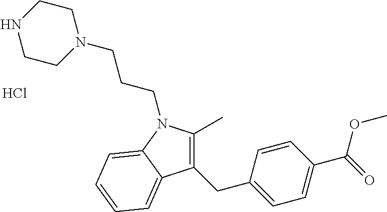 [SMILES: Cl ; COC(=O)c1ccc(Cc2c(C)n(CCCN3CCNCC3)c3ccccc23)cc1]

Tert-butyl 4-(3-(3-(4-(methoxycarbonyl)benzyl)-2-methyl-1H-indol-1-yl)propyl)piperazine-1-carboxylate (0.206 g, 0.41 mmol) and hydrochloric acid (4.0 M, 1,4-dioxane solution, 1.018 mL, 4.07 mmol) were added to 1,4-dioxane (1 mL) at room temperature, and the mixture was stirred at the same temperature for 1 hour. Then, the reaction mixture was concentrated under reduced pressure. To the concentrate, diethyl ether (10 mL) was added, followed by stirring. The precipitated solid was filtered, and then dried to afford compound (0.113 g, 66%) as a purple solid.

### Step 4: (Formula 28) Methyl 4-((1-(3-(4-(2-hydroxy-2-methylpropyl)piperazin-1-yl)propyl)-2-methyl-1H-indol-3-yl)methyl)benzoate

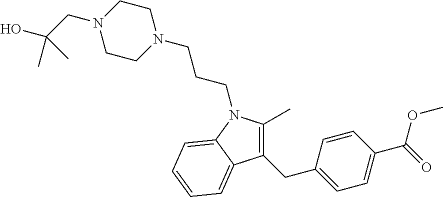 [SMILES: COC(=O)c1ccc(Cc2c(C)n(CCCN3CCN(CC(C)(C)O)CC3)c3ccccc23)cc1]

Methyl 4-((2-methyl-1-(3-(piperazin-1-yl)propyl)-1H-indol-3-yl)methyl)benzoate hydrochloride (0.113 g, 0.24 mmol), 2,2-dimethyloxirane (0.210 mL, 2.36 mmol) and potassium carbonate (0.326 g, 2.36 mmol) were added to ethanol (3 mL), and heated by microwave irradiation at 120° C. for 20 minutes, followed by cooling to room temperature. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 10%) and concentrated to afford the title compound (0.080 g, 69%) as a yellow liquid.

### Step 5: (Formula 29) Methyl 4-((1-(3-(4-(2-fluoro-2-methylpropyl)piperazin-1-yl)propyl)-2-methyl-1H-indol-3-yl)methyl)benzoate

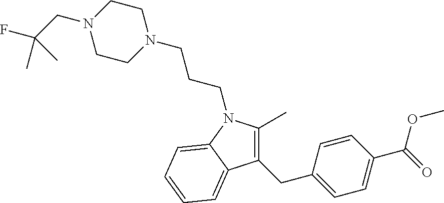 [SMILES: COC(=O)c1ccc(Cc2c(C)n(CCCN3CCN(CC(C)(C)F)CC3)c3ccccc23)cc1]

Methyl 4-((1-(3-(4-(2-hydroxy-2-methylpropyl)piperazin-1-yl)propyl)-2-methyl-1H-indol-3-yl)methyl)benzoate (0.080 g, 0.16 mmol) and diethylaminosulfur trifluoride (0.032 mL, 0.24 mmol) were added to methylene chloride (2 mL) at room temperature, and the mixture was stirred at the same temperature for 1 hour. Then, water was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 50%) and concentrated to afford the title compound (0.031 g, 39%) as a yellow liquid.

### Step 6: (Compound 640) 4-((1-(3-(4-(2-Fluoro-2-methylpropyl)piperazin-1-yl)propyl)-2-methyl-1H-indol-3-yl)methyl)-N-hydroxybenzamide

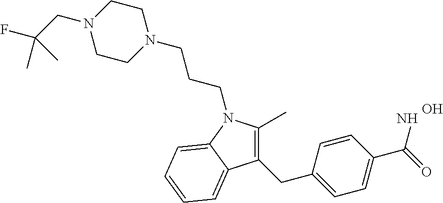

Methyl 4-((1-(3-(4-(2-fluoro-2-methylpropyl)piperazin-1-yl)propyl)-2-methyl-1H-indol-3-yl)methyl)benzoate (0.032 g, 0.07 mmol), hydroxylamine (50 wt % aqueous solution, 0.281 mL, 4.60 mmol) and potassium hydroxide (0.037 g, 0.66 mmol) were added to tetrahydrofuran (1 mL)/methanol (1 mL) at room temperature, and the mixture was stirred at the same temperature for 1 hour. Then, the reaction mixture was concentrated under reduced pressure. To the concentrate, a saturated aqueous solution of sodium hydrogen carbonate (5 mL) was added, followed by stirring. The precipitated solid was filtered, and then dried to afford compound 640 (0.019 g, 60%) as an ivory solid.

¹H-NMR (400 MHz, DMSO-d₆) d 7.58 (d, 2H, J=8.2 Hz), 7.36 (t, 2H, J=7.4 Hz), 7.17 (d, 2H, J=7.6 Hz), 7.02 (t, 1H, J=7.6 Hz), 6.91 (t, 1H, J=7.7 Hz), 4.15 (t, 2H, J=7.0 Hz), 4.02 (s, 2H), 2.45 (s, 2H), 2.42 (s, 3H), 2.39 (s, 2H), 2.35-2.33 (m, 6H), 2.19 (t, 2H, J=6.3 Hz), 1.80 (t, 2H, J=6.8 Hz), 1.33 (s, 3H), 1.27 (s, 3H); MS (ESI) m/z 481.2 (M⁺+1).

### Example 62

### Synthesis of Compound 643

### Step 1: (Formula 31) Methyl 4-((2-methyl-1-((1-((3-methyloxetan-3-yl)methyl)piperidin-4-yl)methyl)-1H-indol-3-yl)methyl)benzoate

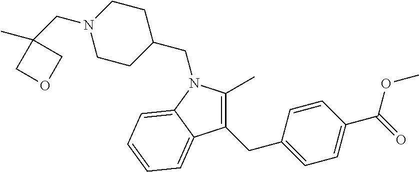

Methyl 4-((2-methyl-1-(piperidin-4-ylmethyl)-1H-indol-3-yl)methyl)benzoate hydrochloride (0.200 g, 0.48 mmol), (3-methyloxetan-3-yl)methyl 4-methylbenzenesulfonate (0.248 g, 0.97 mmol) and diisopropylethylamine (0.257 mL, 1.45 mmol) were added to acetonitrile (5 mL), and heated by microwave irradiation at 120° C. for 1 hour, followed by cooling to room temperature. Then, a saturated aqueous solution of sodium hydrogen carbonate was added to the reaction mixture, followed by extraction with ethyl acetate. The organic layer was dried with anhydrous magnesium sulfate, and then concentrated under reduced pressure. The concentrate was purified by column chromatography (SiO₂, 12 g cartridge; ethyl acetate/hexane=from 70% to 100%) and concentrated to afford the title compound (0.036 g, 16%) as a light yellow liquid.

### Step 2: (Compound 643) N-hydroxy-4-((2-methyl-1-((1-((3-methyloxetan-3-yl)methyl)piperidin-4-yl)methyl)-1H-indol-3-yl)methyl)benzamide

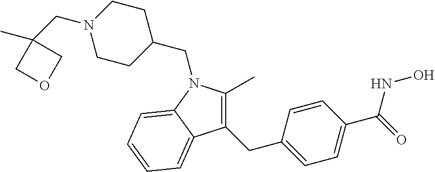 [SMILES: Cc1c(Cc2ccc(C(=O)NO)cc2)c2ccccc2n1CC1CCN(CC2(C)COC2)CC1]

Methyl 4-((2-methyl-1-((1-((3-methyloxetan-3-yl)methyl)piperidin-4-yl)methyl)-1H-indol-3-yl)methyl)benzoate (0.036 g, 0.08 mmol) was added to tetrahydrofuran (1 mL)/methanol (4 mL) at room temperature. To the mixture, hydroxylamine (50 wt % aqueous solution, 0.956 mL, 15.63 mmol) was added, and then potassium hydroxide (0.044 g, 0.78 mmol) was added, followed by stirring at the same temperature for 16 hours. Then, the reaction mixture was concentrated under reduced pressure. To the concentrate, a saturated aqueous solution of sodium hydrogen carbonate (3 mL) was added, followed by stirring. The precipitated solid was filtered, washed with water, and then dried to afford compound 643 (0.022 g, 61%) as a yellow solid.

¹H NMR (400 MHz, DMSO-d₆) δ 11.09 (brs, 1H), 8.99 (brs, 1H), 7.61 (d, 2H, J=8.0 Hz), 7.39-7.34 (m, 2H), 7.25 (d, 2H, J=8.0 Hz), 7.02 (t, 1H, J=7.6 Hz), 6.91 (t, 1H, J=7.3 Hz), 4.30 (d, 2H, J=5.6 Hz), 4.15 (d, 2H, J=5.5 Hz), 4.06 (s, 2H), 4.00 (d, 2H, J=7.4 Hz), 2.55 (m, 1H), 2.41 (s, 2H), 2.40 (s, 3H), 1.81-1.70 (m, 4H), 1.38-1.31 (m, 4H), 1.20 (s, 3H); MS (ESI) m/z 462.2 (M⁺+H).

### Example 63

### Synthesis of Compound 679

### Step 1: (Formula 42) Methyl 4-((1-((1-((4-fluoro-1-methylpiperidin-4-yl)methyl)piperidin-4-yl)methyl)-2-methyl-1H-indol-3-yl)methyl)benzoate

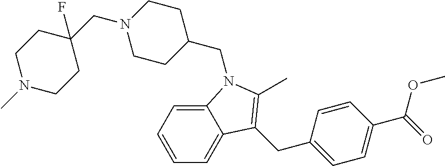

Methyl 4-((1-((1-((4-fluoropiperidin-4-yl)methyl)piperidin-4-yl)methyl)-2-methyl-1H-indol-3-yl)methyl)benzoate hydrochloride (0.130 g, 0.25 mmol) was added to methanol (3 mL) at room temperature. To the mixture, paraformaldehyde (0.037 g, 1.23 mmol) and acetic acid (0.042 mL, 0.74 mmol) were added, followed by stirring for 1 hour. Sodium cyanoborohydride (0.046 g, 0.74 mmol) was added to the reaction solution, followed by stirring at the same temperature for 16 hours. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 15%) and concentrated to afford the title compound (0.032 g, 26%) as a colorless liquid.

### Step 2: (Compound 679) 4-((1-((1-((4-Fluoro-1-methylpiperidin-4-yl)methyl)piperidin-4-yl)methyl)-2-methyl-1H-indol-3-yl)methyl)-N-hydroxybenzamide

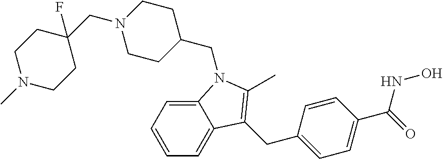 [SMILES: Cc1c(Cc2ccc(C(=O)NO)cc2)c2ccccc2n1CC1CCN(CC2(F)CCN(C)CC2)CC1]

Methyl 4-((1-((1-((4-fluoro-1-methylpiperidin-4-yl)methyl)piperidin-4-yl)methyl)-2-methyl-1H-indol-3-yl)methyl)benzoate (0.032 g, 0.06 mmol), hydroxylamine (50 wt % aqueous solution, 0.039 mL, 0.63 mmol) and potassium hydroxide (0.036 g, 0.63 mmol) were added to tetrahydrofuran (1 mL)/methanol (1 mL) at room temperature, and the mixture was stirred at the same temperature for 1 hour. Then, the reaction mixture was concentrated under reduced pressure. To the concentrate, a saturated aqueous solution of sodium hydrogen carbonate (5 mL) was added, followed by stirring. The precipitated solid was filtered, and then dried to afford compound 679 (0.017 g, 52%) as a white solid.

¹H-NMR (400 MHz, DMSO-d₆) δ 7.58 (d, 2H, J=8.2 Hz), 7.37 (d, 1H, J=8.6 Hz), 7.35 (d, 1H, J=7.6 Hz), 7.14 (d, 2H, J=8.2 Hz), 7.02 (t, 1H, J=7.5 Hz), 6.90 (t, 1H, J=7.4 Hz), 4.01 (s, 2H), 3.99 (s, 2H), 2.84 (d, 2H, J=11.8 Hz), 2.73-2.70 (m, 2H), 2.40 (s, 3H), 2.15 (s, 3H), 2.10-2.08 (m, 2H), 1.96 (t, 2H, J=10.3 Hz), 1.78-1.54 (m, 6H), 1.40-1.24 (m, 5H); MS (ESI) m/z 507.3 (M⁺+1).

### Example 64

### Synthesis of Compound 681

### Step 1: (Formula 42) Methyl 4-((1-((1-((4-fluoro-1-isopropylpiperidin-4-yl)methyl)piperidin-4-yl)methyl)-2-methyl-1H-indol-3-yl)methyl)benzoate

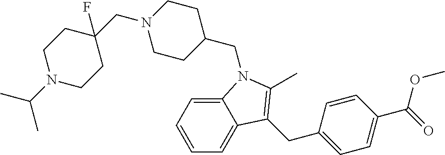 [SMILES: COC(=O)c1ccc(Cc2c(C)n(CC3CCN(CC4(F)CCN(C(C)C)CC4)CC3)c3ccccc23)cc1]

Methyl 4-((1-((1-((4-fluoropiperidin-4-yl)methyl)piperidin-4-yl)methyl)-2-methyl-1H-indol-3-yl)methyl)benzoate hydrochloride (0.130 g, 0.25 mmol) was added to methanol (3 mL) at room temperature. To the mixture, acetone (0.090 mL, 1.23 mmol) and acetic acid (0.042 mL, 0.74 mmol) were added, followed by stirring for 1 hour. Sodium cyanoborohydride (0.046 g, 0.74 mmol) was added to the reaction solution, followed by stirring at the same temperature for 16 hour. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; methanol/methylene chloride=from 0% to 15%) and concentrated to afford the title compound (0.022 g, 17%) as a colorless liquid.

### Step 2: (Compound 68) 4-((1-((1-((4-Fluoro-1-isopropylpiperidin-4-yl)methyl)piperidin-4-yl)methyl)-2-methyl-1H-indol-3-yl)methyl)-N-hydroxybenzamide

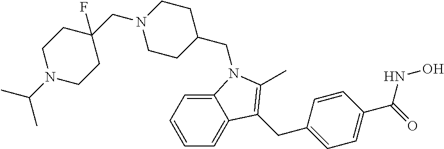 [SMILES: Cc1c(Cc2ccc(C(=O)NO)cc2)c2ccccc2n1CC1CCN(CC2(F)CCN(C(C)C)CC2)CC1]

Methyl 4-((1-((1-((4-fluoro-1-isopropylpiperidin-4-yl)methyl)piperidin-4-yl)methyl)-2-methyl-1H-indol-3-yl)methyl)benzoate (0.022 g, 0.04 mmol), hydroxylamine (50% aqueous solution, 0.025 mL, 0.41 mmol) and potassium hydroxide (0.023 g, 0.41 mmol) were added to tetrahydrofuran (1 mL)/methanol (1 mL) at room temperature, and the mixture was stirred at the same temperature for 1 hour. Then, the reaction mixture was concentrated under reduced pressure. To the concentrate, a saturated aqueous solution of sodium hydrogen carbonate (5 mL) was added, followed by stirring. The precipitated solid was filtered, and then dried to afford compound 681 (0.014 g, 64%) as a white solid.

¹H-NMR (400 MHz, DMSO-d₆) δ 7.57 (d, 2H, J=8.1 Hz), 7.38 (d, 1H, J=8.4 Hz), 7.35 (d, 1H, J=7.8 Hz), 7.13 (d, 2H, J=8.2 Hz), 7.01 (t, 1H, J=7.5 Hz), 6.90 (t, 1H, J=7.5 Hz), 4.01 (s, 2H), 3.99 (s, 2H), 2.84 (d, 2H, J=11.6 Hz), 2.69-2.64 (m, 2H), 2.39 (s, 3H), 2.35-2.30 (m, 4H), 1.96 (t, 2H, J=11.2 Hz), 1.78-1.46 (m, 6H), 1.40-1.22 (m, 4H), 0.95 (d, 2H, J=6.6 Hz); MS (ESI) m/z 535.3 (M⁺+1).

### Example 65

### Synthesis of Compound 695

### Step 1: (Formula 42) Methyl 4-((1-((1-((4-fluoro-1-(isopropylcarbamoyl)piperidin-4-yl)methyl)piperidin-4-yl)methyl)-2-methyl-1H-indol-3-yl)methyl)benzoate

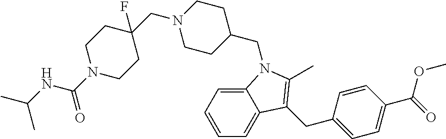 [SMILES: COC(=O)c1ccc(Cc2c(C)n(CC3CCN(CC4(F)CCN(C(=O)NC(C)C)CC4)CC3)c3ccccc23)cc1]

Methyl 4-((1-((1-((4-fluoropiperidin-4-yl)methyl)piperidin-4-yl)methyl)-2-methyl-1H-indol-3-yl)methyl)benzoate hydrochloride (0.100 g, 0.19 mmol), 2-isocyanatopropane (0.020 mL, 0.21 mmol) and triethylamine (0.053 mL, 0.38 mmol) were added to methylene chloride (3 mL) at room temperature, and the mixture was stirred at the same temperature for 2 hours. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 0% to 50%) and concentrated to afford the title compound (0.102 g, 93%) as a yellow liquid.

### Step 2: (Compound 695) 4-Fluoro-4-((4-((3-(4-(hydroxycarbamoyl)benzyl)-2-methyl-1H-indol-1-yl)methyl)piperidin-1-yl)methyl)-N-isopropylpiperidine-1-carboxamide

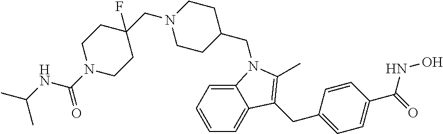 [SMILES: Cc1c(Cc2ccc(C(=O)NO)cc2)c2ccccc2n1CC1CCN(CC2(F)CCN(C(=O)NC(C)C)CC2)CC1]

Methyl 4-((1-((1-((4-fluoro-1-(isopropylcarbamoyl)piperidin-4-yl)methyl)piperidin-4-yl)methyl)-2-methyl-1H-indol-3-yl)methyl)benzoate (0.102 g, 0.18 mmol), hydroxylamine (50% aqueous solution, 0.541 mL, 8.84 mmol) and potassium hydroxide (0.099 g, 1.77 mmol) were added to methanol (1 mL) at room temperature, and the mixture was stirred at the same temperature for 0.5 hours. Then, the reaction mixture was concentrated under reduced pressure. To the concentrate, a saturated aqueous solution of sodium hydrogen carbonate (5 mL) was added, followed by stirring. The precipitated solid was filtered, and then dried to afford compound 695 (0.094 g, 92%) as a light ivory solid.

¹H-NMR (400 MHz, DMSO-d₆) δ 7.57 (d, 2H, J=8.0 Hz), 7.36 (t, 2H, J=8.5 Hz), 7.10 (d, 2H, J=7.8 Hz), 7.02 (t, 1H, J=7.4 Hz), 6.90 (t, 1H, J=7.4 Hz), 4.00 (s, 4H), 2.94 (t, 2H, J=10.9 Hz), 2.84 (d, 2H, J=11.0 Hz), 2.39 (s, 3H), 1.97 (t, 2H, J=10.8 Hz), 1.73-1.68 (m, 4H), 1.59-1.44 (m, 4H), 1.41-1.31 (m, 6H), 1.05 (d, 6H, J=6.5 Hz); MS (ESI) m/z 578.3 (M⁺+1).

### Example 66

### Synthesis of Compound 696

### Step 1: (Formula 42) Methyl 4-((1-((1-((4-fluoro-1-(methylsulfonyl)piperidin-4-yl)methyl)piperidin-4-yl)methyl)-2-methyl-1H-indol-3-yl)methyl)benzoate

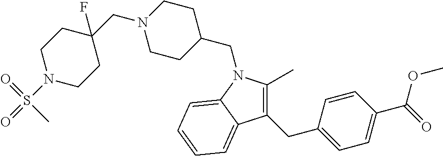 [SMILES: COC(=O)c1ccc(Cc2c(C)n(CC3CCN(CC4(F)CCN(S(C)(=O)=O)CC4)CC3)c3ccccc23)cc1]

Methyl 4-((1-((1-((4-fluoropiperidin-4-yl)methyl)piperidin-4-yl)methyl)-2-methyl-1H-indol-3-yl)methyl)benzoate hydrochloride (0.100 g, 0.19 mmol), methanesulfonyl chloride (0.024 g, 0.21 mmol) and triethylamine (0.052 mL, 0.38 mmol) were added to methylene chloride (3 mL) at room temperature, and the mixture was stirred at the same temperature for 2 hours. Then, water was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 0% to 50%) and concentrated to afford the title compound (0.106 g, 98%) as a yellow liquid.

### Step 2: (Compound 696) 4-((1-((1-((4-Fluoro-1-(methylsulfonyl)piperidin-4-yl)methyl)piperidin-4-yl)methyl)-2-methyl-1H-indol-3-yl)methyl)-N-hydroxybenzamide

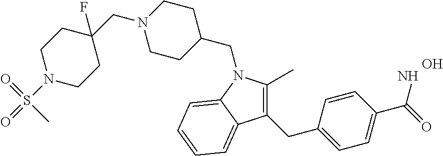

Methyl 4-((1-((1-((4-fluoro-1-(methylsulfonyl)piperidin-4-yl)methyl)piperidin-4-yl)methyl)-2-methyl-1H-indol-3-yl)methyl)benzoate (0.106 g, 0.19 mmol), hydroxylamine (50% aqueous solution, 0.569 mL, 9.30 mmol) and potassium hydroxide (0.104 g, 1.86 mmol) were added to methanol (1 mL) at room temperature, and the mixture was stirred at the same temperature for 30 minutes. Then, the reaction mixture was concentrated under reduced pressure. To the concentrate, a saturated aqueous solution of sodium hydrogen carbonate (5 mL) was added, followed by stirring. The precipitated solid was filtered, and then dried to afford compound 696 (0.101 g, 95%) as an ivory solid.

¹H-NMR (400 MHz, DMSO-d₆) δ 7.59 (d, 2H, J=8.2 Hz), 7.38 (d, 1H, J=8.1 Hz), 7.35 (d, 1H, J=7.8 Hz), 7.18 (d, 2H, J=8.0 Hz), 7.02 (t, 1H, J=7.4 Hz), 6.91 (t, 1H, J=7.5 Hz), 4.03 (s, 2H), 4.01 (d, 2H, J=7.5 Hz), 2.95-2.84 (m, 4H), 2.89 (s, 3H), 2.40 (s, 3H), 2.02-1.88 (m, 5H), 1.80-1.60 (m, 4H), 2.39-1.30 (m, 4H); MS (ESI) m/z 571.2 (M⁺+1).

### Example 67

### Synthesis of Compound 697

### Step 1: (Formula 31) Methyl 4-((1-((1-(3,5-bis(trifluoromethyl)benzyl)piperidin-4-yl)methyl)-2-methyl-1H-indol-3-yl)methyl)benzoate

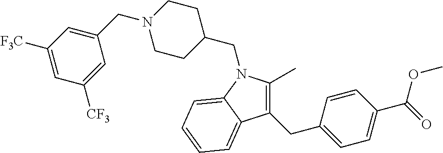 [SMILES: COC(=O)c1ccc(Cc2c(C)n(CC3CCN(Cc4cc(C(F)(F)F)cc(C(F)(F)F)c4)CC3)c3ccccc23)cc1]

Methyl 4-((2-methyl-1-(piperidin-4-ylmethyl)-1H-indol-3-yl)methyl)benzoate hydrochloride (0.150 g, 0.36 mmol), 1-(bromomethyl)-3,5-bis(trifluoromethyl)benzene (0.080 mL, 0.44 mmol) and diisopropylethylamine (0.127 mL, 0.73 mmol) were added to methylene chloride (3 mL) at room temperature, and the mixture was stirred at the same temperature for 1 hour. Then, water was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 0% to 20%) and concentrated to afford the title compound (0.211 g, 96%) as a yellow liquid.

### Step 2: (Compound 697) 4-((1-((1-(3,5-bis(trifluoromethyl)benzyl)piperidin-4-yl)methyl)-2-methyl-1H-indol-3-yl)methyl)-N-hydroxybenzamide

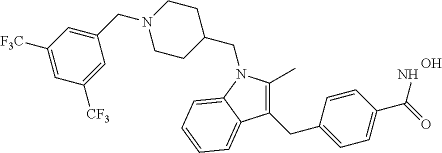

Methyl 4-((1-((1-(3,5-bis(trifluoromethyl)benzyl)piperidin-4-yl)methyl)-2-methyl-1H-indol-3-yl)methyl)benzoate (0.211 g, 0.35 mmol), hydroxylamine (50% aqueous solution, 1.071 mL, 17.51 mmol) and potassium hydroxide (0.196 g, 3.50 mmol) were added to methanol (1 mL) at room temperature, and the mixture was stirred for 1 hour at the same temperature. Then, the reaction mixture was concentrated under reduced pressure. To the concentrate, a saturated aqueous solution of sodium hydrogen carbonate (5 mL) was added, followed by stirring. The precipitated solid was filtered, and then dried to afford compound 697 (0.180 g, 85%) as an ivory solid.

¹H-NMR (400 MHz, DMSO-d₆) δ 7.46-7.32 (m, 3H), 7.01 (q, 2H, J=8.1 Hz), 6.90 (d, 2H, J=7.1 Hz), 6.83 (d, 2H, J=8.3 Hz), 6.41 (d, 2H, J=8.4 Hz), 4.01 (d, 2H, J=7.2 Hz), 3.81 (s, 2H), 3.62 (s, 2H), 2.77 (d, 2H, J=11.6 Hz), 2.37 (s, 3H), 1.91 (t, 2H, J=11.0 Hz), 1.82-1.71 (m, 1H), 1.46-1.32 (m, 4H); MS (ESI) m/z 604.2 (M⁺+1).

### Example 68

### Synthesis of Compound 698

### Step 1: (Formula 36) Methyl 4-((1-((1-((1-hydroxycyclohexyl)methyl)piperidin-4-yl)methyl)-2-methyl-1H-indol-3-yl)methyl)benzoate

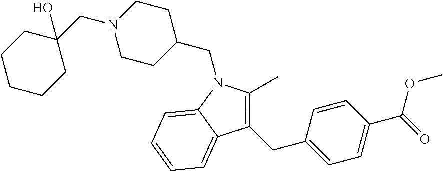 [SMILES: COC(=O)c1ccc(Cc2c(C)n(CC3CCN(CC4(O)CCCCC4)CC3)c3ccccc23)cc1]

Methyl 4-((2-methyl-1-(piperidin-4-ylmethyl)-1H-indol-3-yl)methyl)benzoate hydrochloride (0.200 g, 0.48 mmol), 1-oxaspiro[2.5]octane (0.172 mL, 1.45 mmol) and potassium carbonate (0.335 g, 2.42 mmol) were added to ethanol (3 mL), and heated by microwave irradiation at 120° C. for 30 minutes, followed by cooling to room temperature. Then, water was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 10% to 50%) and concentrated to afford the title compound (0.200 g, 84%) as a yellow liquid.

### Step 2: (Formula 37) Methyl 4-((1-((1-((1-fluorocyclohexyl)methyl)piperidin-4-yl)methyl)-2-methyl-1H-indol-3-yl)methyl)benzoate

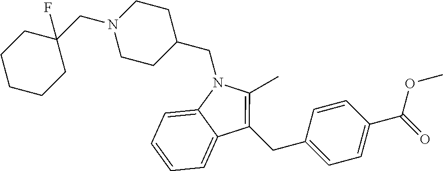 [SMILES: COC(=O)c1ccc(Cc2c(C)n(CC3CCN(CC4(F)CCCCC4)CC3)c3ccccc23)cc1]

Methyl 4-((1-((1-((1-hydroxycyclohexyl)methyl)piperidin-4-yl)methyl)-2-methyl-1H-indol-3-yl)methyl)benzoate (0.200 g, 0.41 mmol) and diethylaminosulfur trifluoride (0.080 mL, 0.61 mmol) were added to methylene chloride (3 mL) at room temperature, and the mixture was stirred at the same temperature for 1 hour. Then, water was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 4 g cartridge; ethyl acetate/hexane=from 0% to 50%) and concentrated to afford the title compound (0.029 g, 15%) as a yellow liquid.

### Step 3: (Compound 698) 4-((1-((1-((1-Fluorocyclohexyl)methyl)piperidin-4-yl)methyl)-2-methyl-1H-indol-3-yl)methyl)-N-hydroxybenzamide

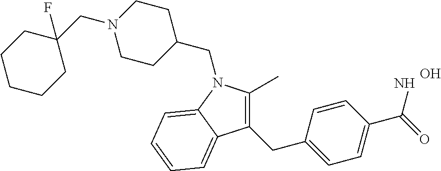

Methyl 4-((1-((1-((1-fluorocyclohexyl)methyl)piperidin-4-yl)methyl)-2-methyl-1H-indol-3-yl)methyl)benzoate (0.029 g, 0.06 mmol), hydroxylamine (50% aqueous solution, 0.181 mL, 2.96 mmol) and potassium hydroxide (0.033 g, 0.59 mmol) were added to methanol (1 mL) at room temperature, and the mixture was stirred at the same temperature for 1 hour. Then, the reaction mixture was concentrated under reduced pressure. To the concentrate, a saturated aqueous solution of sodium hydrogen carbonate (5 mL) was added, followed by stirring. The precipitated solid was filtered, and then dried to afford compound 698 (0.017 g, 59%) as an ivory solid.

¹H-NMR (400 MHz, DMSO-d₆) δ 7.83 (d, 1H, J=8.2 Hz), 7.57 (d, 1H, J=8.2 Hz), 7.40-7.30 (m, 3H), 7.11 (d, 1H, J=8.1 Hz), 7.05-7.00 (m, 1H), 6.93-6.87 (m, 1H), 4.00 (s, 4H), 2.84 (d, 2H, J=11.1 Hz), 1.95 (t, 2H, J=10.5 Hz), 1.77-1.71 (m, 4H), 1.51-1.24 (m, 11H); MS (ESI) m/z 492.2 (M⁺+1).

### Example 69

### Synthesis of Compound 707

### Step 1: (Formula 2) Methyl 4-((4-fluoro-1H-indol-3-yl)methyl)benzoate

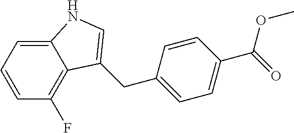 [SMILES: COC(=O)c1ccc(Cc2c[nH]c3cccc(F)c23)cc1]

Methyl 4-(bromomethyl)benzoate (1.400 g, 6.11 mmol) and 4-fluoroindole (0.991 g, 7.33 mmol) were added to water (10 mL), and heated by microwave irradiation at 150° C. for 5 minutes, followed by cooling to room temperature. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 5% to 30%) and concentrated to afford the title compound (0.720 g, 42%) as a white solid.

### Step 2: (Formula 4) Tert-butyl 4-((4-fluoro-3-(4-(methoxycarbonyl)benzyl)-1H-indol-1-yl)methyl)piperidine-1-carboxylate

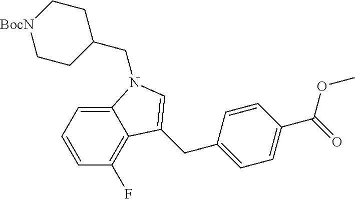 [SMILES: COC(=O)c1ccc(Cc2cn(CC3CCN(C(=O)OC(C)(C)C)CC3)c3cccc(F)c23)cc1]

Methyl 4-((4-fluoro-1H-indol-3-yl)methyl)benzoate (0.720 g, 2.54 mmol) was added to N,N-dimethylformamide (15 mL) at room temperature. To the mixture, sodium hydride (95%, 0.077 g, 3.05 mmol) was added, followed by stirring for 5 minutes. To the reaction solution, tert-butyl 4-((methylsulfonyloxy)methyl)piperidine-1-carboxylate (0.895 g, 3.05 mmol) and potassium iodide (0.506 g, 3.05 mmol) were added, followed by stirring at 60° C. for 2 hours. Then, water was added to the reaction mixture, followed by extraction with ethyl acetate. The organic layer was washed with a saturated aqueous solution of ammonium chloride, dried with anhydrous magnesium sulfate, and then concentrated under reduced pressure. The concentrate was purified by column chromatography (SiO₂, 40 g cartridge; ethyl acetate/hexane=from 10% to 30%) and concentrated to afford the title compound (1.030 g, 84%) as a white solid.

### Step 3: (Formula 5) Methyl 4-((4-fluoro-1-(piperidin-4-ylmethyl)-1H-indol-3-yl)methyl)benzoate hydrochloride

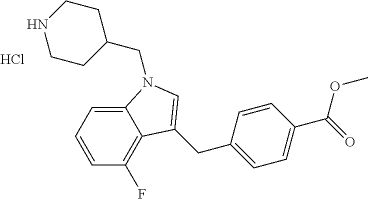 [SMILES: Cl ; COC(=O)c1ccc(Cc2cn(CC3CCNCC3)c3cccc(F)c23)cc1]

Tert-butyl 4-((4-fluoro-3-(4-(methoxycarbonyl)benzyl)-1H-indol-1-yl)methyl)piperidin-1-carboxylate (1.030 g, 2.14 mmol) was added to hydrochloride acid (4.0 M, 1,4-dioxane solution, 8.037 mL, 32.15 mmol), and stirred at room temperature for 1 hour. Then, the reaction mixture was concentrated under reduced pressure. To the concentrate, ethyl acetate (5 mL) and hexane (30 mL) were added, followed by stirring. The precipitated solid was filtered and dried to afford the title compound (0.875 g, 98%) as a light brown solid.

### Step 4: (Formula 6) Methyl 4-((4-fluoro-1-((1-(2-hydroxy-2-methylpropyl)piperidin-4-yl)methyl)-1H-indol-3-yl)methyl)benzoate

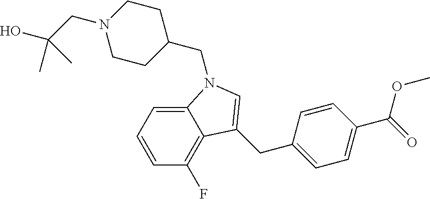 [SMILES: COC(=O)c1ccc(Cc2cn(CC3CCN(CC(C)(C)O)CC3)c3cccc(F)c23)cc1]

Methyl 4-((4-fluoro-1-(piperidin-4-ylmethyl)-1H-indol-3-yl)methyl)benzoate hydrochloride (0.875 g, 2.10 mmol), 2,2-dimethyloxirane (0.757 g, 10.49 mmol) and potassium carbonate (0.870 g, 6.30 mmol) were added to ethanol (10 mL), and heated by microwave irradiation at 110° C. for 20 minutes, followed by cooling to room temperature. Then, the reaction mixture was concentrated under reduced pressure to remove the solvent, and water was added to the concentrate, followed by extraction with ethyl acetate. The organic layer was washed with a saturated aqueous solution of sodium chloride, dried with anhydrous magnesium sulfate, and then concentrated under reduced pressure. The product was used without additional purification (title compound, 0.775 g, 82%, brown liquid).

### Step 5: (Formula 8) Methyl 4-((4-fluoro-1-((1-(2-fluoro-2-methylpropyl)piperidin-4-yl)methyl)-1H-indol-3-yl)methyl)benzoate

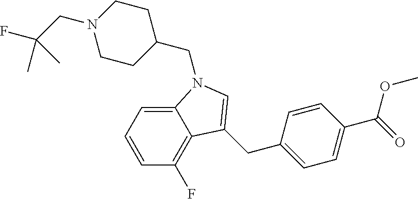 [SMILES: COC(=O)c1ccc(Cc2cn(CC3CCN(CC(C)(C)F)CC3)c3cccc(F)c23)cc1]

Methyl 4-((4-fluoro-1-((1-(2-hydroxy-2-methylpropyl)piperidin-4-yl)methyl)-1H-indol-3-yl)methyl)benzoate (0.775 g, 1.71 mmol) was added to methylene chloride (20 mL) at room temperature. To the mixture, diethylaminosulfur trifluoride (0.226 mL, 1.71 mmol) was added, followed by stirring at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 30% to 60%) and concentrated to afford the title compound (0.441 g, 57%) as a yellow liquid.

### Step 6: (Compound 707) 4-((4-Fluoro-1-((1-(2-fluoro-2-methylpropyl)piperidin-4-yl)methyl)-1H-indol-3-yl)methyl)-N-hydroxybenzamide

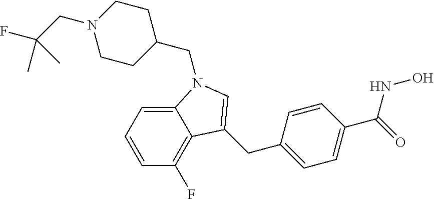 [SMILES: CC(C)(F)CN1CCC(Cn2cc(Cc3ccc(C(=O)NO)cc3)c3c(F)cccc32)CC1]

Methyl 4-((4-fluoro-1-((1-(2-fluoro-2-methylpropyl)piperidin-4-yl)methyl)-1H-indol-3-yl)methyl)benzoate (0.440 g, 0.97 mmol) was added to tetrahydrofuran (3 mL)/methanol (10 mL) at room temperature. To the mixture, hydroxylamine (50% aqueous solution, 2.960 mL, 48.40 mmol) was added, and then potassium hydroxide (0.543 g, 9.68 mmol) was added, followed by stirring at the same temperature for 3 hours. Then, the reaction mixture was concentrated under reduced pressure. To the concentrate, a saturated aqueous solution of sodium hydrogen carbonate (10 mL) was added, followed by stirring. The precipitated solid was filtered, washed with water, and then dried to afford compound 707 (0.426 g, 97%) as a white solid.

¹H NMR (400 MHz, DMSO-d₆) δ 7.63 (d, 2H, J=8.2 Hz), 7.30 (d, 1H, J=8.3 Hz), 7.22 (d, 2H, J=8.2 Hz), 7.18 (s, 1H), 7.05 (m, 1H), 6.69 (m, 1H), 4.11 (s, 2H), 4.02 (d, 2H, J=7.2 Hz), 2.85-2.83 (m, 2H), 2.36 (d, 2H, J=22.8 Hz), 1.99-1.93 (m, 2H), 1.71 (m, 1H), 1.40-1.37 (m, 2H), 1.30-1.22 (m, 8H); MS (ESI) m/z 456.2 (M⁺+H).

### Example 70

### Synthesis of Compound 708

### Step 1: (Formula 2) Methyl 4-((6-fluoro-1H-indol-3-yl)methyl)benzoate)

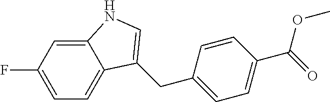 [SMILES: COC(=O)c1ccc(Cc2c[nH]c3cc(F)ccc23)cc1]

Methyl 4-(bromomethyl)benzoate (1.400 g, 6.11 mmol) and 6-fluoroindole (0.991 g, 7.33 mmol) were added to water (10 mL), and heated by microwave irradiation at 150° C. for 5 minutes, followed by cooling to room temperature. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 5% to 30%) and concentrated to afford the title compound (0.670 g, 39%) as a white solid.

### Step 2: (Formula 4) Tert-butyl 4-((6-fluoro-3-(4-(methoxycarbonyl)benzyl)-1H-indol-1-yl)methyl)piperidine-1-carboxylate

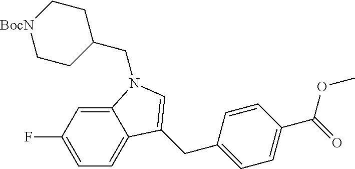 [SMILES: COC(=O)c1ccc(Cc2cn(CC3CCN(C(=O)OC(C)(C)C)CC3)c3cc(F)ccc23)cc1]

Methyl 4-((6-fluoro-1H-indol-3-yl)methyl)benzoate (0.670 g, 2.37 mmol) was added to N,N-dimethylformamide (15 mL) at room temperature. To the mixture, sodium hydride (95%, 0.072 g, 2.84 mmol) was added, followed by stirring for 5 minutes. To the reaction solution, tert-butyl 4-((methylsulfonyloxy)methyl)piperidine-1-carboxylate (0.833 g, 2.84 mmol) and potassium iodide (0.471 g, 2.84 mmol) were added, followed by stirring at 60° C. for 2 hours. Then, water was added to the reaction mixture, followed by extraction with ethyl acetate. The organic layer was washed with a saturated aqueous solution of ammonium chloride, dried with anhydrous magnesium sulfate, and then concentrated under reduced pressure. The concentrate was purified by column chromatography (SiO₂, 40 g cartridge; ethyl acetate/hexane=from 10% to 30%) and concentrated to afford the title compound (0.697 g, 61%) as a white solid.

### Step 3: (Formula 5) Methyl 4-((6-fluoro-1-(piperidin-4-ylmethyl)-1H-indol-3-yl)methyl)benzoate hydrochloride

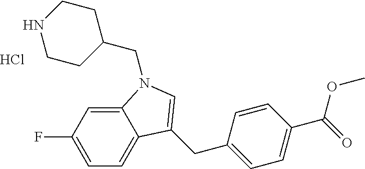 [SMILES: Cl ; COC(=O)c1ccc(Cc2cn(CC3CCNCC3)c3cc(F)ccc23)cc1]

Tert-butyl 4-((6-fluoro-3-(4-(methoxycarbonyl)benzyl)-1H-indol-1-yl)methyl)piperidine-1-carboxylate (0.697 g, 1.45 mmol) was added to hydrochloric acid (4.0 M, 1,4-dioxane solution, 5.439 mL, 21.76 mmol), followed by stirring at room temperature for 1 hour. Then, the reaction mixture was concentrated under reduced pressure. To the concentrate, ethyl acetate (5 mL) and hexane (30 mL) were added, followed by stirring. The precipitated solid was filtered and dried to afford the title compound (0.594 g, 98%) as a white solid.

### Step 4: (Formula 6) Methyl 4-((6-fluoro-1-((1-(2-hydroxy-2-methylpropyl)piperidin-4-yl)methyl)-1H-indol-3-yl)methyl)benzoate

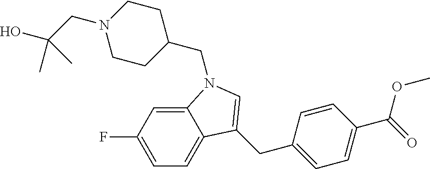 [SMILES: COC(=O)c1ccc(Cc2cn(CC3CCN(CC(C)(C)O)CC3)c3cc(F)ccc23)cc1]

Methyl 4-((6-fluoro-1-(piperidin-4-ylmethyl)-1H-indol-3-yl)methyl)benzoate hydrochloride (0.594 g, 1.43 mmol), 2,2-dimethyloxirane (0.514 g, 7.12 mmol) and potassium carbonate (0.591 g, 4.27 mmol) were added to ethanol (10 mL), and heated by microwave irradiation at 110° C. for 20 minutes, followed by cooling to room temperature. Then, the reaction mixture was concentrated under reduced pressure to remove the solvent. To the concentrate, water was added, followed by extraction with ethyl acetate. The organic layer was washed with a saturated aqueous solution of sodium chloride, dried with anhydrous magnesium sulfate, and then concentrated under reduced pressure. The product was used without additional purification (title compound, 0.641 g, 99%, brown liquid).

### Step 5: (Formula 8) Methyl 4-((6-fluoro-1-((1-(2-fluoro-2-methylpropyl)piperidin-4-yl)methyl)-1H-indol-3-yl)methyl)benzoate

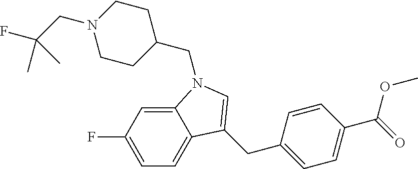 [SMILES: COC(=O)c1ccc(Cc2cn(CC3CCN(CC(C)(C)F)CC3)c3cc(F)ccc23)cc1]

Methyl 4-((6-fluoro-1-((1-(2-hydroxy-2-methylpropyl)piperidin-4-yl)methyl)-1H-indol-3-yl)methyl)benzoate (0.641 g, 1.42 mmol) was added to methylene chloride (20 mL) at room temperature. To the mixture, diethylaminosulfur trifluoride (0.187 mL, 1.42 mmol) was added, followed by stirring at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 12 g cartridge; ethyl acetate/hexane=from 30% to 60%) and concentrated to afford the title compound (0.341 g, 53%) as a light yellow liquid.

### Step 6: (Compound 708) 4-((6-Fluoro-1-((1-(2-fluoro-2-methylpropyl)piperidin-4-yl)methyl)-1H-indol-3-yl)methyl)-N-hydroxybenzamide

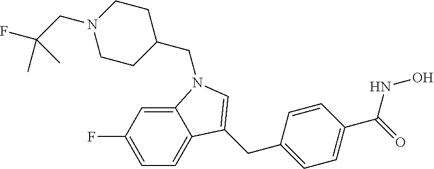 [SMILES: CC(C)(F)CN1CCC(Cn2cc(Cc3ccc(C(=O)NO)cc3)c3ccc(F)cc32)CC1]

Methyl 4-((6-fluoro-1-((1-(2-fluoro-2-methylpropyl)piperidin-4-yl)methyl)-1H-indol-3-yl)methyl)benzoate (0.340 g, 0.75 mmol) was added to tetrahydrofuran (3 mL)/methanol (10 mL) at room temperature. To the mixture, hydroxylamine (50% aqueous solution, 4.575 mL, 74.80 mmol) was added, and then potassium hydroxide (0.420 g, 7.48 mmol) was added, followed by stirring at the same temperature for 3 hours. Then, the reaction mixture was concentrated under reduced pressure. To the concentrate, a saturated aqueous solution of sodium hydrogen carbonate (10 mL) was added, followed by stirring. The precipitated solid was filtered, washed with water, and then dried to afford compound 708 (0.312 g, 92%) as a white solid.

¹H NMR (400 MHz, DMSO-d₆) δ 7.63 (d, 2H, J=8.2 Hz), 7.39-7.33 (m, 2H), 7.27 (d, 2H, J=8.2 Hz), 7.18 (s, 1H), 6.80 (m, 1H), 4.03 (s, 2H), 3.97 (d, 2H, J=7.0 Hz), 2.86-2.83 (m, 2H), 2.36 (d, 2H, J=22.8 Hz), 1.99-1.94 (m, 2H), 1.70 (m, 1H), 1.40-1.38 (m, 2H), 1.30-1.25 (m, 8H); MS (ESI) m/z 456.2 (M⁺+H).

### Example 71

### Synthesis of Compound 713

### Step 1: (Formula 3) Tert-butyl 3-((methylsulfonyloxy)methyl)pyrrolidine-1-carboxylate

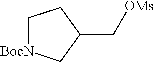 [SMILES: CC(C)(C)OC(=O)N1CCC(COS(C)(=O)=O)C1]

Tert-butyl 3-(hydroxymethyl)pyrrolidin-1-carboxylate (3.600 g, 17.89 mmol), methanesulfonyl chloride (1.523 mL, 19.68 mmol) and triethylamine (4.959 mL, 35.78 mmol) were dissolved in methylene chloride (10 mL) at room temperature, and the solution was stirred at the same temperature for 1 hour. Then, water was added to the reaction mixture, followed by extraction with methylene chloride. The organic layer was washed with a saturated aqueous solution of sodium chloride, dried with anhydrous magnesium sulfate, filtered, and then concentrated under reduced pressure. The concentrate was purified by column chromatography (SiO₂, 40 g cartridge; methanol/methylene chloride=from 0% to 10%) and concentrated to afford the title compound (3.820 g, 76%) as a colorless liquid.

### Step 2: (Formula 4) Tert-butyl 3-((3-(4-(methoxycarbonyl)benzyl)-2-methyl-1H-indol-1-yl)methyl)pyrrolidine-1-carboxylate

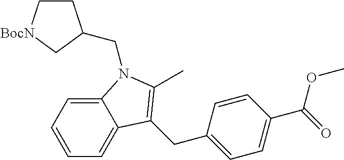 [SMILES: COC(=O)c1ccc(Cc2c(C)n(CC3CCN(C(=O)OC(C)(C)C)C3)c3ccccc23)cc1]

Methyl 4-((2-methyl-1H-indol-3-yl)methyl)benzoate (3.100 g, 11.10 mmol), tert-butyl 3-((methylsulfonyloxy)methyl)pyrrolidine-1-carboxylate (3.720 g, 13.32 mmol), sodium hydride (60%, 0.577 g, 14.43 mmol) and potassium iodide (2.211 g, 13.32 mmol) were dissolved in N,N-dimethylformamide (10 mL) at room temperature, and the solution was stirred at the same temperature for 1 hour. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 0% to 20%) and concentrated to afford the title compound (1.900 g, 37%) as a yellow liquid.

### Step 3: (Formula 5) Methyl 4-((2-methyl-1-(pyrrolidin-3-ylmethyl)-1H-indol-3-yl)methyl)benzoate hydrochloride

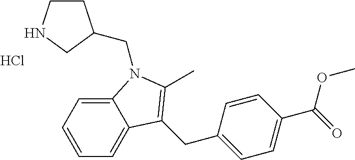 [SMILES: Cl ; COC(=O)c1ccc(Cc2c(C)n(CC3CCNC3)c3ccccc23)cc1]

Tert-butyl 3-((3-(4-(methoxycarbonyl)benzyl)-2-methyl-1H-indol-1-yl)methyl)pyrrolidine-1-carboxylate (1.900 g, 4.11 mmol) and hydrochloric acid (4.0 M, 1,4-dioxane solution, 5.134 mL, 20.54 mmol) were dissolved in 1,4-dioxane (3 mL) at room temperature, and the solution was stirred at the same temperature for 1 hour. Then, the reaction mixture was concentrated under reduced pressure to remove the solvent. To the concentrate, diethyl ether (20 mL) was added, followed by stirring. The precipitated solid was filtered, washed with diethyl ether, and then dried to afford desired compound (1.552 g, 95%) as a pink solid.

### Step 4: (Formula 6) Methyl 4-((1-((1-(2-hydroxy-2-methylpropyl)pyrrolidin-3-yl)methyl)-2-methyl-1H-indol-3-yl)methyl)benzoate

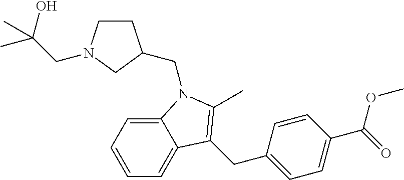 [SMILES: COC(=O)c1ccc(Cc2c(C)n(CC3CCN(CC(C)(C)O)C3)c3ccccc23)cc1]

Methyl 4-((2-methyl-1-(pyrrolidin-3-ylmethyl)-1H-indol-3-yl)methyl)benzoate hydrochloride (0.200 g, 0.50 mmol), 2,2-dimethyloxirane (0.362 g, 5.01 mmol) and potassium carbonate (0.693 g, 5.01 mmol) were added to ethanol (5 mL), and heated by microwave irradiation at 110° C. for 20 minutes, followed by cooling to room temperature. After completion of the reaction, water was added to the reaction mixture, followed by extraction with ethyl acetate. The organic layer was washed with a saturated aqueous solution of sodium chloride, dried with anhydrous magnesium sulfate, filtered, and then concentrated under reduced pressure. The concentrate was purified by column chromatography (SiO₂, 4 g cartridge; ethyl acetate/hexane=from 0% to 20%) and concentrated to afford the title compound (0.187 g, 86%) as a yellow liquid.

### Step 5: (Formula 8) Methyl 4-((1-((1-(2-fluoro-2-methylpropyl)pyrrolidin-3-yl)methyl)-2-methyl-1H-indol-3-yl)methyl)benzoate

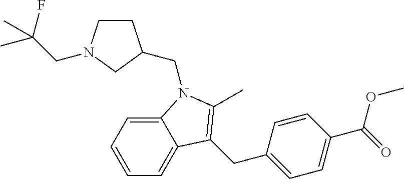 [SMILES: COC(=O)c1ccc(Cc2c(C)n(CC3CCN(CC(C)(C)F)C3)c3ccccc23)cc1]

Methyl 4-((1-((1-(2-hydroxy-2-methylpropyl)pyrrolidin-3-yl)methyl)-2-methyl-1H-indol-3-yl)methyl)benzoate (0.187 g, 0.43 mmol) and diethylaminosulfur trifluoride (0.085 mL, 0.65 mmol) were dissolved in methylene chloride (3 mL) at room temperature, and the solution was stirred at the same temperature for 1 hour. Then, water was added to the reaction mixture, followed by extraction with methylene chloride. The organic layer was washed with a saturated aqueous solution of sodium chloride, dried with anhydrous magnesium sulfate, filtered, and then concentrated under reduced pressure. The concentrate was purified by column chromatography (SiO₂, 4 g cartridge; ethyl acetate/hexane=from 20% to 50%) and concentrated to afford the title compound (0.102 g, 54%) as a yellow liquid.

### Step 6: (Compound 713) 4-((1-((1-(2-Fluoro-2-methylpropyl)pyrrolidin-3-yl)methyl)-2-methyl-1H-indol-3-yl)methyl)-N-hydroxybenzamide

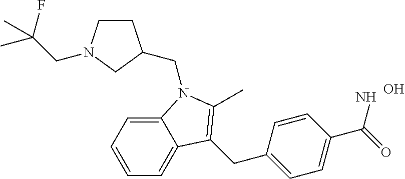

Methyl 4-((1-((1-(2-fluoro-2-methylpropyl)pyrrolidin-3-yl)methyl)-2-methyl-1H-indol-3-yl)methyl)benzoate (0.102 g, 0.23 mmol), hydroxylamine (50% aqueous solution, 0.715 mL, 11.68 mmol) and potassium hydroxide (0.131 g, 2.34 mmol) were dissolved in methanol (1 mL) at room temperature, and the solution was stirred at the same temperature for 1 hour. Then, the reaction mixture was concentrated under reduced pressure. To the concentrate, a saturated aqueous solution of sodium hydrogen carbonate (10 mL) was added, followed by stirring. The precipitated solid was filtered, and then dried to afford compound 713 (0.063 g, 62%) as an ivory solid.

¹H-NMR (400 MHz, DMSO-d₆) δ 7.58 (d, 2H, J=8.2 Hz), 7.38 (d, 1H, J=8.3 Hz), 7.35 (d, 1H, J=8.2 Hz), 7.15 (d, 2H, J=7.8 Hz), 7.02 (t, 1H, J=7.6 Hz), 6.91 (t, 1H, J=7.4 Hz), 4.14-4.04 (m, 2H), 4.02 (s, 2H), 2.82 (q, 2H, J=7.2 Hz), 2.61-2.56 (m, 4H), 2.43 (s, 3H), 2.36 (t, 2H, J=8.2 Hz), 1.86-1.71 (m, 1H), 1.51-1.41 (m, 1H), 1.36 (d, 3H, J=5.8 Hz), 1.30 (d, 3H, J=5.8 Hz); MS (ESI) m/z 438.2 (M⁺+1).

### Example 72

### Synthesis of Compound 728

### Step 1: (Formula 52) Tert-butyl 4-((2-methyl-1H-indol-1-yl)methyl)piperidine-1-carboxylate

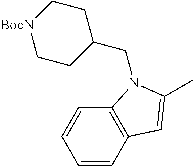 [SMILES: Cc1cc2ccccc2n1CC1CCN(C(=O)OC(C)(C)C)CC1]

2-methylindole (5.000 g, 38.12 mmol), tert-butyl 4-((methylsulfonyloxy)methyl)piperidine-1-carboxylate (13.42 g, 45.74 mmol), sodium hydride (60%, 1.982 g, 49.55 mmol) and potassium iodide (7.593 g, 45.74 mmol) were dissolved in N,N-dimethylformamide (10 mL) at 60° C., and the solution was stirred at the same temperature for 2 hours, and then cooled to room temperature. After completion of the reaction, water was added to the reaction mixture, followed by extraction with ethyl acetate. The organic layer was washed with a saturated aqueous solution of sodium chloride, dried with anhydrous magnesium sulfate, filtered, and then concentrated under reduced pressure. The concentrate was purified by column chromatography (SiO₂, 80 g cartridge; ethyl acetate/hexane=from 10% to 30%) and concentrated to afford the title compound (10.03 g, 80%) as a yellow solid.

Sep 2: (Formula 53) 2-Methyl-1-(piperidin-4-ylmethyl)-1H-indole hydrochloride

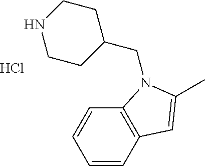 [SMILES: Cl ; Cc1cc2ccccc2n1CC1CCNCC1]

Tert-butyl 4-((2-methyl-1H-indol-1-yl)methyl)piperidine-1-carboxylate (3.000 g, 9.13 mmol) and hydrochloric acid (4.0 M, 1,4-dioxane solution, 11.42 mL, 45.67 mmol) were dissolved in 1,4-dioxane (5 mL) at room temperature, and the solution was stirred at the same temperature for 1 hour. Then, the reaction mixture was concentrated under reduced pressure. To the concentrate, diethyl ether (50 mL) was added, followed by stirring. The precipitated solid was filtered, washed with hexane, and then dried to afford compound (2.067 g, 99%) as a red solid.

### Step 3: (Formula 54) (4-((2-Methyl-1H-indol-1-yl)methyl)piperidin-1-yl)(1-(trifluoromethyl)cyclobutyl)methanone

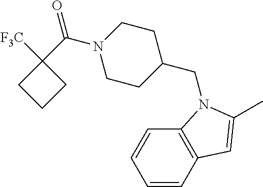 [SMILES: Cc1cc2ccccc2n1CC1CCN(C(=O)C2(C(F)(F)F)CCC2)CC1]

2-methyl-1-(piperidin-4-ylmethyl)-1H-indole hydrochloride (1.200 g, 4.53 mmol), 1-(trifluoromethyl)cyclobutanecarboxylic acid (1.146 mL, 9.06 mmol), 1-ethyl-3-(3-dimethylaminopropyl)carbodiimide (2.606 g, 13.60 mmol), 1-hydroxybenzotriazole anhydride (1.837 g, 13.60 mmol) and diisopropylethylamine (2.343 mL, 13.60 mmol) were dissolved in N,N-dimethylformamide (10 mL) at 60° C., and the solution was stirred at the same temperature for 24 hours, and then cooled to room temperature. After completion of the reaction, water was added to the reaction mixture, followed by extraction with ethyl acetate. The organic layer was washed with a saturated aqueous solution of sodium chloride, dried with anhydrous magnesium sulfate, filtered, and then concentrated under reduced pressure. The concentrate was purified by column chromatography (SiO₂, 40 g cartridge; ethyl acetate/hexane=from 10% to 50%) and concentrated to afford the title compound (0.963 g, 56%) as a yellow liquid.

### Step 4: (Formula 55) 2-methyl-1-((1-((1-(trifluoromethyl)cyclobutyl)methyl)piperidin-4-yl)methyl)-1H-indole

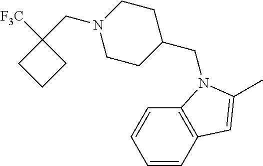 [SMILES: Cc1cc2ccccc2n1CC1CCN(CC2(C(F)(F)F)CCC2)CC1]

(4-((2-methyl-1H-indol-1-yl)methyl)piperidin-1-yl)(1-(trifluoromethyl)cyclobutyl)methanone (0.963 g, 2.55 mmol) was dissolved in tetrahydrofuran (10 mL). At room temperature, lithium aluminum anhydride (1.0 M tetrahydrofuran solution, 7.634 mL, 7.63 mmol) was slowly added to the solution, followed by stirring for 3 minutes. To the reaction mixture, methanol (10.31 mL, 254.47 mmol) was added, followed by stirring for 30 minutes. After completion of the reaction, water was added to the reaction mixture, followed by extraction with ethyl acetate. The organic layer was washed with a saturated aqueous solution of sodium chloride, dried with anhydrous magnesium sulfate, filtered, and then concentrated under reduced pressure. The concentrate was purified by column chromatography (SiO₂, 40 g cartridge; ethyl acetate/hexane=from 10% to 30%) and concentrated to afford the title compound (0.430 g, 46%) as a white solid.

### Step 5: (Formula 56) Methyl 4-((2-methyl-1-((1-((1-(trifluoromethyl)cyclobutyl)methyl)piperidin-4-yl)methyl)-1H-indol-3-yl)methyl)benzoate

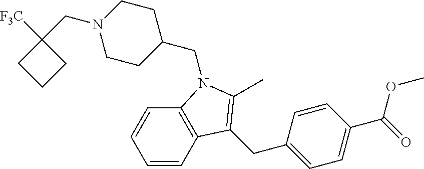 [SMILES: COC(=O)c1ccc(Cc2c(C)n(CC3CCN(CC4(C(F)(F)F)CCC4)CC3)c3ccccc23)cc1]

(4-((2-methyl-1H-indol-1-yl)methyl)piperidin-1-yl)(1-(trifluoromethyl)cyclobutyl)methanone (0.430 g, 1.14 mmol) and methyl 4-(bromomethyl)benzoate (0.286 g, 1.25 mmol) were added to water (5 mL), and heated by microwave irradiation at 150° C. for 10 minutes, followed by cooling to room temperature. After completion of the reaction, water was added to the reaction mixture, followed by extraction with methylene chloride. The organic layer was washed with a saturated aqueous solution of sodium chloride, dried with anhydrous magnesium sulfate, filtered, and then concentrated under reduced pressure. The concentrate was purified by column chromatography (SiO₂, 40 g cartridge; ethyl acetate/hexane=from 0% to 30%) and concentrated to afford the title compound (0.174 g, 30%) as a colorless liquid.

### Step 6: (Compound 728) N-hydroxy-4-((2-methyl-1-((1-((1-(trifluoromethyl)cyclobutyl)methyl)piperidin-4-yl)methyl)-1H-indol-3-yl)methyl)benzamide

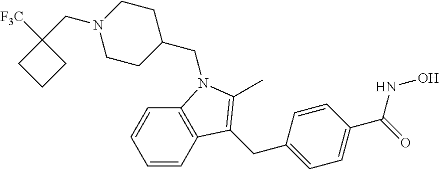 [SMILES: Cc1c(Cc2ccc(C(=O)NO)cc2)c2ccccc2n1CC1CCN(CC2(C(F)(F)F)CCC2)CC1]

Methyl 4-((2-methyl-1-((1-((1-(trifluoromethyl)cyclobutyl)methyl)piperidin-4-yl)methyl)-1H-indol-3-yl)methyl)benzoate (0.174 g, 0.34 mmol), hydroxylamine (50% aqueous solution, 0.208 mL, 3.40 mmol) and potassium hydroxide (0.191 g, 3.40 mmol) were dissolved in methanol (2 mL)/tetrahydrofuran (2 mL) at room temperature, and the solution was stirred at the same temperature for 2 hours. Then, the reaction mixture was concentrated under reduced pressure to remove the solvent. To the concentrate, a saturated aqueous solution of sodium hydrogen carbonate (20 mL) was added, followed by stirring. The precipitated solid was filtered, washed with hexane, and then dried to afford compound 728 (0.174 g, 100%) as a white solid.

¹H-NMR (400 MHz, CH₃OD) d 7.57 (d, 2H, J=8.2 Hz), 7.27 (t, 2H, J=8.0 Hz), 7.14 (d, 2H, J=8.3 Hz), 7.02 (t, 1H, J=7.5 Hz), 6.88 (t, 1H, J=7.5 Hz), 4.06 (s, 2H), 3.99 (d, 2H, J=7.4 Hz), 2.81 (d, 2H, J=11.8 Hz), 2.48 (s, 2H), 2.35 (s, 3H), 2.21-2.14 (m, 2H), 2.10-2.01 (m, 4H), 1.97-1.87 (m, 2H), 1.86-1.78 (m, 1H), 1.48-1.38 (m, 4H); MS (ESI) m/z 514.3 (M⁺+1).

### Example 73

### Synthesis of Compound 747

### 4-((1-((1-(2-Fluoro-2-methylpropyl)piperidin-4-yl)methyl)-1H-pyrrolo[2,3-b]pyridin-3-yl)(hydroxy)methyl)-N-hydroxybenzamide

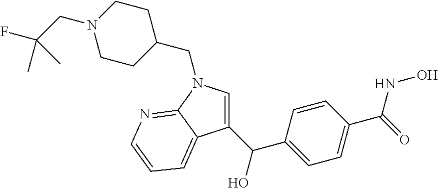 [SMILES: CC(C)(F)CN1CCC(Cn2cc(C(O)c3ccc(C(=O)NO)cc3)c3cccnc32)CC1]

Methyl 4-((1-((1-(2-fluoro-2-methylpropyl)piperidin-4-yl)methyl)-1H-pyrrolo[2,3-b]pyridin-3-yl)(hydroxy)methyl)benzoate (0.100 g, 0.22 mmol) was dissolved in methanol (5 mL) at room temperature. To the solution, hydroxylamine (50% aqueous solution, 1.349 mL, 22.05 mmol) and potassium hydroxide (0.124 g, 2.21 mmol) were added, followed by stirring at the same temperature for 3 hours. Then, the reaction mixture was concentrated under reduced pressure to remove the solvent. To the concentrate, a saturated aqueous solution of sodium hydrogen carbonate was added, followed by extraction with ethyl acetate. The organic layer was washed with a saturated aqueous solution of sodium chloride, dried with anhydrous magnesium sulfate, filtered, and then concentrated under reduced pressure. The product was used without additional purification (Compound 747, 0.062 g, 62%, light yellow solid).

¹H NMR (400 MHz, DMSO-d₆) δ 11.15 (s, 1H), 9.00 (s, 1H), 8.19 (d, 1H, J=3.4 Hz), 7.81 (d, 1H, J=7.9 Hz), 7.70 (d, 2H, J=8.2 Hz), 7.51 (d, 2H, J=8.1 Hz), 7.36 (s, 1H), 6.99 (m, 1H), 5.98 (d, 1H, J=4.3 Hz), 5.86 (d, 1H, J=4.3 Hz), 4.09 (d, 2H, J=7.1 Hz), 2.86-2.83 (m, 2H), 2.37 (d, 2H, J=22.6 Hz), 2.00-1.94 (m, 2H), 1.79 (m, 1H), 1.41-1.39 (m, 2H), 1.31 (s, 3H), 1.28-1.25 (m, 5H); MS (ESI) m/z 455.2 (M⁺+H).

### Example 74

### Synthesis of Compound 748

### Step 1: (Formula 58) 3-Bromo-1H-pyrrolo[2,3-b]pyridine

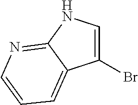

7-azaindole (5.000 g, 42.32 mmol) was added to carbon tetrachloride (150 mL) at 0° C. To the mixture, bromine (2.182 mL, 42.32 mmol) was added, followed by stirring at the same temperature for 2 hours. Then, 0.5 N hydrochloric acid aqueous solution (500 mL) was added to the reaction mixture, followed by extraction. To the water layer, 0.5 N sodium hydroxide aqueous solution (700 mL) was added, followed by stirring. The precipitated solid was filtered, washed with water, and then dried to afford the title compound (4.820 g, 58%) as a brown solid.

### Step 2: (Formula 59) Tert-butyl 4-((3-bromo-1H-pyrrolo [2,3-b]pyridin-1-yl)methyl)piperidine-1-carboxylate

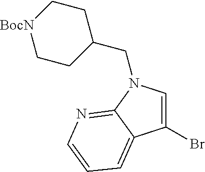 [SMILES: CC(C)(C)OC(=O)N1CCC(Cn2cc(Br)c3cccnc32)CC1]

3-bromo-1H-pyrrolo[2,3-b]pyridine (2.000 g, 10.15 mmol) was added to N,N-dimethylformamide (50 mL). To the mixture, sodium hydride (95%, 0.308 g, 12.18 mmol) was added, followed by stirring at room temperature for 10 minutes. Then, tert-butyl 4-((methylsulfonyloxy)methyl)piperidine-1-carboxylate (3.574 g, 12.18 mmol) and potassium iodide (2.022 g, 12.18 mmol) were added to the reaction solution, followed by stirring at 60° C. for 2 hours. Then, a saturated aqueous solution of ammonium chloride was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 20% to 50%) and concentrated to afford the title compound (2.900 g, 73%) as a light yellow liquid.

### Step 3: (Formula 60) 3-Bromo-1-(piperidin-4-ylmethyl)-1H-pyrrolo[2,3-b]pyridine hydrochloride

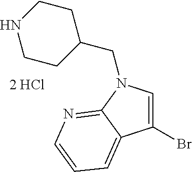 [SMILES: Brc1cn(CC2CCNCC2)c2ncccc12]

Tert-butyl 4-((3-bromo-1H-pyrrolo[2,3-b]pyridin-1-yl)methyl)piperidine-1-carboxylate (2.900 g, 7.36 mmol) was added to hydrochloric acid (4.0 M, 1,4-dioxane solution, 14.709 mL, 58.84 mmol) and stirred at room temperature for 1 hour. To the reaction mixture, ethyl acetate (20 mL) and hexane (100 mL) were added, followed by stirring. The precipitated solid was filtered and dried to afford the title compound (2.680 g, 99%) as a white solid.

### Step 4: (Formula 61) 1-(4-((3-Bromo-1H-pyrrolo[2,3-b]pyridin-1-yl)methyl)piperidin-1-yl)-2-methylpropan-2-ol

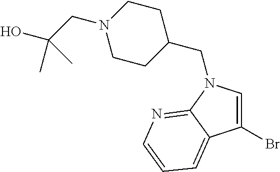 [SMILES: CC(C)(O)CN1CCC(Cn2cc(Br)c3cccnc32)CC1]

3-bromo-1-(piperidin-4-ylmethyl)-1H-pyrrolo[2,3-b]pyridine hydrochloride (2.680 g, 7.30 mmol), 2,2-dimethyloxirane (3.290 mL, 36.50 mmol) add potassium carbonate (5.045 g, 36.50 mmol) were added to water (10 mL)/ethanol (20 mL), and heated by microwave irradiation at 110° C. for 20 hours, followed by cooling to room temperature. Then, water was added to the reaction mixture, followed by extraction with ethyl acetate. The organic layer was washed with a saturated aqueous solution of sodium chloride, dried with anhydrous magnesium sulfate, and then concentrated under reduced pressure. The product was used without additional purification (title compound, 2.130 g, 80%).

### Step 5: (Formula 62) 3-Bromo-1-((1-(2-fluoro-2-methylpropyl)piperidin-4-yl)methyl)-1H-pyrrolo[2,3-b]pyridine

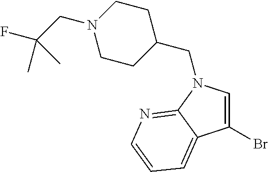 [SMILES: CC(C)(F)CN1CCC(Cn2cc(Br)c3cccnc32)CC1]

1-(4-((3-bromo-1H-pyrrolo[2,3-b]pyridin-1-yl)methyl)piperidin-1-yl)-2-methylpropan-2-ol (2.130 g, 5.82 mmol) was added to methylene chloride (30 mL) at room temperature. To the mixture, diethylaminosulfur trifluoride (0.845 mL, 6.40 mmol) was added, followed by stirring at the same temperature for 2 hours. Then, a saturated aqueous solution of sodium hydrogen carbonate was added to the reaction mixture, followed by extraction with methylene chloride. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 30% to 60%) and concentrated to afford the title compound (1.140 g, 53%) as a colorless liquid.

### Step 6: (Formula 63) (1-((1-(2-Fluoro-2-methylpropyl)piperidin-4-yl)methyl)-1H-pyrrolo[2,3-b]pyridin-3-yl)lithium

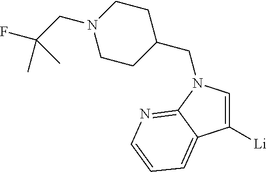

3-bromo-1-((1-(2-fluoro-2-methylpropyl)piperidin-4-yl)methyl)-1H-pyrrolo[2,3-b]pyridine (1.140 g, 3.10 mmol) was dissolved in tetrahydrofuran (20 mL). At a temperature of −78° C., n-butyllithium (1.6 M hexane solution, 2.128 mL, 3.41 mmol) was added slowly to the solution, followed by stirring for 10 minutes. The product was used without additional purification (title compound, 0.913 g, 100%, 0.16 M yellow solution).

### Step 7: (Formula 64) Methyl 4-((1-((1-(2-fluoro-2-methylpropyl)piperidin-4-yl)methyl)-1H-pyrrolo[2,3-b]pyridin-3-yl)(hydroxy)methyl)benzoate

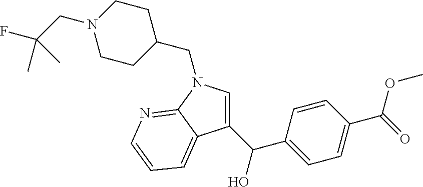 [SMILES: COC(=O)c1ccc(C(O)c2cn(CC3CCN(CC(C)(C)F)CC3)c3ncccc23)cc1]

Methyl 4-formylbenzoate (0.761 g, 4.64 mmol) was dissolved in tetrahydrofuran (10 mL) at −78° C. To the solution, (1-((1-(2-fluoro-2-methylpropyl)piperidin-4-yl)methyl)-1H-pyrrolo[2,3-b]pyridin-3-yl)lithium (0.16 M tetrahydrofuran solution, 19.946 mL, 3.09 mmol) was added, followed by stirring at the same temperature for 1 hour. Then, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, and then concentrated under reduced pressure. The concentrate was purified by column chromatography (SiO₂, 40 g cartridge; ethyl acetate/hexane=from 20% to 60%) and concentrated to afford the title compound (0.884 g, 63%) as a light yellow solid.

### Step 8: (Formula 65) Methyl 4-(chloro(1-((1-(2-fluoro-2-methylpropyl)piperidin-4-yl)methyl)-1H-pyrrolo[2,3-b]pyridin-3-yl)methyl)benzoate

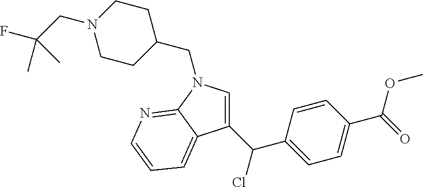 [SMILES: COC(=O)c1ccc(C(Cl)c2cn(CC3CCN(CC(C)(C)F)CC3)c3ncccc23)cc1]

Methyl 4-((1-((1-(2-fluoro-2-methylpropyl)piperidin-4-yl)methyl)-1H-pyrrolo[2,3-b]pyridin-3-yl)(hydroxy)methyl)benzoate (0.770 g, 1.70 mmol) was dissolved in methylene chloride (10 mL) at room temperature. To the solution, methanesulfonyl chloride (0.233 g, 2.04 mmol) and diisopropylethylamine (0.445 mL, 2.55 mmol) were added, followed by stirring at 40° C. for 16 hours. Then, the temperature was lowered to room temperature to terminate the reaction. The reaction mixture was evaporated under reduced pressure to remove the solvent, the residue was used without additional purification (title compound, 0.800 g, 100%, brown liquid).

### Step 9: (Formula 66) Methyl 4-((1-((1-(2-fluoro-2-methylpropyl)piperidin-4-yl)methyl)-1H-pyrrolo[2,3-b]pyridin-3-yl)methyl)benzoate

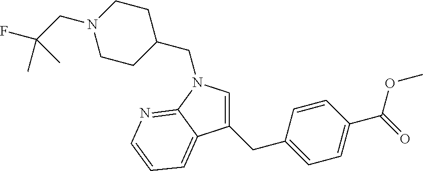 [SMILES: COC(=O)c1ccc(Cc2cn(CC3CCN(CC(C)(C)F)CC3)c3ncccc23)cc1]

Methyl 4-(chloro(1-((1-(2-fluoro-2-methylpropyl)piperidin-4-yl)methyl)-1H-pyrrolo[2,3-b]pyridin-3-yl)methyl)benzoate (0.800 g, 1.70 mmol) and zinc (0.222 g, 3.39 mmol) were added to acetic acid (5 mL) at room temperature, and the mixture was stirred at 90° C. for 2 hour, and then cooled to room temperature. After completion of the reaction, the reaction mixture was concentrated under reduced pressure to remove the solvent. To the concentrate, a saturated aqueous solution of sodium hydrogen carbonate was added, followed by extraction with ethyl acetate. The organic layer was washed with a saturated aqueous solution of sodium chloride, dried with anhydrous magnesium sulfate, filtered, and then concentrated under reduced pressure. The concentrate was purified by column chromatography (SiO₂, 12 g cartridge; ethyl acetate/hexane=from 30% to 60%) and concentrated to afford the title compound (0.246 g, 33%) as a yellow liquid.

### Step 10: (Formula 748) 4-((1-((1-(2-Fluoro-2-methylpropyl)piperidin-4-yl)methyl)-1H-pyrrolo[2,3-b]pyridin-3-yl)methyl)-N-hydroxybenzamide

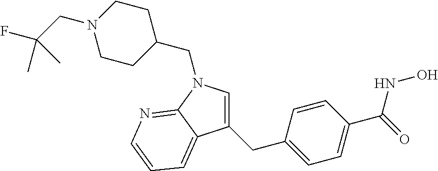 [SMILES: CC(C)(F)CN1CCC(Cn2cc(Cc3ccc(C(=O)NO)cc3)c3cccnc32)CC1]

Methyl 4-((1-((1-(2-fluoro-2-methylpropyl)piperidin-4-yl)methyl)-1H-pyrrolo[2,3-b]pyridin-3-yl)methyl)benzoate (0.246 g, 0.56 mmol) was dissolved in methanol (10 mL) at room temperature. To the solution, hydroxylamine (50% aqueous solution, 2.407 mL, 39.36 mmol) and potassium hydroxide (0.315 g, 5.62 mmol) were added, followed by stirring at the same temperature for 3 hours. Then, the reaction mixture was concentrated under reduced pressure to remove the solvent. To the concentrate, a saturated aqueous solution of sodium hydrogen carbonate (5 mL) was added, followed by stirring. The precipitated solid was filtered, washed with water, and dried to afford compound 748 (0.183 g, 74%) as a light yellow solid.

¹H NMR (400 MHz, DMSO-d₆) δ 8.20 (m, 1H), 7.81 (d, 1H, J=7.7 Hz), 7.65 (d, 2H, J=8.2 Hz), 7.38 (s, 1H), 7.30 (d, 2H, J=7.9 Hz), 7.00 (m, 1H), 4.10 (d, 2H, J=7.4 Hz), 4.07 (s, 2H), 2.85-2.82 (m, 2H), 2.37 (d, 2H, J=22.8 Hz), 2.00-1.95 (m, 2H), 1.82 (m, 1H), 1.40-1.38 (m, 2H), 1.30 (s, 3H), 1.27-1.22 (m, 5H); MS (ESI) m/z 439.2 (M⁺+H).

### Example 75

### Synthesis of Compound 749

### Step 1: (Formula 51) Methyl 4-((1-((1-((3-fluorooxetan-3-yl)methyl)azetidin-3-yl)methyl)-2-methyl-1H-indol-3-yl)methyl)benzoate

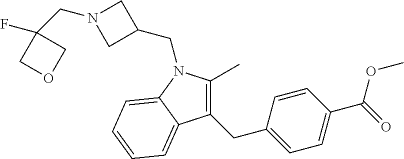 [SMILES: COC(=O)c1ccc(Cc2c(C)n(CC3CN(CC4(F)COC4)C3)c3ccccc23)cc1]

Methyl 4-((1-(azetidin-3-ylmethyl)-2-methyl-1H-indol-3-yl)methyl)benzoate hydrochloride (0.200 g, 0.52 mmol), 3-(bromomethyl)-3-fluorooxetane (0.263 g, 1.56 mmol) and diisopropylethylamine (0.272 mL, 1.56 mmol) were added to acetonitrile (3 mL), and heated by microwave irradiation at 120° C. for 1 hour, followed by cooling to room temperature. After completion of the reaction, a saturated aqueous solution of sodium hydrogen carbonate was added to the reaction mixture, followed by extraction with ethyl acetate. The organic layer was washed with a saturated aqueous solution of sodium chloride, dried with anhydrous magnesium sulfate, filtered, and then concentrated under reduced pressure. The concentrate was purified by column chromatography (SiO₂, 12 g cartridge; ethyl acetate/hexane=from 40% to 70%) and concentrated to afford the title compound (0.083 g, 37%) as a yellow solid.

### Step 2: (Compound 749) 4-((1-((1-((3-Fluorooxetan-3-yl)methyl)azetidin-3-yl)methyl)-2-methyl-1H-indol-3-yl)methyl)-N-hydroxybenzamide

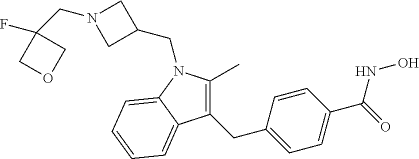 [SMILES: Cc1c(Cc2ccc(C(=O)NO)cc2)c2ccccc2n1CC1CN(CC2(F)COC2)C1]

Methyl 4-((1-((1-((3-fluorooxetan-3-yl)methyl)azetidin-3-yl)methyl)-2-methyl-1H-indol-3-yl)methyl)benzoate (0.083 g, 0.19 mmol) was dissolved in methanol (10 mL) at room temperature. To the solution, hydroxylamine (50% aqueous solution, 1.163 mL, 19.01 mmol) and potassium hydroxide (0.107 g, 1.90 mmol) were added, followed by stirring at the same temperature for 3 hours. Then, the reaction mixture was concentrated under reduced pressure to remove the solvent. To the concentrate, a saturated aqueous solution of sodium hydrogen carbonate (20 mL) was added, followed by stirring. The precipitated solid was filtered, washed with water, and then dried to afford compound 749 (0.072 g, 87%) as a light brown solid.

¹H NMR (400 MHz, DMSO-d₆) δ 7.60 (d, 2H, J=8.2 Hz), 7.40 (d, 1H, J=8.2 Hz), 7.35 (d, 1H, J=7.8 Hz), 7.23 (d, 2H, J=8.2 Hz), 7.04 (t, 1H, J=7.1 Hz), 6.92 (t, 1H, J=7.4 Hz), 4.57 (d, 1H, J=7.8 Hz), 4.53-4.50 (m, 2H), 4.46 (d, 1H, J=8.3 Hz), 4.31 (d, 2H, J=7.1 Hz), 4.05 (s, 2H), 3.27 (t, 2H, J=7.0 Hz), 3.00 (t, 2H, J=6.4 Hz), 2.86-2.80 (m, 3H), 2.41 (s, 3H); MS (ESI) m/z 438.1 (M⁺+H).

### Example 76

### Synthesis of Compound 750

### Step 1: (Formula 3) Tert-butyl 3-((methylsulfonyloxy)methyl)azetidine-1-carboxylate

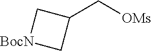 [SMILES: CC(C)(C)OC(=O)N1CC(COS(C)(=O)=O)C1]

Tert-butyl 3-(hydroxymethyl)azetidine-1-carboxylate (1.000 g, 5.34 mmol) was dissolved in methylene chloride (20 mL) at room temperature. To the solution, methanesulfonyl chloride (0.537 mL, 6.94 mmol) and triethylamine (1.095 mL, 8.01 mmol) were added, followed by stirring at the same temperature for 16 hours. Then, a saturated aqueous solution of ammonium chloride was added to the reaction mixture, followed by extraction with methylene chloride. The organic layer was washed with a saturated aqueous solution of sodium chloride, dried with anhydrous magnesium sulfate, filtered, and then concentrated under reduced pressure. The concentrate was purified by column chromatography (SiO₂, 12 g cartridge; ethyl acetate/hexane=from 50% to 70%) and concentrated to afford the title compound (1.380 g, 97%) as a yellow liquid.

### Step 2: (Formula 4) Tert-butyl 3-((3-(4-(methoxycarbonyl)benzyl)-2-methyl-1H-indol-1-yl)methyl)azetidine-1-carboxylate

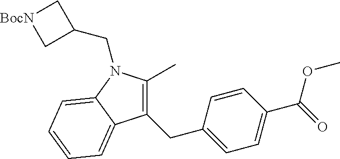 [SMILES: COC(=O)c1ccc(Cc2c(C)n(CC3CN(C(=O)OC(C)(C)C)C3)c3ccccc23)cc1]

Methyl 4-((2-methyl-1H-indol-3-yl)methyl)benzoate (1.200 g, 4.30 mmol) was dissolved in N,N-dimethylformamide (30 mL) at room temperature. To the solution, sodium hydride (95%, 0.130 g, 5.16 mmol) was added, followed by stirring at the same temperature for 5 minutes. To the reaction mixture, tert-butyl 3-((methylsulfonyloxy)methyl)azetidine-1-carboxylate (1.368 g, 5.16 mmol) and potassium iodide (0.856 g, 5.16 mmol) were added, followed by stirring at 60° C. for 2 hours. Then, the reaction mixture was cooled to room temperature to terminate the reaction. Then, a saturated aqueous solution of ammonium chloride was added to the reaction mixture, followed by extraction with ethyl acetate. The organic layer was washed with a saturated aqueous solution of sodium hydrogen carbonate, dried with anhydrous magnesium sulfate, filtered, and then concentrated under reduced pressure. The concentrate was purified by column chromatography (SiO₂, 40 g cartridge; ethyl acetate/hexane=from 10% to 30%) and concentrated to afford the title compound (1.100 g, 57%) as a light yellow solid.

### Step 3: (Formula 5) Methyl 4-((1-(azetidin-3-ylmethyl)-2-methyl-1H-indol-3-yl)methyl)benzoate hydrochloride

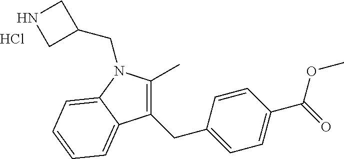 [SMILES: Cl ; COC(=O)c1ccc(Cc2c(C)n(CC3CNC3)c3ccccc23)cc1]

Tert-butyl 3-((3-(4-(methoxycarbonyl)benzyl)-2-methyl-1H-indol-1-yl)methyl)azetidine-1-carboxylate (1.100 g, 2.45 mmol) was dissolved in 1,4-dioxane (5 mL) at room temperature. To the solution, hydrochloric acid (4.0 M, 1,4-dioxane solution, 9.196 mL, 36.79 mmol) was added, followed by stirring at the same temperature for 1 hour. Then, the reaction mixture was concentrated under reduced pressure to remove the solvent. To the concentrate, ethyl acetate (20 mL) and hexane (100 mL) were added, followed by stirring. The precipitated solid was filtered, washed with hexane, and then dried to afford the title compound (0.930 g, 99%) as a pink solid.

### Step 4: (Formula 6) Methyl 4-((1-((1-(2-hydroxy-2-methylpropyl)azetidin-3-yl)methyl)-2-methyl-1H-indol-3-yl)methyl)benzoate

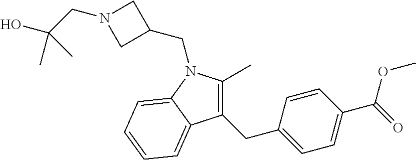 [SMILES: COC(=O)c1ccc(Cc2c(C)n(CC3CN(CC(C)(C)O)C3)c3ccccc23)cc1]

Methyl 4-((1-(azetidin-3-ylmethyl)-2-methyl-1H-indol-3-yl)methyl)benzoate hydrochloride (0.700 g, 1.82 mmol), 2,2-dimethyloxirane (1.311 g, 18.19 mmol) and potassium carbonate (1.257 g, 9.09 mmol) were added to ethanol (10 mL), and heated by microwave irradiation at 110° C. for 1 hour, followed by cooling to room temperature. After completion of the reaction, the reaction mixture was concentrated under reduced pressure to remove the solvent, and water was added to the concentrate, followed by extraction with ethyl acetate. The organic layer was washed with a saturated aqueous solution of sodium chloride, dried with anhydrous magnesium sulfate, filtered, and then concentrated under reduced pressure. The concentrate was purified by column chromatography (SiO₂, 12 g cartridge; ethyl acetate/hexane=from 40% to 70%) and concentrated to afford the title compound (0.442 g, 58%) as a brown liquid.

### Step 5: (Formula 8) Methyl 4-((1-((1-(2-fluoro-2-methylpropyl)azetidin-3-yl)methyl)-2-methyl-1H-indol-3-yl)methyl)benzoate

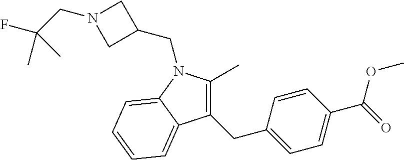 [SMILES: COC(=O)c1ccc(Cc2c(C)n(CC3CN(CC(C)(C)F)C3)c3ccccc23)cc1]

Methyl 4-((1-((1-(2-hydroxy-2-methylpropyl)azetidin-3-yl)methyl)-2-methyl-1H-indol-3-yl)methyl)benzoate (0.442 g, 1.05 mmol) was dissolved in methylene chloride (20 mL) at room temperature. To the solution, diethylaminosulfur trifluoride (0.167 mL, 1.26 mmol) was added, followed by stirring at the same temperature for 2 hours. Then, the reaction mixture was concentrated under reduced pressure to remove the solvent, and a saturated aqueous solution of sodium hydrogen carbonate was added to the concentrate, followed by extraction with methylene chloride. The organic layer was washed with a saturated aqueous solution of sodium chloride, dried with anhydrous magnesium sulfate, filtered, and then concentrated under reduced pressure. The concentrate was purified by column chromatography (SiO₂, 12 g cartridge; ethyl acetate/hexane=from 30% to 60%) and concentrated to afford the title compound (0.148 g, 33%) as a yellow liquid.

### Step 6: (Compound 750) 4-((1-((1-(2-Fluoro-2-methylpropyl)azetidin-3-yl)methyl)-2-methyl-1H-indol-3-yl)methyl)-N-hydroxybenzamide

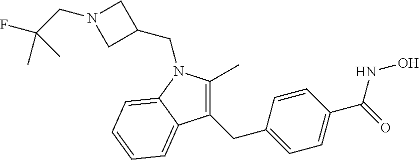 [SMILES: Cc1c(Cc2ccc(C(=O)NO)cc2)c2ccccc2n1CC1CN(CC(C)(C)F)C1]

Methyl 4-((1-((1-(2-fluoro-2-methylpropyl)azetidin-3-yl)methyl)-2-methyl-1H-indol-3-yl)methyl)benzoate (0.148 g, 0.35 mmol) was dissolved in methanol (10 mL) at room temperature. To the solution, hydroxylamine (50% aqueous solution, 2.142 mL, 35.03 mmol) and potassium hydroxide (0.197 g, 3.50 mmol) were added, followed by stirring at the same temperature for 3 hours. Then, the reaction mixture was concentrated under reduced pressure to remove solvent. To the concentrate, a saturated aqueous solution of sodium hydrogen carbonate (10 mL) was added, followed by stirring. The precipitated solid was filtered, washed with water, and then dried to afford compound 750 (0.126 g, 85%) as a white solid.

¹H NMR (400 MHz, DMSO-d₆) δ 7.60 (d, 2H, J=8.2 Hz), 7.40 (d, 1H, J=8.1 Hz), 7.35 (d, 1H, J=8.0 Hz), 7.22 (d, 2H, J=8.3 Hz), 7.04 (t, 1H, J=7.6 Hz), 6.92 (t, 1H, J=7.4 Hz), 4.32 (d, 2H, J=7.3 Hz), 4.05 (s, 2H), 3.22 (t, 2H, J=7.0 Hz), 2.95 (t, 2H, J=6.4 Hz), 2.78 (m, 1H), 2.46 (d, 2H, J=21.4 Hz), 2.42 (s, 3H), 1.28 (s, 3H), 1.23 (s, 3H); MS (ESI) m/z 424.2 (M⁺+H).

The structures of the above-described compounds are shown in Tables 11 to 16 below.

TABLE 11

| Com- | |
|---|---|
| pound | structure |
| 153 | 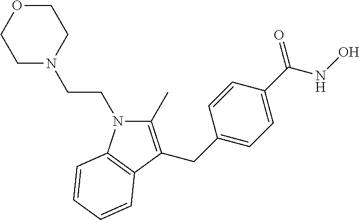 [SMILES: Cc1c(Cc2ccc(C(=O)NO)cc2)c2ccccc2n1CCN1CCOCC1] |
| 154 | 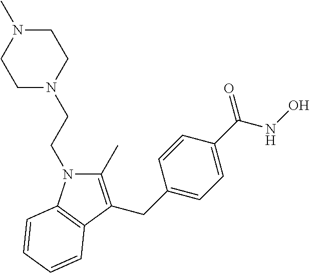 [SMILES: Cc1c(Cc2ccc(C(=O)NO)cc2)c2ccccc2n1CCN1CCN(C)CC1] |
| 155 | 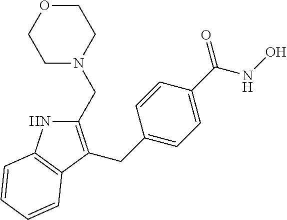 [SMILES: O=C(NO)c1ccc(Cc2c(CN3CCOCC3)[nH]c3ccccc23)cc1] |
| 196 | 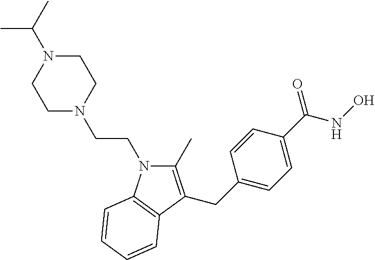 [SMILES: Cc1c(Cc2ccc(C(=O)NO)cc2)c2ccccc2n1CCN1CCN(C(C)C)CC1] |
| 197 | 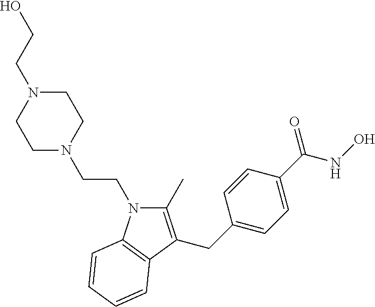 [SMILES: Cc1c(Cc2ccc(C(=O)NO)cc2)c2ccccc2n1CCN1CCN(CCO)CC1] |
| 198 | 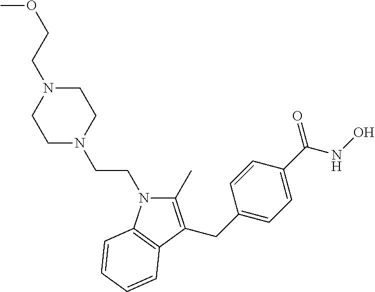 [SMILES: COCCN1CCN(CCn2c(C)c(Cc3ccc(C(=O)NO)cc3)c3ccccc32)CC1] |
| 199 | 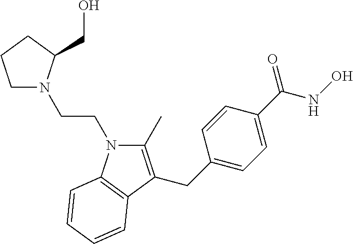 [SMILES: Cc1c(Cc2ccc(C(=O)NO)cc2)c2ccccc2n1CCN1CCC[C@H]1CO] |
| 200 | 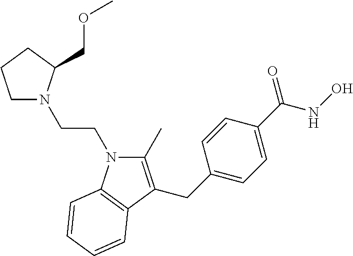 [SMILES: COC[C@@H]1CCCN1CCn1c(C)c(Cc2ccc(C(=O)NO)cc2)c2ccccc21] |
| 201 | 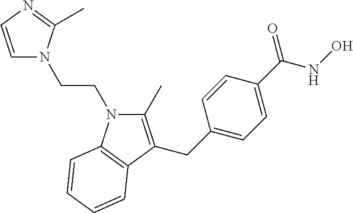 [SMILES: Cc1nccn1CCn1c(C)c(Cc2ccc(C(=O)NO)cc2)c2ccccc21] |
| 243 | 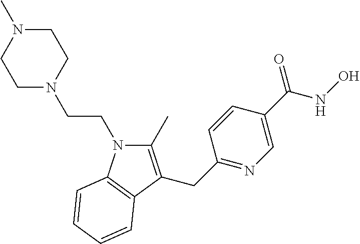 [SMILES: Cc1c(Cc2ccc(C(=O)NO)cn2)c2ccccc2n1CCN1CCN(C)CC1] |
| 244 | 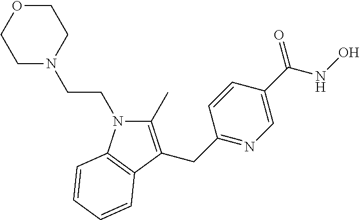 [SMILES: Cc1c(Cc2ccc(C(=O)NO)cn2)c2ccccc2n1CCN1CCOCC1] |
| 528 | 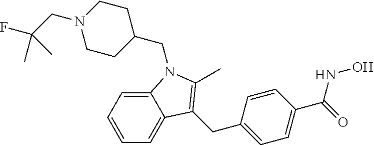 [SMILES: Cc1c(Cc2ccc(C(=O)NO)cc2)c2ccccc2n1CC1CCN(CC(C)(C)F)CC1] |

TABLE 12

| Compound | structure |
|---|---|
| 550 | 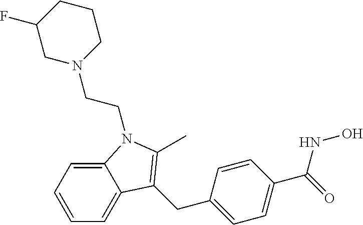 [SMILES: Cc1c(Cc2ccc(C(=O)NO)cc2)c2ccccc2n1CCN1CCCC(F)C1] |
| 551 | 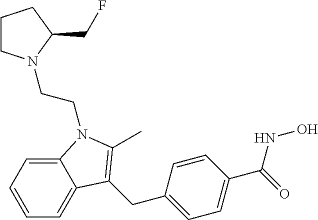 [SMILES: Cc1c(Cc2ccc(C(=O)NO)cc2)c2ccccc2n1CCN1CCC[C@H]1CF] |
| 553 | 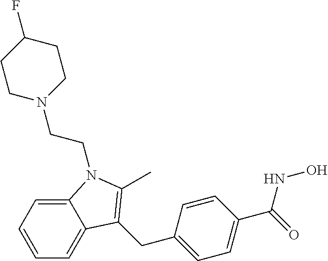 [SMILES: Cc1c(Cc2ccc(C(=O)NO)cc2)c2ccccc2n1CCN1CCC(F)CC1] |
| 556 | 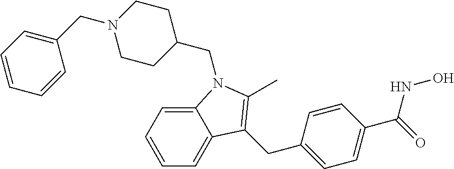 [SMILES: Cc1c(Cc2ccc(C(=O)NO)cc2)c2ccccc2n1CC1CCN(Cc2ccccc2)CC1] |
| 558 | 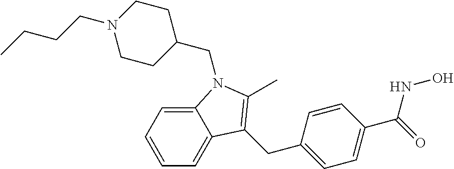 [SMILES: CCCCN1CCC(Cn2c(C)c(Cc3ccc(C(=O)NO)cc3)c3ccccc32)CC1] |
| 559 | 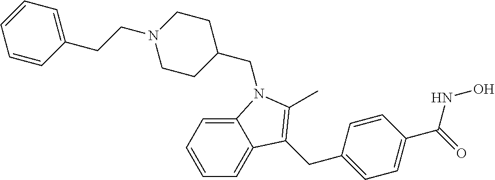 [SMILES: Cc1c(Cc2ccc(C(=O)NO)cc2)c2ccccc2n1CC1CCN(CCc2ccccc2)CC1] |
| 562 | 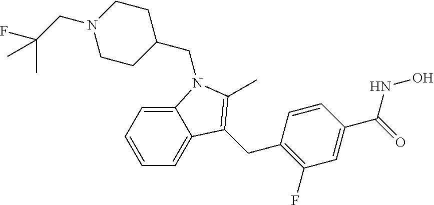 [SMILES: Cc1c(Cc2ccc(C(=O)NO)cc2F)c2ccccc2n1CC1CCN(CC(C)(C)F)CC1] |
| 563 | 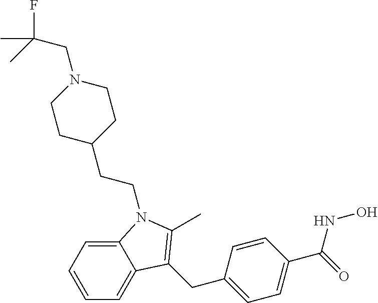 [SMILES: Cc1c(Cc2ccc(C(=O)NO)cc2)c2ccccc2n1CCC1CCN(CC(C)(C)F)CC1] |
| 564 | 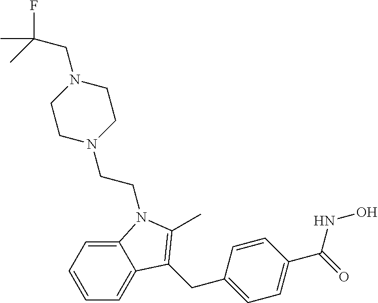 [SMILES: Cc1c(Cc2ccc(C(=O)NO)cc2)c2ccccc2n1CCN1CCN(CC(C)(C)F)CC1] |
| 581 | 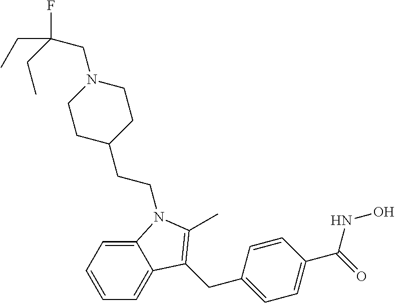 [SMILES: CCC(F)(CC)CN1CCC(CCn2c(C)c(Cc3ccc(C(=O)NO)cc3)c3ccccc32)CC1] |
| 582 | 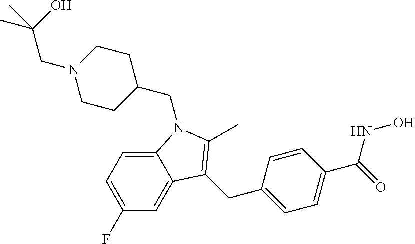 [SMILES: Cc1c(Cc2ccc(C(=O)NO)cc2)c2cc(F)ccc2n1CC1CCN(CC(C)(C)O)CC1] |
| 584 | 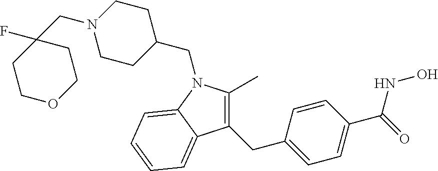 [SMILES: Cc1c(Cc2ccc(C(=O)NO)cc2)c2ccccc2n1CC1CCN(CC2(F)CCOCC2)CC1] |

TABLE 13

| Com- | |
|---|---|
| pound | structure |
| 585 | 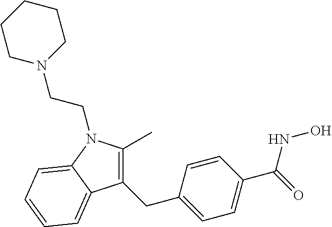 [SMILES: Cc1c(Cc2ccc(C(=O)NO)cc2)c2ccccc2n1CCN1CCCCC1] |
| 586 | 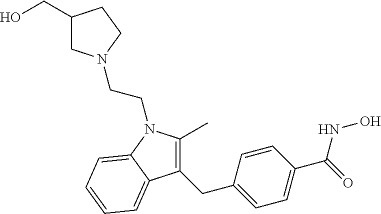 [SMILES: Cc1c(Cc2ccc(C(=O)NO)cc2)c2ccccc2n1CCN1CCC(CO)C1] |
| 587 | 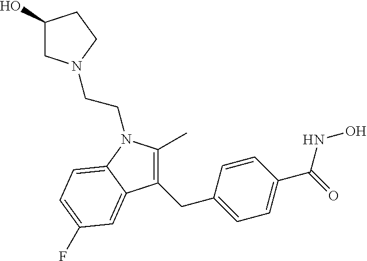 [SMILES: Cc1c(Cc2ccc(C(=O)NO)cc2)c2cc(F)ccc2n1CCN1CC[C@H](O)C1] |
| 588 | 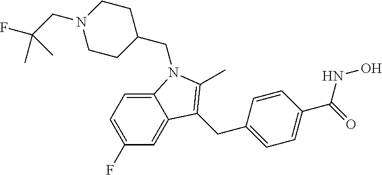 [SMILES: Cc1c(Cc2ccc(C(=O)NO)cc2)c2cc(F)ccc2n1CC1CCN(CC(C)(C)F)CC1] |
| 589 | 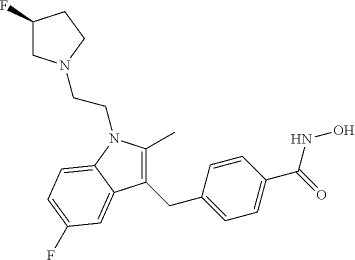 [SMILES: Cc1c(Cc2ccc(C(=O)NO)cc2)c2cc(F)ccc2n1CCN1CC[C@H](F)C1] |
| 590 | 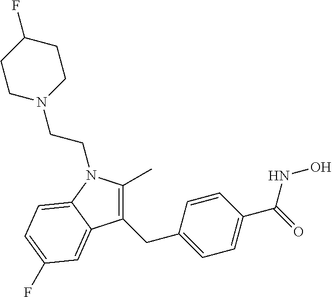 [SMILES: Cc1c(Cc2ccc(C(=O)NO)cc2)c2cc(F)ccc2n1CCN1CCC(F)CC1] |
| 591 | 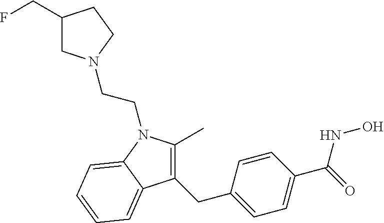 [SMILES: Cc1c(Cc2ccc(C(=O)NO)cc2)c2ccccc2n1CCN1CCC(CF)C1] |
| 592 | 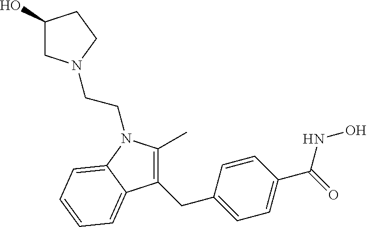 [SMILES: Cc1c(Cc2ccc(C(=O)NO)cc2)c2ccccc2n1CCN1CC[C@H](O)C1] |
| 593 | 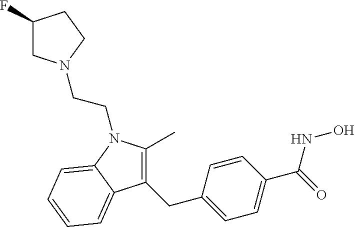 [SMILES: Cc1c(Cc2ccc(C(=O)NO)cc2)c2ccccc2n1CCN1CC[C@H](F)C1] |
| 594 | 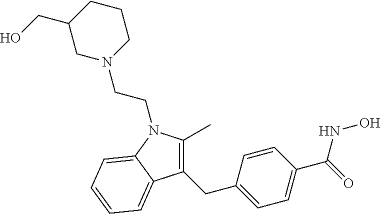 [SMILES: Cc1c(Cc2ccc(C(=O)NO)cc2)c2ccccc2n1CCN1CCCC(CO)C1] |
| 600 | 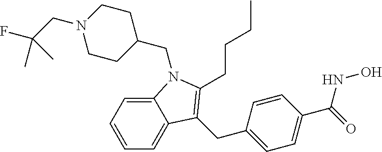 [SMILES: CCCCc1c(Cc2ccc(C(=O)NO)cc2)c2ccccc2n1CC1CCN(CC(C)(C)F)CC1] |
| 601 | 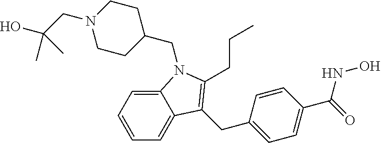 [SMILES: CCCc1c(Cc2ccc(C(=O)NO)cc2)c2ccccc2n1CC1CCN(CC(C)(C)O)CC1] |

TABLE 14

| Compound | structure |
|---|---|
| 602 | 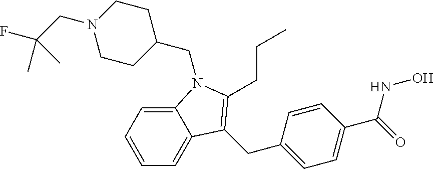 [SMILES: CCCc1c(Cc2ccc(C(=O)NO)cc2)c2ccccc2n1CC1CCN(CC(C)(C)F)CC1] |
| 603 | 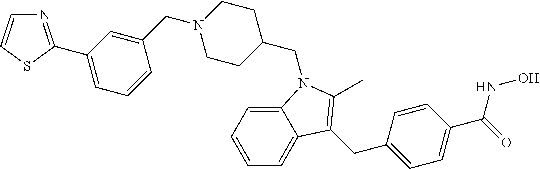 [SMILES: Cc1c(Cc2ccc(C(=O)NO)cc2)c2ccccc2n1CC1CCN(Cc2cccc(-c3nccs3)c2)CC1] |
| 608 | 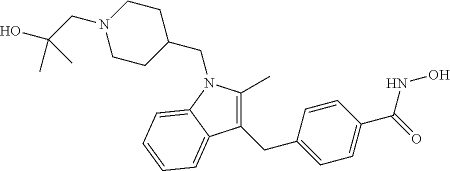 [SMILES: Cc1c(Cc2ccc(C(=O)NO)cc2)c2ccccc2n1CC1CCN(CC(C)(C)O)CC1] |
| 609 | 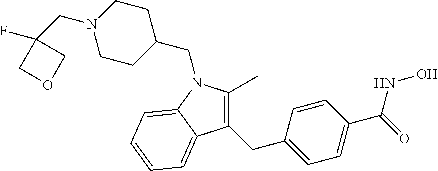 [SMILES: Cc1c(Cc2ccc(C(=O)NO)cc2)c2ccccc2n1CC1CCN(CC2(F)COC2)CC1] |
| 610 | 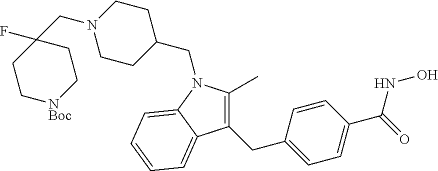 [SMILES: Cc1c(Cc2ccc(C(=O)NO)cc2)c2ccccc2n1CC1CCN(CC2(F)CCN(C(=O)OC(C)(C)C)CC2)CC1] |
| 611 | 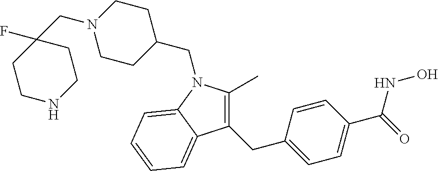 [SMILES: Cc1c(Cc2ccc(C(=O)NO)cc2)c2ccccc2n1CC1CCN(CC2(F)CCNCC2)CC1] |
| 612 | 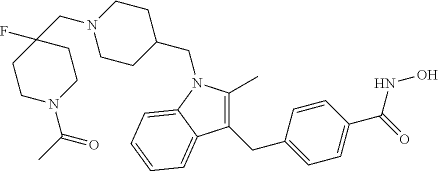 [SMILES: CC(=O)N1CCC(F)(CN2CCC(Cn3c(C)c(Cc4ccc(C(=O)NO)cc4)c4ccccc43)CC2)CC1] |
| 613 | 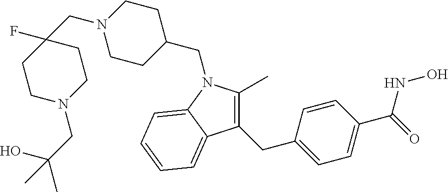 [SMILES: Cc1c(Cc2ccc(C(=O)NO)cc2)c2ccccc2n1CC1CCN(CC2(F)CCN(CC(C)(C)O)CC2)CC1] |
| 614 | 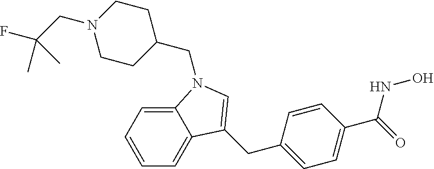 [SMILES: CC(C)(F)CN1CCC(Cn2cc(Cc3ccc(C(=O)NO)cc3)c3ccccc32)CC1] |
| 615 | 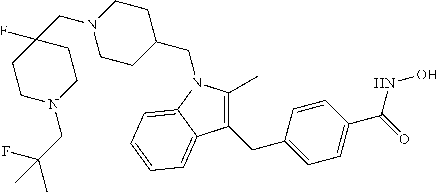 [SMILES: Cc1c(Cc2ccc(C(=O)NO)cc2)c2ccccc2n1CC1CCN(CC2(F)CCN(CC(C)(C)F)CC2)CC1] |
| 616 | 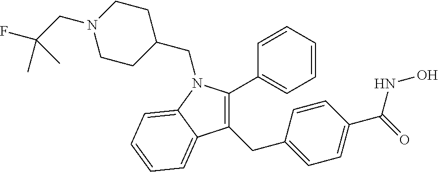 [SMILES: CC(C)(F)CN1CCC(Cn2c(-c3ccccc3)c(Cc3ccc(C(=O)NO)cc3)c3ccccc32)CC1] |
| 619 | 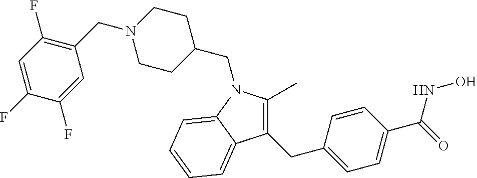 [SMILES: Cc1c(Cc2ccc(C(=O)NO)cc2)c2ccccc2n1CC1CCN(Cc2cc(F)c(F)cc2F)CC1] |
| 620 | 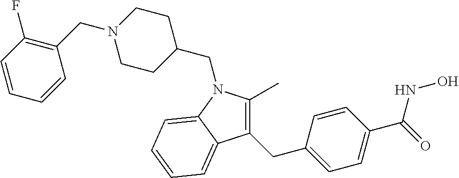 [SMILES: Cc1c(Cc2ccc(C(=O)NO)cc2)c2ccccc2n1CC1CCN(Cc2ccccc2F)CC1] |
| 621 | 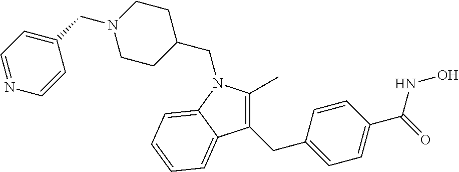 |
| 622 | 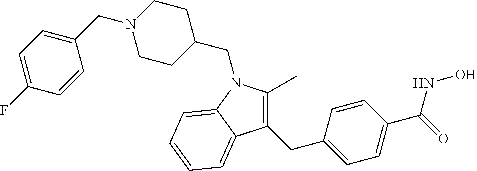 [SMILES: Cc1c(Cc2ccc(C(=O)NO)cc2)c2ccccc2n1CC1CCN(Cc2ccc(F)cc2)CC1] |
| 623 | 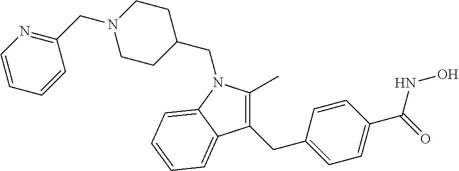 [SMILES: Cc1c(Cc2ccc(C(=O)NO)cc2)c2ccccc2n1CC1CCN(Cc2ccccn2)CC1] |

TABLE 15

| Compound | structure |
|---|---|
| 624 | 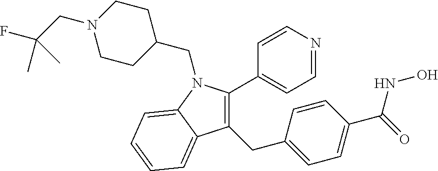 [SMILES: CC(C)(F)CN1CCC(Cn2c(-c3ccncc3)c(Cc3ccc(C(=O)NO)cc3)c3ccccc32)CC1] |
| 625 | 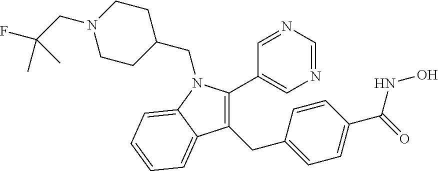 [SMILES: CC(C)(F)CN1CCC(Cn2c(-c3cncnc3)c(Cc3ccc(C(=O)NO)cc3)c3ccccc32)CC1] |
| 626 | 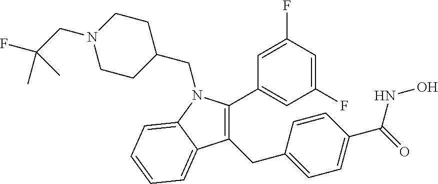 [SMILES: CC(C)(F)CN1CCC(Cn2c(-c3cc(F)cc(F)c3)c(Cc3ccc(C(=O)NO)cc3)c3ccccc32)CC1] |
| 627 | 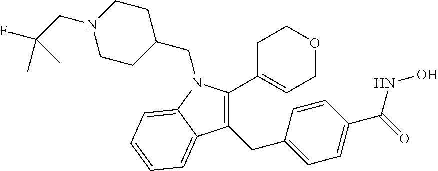 [SMILES: CC(C)(F)CN1CCC(Cn2c(C3=CCOCC3)c(Cc3ccc(C(=O)NO)cc3)c3ccccc32)CC1] |
| 631 | 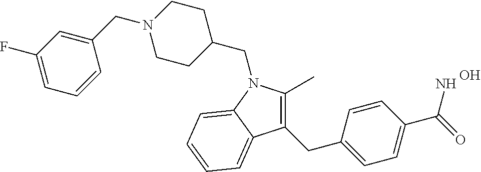 |
| 632 | 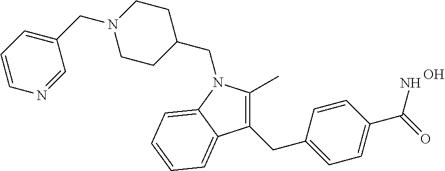 |
| 633 | 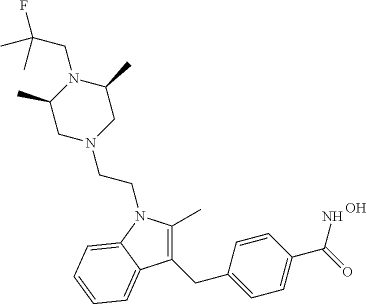 |
| 639 | 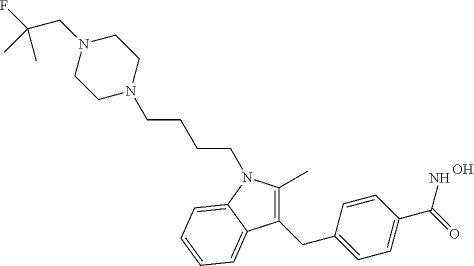 |
| 640 | 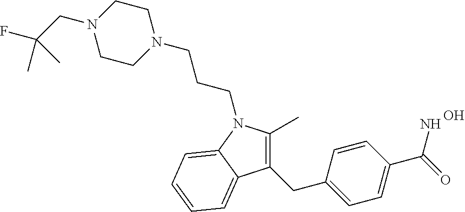 |
| 643 | 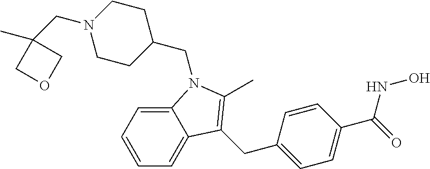 [SMILES: Cc1c(Cc2ccc(C(=O)NO)cc2)c2ccccc2n1CC1CCN(CC2(C)COC2)CC1] |
| 679 | 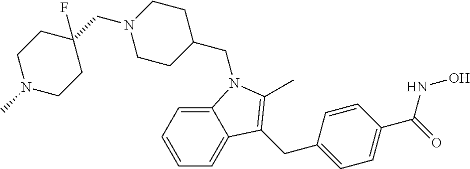 |
| 681 | 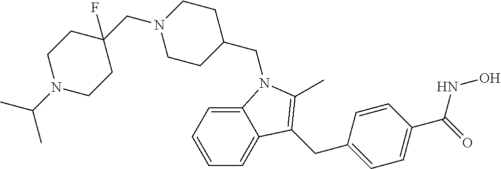 [SMILES: Cc1c(Cc2ccc(C(=O)NO)cc2)c2ccccc2n1CC1CCN(CC2(F)CCN(C(C)C)CC2)CC1] |
| 695 | 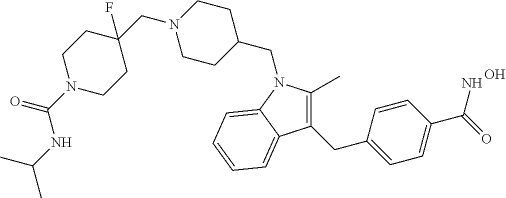 |
| 696 | 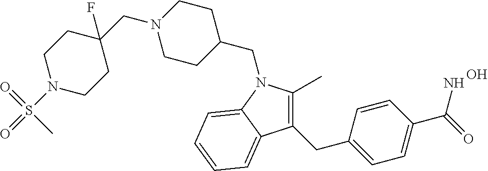 |

TABLE 16

| Compound | structure |
|---|---|
| 697 | 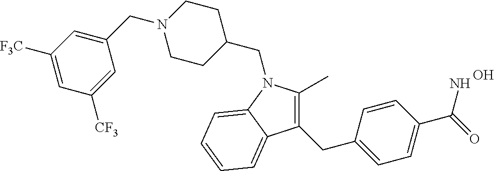 |
| 698 | 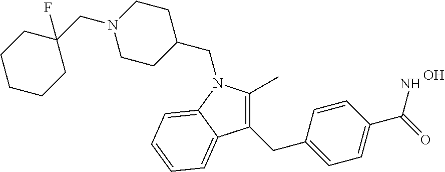 |
| 707 | 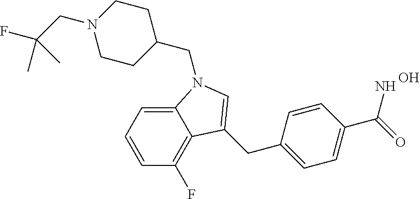 |
| 708 | 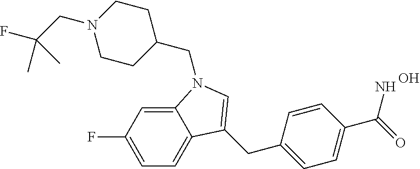 |
| 713 | 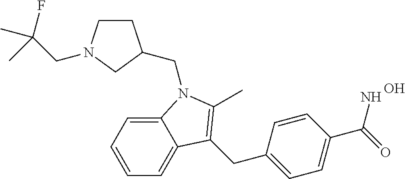 |
| 728 | 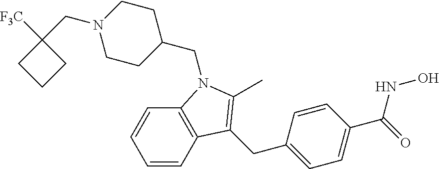 [SMILES: Cc1c(Cc2ccc(C(=O)NO)cc2)c2ccccc2n1CC1CCN(CC2(C(F)(F)F)CCC2)CC1] |
| 747 | 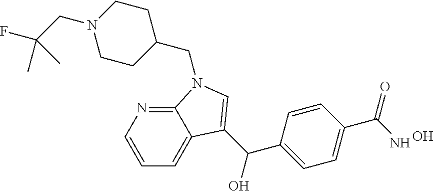 |
| 748 | 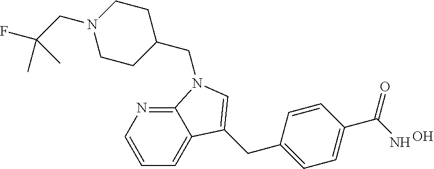 |
| 749 | 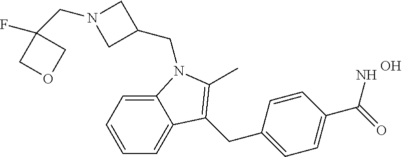 |
| 750 | 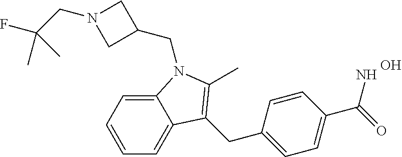 |

Compound 153
N-hydroxy-4-((2-methyl-1-(2-morpholinoethyl)-1H-indol-3-yl)methyl)benzamide;

Compound 154:
N-hydroxy-4-((2-methyl-1-(2-(4-methylpiperazin-1-yl)ethyl)-1H-indol-3-yl)methyl)benzamide;

Compound 155:
N-hydroxy-4-((2-(morpholinomethyl)-1H-indol-3-yl)methyl)benzamide;

Compound 196:
N-hydroxy-4-((1-(2-(4-isopropylpiperazin-1-yl)ethyl)-2-methyl-1H-indol-3-yl)methyl)benzamide;

Compound 197:
N-hydroxy-4-((1-(2-(4-(2-hydroxyethyl)piperazin-1-yl)ethyl)-2-methyl-1H-indol-3-yl)methyl)benzamide;

Compound 198:
N-hydroxy-4-((1-(2-(4-(2-methoxyethyl)piperazin-1-yl)ethyl)-2-methyl-1H-indol-3-yl)methyl)benzamide;

Compound 199:
(S)—N-hydroxy-4-((1-(2-(2-(hydroxymethyl)pyrrolidin-1-yl)ethyl)-2-methyl-1H-indol-3-yl)methyl)benzamide;

Compound 200:
(S)—N-hydroxy-4-((1-(2-(2-(methoxymethyl)pyrrolidin-1-yl)ethyl)-2-methyl-1H-indol-3-yl)methyl)benzamide;

Compound 201:
N-hydroxy-4-((2-methyl-1-(2-(2-methyl-1H-imadazol-1-yl)ethyl)-1H-indol-3-yl)methyl)benzamide;

Compound 243:
N-hydroxy-6-((2-methyl-1-(2-(4-methylpeperazin-1-yl)ethyl)-1H-indol-3-yl)methyl)nicotinamide;

Compound 244:
N-hydroxy-6-((2-methyl-1-(2-morpholinoethyl)-1H-indol-3-yl)methyl)nocotinamide;

Compound 528:
4-((1-((1-(2-fluoro-2-methylpropyl)piperidin-4-yl)methyl)-2-methyl-1H-indol-3-yl)methyl)-N-hydroxybenzamide;

Compound 550:
4-((1-(2-(3-fluoropiperidin-1-yl)ethyl)-2-methyl-1H-indol-3-yl)methyl)-N-hydroxybenzamide;

Compound 551:
(S)-4-((1-(2-(2-(fluoromethyl)pyrrolidin-1-yl)ethyl)-2-methyl-1H-indol-3-yl)methyl)-N-hydroxybenzamide;

Compound 553:
4-((1-(2-(4-fluoropiperidin-1-yl)ethyl)-2-methyl-1H-indol-3-yl)methyl)-N-hydroxybenzamide;

Compound 556:
4-((1-((1-benzylpiperidin-4-yl)methyl)-2-methyl-1H-indol-3-yl)methyl)-N-hydroxybenzamide;

Compound 558:
4-((1-((1-butylpiperidin-4-yl)methyl)-2-methyl-1H-indol-3-yl)methyl)-N-hydroxybenzamide;

Compound 559:
N-hydroxy-4-((2-methyl-1-((1-phenethylpiperidin-4-yl)methyl)-1H-indol-3-yl)methyl)benzamide;

Compound 562:
3-fluoro-4-((1-((1-(2-fluoro-2-methylpropyl)piperidin-4-yl)methyl)-2-methyl-1H-indol-3-yl)methyl)-N-hydroxybenzamide;

Compound 563:
4-((1-(2-(1-(2-fluoro-2-methylpropyl)piperidin-4-yl)ethyl)-2-methyl-1H-indol-3-yl)methyl)-N-hydroxybenzamide;

Compound 564:
4-((1-(2-(4-(2-fluoro-2-methylpropyl)piperazin-1-yl)ethyl)-2-methyl-1H-indol-3-yl)methyl)-N-hydroxybenzamide;

Compound 581:
4-((1-(2-(1-(2-ethyl-2-fluorobutyl)piperidin-4-yl)ethyl)-2-methyl-1H-indol-3-yl)methyl)-N-hydroxybenzamide;

Compound 582:
4-((5-fluoro-1-((1-(2-hydroxy-2-methylpropyl)piperidin-4-yl)methyl)-2-methyl-1H-indol-3-yl)methyl)-N-hydroxybenzamide;

Compound 584:
4-((1-((1-((4-fluoro-tetrahydro-2H-pyran-4-yl)methyl)piperidin-4-yl)methyl)-2-methyl-1H-indol-3-yl)methyl)-N-hydroxybenzamide;

Compound 585:
4-((1-((1-((4-fluoro-tetrahydro-2H-pyran-4-yl)methyl)piperidin-4-yl)methyl)-2-methyl-1H-indol-3-yl)methyl)-N-hydroxybenzamide;

Compound 586:
N-hydroxy-4-((1-(2-(3-(hydroxymethyl)pyrroldin-1-yl)ethyl)-2-methyl-1H-indol-3-yl)methyl)benzamide;

Compound 587:
(S)-4-((5-fluoro-1-(2-(3-hydroxypyrrolidin-1-yl)ethyl)-2-methyl-1H-indol-3-yl)methyl)-N-hydroxybenzamide;

Compound 588:
4-((5-fluoro-1-((1-(2-fluoro-2-methylpropyl)piperidin-4-yl)methyl)-2-methyl-1H-indol-3-yl)methyl)-N-hydroxybenzamide;

Compound 589:
(S)-4-((5-fluoro-1-(2-(3-fluoropyrrolidin-1-yl)ethyl)-2-methyl-1H-indol-3-yl)methyl)-N-hydroxybenzamide;

Compound 590:
4-((5-fluoro-1-(2-(4-fluoropiperidin-1-yl)ethyl)-2-methyl-1H-indol-3-yl)methyl)-N-hydroxybenzamide;

Compound 591:
4-((1-(2-(3-(fluoromethyl)pyrrolidin-1-yl)ethyl)-2-methyl-1H-indol-3-yl)methyl)-N-hydroxybenzamide;

Compound 592:
(S)—N-hydroxy-4-((1-(2-(3-hydroxypyrrolidin-1-yl)ethyl)-2-methyl-1H-indol-3-yl)methyl)benzamide;

Compound 593:
(S)-4-((1-(2-(3-fluoropyrrolidin-1-yl)ethyl)-2-methyl-1H-indol-3-yl)methyl)-N-hydroxybenzamide;

Compound 594:
N-hydroxy-4-((1-(2-(3-(hydroxymethyl)piperidin-1-yl)ethyl)-2-methyl-1H-indol-3-yl)methyl)benzamide;

Compound 600:
4-((2-butyl-1-((1-(2-fluoro-2-methylpropyl)piperidin-4-yl)methyl)-1H-indol-3-yl)methyl)-N-hydroxybenzamide;

Compound 601:
N-hydroxy-4-((1-((1-(2-hydroxy-2-methylpropyl)piperidin-4-yl)methyl)-2-propyl-1H-indol-3-yl)methyl)benzamide;

Compound 602:
4-((1-((1-(2-fluoro-2-methylpropyl)piperidin-4-yl)methyl)-2-propyl-1H-indol-3-yl)methyl)-N-hydroxybenzamide;

Compound 603:
N-hydroxy-4-((2-methyl-1-((1-(3-(thiazol-2-yl)benzyl)piperidin-4-yl)methyl)-1H-indol-3-yl)methyl)benzamide;

Compound 608:
N-hydroxy-4-((1-((1-(2-hydroxy-2-methylpropyl)piperidin-4-yl)methyl)-2-methyl-1H-indol-3-yl)methyl)benzamide;

Compound 609:
4-((1-((1-((3-fluoroxetan-3-yl)methyl)piperidin-4-yl)methyl)-2-methyl-1H-indol-3-yl)methyl)-N-hydroxybenzamide;

Compound 610:
tert-butyl 4-fluoro-4-((4-((3-(4-(hydroxycarbamoyl)benzyl)-2-methyl-1H-indol-1-yl)methyl)piperidin-1-yl)methyl)piperidine-1-carboxylate;

Compound 611:
4-((1-((1-((4-fluoropiperidin-4-yl)methyl)piperidin-4-yl)methyl)-2-methyl-1H-indol-3-yl)methyl)-N-hydroxybenzamide;

Compound 612:
4-((1-((1-((1-acetyl-4-fluoropiperidin-4-yl)methyl)piperidin-4-yl)methyl)-2-methyl-1H-indol-3-yl)methyl)-N-hydroxybenzamide;

Compound 613:
4-((1-((1-((4-fluoro-1-(2-hydroxy-2-methylpropyl)piperidin-4-yl)methyl)piperidin-4-yl)methyl)-2-methyl-1H-indol-3-yl)methyl)-N-hydroxybenzamide;

Compound 614:
4-((1-((1-(2-fluoro-2-methylpropyl)piperidin-4-yl)methyl)-1H-indol-3-yl)methyl)-N-hydroxybenzamide;

Compound 615:
4-((1-((1-((4-fluoro-1-(2-fluoro-2-methylpropyl)piperidin-4-yl)methyl)piperidin-4-yl)methyl)-2-methyl-1H-indol-3-yl)methyl)-N-hydroxybenzamide;

Compound 616:
4-((1-((1-(2-fluoro-2-methylpropyl)piperidin-4-yl)methyl)-2-phenyl-1H-indol-3-yl)methyl)-N-hydroxybenzamide;

Compound 619:
N-hydroxy-4-((2-methyl-1-((1-(2,4,5-trifluorobenzyl)piperidin-4-yl)methyl)-1H-indol-3-yl)methyl)benzamide;

Compound 620:
4-((1-((1-(2-fluorobenzyl)piperidin-4-yl)methyl)-2-methyl-1H-indol-3-yl)methyl)-N-hydroxybenzamide;

Compound 621:
N-hydroxy-4-((2-methyl-1-((1-(pyridin-4-ylmethyl)piperidin-4-yl)methyl)-1H-indol-3-yl)methyl)benzamide;

Compound 622:
4-((1-((1-(4-fluorobenzyl)piperidin-4-yl)methyl)-2-methyl-1H-indol-3-yl)methyl)-N-hydroxybenzamide;

Compound 623:
N-hydroxy-4-((2-methyl-1-((1-(pyridin-2-ylmethyl)piperidin-4-yl)methyl)-1H-indol-3-yl)methyl)benzamide;

Compound 624:
4-((1-((1-(2-fluoro-2-methylpropyl)piperidin-4-yl)methyl)-2-(pyridin-4-yl)-1H-indol-3-yl)methyl)-N-hydroxybenzamide;

Compound 625:
4-((1-((1-(2-fluoro-2-methylpropyl)piperidin-4-yl)methyl)-2-(pyrimidin-5-yl)-1H-indol-3-yl)methyl)-N-hydroxybenzamide;

Compound 626:
4-((2-(3,5-difluorophenyl)-1-((1-(2-fluoro-2-methylpropyl)piperidin-4-yl)methyl)-1H-indol-3-yl)methyl)-N-hydroxybenzamide;

Compound 627:
4-((2-(3,6-dihydro-2H-pyran-4-yl)-1-((1-(2-fluoro-2-methylpropyl)piperidin-4-yl)methyl)-1H-indol-3-yl)methyl)-N-hydroxybenzamide;

Compound 631:
4-((1-((1-(3-fluorobenzyl)piperidin-4-yl)methyl)-2-methyl-1H-indol-3-yl)methyl)-N-hydroxybenzamide;

Compound 632:
N-hydroxy-4-((2-methyl-1-((1-(pyridin-3-ylmethyl)piperidin-4-yl)methyl)-1H-indol-3-yl)methyl)benzamide;

Compound 633:
4-((1-(2-((3S,5R)-4-(2-fluoro-2-methylpropyl)-3,5-dimethylpiperazin-1-yl)ethyl)-2-methyl-1H-indol-3-yl)methyl)-N-hydroxybenzamide;

Compound 639:
4-((1-(4-(4-(2-fluoro-2-methylpropyl)piperazin-1-yl)butyl)-2-methyl-1H-indol-3-yl)methyl)-N-hydroxybenzamide;

Compound 640:
4-((1-(3-(4-(2-fluoro-2-methylpropyl)piperazin-1-yl)propyl)-2-methyl-1H-indol-3-yl)methyl)-N-hydroxybenzamide;

Compound 643:
N-hydroxy-4-((2-methyl-1-((1-((3-methyloxetan-3-yl)methyl)piperidin-4-yl)methyl)-1H-indol-3-yl)methyl)benzamide;

Compound 679:
4-((1-((1-((4-fluoro-1-methylpiperidin-4-yl)methyl)piperidin-4-yl)methyl)-2-methyl-1H-indol-3-yl)methyl)-N-hydroxybenzamide;

Compound 681:
4-((1-((1-((4-fluoro-1-isopropylpiperidin-4-yl)methyl)piperidin-4-yl)methyl)-2-methyl-1H-indol-3-yl)methyl)-N-hydroxybenzamide;

Compound 695:
4-fluoro-4-((4-((3-(4-(hydroxycarbamoyl)benzyl)-2-methyl-1H-indol-1-yl)methyl)piperidin-1-yl)methyl)-N-isopropylpiperidine-1-carboxamide;

Compound 696:
4-((1-((1-((4-fluoro-1-(methylsulfonyl)piperidin-4-yl)methyl)piperidin-4-yl)methyl)-2-methyl-1H-indol-3-yl)methyl)-N-hydroxybenzamide;

Compound 697:
4-((1-((1-(3,5-bis(trifluoromethyl)benzyl)piperidin-4-yl)methyl)-2-methyl-1H-indol-3-yl)methyl)-N-hydroxybenzamide;

Compound 698:
4-((1-((1-((1-fluorocyclohexyl)methyl)piperidin-4-yl)methyl)-2-methyl-1H-indol-3-yl)methyl)-N-hydroxybenzamide;

Compound 707:
4-((4-fluoro-1-((1-(2-fluoro-2-methylpropyl)piperidin-4-yl)methyl)-1H-indol-3-yl)methyl)-N-hydroxybenzamide;

Compound 708:
4-((6-fluoro-1-((1-(2-fluoro-2-methylpropyl)piperidin-4-yl)methyl)-1H-indol-3-yl)methyl)-N-hydroxybenzamide;

Compound 713:
4-((1-((1-(2-fluoro-2-methylpropyl)pyrrolidin-3-yl)methyl)-2-methyl-1H-indol-3-yl)methyl)-N-hydroxybenzamide;

Compound 728:
N-hydroxy-4-((2-methyl-1-((1-((1-(trifluoromethyl)cyclobutyl)methyl)piperidin-4-yl)methyl)-1H-indol-3-yl)methyl)benzamide;

Compound 747:
4-((1-((1-(2-fluoro-2-methylpropyl)piperidin-4-yl)methyl)-1H-pyrrolo[2,3-b]pyridin-3-yl)(hydroxy)methyl)-N-hydroxybenzamide;

Compound 748:
4-((1-((1-(2-fluoro-2-methylpropyl)piperidin-4-yl)methyl)-1H-pyrrolo[2,3-b]pyridin-3-yl)methyl)-N-hydroxybenzamide;

Compound 749:
4-((1-((1-((3-fluorooxetan-3-yl)methyl)azetidin-3-yl)methyl)-2-methyl-1H-indol-3-yl)methyl)-N-hydroxybenzamide;

Compound 750:
4-((1-((1-(2-fluoro-2-methylpropyl)azetidin-3-yl)methyl)-2-methyl-1H-indol-3-yl)methyl)-N-hydroxybenzamide.

### Measurement of Activities of Compounds According to the Present Invention

### Experimental Protocol

### Experimental Example 1

### Experiment on Inhibition of HDAC enzyme activity (in vitro)

1. Experiment on Activities in HDAC Enzymes (HDAC1 and HDAC6)

Assays were performed using a HDAC1 fluorimetric drug discovery assay kit (Enzolifesciences: BML-AK511) and a HDAC6 human recombinant (Calbiochem: 382180). The assay mixture of the kit was treated with each of the compounds of the Examples at concentrations 100, 1000 and 10000 nM for HDAC1 assay and 0.1, 1, 10, 100 and 1000 nM for HDAC6 assay. The treated assay mixture was allowed to react at 37° C. for 60 minutes, and then was treated with a developer and allowed to stand at 37° C. for 30 minutes, followed by quantification of the fluorescence. The results of the experiment are shown in Table 17 below. In Table 17, lower HDAC6 values (μM) indicate higher HDAC6 inhibitory activities of the compounds. Also, because selective HDAC6 inhibitors should not inhibit HDAC1 (μM), higher HDAC1 values indicate higher HDAC6 selectivity of the compounds.

2. Results of Measurement of the Abilities to Inhibit the Activities of HDAC Enzymes (HDAC1 and HDAC6)

TABLE 17

| Compounds | HDAC6 (μM) | HDAC1 (μM) |
|---|---|---|
| Compound 153 | 0.14 | 4.38 |
| Compound 154 | 0.008 | 2.22 |
| Compound 155 | 0.024 | 3.86 |
| Compound 196 | 0.006 | 1.61 |
| Compound 197 | 0.008 | 1.81 |
| Compound 198 | 0.005 | 1.89 |
| Compound 199 | 0.004 | 1.26 |
| Compound 200 | 0.008 | 2.61 |
| Compound 201 | 0.009 | 2.47 |
| Compound 243 | 0.340 | 11.4 |
| Compound 244 | 0.236 | 10.9 |
| Compound 528 | 0.074 | 8.00 |
| Compound 550 | 0.068 | 16.9 |
| Compound 551 | 0.082 | 20.7 |
| Compound 553 | 0.039 | 8.10 |
| Compound 556 | 0.108 | 13.3 |
| Compound 558 | 0.080 | 6.11 |
| Compound 559 | 0.273 | 13.2 |
| Compound 562 | 0.087 | 12.8 |
| Compound 563 | 0.307 | 18.8 |
| Compound 564 | 0.025 | 0.37 |
| Compound 581 | 0.870 | >10 |
| Compound 582 | 0.027 | 2.46 |
| Compound 584 | 0.007 | 11.3 |
| Compound 585 | 0.084 | 7.75 |
| Compound 586 | 0.016 | 2.81 |
| Compound 587 | 0.030 | 2.81 |
| Compound 588 | 0.378 | 6.73 |
| Compound 589 | 0.063 | 4.64 |
| Compound 590 | 0.049 | 6.12 |
| Compound 591 | 0.031 | 6.61 |
| Compound 592 | 0.016 | 4.95 |
| Compound 593 | 0.032 | 7.14 |
| Compound 594 | 0.017 | 3.14 |
| Compound 601 | 0.098 | 5.60 |
| Compound 602 | 0.300 | >10 |
| Compound 603 | 0.206 | >10 |
| Compound 608 | 0.007 | 1.07 |
| Compound 609 | 0.005 | 3.63 |
| Compound 610 | 0.079 | >10 |
| Compound 611 | 0.024 | 2.93 |
| Compound 612 | 0.061 | 9.10 |
| Compound 613 | 0.069 | 6.74 |
| Compound 614 | 0.150 | 14.8 |
| Compound 615 | 0.007 | >10 |
| Compound 616 | 0.821 | 17.4 |
| Compound 619 | 0.770 | >10 |
| Compound 620 | 0.306 | >10 |
| Compound 621 | 0.089 | 10.5 |
| Compound 622 | 0.090 | 19.2 |
| Compound 623 | 0.028 | 1.48 |
| Compound 624 | 0.107 | 5.01 |
| Compound 625 | 0.071 | 5.72 |
| Compound 626 | 0.587 | >10 |
| Compound 627 | 0.352 | 11.9 |
| Compound 631 | 1.36 | >10 |
| Compound 632 | 0.063 | 1.79 |
| Compound 633 | 0.075 | 1.76 |
| Compound 639 | 0.076 | 4.98 |
| Compound 640 | 0.115 | 7.60 |
| Compound 643 | 0.028 | 4.58 |
| Compound 679 | 0.013 | 0.832 |
| Compound 681 | 0.037 | 0.802 |
| Compound 695 | 0.024 | 11.5 |
| Compound 696 | 0.123 | >10 |
| Compound 698 | 0.430 | >10 |
| Compound 707 | 0.171 | 12.0 |
| Compound 708 | 0.432 | 14.9 |
| Compound 713 | 0.246 | >10 |
| Compound 728 | 1.19 | >10 |
| Compound 747 | 0.334 | 6.10 |
| Compound 748 | 0.140 | 3.88 |
| Compound 749 | 0.092 | 8.59 |
| Compound 750 | 0.114 | 10.7 |

3. Western Blot Analysis

The degrees of acetylation of Histone H3 and H4 (substrates of HDAC class 1) and tubulin (representative substrate of HDAC6) were examined by Western blot analysis in order to confirm the ability of compound 528 to selectively inhibit HDAC6 in cells.

Specifically, RPMI8226 cells were seeded into a six-well plate at a density of 1.0×10⁶ cells/well, and then treated with varying concentrations of compound 528. After 24 hours, protein was extracted from the cells using RIPA buffer and quantified by the Bradford method. 25 μg of the protein was added to sample buffer and electrophoresed on 4-12% gradient gel, and the gel was transferred to a nitrocellulose membrane for 50 minutes. The membrane was blocked in 5% skim milk solution for 1 hour. Anti-acetyl H3 antibody (1:2,000), anti-acetyl H4 antibody (1:5,000), anti-acetyl tubulin antibody (1:5,000) and anti-β-actin antibody (1:10,000) were added to 5% skim milk, and the membrane was immersed in the skim milk and allowed to react at 4° C. for 16 hours, after which it was washed three times with 1×TBS-T for 10 minutes each washing. IgG-HRP antibody (1:5,000) was added to the 5% skim milk, and the membrane was immersed in the skim milk and allowed to react at room temperature for 40 minutes, after which it was washed three times with 1×TBS-T for 10 minutes each washing. Detection was performed by LAS 3000 using ECL solution.

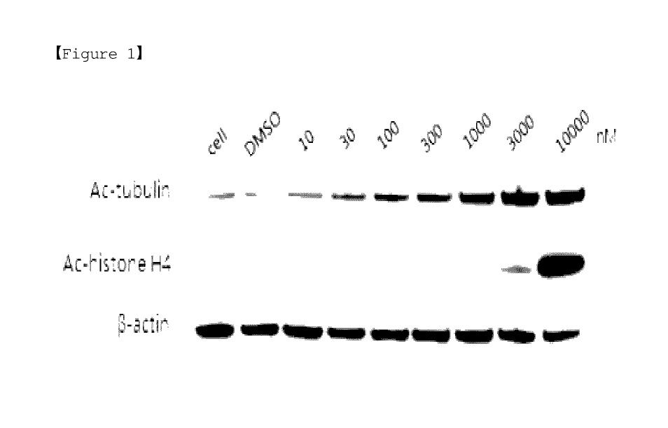

The results are shown in FIG. 1.

As can be seen in FIG. 1, tubulin acetylation (HDAC6) appeared at a compound concentration as low as 100 nM, suggesting that compound 528 has high activity even at low concentrations. However, histone acetylation (HDAC1) appeared at a compound concentration of at least 3 uM, suggesting that compound 528 has little or no activity against HDAC1 at low concentrations. This difference in concentration of the compound between the expressions of tubulin and histone in cells demonstrates that the compound selectively inhibits HDAC6 in cells.

From this difference in concentration of the compound between the expressions of tubulin and histone in cells, it can be seen that the novel indole derivative compounds having a carbon-carbon bond according to the present invention have high selectivity for HDAC6 in cells.

### Experimental Example 2

### Experiment on Distribution of Compound 528 in Brain Tissue of Mice (In Vivo)

The distribution of the compound of the present invention in the brain tissue of ICR mice was examined. Specifically, the compound of the present invention was administered orally (PO) to ICR mice at a dose of 50 mg/kg. At 0.5, 1 and 2 hours after administration, blood was collected from the orbital venous plexus of the mice. After the mice were euthanized, the brain tissue (including cerebral, cerebellar and medullar tissues) was extracted.

The blood was centrifuged at 13000 rpm for 4 minutes, and then 30 μL of plasma was collected, and an organic solvent (90 wt % acetonitrile in methanol) was added thereto to remove protein. Then, the solution was centrifuged, and the supernatant was collected.

In addition, saline was added to the isolated brain tissue, and homogenized using a homogenizer. From the homogenized dilution, 100 μL of a sample was taken, and an organic solvent (90 wt % acetonitrile in methanol) was added thereto to remove protein, after which the solution was centrifuged, and the supernatant was collected. The concentrations of the compound of the present invention in the pretreated plasma and brain tissue solutions were analyzed by LC-MS/MS.

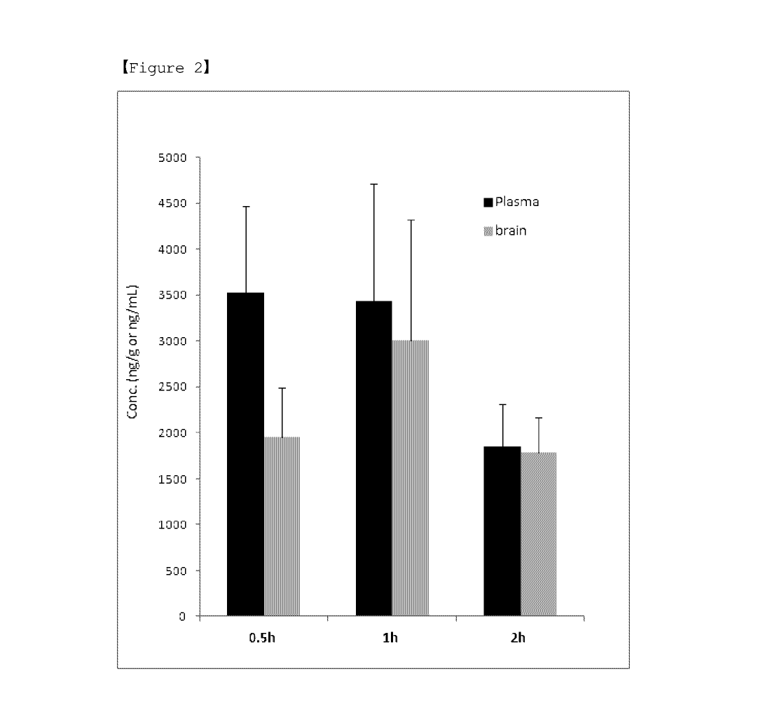

The measured concentrations of the compound in the plasma and brain tissues are shown in FIG. 2. As can be seen in FIG. 2, the concentration of the compound of the present invention in the plasma tissue was 50% or higher of that in the brain tissue. In other words, a large amount of compound 528 was distributed in the brain, suggesting that compound 528 can be used for brain diseases.

### INDUSTRIAL APPLICABILITY

As described above, the novel indole derivative compounds of the present invention have the effect of inhibiting histone deacetylase (HDAC), and thus are useful as agents for preventing or treating a disease associated with histone deacetylase (HDAC) activity.

1. An indole derivative compound represented by the following formula I, an isomer thereof, a pharmaceutically acceptable salt thereof, or a hydrate or solvate thereof: 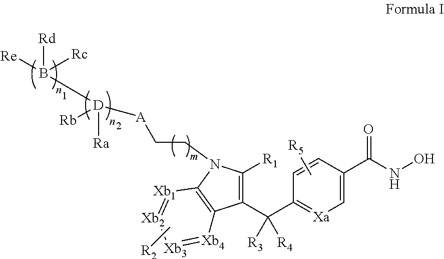
wherein
R₁ is hydrogen, halogen, a straight or branched C₁₋₅ alkyl, —NH₂, —OH, a straight or branched chain C₁₋₅ alkoxy, —CF₃, aryl, a 4- to 6-membered heteroaryl containing one or two heteroatoms selected from N, O and S, 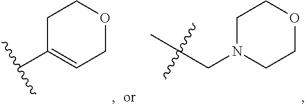 [SMILES: *C1=CCOCC1 ; *CN1CCOCC1]
wherein the aryl and heteroaryl may each independently be unsubstituted or substituted with halogen, a straight or branched chain C₁₋₅ alkoxy or a straight or branched chain C₁₋₅ alkyl;
R₂ is hydrogen, halogen, a straight or branched chain C₁₋₅ alkyl, —NH₂, —OH, a straight or branched chain C₂₋₁₀ alkylalkoxy (—C₁₋₅—O—C₁₋₅ alkyl), a straight or branched chain C₁₋₅ alkoxy, —CF₃, a straight or branched C₁₋₅ alkyl-halogen, or a straight or branched chain C₁₋₅ alkyl hydroxide (—C₁₋₅ alkyl-OH);
R₃ and R₄ are each independently hydrogen or —OH;
R₅ is hydrogen, halogen, —CF₃, or a straight or branched chain C₁₋₃ alkyl;
n₁ and n₂ are each independently 0, 1 or 2;
A is 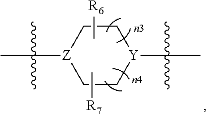
a 3- to 8-membered C₂₋₁₂ heterocycloalkyl containing one or two heteroatoms selected from N, O and S, or a 3- to 8-membered C₂₋₁₂ heteroaryl containing one or two heteroatoms selected from N, O and S, wherein Y is C or N, Z is C, O or N, R₆ and R₇ are each independently hydrogen, halogen, a straight or branched chain C₁₋₅ alkyl, —NH₂, —OH, a straight or branched chain C₂₋₁₀ alkylalkoxy (—C₁₋₅—O—C₁₋₅ alkyl), a straight or branched chain C₁₋₅ alkoxy, —CF₃, a straight or branched chain C₁₋₅ alkyl-halogen, or a straight or branched chain C₁₋₅ alkyl hydroxide (—C₁₋₅ alkyl-OH), and n3 and n4 are each independently 0, 1 or 2;
Xa, Xb₁, Xb₂, Xb₃ and Xb₄ are each independently C or N;
B and D are each independently —H, C or halogen, and when B and D are H or halogen, Ra, Rb, Rc, Rd or Re, linked to B and D, does not exist;
m is 0, 1 or 2;
Ra is hydrogen, halogen, a straight or branched chain C₁₋₅ alkyl, a C₃₋₁₂ cycloalkyl, phenyl, —OH, a 5- or 6-membered heteroaryl containing one or two heteroatoms selected from N and O, or ═O;
Rb does not exist or is hydrogen, halogen, a straight or branched chain C₁₋₅ alkyl, —OH, a straight or branched chain C₁₋₅ alkoxy, a C₃₋₁₂ cycloalkyl, a 5- or 6-membered heteroaryl containing one or two heteroatoms selected from N and O, or phenyl;
the straight or branched chain C₁₋₅ alkyl, C₃₋₁₂ cycloalkyl and phenyl in Ra or Rb may each independently be unsubstituted or substituted with halogen, —CN, thiazole, a straight or branched chain C₁₋₅ alkoxy or a straight or branched chain C₁₋₅ alkyl;
Rc is —H, halogen, a straight or branched chain C₁₋₅ alkyl, a straight or branched chain C₁₋₅ alkoxy, —CO(O)C₁₋₅ alkyl, aryl, a 4- to 6-membered heteroaryl containing one or two heteroatoms selected from N, O and S, a C₃₋₁₂ cycloalkyl, —OH, or phenyl;
Rd is hydrogen, halogen, a straight or branched chain C₁₋₅ alkyl, a straight or branched chain C₁₋₅ alkoxy, a C₃₋₁₂ cycloalkyl, —OH, or phenyl;
Rc and Rd may be linked together to form a C₃₋₈ cycloalkyl or a 4- to 6-membered cycloheteroalkyl containing one or two heteroatoms selected from N, O and S;
Re is hydrogen, halogen, —CF₃, a straight or branched chain C₁₋₃ perfluoroalkyl, a straight or branched chain C₁₋₅ alkyl, a straight or branched chain C₁₋₅ alkoxy, a 4- to 6-membered heterocycloalkyl containing one or two heteroatoms selected from N, O and S, a C₃₋₁₂ cycloalkyl, aryl, a 4- to 6-membered heteroaryl containing one or two heteroatoms selected from N, O and S, NH₂, —OH, a straight or branched chain —NHC₁₋₅ alkyl, —N-(straight or branched chain C₁₋₅ alkyl)₂, or an aryl substituted with a straight or branched chain C₁₋₅ alkyl;
wherein the straight or branched chain C₁₋₅ alkyl, C₃₋₁₂ cycloalkyl, 4- to 6-membered cycloheteroalkyl containing one or two heteroatoms selected from N, O and S, aryl, and 4- to 6-membered heteroaryl containing one or two heteroatoms selected from N, O and S in Rc, Rd or Re, or the cycloalkyl or cycloheteroalkyl formed by linkage between Rc and Rd, may each independently be unsubstituted or substituted with halogen, —CF₃, —CN, thiazole, a straight or branched chain C₁₋₅ alkoxy, a straight or branched chain C₁₋₅ alkyl, a straight or branched chain —C(═O)C₁₋₅ alkyl, a straight or branched chain —C(═O)OC₁₋₅ alkyl, a straight or branched chain —C(═O)OC₁₋₅alkyl, a straight or branched chain —C(═O)NHC₁₋₅alkyl, a straight or branched chain SO₂C₁₋₅ alkyl, —CF₃

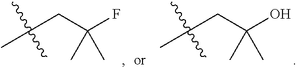

2. The compound represented by formula I, isomer thereof, pharmaceutically acceptable salt thereof, or hydrate or solvate thereof according to claim 1, wherein A in formula I is

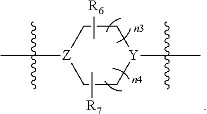

3. The compound represented by formula I, isomer thereof, pharmaceutically acceptable salt thereof, or hydrate or solvate thereof according to claim 1, wherein R₃ and R₄ in formula I are each independently hydrogen.

4. The compound represented by formula I, isomer thereof, pharmaceutically acceptable salt thereof, or hydrate or solvate thereof according to claim 1, wherein the halogen is —F.

5. The compound represented by formula I, isomer thereof, pharmaceutically acceptable salt thereof, or hydrate or solvate thereof according to claim 1, wherein R₁ in formula I is —H, methyl, ethyl, propyl, butyl, phenyl, 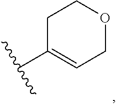 [SMILES: *C1=CCOCC1]
pyridinyl or pyrimidinyl, in which the phenyl, pyridinyl or pyrimidinyl is unsubstituted or substituted with one or two —F or —CF₃ groups.

6. The compound represented by formula I, isomer thereof, pharmaceutically acceptable salt thereof, or hydrate or solvate thereof according to claim 1, wherein B and D in formula I are each independently carbon (C).

7. The compound represented by formula I, isomer thereof, pharmaceutically acceptable salt thereof, or hydrate or solvate thereof according to claim 1, wherein the cycloheteroalkyl formed by linkage between Rc and Rd is

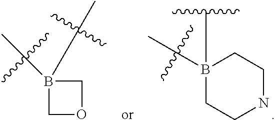

8. The compound represented by formula I, isomer thereof, pharmaceutically acceptable salt thereof, or hydrate or solvate thereof according to claim 1, wherein the compound represented by formula I is selected from the group consisting of the following compounds:
N-hydroxy-4-((2-methyl-1-(2-morpholinoethyl)-1H-indol-3-yl)methyl)benzamide;
N-hydroxy-4-((2-methyl-1-(2-(4-methylpiperazin-1-yl)ethyl)-1H-indol-3-yl)methyl)benzamide;
N-hydroxy-4-((2-(morpholinomethyl)-1H-indol-3-yl)methyl)benzamide;
N-hydroxy-4-((1-(2-(4-isopropylpiperazin-1-yl)ethyl)-2-methyl-1H-indol-3-yl)methyl)benzamide;
N-hydroxy-4-((1-(2-(4-(2-hydroxyethyl)piperazin-1-yl)ethyl)-2-methyl-1H-indol-3-yl)methyl)benzamide;
N-hydroxy-4-((1-(2-(4-(2-methoxyethyl)piperazin-1-yl)ethyl)-2-methyl-1H-indol-3-yl)methyl)benzamide;
(S)—N-hydroxy-4-((1-(2-(2-(hydroxymethyl)pyrrolidin-1-yl)ethyl)-2-methyl-1H-indol-3-yl)methyl)benzamide;
(S)—N-hydroxy-4-((1-(2-(2-(methoxymethyl)pyrrolidin-1-yl)ethyl)-2-methyl-1H-indol-3-yl)methyl)benzamide;
N-hydroxy-4-((2-methyl-1-(2-(2-methyl-1H-imadazol-1-yl)ethyl)-1H-indol-3-yl)methyl)benzamide;
N-hydroxy-6-((2-methyl-1-(2-(4-methylpeperazin-1-yl)ethyl)-1H-indol-3-yl)methyl)nicotinamide;
N-hydroxy-6-((2-methyl-1-(2-morpholinoethyl)-1H-indol-3-yl)methyl)nocotinamide;
4-((1-((1-(2-fluoro-2-methylpropyl)piperidin-4-yl)methyl)-2-methyl-1H-indol-3-yl)methyl)-N-hydroxybenzamide;
4-((1-(2-(3-fluoropiperidin-1-yl)ethyl)-2-methyl-1H-indol-3-yl)methyl)-N-hydroxybenzamide;
(S)-4-((1-(2-(2-(fluoromethyl)pyrrolidin-1-yl)ethyl)-2-methyl-1H-indol-3-yl)methyl)-N-hydroxybenzamide;
4-((1-(2-(4-fluoropiperidin-1-yl)ethyl)-2-methyl-1H-indol-3-yl)methyl)-N-hydroxybenzamide;
4-((1-((1-benzylpiperidin-4-yl)methyl)-2-methyl-1H-indol-3-yl)methyl)-N-hydroxybenzamide;
4-((1-((1-butylpiperidin-4-yl)methyl)-2-methyl-1H-indol-3-yl)methyl)-N-hydroxybenzamide;
N-hydroxy-4-((2-methyl-1-((1-phenethylpiperidin-4-yl)methyl)-1H-indol-3-yl)methyl)benzamide;
3-fluoro-4-((1-((1-(2-fluoro-2-methylpropyl)piperidin-4-yl)methyl)-2-methyl-1H-indol-3-yl)methyl)-N-hydroxybenzamide;
4-((1-(2-(1-(2-fluoro-2-methylpropyl)piperidin-4-yl)ethyl)-2-methyl-1H-indol-3-yl)methyl)-N-hydroxybenzamide;
4-((1-(2-(4-(2-fluoro-2-methylpropyl)piperazin-1-yl)ethyl)-2-methyl-1H-indol-3-yl)methyl)-N-hydroxybenzamide;
4-((1-(2-(1-(2-ethyl-2-fluorobutyl)piperidin-4-yl)ethyl)-2-methyl-1H-indol-3-yl)methyl)-N-hydroxybenzamide;
4-((5-fluoro-1-((1-(2-hydroxy-2-methylpropyl)piperidin-4-yl)methyl)-2-methyl-1H-indol-3-yl)methyl)-N-hydroxybenzamide;
4-((1-((1-((4-fluoro-tetrahydro-2H-pyran-4-yl)methyl)piperidin-4-yl)methyl)-2-methyl-1H-indol-3-yl)methyl)-N-hydroxybenzamide;
4-((1-((1-((4-fluoro-tetrahydro-2H-pyran-4-yl)methyl)piperidin-4-yl)methyl)-2-methyl-1H-indol-3-yl)methyl)-N-hydroxybenzamide;
N-hydroxy-4-((1-(2-(3-(hydroxymethyl)pyrroldin-1-yl)ethyl)-2-methyl-1H-indol-3-yl)methyl)benzamide;
(S)-4-((5-fluoro-1-(2-(3-hydroxypyrrolidin-1-yl)ethyl)-2-methyl-1H-indol-3-yl)methyl)-N-hydroxybenzamide;
4-((5-fluoro-1-((1-(2-fluoro-2-methylpropyl)piperidin-4-yl)methyl)-2-methyl-1H-indol-3-yl)methyl)-N-hydroxybenzamide;
(S)-4-((5-fluoro-1-(2-(3-fluoropyrrolidin-1-yl)ethyl)-2-methyl-1H-indol-3-yl)methyl)-N-hydroxybenzamide;
4-((5-fluoro-1-(2-(4-fluoropiperidin-1-yl)ethyl)-2-methyl-1H-indol-3-yl)methyl)-N-hydroxybenzamide;
4-((1-(2-(3-(fluoromethyl)pyrrolidin-1-yl)ethyl)-2-methyl-1H-indol-3-yl)methyl)-N-hydroxybenzamide;
(S)—N-hydroxy-4-((1-(2-(3-hydroxypyrrolidin-1-yl)ethyl)-2-methyl-1H-indol-3-yl)methyl)benzamide;
(S)-4-((1-(2-(3-fluoropyrrolidin-1-yl)ethyl)-2-methyl-1H-indol-3-yl)methyl)-N-hydroxybenzamide;
N-hydroxy-4-((1-(2-(3-(hydroxymethyl)piperidin-1-yl)ethyl)-2-methyl-1H-indol-3-yl)methyl)benzamide;
4-((2-butyl-1-((1-(2-fluoro-2-methylpropyl)piperidin-4-yl)methyl)-1H-indol-3-yl)methyl)-N-hydroxybenzamide;
N-hydroxy-4-((1-((1-(2-hydroxy-2-methylpropyl)piperidin-4-yl)methyl)-2-propyl-1H-indol-3-yl)methyl)benzamide;
4-((1-((1-(2-fluoro-2-methylpropyl)piperidin-4-yl)methyl)-2-propyl-1H-indol-3-yl)methyl)-N-hydroxybenzamide;
N-hydroxy-4-((2-methyl-1-((1-(3-(thiazol-2-yl)benzyl)piperidin-4-yl)methyl)-1H-indol-3-yl)methyl)benzamide;
N-hydroxy-4-((1-((1-(2-hydroxy-2-methylpropyl)piperidin-4-yl)methyl)-2-methyl-1H-indol-3-yl)methyl)benzamide;
4-((1-((1-((3-fluoroxetan-3-yl)methyl)piperidin-4-yl)methyl)-2-methyl-1H-indol-3-yl)methyl)-N-hydroxybenzamide;
tert-butyl4-fluoro-4-((4-((3-(4-(hydroxycarbamoyl)benzyl)-2-methyl-1H-indol-1-yl)methyl)piperidin-1-yl)methyl)piperidine-1-carboxylate;
4-((1-((1-((4-fluoropiperidin-4-yl)methyl)piperidin-4-yl)methyl)-2-methyl-1H-indol-3-yl)methyl)-N-hydroxybenzamide;
4-((1-((1-((1-acetyl-4-fluoropiperidin-4-yl)methyl)piperidin-4-yl)methyl)-2-methyl-1H-indol-3-yl)methyl)-N-hydroxybenzamide;
4-((1-((1-((4-fluoro-1-(2-hydroxy-2-methylpropyl)piperidin-4-yl)methyl)piperidin-4-yl)methyl)-2-methyl-1H-indol-3-yl)methyl)-N-hydroxybenzamide;
4-((1-((1-(2-fluoro-2-methylpropyl)piperidin-4-yl)methyl)-1H-indol-3-yl)methyl)-N-hydroxybenzamide;
4-((1-((1-((4-fluoro-1-(2-fluoro-2-methylpropyl)piperidin-4-yl)methyl)piperidin-4-yl)methyl)-2-methyl-1H-indol-3-yl)methyl)-N-hydroxybenzamide;
4-((1-((1-(2-fluoro-2-methylpropyl)piperidin-4-yl)methyl)-2-phenyl-1H-indol-3-yl)methyl)-N-hydroxybenzamide;
N-hydroxy-4-((2-methyl-1-((1-(2,4,5-trifluorobenzyl)piperidin-4-yl)methyl)-1H-indol-3-yl)methyl)benzamide;
4-((1-((1-(2-fluorobenzyl)piperidin-4-yl)methyl)-2-methyl-1H-indol-3-yl)methyl)-N-hydroxybenzamide;
N-hydroxy-4-((2-methyl-1-((1-(pyridin-4-ylmethyl)piperidin-4-yl)methyl)-1H-indol-3-yl)methyl)benzamide;
4-((1-((1-(4-fluorobenzyl)piperidin-4-yl)methyl)-2-methyl-1H-indol-3-yl)methyl)-N-hydroxybenzamide;
N-hydroxy-4-((2-methyl-1-((1-(pyridin-2-ylmethyl)piperidin-4-yl)methyl)-1H-indol-3-yl)methyl)benzamide;
4-((1-((1-(2-fluoro-2-methylpropyl)piperidin-4-yl)methyl)-2-(pyridin-4-yl)-1H-indol-3-yl)methyl)-N-hydroxybenzamide;
4-((1-((1-(2-fluoro-2-methylpropyl)piperidin-4-yl)methyl)-2-(pyrimidin-5-yl)-1H-indol-3-yl)methyl)-N-hydroxybenzamide;
4-((2-(3,5-difluorophenyl)-1-((1-(2-fluoro-2-methylpropyl)piperidin-4-yl)methyl)-1H-indol-3-yl)methyl)-N-hydroxybenzamide;
4-((2-(3,6-dihydro-2H-pyran-4-yl)-1-((1-(2-fluoro-2-methylpropyl)piperidin-4-yl)methyl)-1H-indol-3-yl)methyl)-N-hydroxybenzamide;
4-((1-((1-(3-fluorobenzyl)piperidin-4-yl)methyl)-2-methyl-1H-indol-3-yl)methyl)-N-hydroxybenzamide;
N-hydroxy-4-((2-methyl-1-((1-(pyridin-3-ylmethyl)piperidin-4-yl)methyl)-1H-indol-3-yl)methyl)benzamide;
4-((1-(2-((3S,5R)-4-(2-fluoro-2-methylpropyl)-3,5-dimethylpiperazin-1-yl)ethyl)-2-methyl-1H-indol-3-yl)methyl)-N-hydroxybenzamide;
4-((1-(4-(4-(2-fluoro-2-methylpropyl)piperazin-1-yl)butyl)-2-methyl-1H-indol-3-yl)methyl)-N-hydroxybenzamide;
4-((1-(3-(4-(2-fluoro-2-methylpropyl)piperazin-1-yl)propyl)-2-methyl-1H-indol-3-yl)methyl)-N-hydroxybenzamide;
N-hydroxy-4-((2-methyl-1-((1-((3-methyloxetan-3-yl)methyl)piperidin-4-yl)methyl)-1H-indol-3-yl)methyl)benzamide;
4-((1-((1-((4-fluoro-1-methylpiperidin-4-yl)methyl)piperidin-4-yl)methyl)-2-methyl-1H-indol-3-yl)methyl)-N-hydroxybenzamide;
4-((1-((1-((4-fluoro-1-isopropylpiperidin-4-yl)methyl)piperidin-4-yl)methyl)-2-methyl-1H-indol-3-yl)methyl)-N-hydroxybenzamide;
4-fluoro-4-((4-((3-(4-(hydroxycarbamoyl)benzyl)-2-methyl-1H-indol-1-yl)methyl)piperidin-1-yl)methyl)-N-isopropylpiperidine-1-carboxamide;
4-((1-((1-((4-fluoro-1-(methylsulfonyl)piperidin-4-yl)methyl)piperidin-4-yl)methyl)-2-methyl-1H-indol-3-yl)methyl)-N-hydroxybenzamide;
4-((1-((1-(3,5-bis(trifluoromethyl)benzyl)piperidin-4-yl)methyl)-2-methyl-1H-indol-3-yl)methyl)-N-hydroxybenzamide;
4-((1-((1-((1-fluorocyclohexyl)methyl)piperidin-4-yl)methyl)-2-methyl-1H-indol-3-yl)methyl)-N-hydroxybenzamide;
4-((4-fluoro-1-((1-(2-fluoro-2-methylpropyl)piperidin-4-yl)methyl)-1H-indol-3-yl)methyl)-N-hydroxybenzamide;
4-((6-fluoro-1-((1-(2-fluoro-2-methylpropyl)piperidin-4-yl)methyl)-1H-indol-3-yl)methyl)-N-hydroxybenzamide;
4-((1-((1-(2-fluoro-2-methylpropyl)pyrrolidin-3-yl)methyl)-2-methyl-1H-indol-3-yl)methyl)-N-hydroxybenzamide;
N-hydroxy-4-((2-methyl-1-((1-((1-(trifluoromethyl)cyclobutyl)methyl)piperidin-4-yl)methyl)-1H-indol-3-yl)methyl)benzamide;
4-((1-((1-(2-fluoro-2-methylpropyl)piperidin-4-yl)methyl)-1H-pyrrolo[2,3-b]pyridin-3-yl)(hydroxy)methyl)-N-hydroxybenzamide;
4-((1-((1-(2-fluoro-2-methylpropyl)piperidin-4-yl)methyl)-1H-pyrrolo[2,3-b]pyridin-3-yl)methyl)-N-hydroxybenzamide;
4-((1-((1-((3-fluorooxetan-3-yl)methyl)azetidin-3-yl)methyl)-2-methyl-1H-indol-3-yl)methyl)-N-hydroxybenzamide; and
4-((1-((1-(2-fluoro-2-methylpropyl)azetidin-3-yl)methyl)-2-methyl-1H-indol-3-yl)methyl)-N-hydroxybenzamide.

9. The compound represented by formula I, isomer thereof, pharmaceutically acceptable salt thereof, or hydrate or solvate thereof according to claim 1, wherein the compound represented by formula I is 4-((1-((1-(2-fluoro-2-methylpropyl)piperidin-4-yl)methyl)-2-methyl-1H-indol-3-yl)methyl)-N-hydroxybenzamide.

10. The compound represented by formula I, isomer thereof, pharmaceutically acceptable salt thereof, or hydrate or solvate thereof according to claim 1, wherein the compound represented by formula I is 4-((1-((1-((3-fluorooxetan-3-yl)methyl)piperidin-4-yl)methyl)-2-methyl-1H-indol-3-yl)methyl)-N-hydroxybenzamide.

11. A pharmaceutical composition comprising a compound represented by formula I, isomer thereof, pharmaceutically acceptable salt thereof, or hydrate or solvate thereof according to claim 1, together with a pharmaceutically acceptable carrier.

12. The pharmaceutical composition of claim 11, which is for prevention or treatment of a disease associated with histone deacetylase (HDAC) activity.

13. The pharmaceutical composition of claim 12, wherein the disease associated with histone deacetylase (HDAC) activity is cell proliferative disease, autosomal dominant disease, fibrosis, autoimmune disease, diabetes, acute neurological disease, chronic neurological disease, hypertrophy, congestive heart failure, amyotrophic lateral sclerosis, glaucoma, angiogenesis-related ocular disease, or Alzheimer's disease.

14. A method for preventing or treating a disease associated with histone deacetylase (HDAC) activity, the method comprising administering, to mammals including humans, a composition comprising as an active ingredient a compound of formula I, isomer thereof, pharmaceutically acceptable salt thereof, or hydrate or solvate thereof according to claim 1.

15. (canceled)
